(19) United States
(12) Patent Application Publication
(10) Pub. No.: US 2024/0166647 A1
(43) Pub. Date: May 23, 2024

(54) Cereblon Ligands

(71) Applicant: THE REGENTS OF THE UNIVERSITY OF MICHIGAN, Ann Arbor, MI (US)

(72) Inventors: Shaomeng Wang, Superior Township, MI (US); Tianfeng Xu, Ypsilanti, MI (US); Dimin Wu, Ann Arbor, MI (US); Zhixiang Chen, Ann Arbor, MI (US); Xin Han, Ann Arbor, MI (US); Weiguo Xiang, Ypsilanti, MI (US); Rohan Rej, Ann Arbor, MI (US); Angelo Aguilar, Ann Arbor, MI (US); Longchuan Bai, Ann Arbor, MI (US)

(21) Appl. No.: 18/279,225

(22) PCT Filed: Mar. 3, 2022
(86) PCT No.: PCT/US22/18609

§ 371 Date: Aug. 29, 2023

Related U.S. Application Data
(60) Provisional application No. 63/156,294, filed on Mar. 3, 2021.

(51) Int. Cl. C07D 471/04; C07D 471/14; C07D 487/04; C07D 487/10; C07D 498/04; C07D 498/14

(52) U.S. Cl. CPC C07D 471/04; C07D 471/14; C07D 487/04; C07D 487/10; C07D 498/04; C07D 498/14

(57) ABSTRACT

The present disclosure provides compounds of Formula (I), wherein A, A¹, A², A³, R³, Z and Z¹ are as defined in the specification, and the salts and solvates thereof. The present disclosure also relates to uses of the compounds as cereblon (CRBN) ubiquitination inhibitors, as synthetic intermediates that can be used to prepare PROTAC molecules, or as PROTAC molecules. The present disclosure also relates to uses of the compounds, e.g., in treating or preventing cancer and other diseases.

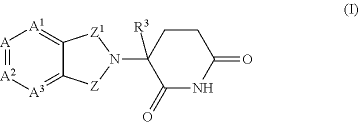

### RELATED APPLICATION

The application claims priority to, and the benefit of, U.S. Provisional Application No. 63/156,294, filed on Mar. 3, 2021, the content of which is incorporated herein by reference in its entirety.

### BACKGROUND

Cereblon (CRBN), a component of the DDB1-CUL4a-Roc1 ubiquitin ligase complex, is a molecular target of immunomodulatory agents such as thalidomide, lenalidomide, and pomalidomide. Lopez-Girona et al., Leukemia 26:2326-2335 (2012). Inhibition of CRBN ubiquitination by these agents may allow CRBN to accumulate, leading to the increased cullin-4 RING E3 ligase-mediated degradation of target proteins. Liu et al., FASEB J 12:4829-4839 (2015). There exists a need for new immunomodulatory agents for the treatment of cancer and other diseases. See Boichenko et al., ACS Omega 3:11163-11171 (2018).

Proteolysis Targeting Chimera (PROTAC) molecules are heterobifunctional conmpounds that that simultaneously bind to a target protein and to an E3 ligase complex, resulting in the transfer of ubiquitin and initiating a process ultimately causing the proteasomal degradation of the target protein. Benowitz et al., Expert Opinion on Therapeutic Patents 31:1-23 (2021). There exists a need for new PROTAC molecules for the treatment of cancer and other diseases.

### SUMMARY

In some aspects, the present disclosure provides compounds represented by any one of Formulae I-XXXIX, below, and the pharmaceutically acceptable salts and solvates, e.g., hydrates, thereof.

Compounds of any one of Formulae I-XXXIX, and the pharmaceutically acceptable salts and solvates thereof, wherein R^{1a}, R^{1b}, R^{1d}, R^{1e}, R^{1g}, R^{1h}, R^{1j}, R^{2e}, R^{2f}, R^{2g}, or R^{2h} is not Q-L- are collectively referred to as “Cereblon Ligands.” Cereblon Ligands bind to the protein cereblon and thus can be used as immunomodulatory agents. Cereblon Ligands can also be used as synthetic intermediates to prepare PROTAC molecules for targeted protein degradation via proximity-induced ubiquitination. See, e.g., PCT/US2020/048186.

Compounds of any one of Formulae I-XXXIX, and the pharmaceutically acceptable salts and solvates thereof, wherein R^{1a}, R^{1b}, R^{1d}, R^{1e}, R^{1g}, R^{1h}, R^{1j}, R^{2e}, R^{2f}, R^{2g}, or R^{2h} is Q-L- are collectively referred to as “PROTAC Molecules.”

In some aspects, the present disclosure provides methods of treating or preventing a condition or disease by administering a therapeutically effective amount of a Cereblon Ligand to a subject, e.g., a human patient, in need thereof. The disease or condition of interest that is treatable or preventable by inhibition CRBN ubiquitination is, for example, cancer or other proliferative disorder, or an inflammatory disease. Also provided are methods of preventing the proliferation of unwanted proliferating cells, such as in cancer, in a subject comprising administering a therapeutically effective amount of a Cereblon Ligand to a subject at risk of developing a condition characterized by unwanted proliferating cells. In some embodiments, Cereblon Ligands may reduce the proliferation of unwanted cells by modulating the function of CRBN in those cells. In some embodiments, Cereblon Ligands are administered in combination with an optional therapeutic agent.

In some aspects, the present disclosure provides a method of inhibiting CRBN ubiquitination in a subject, comprising administering to the subject a therapeutically effective amount of a Cereblon Ligand.

In some aspects, the present disclosure provides a pharmaceutical composition comprising a Cereblon Ligand and an excipient and/or pharmaceutically acceptable carrier.

In some aspects, the present disclosure provides a composition comprising a Cereblon Ligand and an excipient and/or pharmaceutically acceptable carrier for use treating or preventing diseases or conditions wherein the inhibition of CRBN ubiquitination provides a benefit, e.g., cancer.

In some aspects, the present disclosure provides a Cereblon Ligand for use in the treatment or prevention of a disease or condition of interest, e.g., cancer.

In some aspects, the present disclosure provides a use of a Cereblon Ligand for the manufacture of a medicament for treating a disease or condition of interest, e.g., cancer.

In some aspects, the present disclosure provides a kit comprising a Cereblon Ligand, and, optionally, a packaged composition comprising an optional therapeutic agent useful in the treatment of a disease or condition of interest, and a package insert containing directions for use in the treatment of a disease or condition, e.g., cancer.

In some aspects, the present disclosure provides methods of treating or preventing a condition or disease, e.g., cancer, in a subject comprising administering a therapeutically effective amount of a PROTAC Molecule to the subject.

In some aspects, the present disclosure provides methods of degrading a target protein, e.g., BET bromodomain protein, in a subject, comprising administering to the subject a therapeutically effective amount of a PROTAC Molecule to the subject.

In some aspects, the present disclosure provides a pharmaceutical composition comprising a PROTAC Molecule and an excipient and/or pharmaceutically acceptable carrier.

In some aspects, the present disclosure provides a composition comprising a PROTAC Molecule and an excipient and/or pharmaceutically acceptable carrier for use treating or preventing diseases or conditions wherein the inhibition of CRBN ubiquitination provides a benefit, e.g., cancer.

In some aspects, the present disclosure provides a PROTAC Molecule for use in the treatment or prevention of a disease or condition of interest, e.g., cancer.

In some aspects, the present disclosure provides a use of a PROTAC Molecule for the manufacture of a medicament for treating a disease or condition of interest, e.g., cancer.

In some aspects, the present disclosure provides a kit comprising a PROTAC Molecule, and, optionally, a packaged composition comprising an optional therapeutic agent useful in the treatment of a disease or condition of interest, and a package insert containing directions for use in the treatment of a disease or condition, e.g., cancer.

In some aspects, the present disclosure provides methods of preparing Cereblon Ligands.

In some aspects, the present disclosure provides methods of preparing PROTAC molecules.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

It is to be understood that both the foregoing summary and the following detailed description are exemplary and explanatory only, and are not restrictive of the invention as claimed.

### DETAILED DESCRIPTION

The present disclosure relates to cereblon (CRBN) ubiquitination inhibitors and PROTAC molecules, and therapeutic methods of treating conditions and diseases, e.g., cancer, wherein the inhibition of CRBN ubiquitination or targeted protein degradation provides a benefit.

Compounds of the Disclosure

In some aspects, the disclosure provides a compound of Formula I:

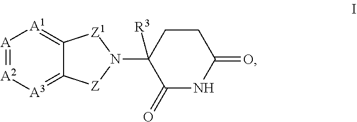

or a pharmaceutically acceptable salt or solvate thereof, wherein:
(a) A¹ is selected from —CR^{2a}═ and —N═;
A is —CR^{2b}═;
A² is —CR^{2c}═;
R^{2b} and R^{2c} are taken together to form a ring comprising a:
   (i) —(CH₂)ₘ—X—(CH₂)ₙ— radical;
   (ii) —C(═O)—N(R^{1d})—(CH₂)_{q}— radical;
   (iii) —(CH₂)ₜ—N(R^{1e})—(CH₂)₂—Y—(CH₂)ᵤ— radical;
   (iv) -E¹=E-E²=E³- radical;
   (v)=A⁴-N(R^{1g})—CR^{2k}=radical; or
   (vi) -E⁴=CR^{1j}-E⁵- radical;
   R^{2a} is selected from hydrogen, halo, C₁-C₃ alkyl, amino, and C₁-C₃ alkoxy; and
   R^{2d} is selected from hydrogen, halo, C₁-C₃ alkyl, amino, and C₁-C₃ alkoxy;
   A³ is selected from —CR^{2d}═ and —N═; or
(b) A¹ is —CR^{2a}═;
   A is —CR^{2b}—;
   R^{2a} and R^{2b} are taken together to form a ring comprising a:
      (i) —(CH₂)ₘ—X—(CH₂)ₙ— radical;
      (ii) —C(═O)—N(R^{1d})—(CH₂)_{q}— radical;
      (iii) —(CH₂)ₜ—N(R^{1e})—(CH₂)₂—Y—(CH₂)ᵤ— radical;
      (iv) -E¹=E-E²=E³- radical;
      (v)=A⁴-N(R^{1g})—CR^{2k}=radical; or
      (vi) -E⁴=CR^{1j}-E⁵- radical;
   A² is selected from —CR^{2c}═ and —N═;
   A³ is selected from —CR^{2d}═ and —N═;
   R^{2e} is selected from hydrogen, halo, C₁-C₃ alkyl, amino, and C₁-C₃ alkoxy; and
   R^{2d} is selected from hydrogen, halo, C₁-C₃ alkyl, amino, and C₁-C₃ alkoxy;
   X is selected from —N(R^{1a})— and —CR^{1b}R^{1c}—
   R^{1a} is selected from hydrogen, C₁-C₃ alkyl, C₁-C₃ haloalkyl, cycloalkyl, aryl, heterocyclo, heteroaryl, (cycloalkyl)alkyl, (aryl)alkyl, (heterocyclo)alkyl, (heteroaryl)alkyl, —C(═O)-(cycloalkyl), —C(═O)-(aryl), —C(═O)-(heterocyclo), —C(═O)-(heteroaryl), and Q-L-, wherein the C₁-C₃ alkyl, C₁-C₃ haloalkyl, cycloalkyl, aryl, heterocyclo, heteroaryl, (cycloalkyl)alkyl, (aryl)alkyl, (heterocyclo)alkyl, (heteroaryl)alkyl, —C(═O)-(cycloalkyl), —C(═O)-(aryl), —C(═O)-(heterocyclo), —C(═O)-(heteroaryl) is optionally substituted with one or more R^{1aS};
      each R^{1aS} is independently selected from oxo, halo, C₁-C₃ alkyl, C₁-C₃ haloalkyl, C₁-C₃ alkoxy, —C(═O)—OH, —C(═O)—(C₁-C₃ alkyl), —C(═O)O—(C₁-C₄ alkyl), —NH₂, —NH(C₁-C₃ alkyl), —N(C₁-C₃ alkyl)₂, cycloalkyl, aryl, heterocyclo, heteroaryl, (cycloalkyl)alkyl, (aryl)alkyl, (heterocyclo)alkyl, (heteroaryl)alkyl, —C(═O)-(cycloalkyl), —C(═O)-(aryl), —C(═O)-(heterocyclo), and —C(═O)-(heteroaryl), wherein the C₁-C₃ alkyl, C₁-C₃ haloalkyl, C₁-C₃ alkoxy, cycloalkyl, aryl, heterocyclo, heteroaryl, (cycloalkyl)alkyl, (aryl)alkyl, (heterocyclo)alkyl, (heteroaryl)alkyl, —C(═O)-(cycloalkyl), —C(═O)-(aryl), —C(═O)-(heterocyclo), —C(═O)-(heteroaryl) is optionally substituted with one or more R^{1aSS};
      each R^{1aSS} is independently selected from oxo, halo, C₁-C₃ alkyl, C₁-C₃ haloalkyl, C₁-C₃ alkoxy, —C(═O)—OH, —C(═O)—(C₁-C₃ alkyl), —NH₂, —NH(C₁-C₃ alkyl), and —N(C₁-C₃ alkyl)₂;
   R^{1b} is selected from hydrogen, —CHO, —C(═O)OH, hydroxy, (hydroxy)C₁-C₃ alkyl, amino, (amino)alkyl, (heterocyclo)alkyl, and Q-L-, wherein the (heterocyclo)alkyl is optionally substituted with one or more R^{1bS};
      each R^{1bS} is independently (aryl)alkyl optionally substituted with one or more halo;
   R^{1c} is hydrogen; or
   R^{1b} and R^{1c} taken together with the carbon atom to which they are attached form a —C(═O)—; or
   R^{1b} and R^{1c} taken together with the carbon atom to which they are attached form:

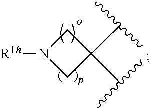

R^{1d} is selected from hydrogen, heterocyclo, (heterocyclo)alkyl, and Q-L-, wherein the heterocyclo or (heterocyclo)alkyl is optionally substituted with one or more R^{1dS}.
   each R^{1dS} is independently selected from C₁-C₃ alkyl, —C(═O)—(C₁-C₃ alkyl), and (aryl)alkyl, wherein the (aryl)alkyl is optionally substituted with one or more halo;
R^{1e} is selected from hydrogen and Q-L-;
R^{1g} is selected from hydrogen and Q-L-;
R^{1h} is selected from hydrogen, (aryl)alkyl, and Q-L-, wherein the (aryl)alkyl is optionally substituted with one or more halo;
Y is selected from —O—, —S—, and —N(R^{1f})—
R^{1f} is selected from hydrogen and C₁-C₃ alkyl;
E¹ is selected from —CR^{2e}═ and —N═;
E is selected from —CR^{2f}═ and —N═;
E² is selected from —CR^{2g}═ and —N═;
E³ is selected from —CR^{2h}═ and —N═;
R^{2e}, R^{2f}, R^{2g}, and R^{2h} are independently selected from hydrogen, halo, hydroxy, amino, —CHO, C₁-C₃ alkyl, and C₁-C₃ alkoxy; or
R^{2e} is Q-L-; and, if present, R^{2f}, R^{2g}, and R^{2h} are independently selected from hydrogen, halo, hydroxy, amino, —CHO, C₁-C₃ alkyl, and C₁-C₃ alkoxy; or
R^{2f} is Q-L-; and, if present, R^{2e}, R^{2g}, and R^{2h} are independently selected from hydrogen, halo, hydroxy, amino, —CHO, C₁-C₃ alkyl, and C₁-C₃ alkoxy; or
R^{2g} is Q-L-; and, if present, R^{2e}, R^{2f}, and R^{2h} are independently selected from hydrogen, halo, hydroxy, amino, —CHO, C₁-C₃ alkyl, and C₁-C₃ alkoxy; or
R^{2h} is Q-L-; and, if present, R^{2e}, R^{2f}, and R^{2g} are independently selected from hydrogen, halo, hydroxy, amino, —CHO, C₁-C₃ alkyl, and C₁-C₃ alkoxy;
E⁴ is selected from ═C(H)— and ═N—;
E⁵ is selected from —O—, —S—, and —N(R^{2m})—;
R^{2m} is selected from hydrogen and C₁-C₄ alkyl;
R^{1j} is selected from hydrogen, C₁-C₄ alkyl, (hydroxy)alkyl, (heterocyclo)alkyl, and Q-L-;
A⁴ is selected from —CR^{2j}'² and —N═;
R^{2j} is selected from hydrogen and C₁-C₃ alkyl;
R^{2k} is selected from hydrogen and C₁-C₃ alkyl;
m is 1, 2, or 3;
n is 1, 2, or 3;
is 1, 2, or 3;
p is 1, 2, or 3;
q is 1 or 2;
t is 0 or 1;
u is 0 or 1;
R³ is selected from hydrogen, deuterium, fluoro, and C₁-C₃ alkyl;
Z and Z¹ are —C(═O)—; or
Z is —C(═O)— and Z¹ is —CR^{4a}R^{4b}—; or
Z is —CR^{4a}R^{4b}— and Z¹ is —C(═O)—; or
Z is —N═C(CH₃)— and Z¹ is —C(═O)—; or
Z is —C(═O)— and Z¹ is —N═C(CH₃)—; or
Z is a bond and Z¹ is —N(R²ⁿ)C(═O)—; or
Z is —N(R²ⁿ)C(═O) and Z is a bond;
R²ⁿ is selected from hydrogen and C₁-C₄ alkyl;
R^{4a} and R^{4b} are independently selected from hydrogen and C₁-C₃ alkyl; or
R^{4a} and R^{4b} taken together with the carbon to which they are attached form a C₃-C₆ cycloalkyl,
Q is a small molecule that binds to a target protein of interest;
L is -J¹-J²-J³-J⁴-J⁵-, wherein J¹ is attached to Q;
J¹ is selected from alkylenyl, cycloalkylenyl, and heterocyclenyl; or J¹ is absent;
J² is selected from —C(═O)—, —(CH₂)_{w}—, —CH═CH—, and —C≡C—;
w is 0, 1, 2, or 3;
J³ is selected from alkylenyl, heteroalkylenyl, cycloalkylenyl, heterocyclenyl, phenylenyl, and heteroarylenyl; or J³ is absent;
J⁴ is selected from alkylenyl, cycloalkylenyl, and heterocyclenyl; or J⁴ is absent;
J⁵ is selected from —O—, —N(H)—, —C≡C—, —(CH₂)ₓ— and —C(═O)—; and
x is 0, 1, 2, or 3.

In some aspects, the disclosure provides a compound of Formula I:

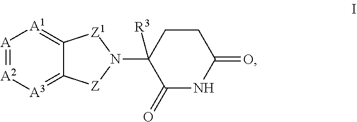

or a pharmaceutically acceptable salt or solvate thereof, wherein:
(a) A¹ is selected from —CR^{2a}═ and —N═;
   A is —CR^{2b}—.
   A² is —CR^{2c}—;
   R^{2b} and R^{2c} are taken together to form a ring comprising a:
      (i) —(CH₂)ₘ—X—(CH₂)ₙ— radical;
      (ii) —C(═O)—N(R^{1d})—(CH₂)_{q}— radical;
      (iii) —(CH₂)ₜ—N(R^{1e})—(CH₂)₂—Y—(CH₂)ᵤ— radical;
      (iv) -E¹=E-E²=E³- radical;
      (v)=A⁴-N(R^{1g})—CR^{2k}=radical; or
      (vi) -E⁴=CR^{1j}-E⁵- radical;
   R^{2a} is selected from hydrogen, halo, C₁-C₃ alkyl, amino, and C₁-C₃ alkoxy; and
   R^{2d} is selected from hydrogen, halo, C₁-C₃ alkyl, amino, and C₁-C₃ alkoxy;
   A³ is selected from —CR^{2d}═ and —N═; or
(b) A¹ is —CR^{2a}—;
   A is —CR^{2b}—.
   R^{2a} and R^{2b} are taken together to form a ring comprising a:
      (i) —(CH₂)ₘ—X—(CH₂)ₙ— radical;
      (ii) —C(═O)—N(R^{1d})—(CH₂)_{q}— radical;
      (iii) —(CH₂)ₜ—N(R^{1e})—(CH₂)₂—Y—(CH₂)ᵤ— radical;
      (iv) -E¹=E-E²=E³- radical;
      (v)=A⁴-N(R^{1g})—CR^{2k}=radical; or
      (vi) -E⁴=CR^{1j}-E⁵- radical;
   A² is selected from —CR^{2c}═ and —N═;
   A³ is selected from —CR^{2d}═ and —N═;
   R^{2e} is selected from hydrogen, halo, C₁-C₃ alkyl, amino, and C₁-C₃ alkoxy; and
   R^{2d} is selected from hydrogen, halo, C₁-C₃ alkyl, amino, and C₁-C₃ alkoxy;
   X is selected from —N(R^{1a})— and —CR^{1b}R^{1c}—
   R^{1a} is selected from hydrogen and Q-L-;
   R^{1b} is selected from hydrogen, —CHO, —C(═O)OH, hydroxy, (hydroxy)C₁-C₃ alkyl, amino, (amino)alkyl, and Q-L-;
   R^{1c} is hydrogen; or
   R^{1b} and R^{1c} taken together with the carbon atom to which they are attached form a —C(═O)—; or
   R^{1b} and R^{1c} taken together with the carbon atom to which they are attached form:

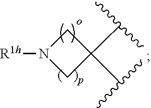

R^{1d} is selected from hydrogen and Q-L-;
R^{1e} is selected from hydrogen and Q-L-;
R^{1g} is selected from hydrogen and Q-L-;
R^{1h} is selected from hydrogen and Q-L-;
Y is selected from —O—, —S—, and —N(R^{1f})—
R^{1f} is selected from hydrogen and C₁-C₃ alkyl;
E¹ is selected from —CR^{2e}═ and —N═;
E is selected from —CR^{2f}═ and —N═;
E² is selected from —CR^{2g}═ and —N═;
E³ is selected from —CR^{2h}═ and —N═;
R^{2e}, R^{2f}, R^{2g}, and R^{2h} are independently selected from hydrogen, halo, hydroxy, amino, —CHO, C₁-C₃ alkyl, and C₁-C₃ alkoxy; or
R^{2e} is Q-L-; and, if present, R^{2f}, R^{2g}, and R^{2h} are independently selected from hydrogen, halo, hydroxy, amino, —CHO, C₁-C₃ alkyl, and C₁-C₃ alkoxy; or
R^{2f} is Q-L-; and, if present, R^{2e}, R^{2g}, and R^{2h} are independently selected from hydrogen, halo, hydroxy, amino, —CHO, C₁-C₃ alkyl, and C₁-C₃ alkoxy; or
R^{2g} is Q-L-; and, if present, R^{2e}, R^{2f}, and R^{2h} are independently selected from hydrogen, halo, hydroxy, amino, —CHO, C₁-C₃ alkyl, and C₁-C₃ alkoxy; or
R^{2h} is Q-L-; and, if present, R^{2e}, R^{2f}, and R^{2g} are independently selected from hydrogen, halo, hydroxy, amino, —CHO, C₁-C₃ alkyl, and C₁-C₃ alkoxy;
E⁴ is selected from ═C(H)— and ═N—;
E⁵ is selected from —O—, —S—, and —N(R^{2m})—;
R^{2m} is selected from hydrogen and C₁-C₄ alkyl;
R^{1j} is selected from hydrogen, C₁-C₄ alkyl, (hydroxy)alkyl, (heterocyclo)alkyl, and Q-L-;
A⁴ is selected from —CR^{2j}═ and —N═;
R^{2j} is selected from hydrogen and C₁-C₃ alkyl;
R^{2k} is selected from hydrogen and C₁-C₃ alkyl;
m is 1, 2, or 3;
n is 1, 2, or 3;
is 1, 2, or 3;
p is 1, 2, or 3;
q is 1 or 2;
t is 0 or 1;
u is 0 or 1;
R³ is selected from hydrogen, deuterium, fluoro, and C₁-C₃ alkyl;
Z and Z¹ are —C(═O)—; or
Z is —C(═O)— and Z¹ is —CR^{4a}R^{4b}—; or
Z is —CR^{4a}R^{4b}— and Z¹ is —C(═O)—; or
Z is —N═C(CH₃)— and Z¹ is —C(═O)—; or
Z is —C(═O)— and Z¹ is —N═C(CH₃)—; or
Z is a bond and Z¹ is —N(R²ⁿ)C(═O)—; or
Z is —N(R²ⁿ)C(═O) and Z is a bond;
R²ⁿ is selected from hydrogen and C₁-C₄ alkyl;
R^{4a} and R^{4b} are independently selected from hydrogen and C₁-C₃ alkyl; or
R^{4a} and R^{4b} taken together with the carbon to which they are attached form a C₃-C₆ cycloalkyl,
Q is a small molecule that binds to a target protein of interest;
L is -J¹-J²-J³-J⁴-J⁵-, wherein J¹ is attached to Q;
J¹ is selected from alkylenyl, cycloalkylenyl, and heterocyclenyl; or J¹ is absent;
J² is selected from —C(═O)—, —(CH₂)_{w}—, —CH═CH—, and —C≡C—;
w is 0, 1, 2, or 3;
J³ is selected from alkylenyl, heteroalkylenyl, cycloalkylenyl, heterocyclenyl, phenylenyl, and heteroarylenyl; or J³ is absent;
J⁴ is selected from alkylenyl, cycloalkylenyl, and heterocyclenyl; or J⁴ is absent;
J⁵ is selected from —O—, —N(H)—, —C≡C—, —(CH₂)ₓ— and —C(═O)—; and
x is 0, 1, 2, or 3.

### Embodiments of Option (a)

In some embodiments, A¹ is —CR^{2a}═.

In some embodiments, A¹ is and —N═.

In some embodiments, A is —CR^{2b}═, A² is —CR^{2c}═, wherein R^{2b} and R^{2c} are taken together to form a ring comprising —(CH₂)ₘ—X—(CH₂)ₙ—.

In some embodiments, A is —CR^{2b}═, A² is —CR^{2c}═, wherein R^{2b} and R^{2c} are taken together to form a ring comprising —C(═O)—N(R^{1d})—(CH₂)_{q}—.

In some embodiments, A is —CR^{2b}═, A² is —CR^{2c}═, wherein R^{2b} and R^{2c} are taken together to form a ring comprising —(CH₂)ₜ—N(R^{1e})—(CH₂)₂—Y—(CH₂)ᵤ—.

In some embodiments, A is —CR^{2b}═, A² is —CR^{2c}═, wherein R^{2b} and R^{2c} are taken together to form a ring comprising -E¹=E-E²=E³-.

In some embodiments, A is —CR^{2b}═, A² is —CR^{2c}═, wherein R^{2b} and R^{2c} are taken together to form a ring comprising =A⁴-N(R^{1g})—CR^{2k}═.

In some embodiments, A is —CR^{2b}═, A² is —CR^{2c}═, wherein R^{2b} and R^{2c} are taken together to form a ring comprising -E4=CR^{1j}-E⁵-.

In some embodiments, R^{2a} is hydrogen.

In some embodiments, R^{2a} is halo (e.g., F, Cl, or Br).

In some embodiments, R^{2a} is C₁-C₃ alkyl (e.g., methyl, ethyl, or butyl).

In some embodiments, R^{2a} is amino.

In some embodiments, R^{2a} is C₁-C₃ alkoxy.

In some embodiments, R^{2d} is hydrogen.

In some embodiments, R^{2d} halo (e.g., F, Cl, or Br).

In some embodiments, R^{2d} is C₁-C₃ alkyl (e.g., methyl, ethyl, or butyl).

In some embodiments, R^{2d} is amino.

In some embodiments, R^{2d} is C₁-C₃ alkoxy;

In some embodiments, A³ is —CR^{2d}═.

In some embodiments, A³ is —N═.

### Embodiments of Option (b)

In some embodiments, A¹ is —CR^{2a}═, A is —CR^{2b}═, wherein R^{2a} and R^{2b} are taken together to form a ring comprising —(CH₂)ₘ—X—(CH₂)ₙ— radical.

In some embodiments, A¹ is —CR^{2a}═, A is —CR^{2b}═, wherein R^{2a} and R^{2b} are taken together to form a ring comprising —C(═O)—N(R^{1d})—(CH₂)_{q}—.

In some embodiments, A¹ is —CR^{2a}═, A is —CR^{2b}═, wherein R^{2a} and R^{2b} are taken together to form a ring comprising —(CH₂)ₜ—N(R^{1e})—(CH₂)₂—Y—(CH₂)ᵤ—.

In some embodiments, A¹ is —CR^{2a}═, A is —CR^{2b}═, wherein R^{2a} and R^{2b} are taken together to form a ring comprising -E¹=E-E²=E³-.

In some embodiments, A¹ is —CR^{2a}═, A is —CR^{2b}═, wherein R^{2a} and R^{2b} are taken together to form a ring comprising=A⁴-N(R^{1g})—CR^{2k}═.

In some embodiments, A¹ is —CR^{2a}═, A is —CR^{2b}═, wherein R^{2a} and R^{2b} are taken together to form a ring comprising -E⁴=CR^{1j}-E⁵-.

In some embodiments, A² is —CR²c.

In some embodiments, A² is —N═.

In some embodiments, A³ is —CR^{2a}—.

In some embodiments, A³ is —N═.

In some embodiments, R^{2e} is hydrogen.

In some embodiments, R^{2e} is halo.

In some embodiments, R^{2e} is C₁-C₃ alkyl.

In some embodiments, R^{2e} is amino.

In some embodiments, R^{2e} is C₁-C₃ alkoxy.

In some embodiments, R^{2d} is hydrogen.

In some embodiments, R^{2d} is halo.

In some embodiments, R^{2d} is C₁-C₃ alkyl.

In some embodiments, R^{2d} is amino.

In some embodiments, R^{2d} is C₁-C₃ alkoxy.

### Embodiments of the Compounds

In some embodiments, X is —N(R^{1a})—.

In some embodiments, X is —CR^{1b}R^{1c}—.

In some embodiments, R^{1a} is hydrogen.

In some embodiments, R^{1a} is C₁-C₃ alkyl optionally substituted with one or more R^{1aS}.

In some embodiments, R^{1a} is C₁-C₃ haloalkyl optionally substituted with one or more R^{1aS}.

In some embodiments, R^{1a} is cycloalkyl optionally substituted with one or more R^{1aS}.

In some embodiments, R^{1a} is aryl optionally substituted with one or more R^{1aS}.

In some embodiments, R^{1a} is heterocyclo optionally substituted with one or more R^{1aS}.

In some embodiments, R^{1a} is heteroaryl optionally substituted with one or more R^{1aS}.

In some embodiments, R^{1a} is (cycloalkyl)alkyl optionally substituted with one or more R^{1aS}.

In some embodiments, R^{1a} is (aryl)alkyl optionally substituted with one or more R^{1aS}.

In some embodiments, R^{1a} is (heterocyclo)alkyl optionally substituted with one or more R^{1aS}.

In some embodiments, R^{1a} is (heteroaryl)alkyl optionally substituted with one or more R^{1aS}.

In some embodiments, R^{1a} is —C(═O)-(cycloalkyl) optionally substituted with one or more R^{1aS}.

In some embodiments, R^{1a} is —C(═O)-(aryl) optionally substituted with one or more R^{1aS}.

In some embodiments, R^{1a} is —C(═O)-(heterocyclo) optionally substituted with one or more R^{1aS}.

In some embodiments, Ria is —C(═O)-(heteroaryl) optionally substituted with one or more R^{1aS}.

In some embodiments, R^{1a} is Q-L-.

In some embodiments, at least one R^{1aS} is oxo.

In some embodiments, at least one R^{1aS} is halo.

In some embodiments, at least one R^{1aS} is C₁-C₃ alkyl optionally substituted with one or more R^{1aS}.

In some embodiments, at least one R^{1aS} is C₁-C₃ haloalkyl optionally substituted with one or more R^{1aS}.

In some embodiments, at least one R^{1aS} is C₁-C₃ alkoxy optionally substituted with one or more R^{1aS}.

In some embodiments, at least one R^{1aS} is —C(═O)—OH.

In some embodiments, at least one R^{1aS} is —C(═O)—(C₁-C₃ alkyl).

In some embodiments, at least one R^{1aS} is —C(═O)O—(C₁-C₄ alkyl),

In some embodiments, at least one R^{1aS} is —NH₂

In some embodiments, at least one R^{1aS} is —NH(C₁-C₃ alkyl)

In some embodiments, at least one R^{1aS} is —N(C₁-C₃ alkyl)₂

In some embodiments, at least one R^{1aS} is cycloalkyl optionally substituted with one or more R^{1aSS}.

In some embodiments, at least one R^{1aS} is aryl optionally substituted with one or more R^{1aSS}.

In some embodiments, at least one R^{1aS} is heterocyclo optionally substituted with one or more R^{1aSS}.

In some embodiments, at least one R^{1aS} is heteroaryl optionally substituted with one or more R^{1aSS}.

In some embodiments, at least one R^{1aS} is (cycloalkyl)alkyl optionally substituted with one or more R^{1aSS}.

In some embodiments, at least one R^{1aS} is (aryl)alkyl optionally substituted with one or more R^{1aSS}.

In some embodiments, at least one R^{1aS} is (heterocyclo)alkyl optionally substituted with one or more R^{1aSS}.

In some embodiments, at least one R^{1aS} is (heteroaryl)alkyl optionally substituted with one or more R^{1aSS}.

In some embodiments, at least one R^{1aS} is —C(═O)-(cycloalkyl) optionally substituted with one or more R^{1aSS}.

In some embodiments, at least one R^{1aS} is —C(═O)-(aryl) optionally substituted with one or more R^{1aSS}.

In some embodiments, at least one R^{1aS} is —C(═O)-(heterocyclo) optionally substituted with one or more R^{1aSS}.

In some embodiments, at least one R^{1aS} is —C(═O)-(heteroaryl) optionally substituted with one or more R^{1aSS}.

In some embodiments, at least one R^{1aSS} is oxo.

In some embodiments, at least one R^{1aSS} is halo.

In some embodiments, at least one R^{1aSS} is C₁-C₃ alkyl.

In some embodiments, at least one R^{1aSS} is C₁-C₃ haloalkyl.

In some embodiments, at least one R^{1aSS} is C₁-C₃ alkoxy.

In some embodiments, at least one R^{1aSS} is —C(═O)—OH.

In some embodiments, at least one R^{1aSS} is —C(═O)—(C₁-C₃ alkyl).

In some embodiments, at least one R^{1aSS} is —NH₂.

In some embodiments, at least one R^{1aSS} is —NH(C₁-C₃ alkyl).

In some embodiments, at least one R^{1aSS} is —N(C₁-C₃ alkyl)₂.

In some embodiments, R^{1b} is hydrogen.

In some embodiments, R^{1b} is —CHO.

In some embodiments, R^{1b} is —C(═O)OH.

In some embodiments, R^{1b} is hydroxy.

In some embodiments, R^{1b} is (hydroxy)C₁-C₃ alkyl.

In some embodiments, R^{1b} is amino.

In some embodiments, R^{1b} is (amino)alkyl.

In some embodiments, R^{1b} is (heterocyclo)alkyl, optionally substituted with one or more R^{1bS}.

In some embodiments, R^{1b} is Q-L-.

In some embodiments, at least one R^{1bS} is (aryl)alkyl optionally substituted with one or more halo.

In some embodiments, R^{1c} is hydrogen.

In some embodiments, R^{1b} and R^{1c} taken together with the carbon atom to which they are attached form a —C(═O)—.

In some embodiments, R^{1b} and R^{1c} taken together with the carbon atom to which they are attached form

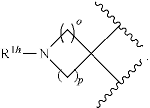

In some embodiments, R^{1d} is hydrogen.

In some embodiments, R^{1d} is heterocyclo optionally substituted with one or more R^{1dS}.

In some embodiments, R^{1d} is (heterocyclo)alkyl optionally substituted with one or more R^{1dS}.

In some embodiments, R^{1d} is Q-L-.

In some embodiments, at least one R^{1dS} is C₁-C₃ alkyl.

In some embodiments, at least one R^{1dS} is —C(═O)—(C₁-C₃ alkyl).

In some embodiments, at least one R^{1dS} is (aryl)alkyl optionally substituted with one or more halo.

In some embodiments, R^{1e} is hydrogen.

In some embodiments, R^{1e} is Q-L-.

In some embodiments, R^{1g} is hydrogen.

In some embodiments, R^{1g} is Q-L-.

In some embodiments, R^{1h} is hydrogen.

In some embodiments, R^{1h} is (aryl)alkyl optionally substituted with one or more halo.

In some embodiments, R^{1h} is Q-L-.

In some embodiments, Y is —O—.

In some embodiments, Y is —S—.

In some embodiments, Y is —N(R^{1f})—.

In some embodiments, R¹ is hydrogen.

In some embodiments, R¹ is C₁-C₃ alkyl.

In some embodiments, E¹ is —CR^{2e}.

In some embodiments, E¹ is —N═.

In some embodiments, E is —CR²f=.

In some embodiments, E is —N═.

In some embodiments, E² is —CR^{2g}.

In some embodiments, E² is —N═.

In some embodiments, E³ is —CR^{2h}═.

In some embodiments, E³ is —N═;

In some embodiments, R^{2e}, R^{2f}, R^{2g}, and R^{2h} are independently selected from hydrogen, halo, hydroxy, amino, —CHO, C₁-C₃ alkyl, and C₁-C₃ alkoxy.

In some embodiments, R^{2e} is Q-L-; and, if present, R^{2f}, R^{2g}, and R^{2h} are independently selected from hydrogen, halo, hydroxy, amino, —CHO, C₁-C₃ alkyl, and C₁-C₃ alkoxy.

In some embodiments, R^{2f} is Q-L-; and, if present, R^{2e}, R^{2g}, and R^{2h} are independently selected from hydrogen, halo, hydroxy, amino, —CHO, C₁-C₃ alkyl, and C₁-C₃ alkoxy.

In some embodiments, R^{2g} is Q-L-; and, if present, R^{2e}, R^{2f}, and R^{2h} are independently selected from hydrogen, halo, hydroxy, amino, —CHO, C₁-C₃ alkyl, and C₁-C₃ alkoxy.

In some embodiments, R^{2h} is Q-L-; and, if present, R^{2e}, R^{2f}, and R^{2g} are independently selected from hydrogen, halo, hydroxy, amino, —CHO, C₁-C₃ alkyl, and C₁-C₃ alkoxy.

In some embodiments, E⁴ is ═C(H)—.

In some embodiments, E⁴ is ═N—.

In some embodiments, E⁵ is —O—.

In some embodiments, E⁵ is —S—.

In some embodiments, E⁵ is —N(R^{2m})—.

In some embodiments, R^{2m} is hydrogen.

In some embodiments, R^{2m} is C₁-C₄ alkyl.

In some embodiments, R^{1j} is hydrogen.

In some embodiments, R^{1j} is C₁-C₄ alkyl.

In some embodiments, R^{1j} is (hydroxy)alkyl.

In some embodiments, R^{1j} is (heterocyclo)alkyl.

In some embodiments, R^{1j} is Q-L-.

In some embodiments, A⁴ is —CR^{2j}═.

In some embodiments, A⁴ is —N═.

In some embodiments, R^{2j} is hydrogen.

In some embodiments, R^{2j} is C₁-C₃ alkyl.

In some embodiments, R^{2k} is hydrogen.

In some embodiments, R^{2k} is C₁-C₃ alkyl.

In some embodiments, m is 1.

In some embodiments, m is 2.

In some embodiments, m is 3.

In some embodiments, n is 1.

In some embodiments, n is 2.

In some embodiments, n is 3.

In some embodiments, o is 1.

In some embodiments, o is 2.

In some embodiments, o is 3.

In some embodiments, p is 1.

In some embodiments, p is 2.

In some embodiments, p is 3.

In some embodiments, q is 1.

In some embodiments, q is 2.

In some embodiments, t is 0.

In some embodiments, t is 1.

In some embodiments, u is 0.

In some embodiments, u is 1.

In some embodiments, R³ is hydrogen.

In some embodiments, R³ is deuterium.

In some embodiments, R³ is fluoro.

In some embodiments, R³ is C₁-C₃ alkyl.

In some embodiments, Z and Z¹ are —C(═O)—.

In some embodiments, Z is —C(═O)— and Z¹ is —CR^{4a}R^{4b}—.

In some embodiments, Z is —CR^{4a}R^{4b}— and Z¹ is —C(═O)—.

In some embodiments, Z is —N═C(CH₃)— and Z¹ is —C(═O)—.

In some embodiments, Z is —C(═O)— and Z¹ is —N═C(CH₃)—.

In some embodiments, Z is a bond and Z¹ is —N(R²ⁿ)C(═O)—.

In some embodiments, Z is —N(R²ⁿ)C(═O) and Z is a bond.

In some embodiments, R²ⁿ is hydrogen.

In some embodiments, R²ⁿ is C₁-C₄ alkyl.

In some embodiments, R^{4a} and R^{4b} are independently selected from hydrogen and C₁-C₃alkyl.

In some embodiments, R^{4a} and R^{4b} taken together with the carbon to which they are attached form a C₃-C₆ cycloalkyl.

In some embodiments, Q is a small molecule that binds to a target protein of interest.

In some embodiments, L is -J¹-J²-J³-J⁴-J⁵-, wherein J¹ is attached to Q;

In some embodiments, J¹ is alkylenyl.

In some embodiments, J¹ is cycloalkylenyl.

In some embodiments, J¹ is heterocyclenyl.

In some embodiments, J¹ is absent.

In some embodiments, J² is —C(═O)—.

In some embodiments, J² is —(CH₂)_{w}—.

In some embodiments, J² is —CH═CH—.

In some embodiments, J² is —C≡C—.

In some embodiments, w is 0.

In some embodiments, w is 1.

In some embodiments, w is 2.

In some embodiments, w is 3.

In some embodiments, J³ is alkylenyl.

In some embodiments, J³ is heteroalkylenyl.

In some embodiments, J³ is cycloalkylenyl.

In some embodiments, J³ is heterocyclenyl.

In some embodiments, J³ is phenylenyl.

In some embodiments, J³ is heteroarylenyl.

In some embodiments, J³ is absent.

In some embodiments, J⁴ is alkylenyl.

In some embodiments, J⁴ is cycloalkylenyl.

In some embodiments, J⁴ is heterocyclenyl.

In some embodiments, J⁴ is absent.

In some embodiments, J⁵ is —O—.

In some embodiments, J⁵ is —N(H)—.

In some embodiments, J⁵ is —C≡C—.

In some embodiments, J⁵ is —(CH₂)ₓ—.

In some embodiments, J⁵ is —C(═O)—.

In some embodiments, x is 0.

In some embodiments, x is 1.

In some embodiments, x is 2.

In some embodiments, x is 3.

In some embodiments, the compound is not a compound of any one of Formulae A-F:

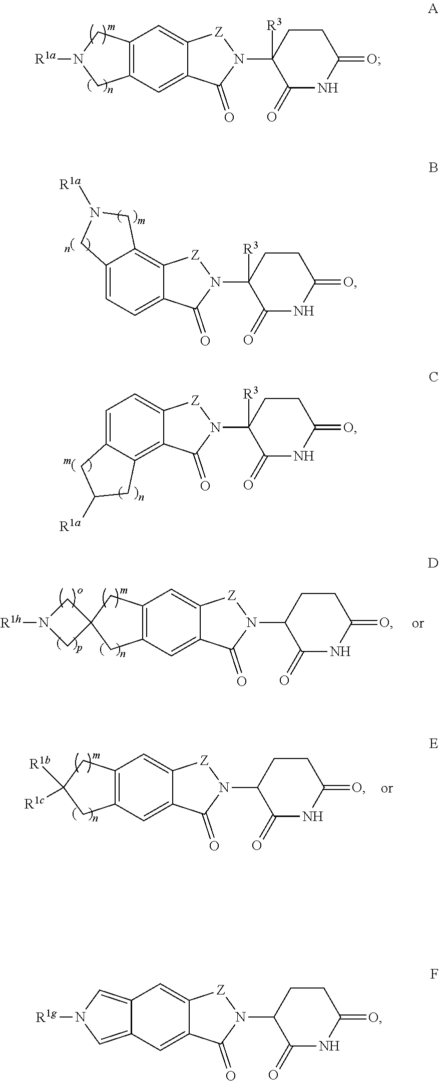

wherein:
R^{1a}, R^{1h}, R^{1g}, m, n, o, and p are as defined in connection with Formula I;
R³ is selected from hydrogen, fluoro, methyl, and deuterium;
Z is selected from —C(═O)— and —CH₂—;
R^{1b} is selected from —OH, —CH₂OH, —CHO, —C(═O)OH, and Q-L; and
R^{1c} is hydrogen; or
R^{1b} and R^{1c} taken together with the carbon atom to which they are attached form a —C(═O)—.

In some embodiments, the compound is not any of the following compounds:

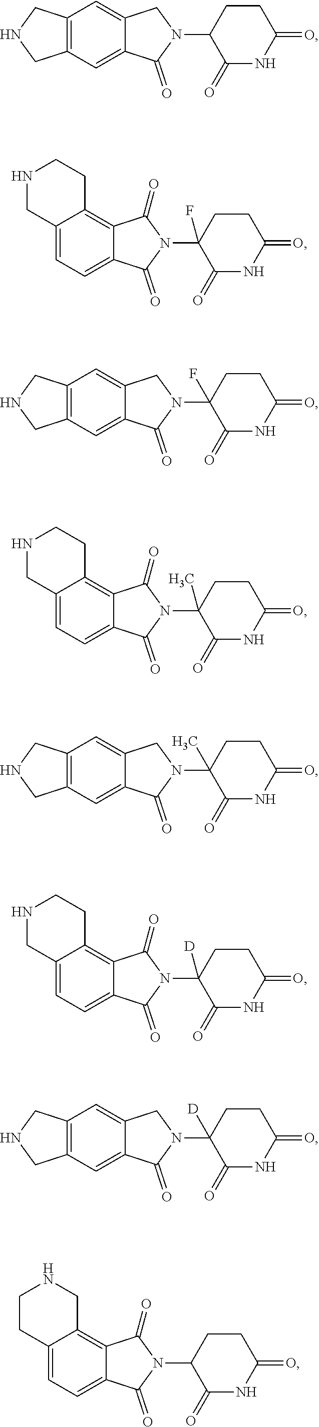

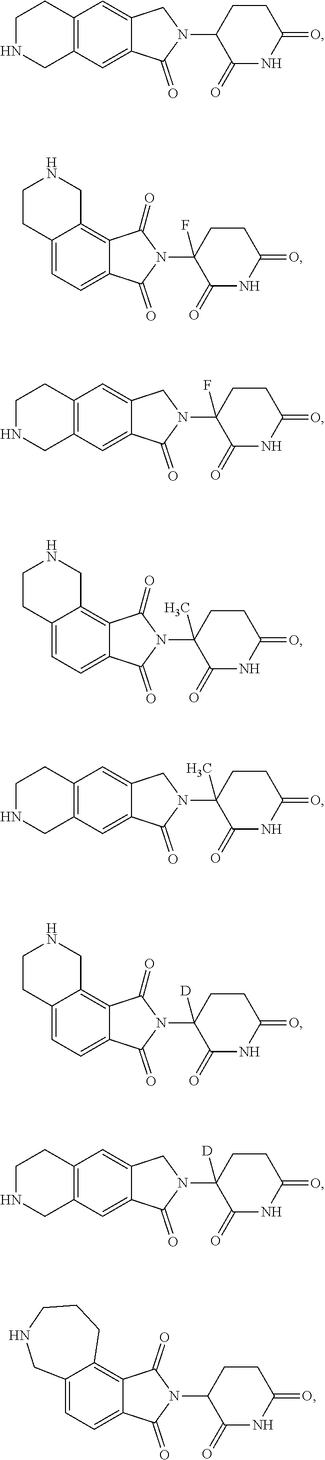

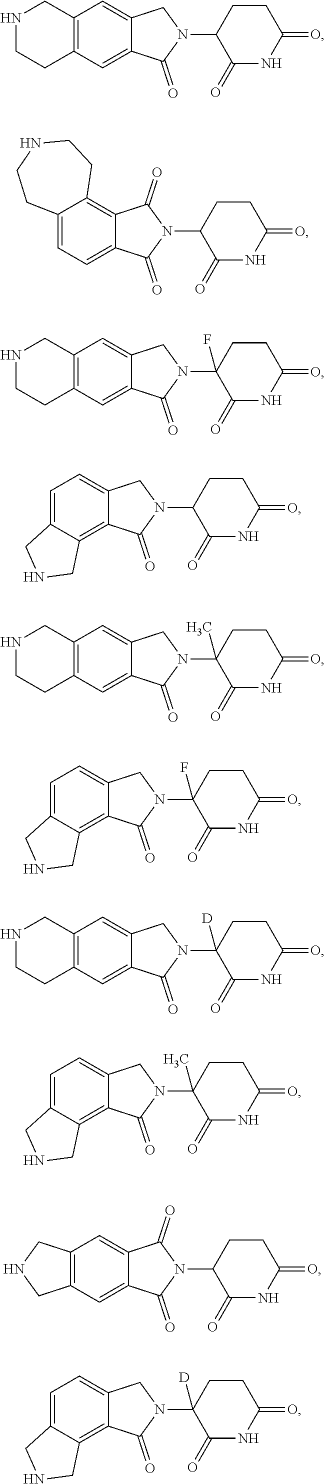

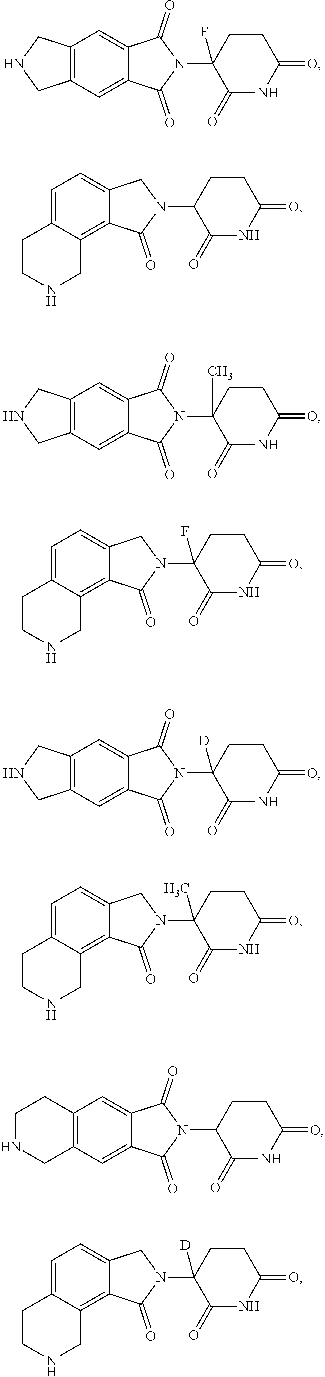

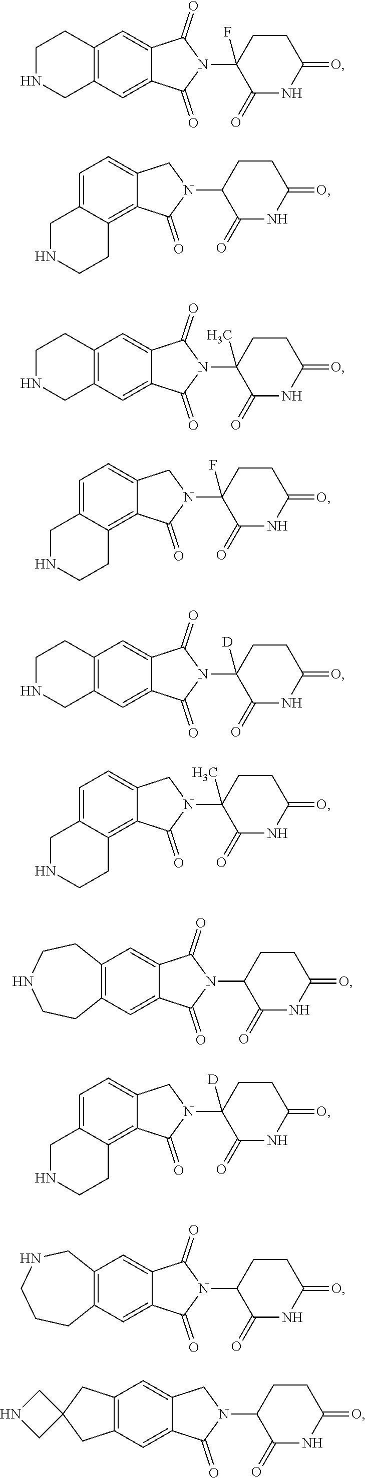

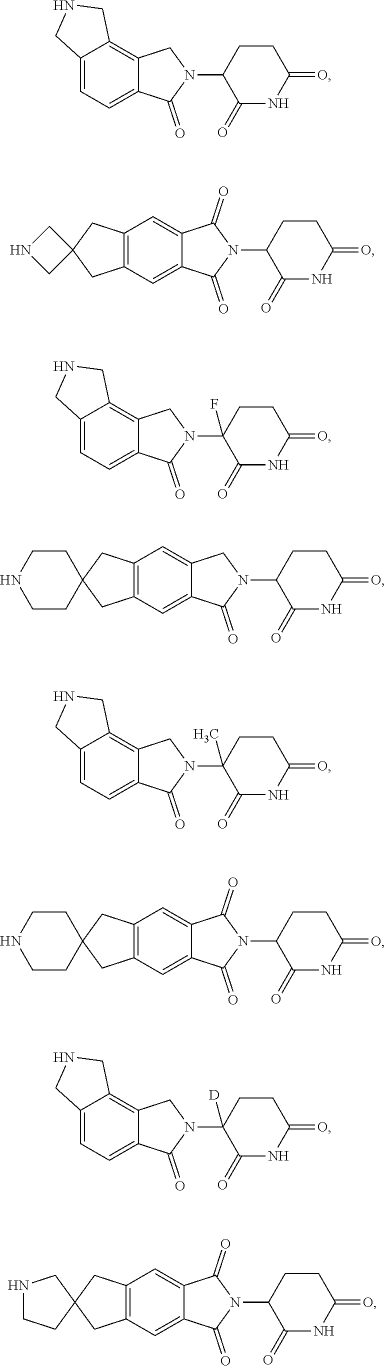

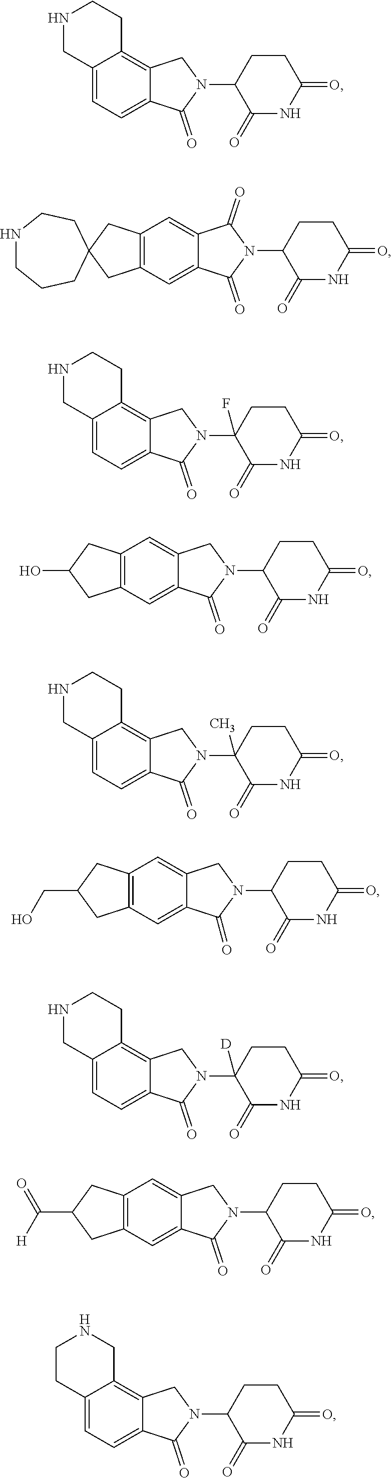

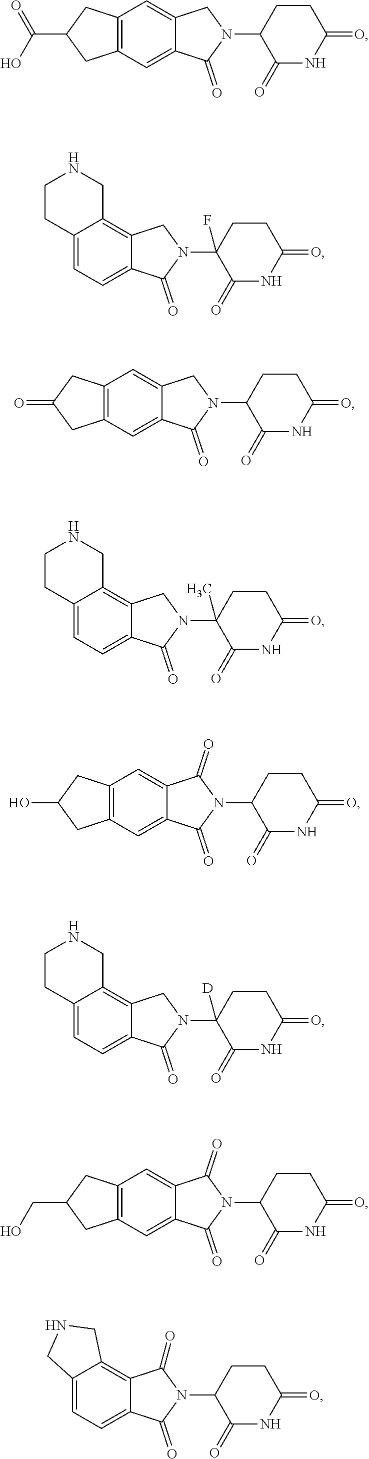

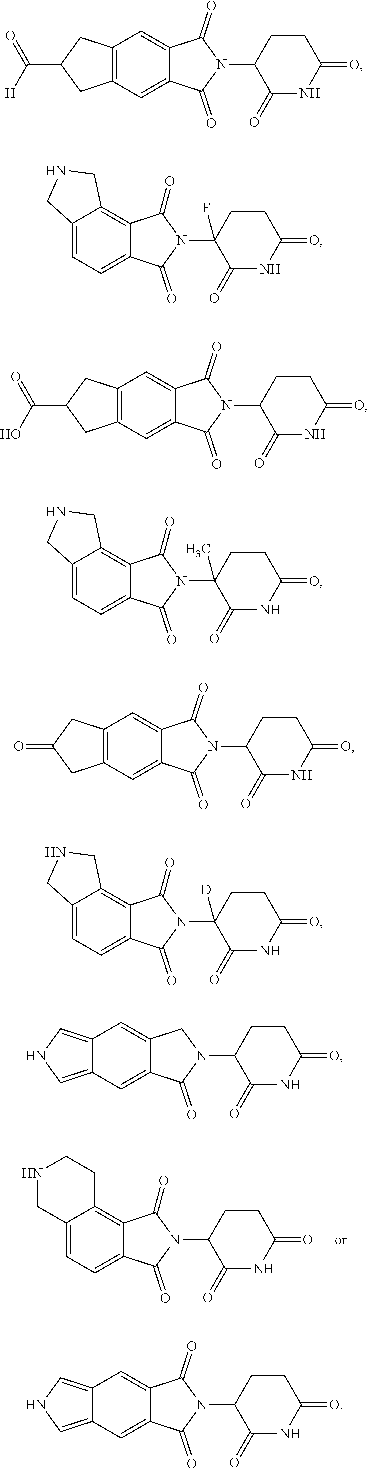

In some embodiments, the compound is of Formula II:

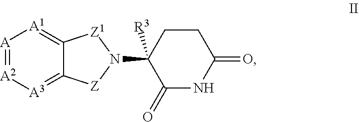

or a pharmaceutically acceptable salt or solvate thereof, wherein A, A¹, A², A³, Z, Z¹ and R³ are as defined in connection with Formula I.

In some embodiments, the compound is of Formula III:

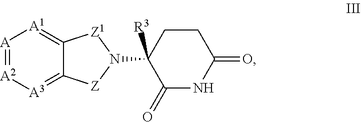

or a pharmaceutically acceptable salt or solvate thereof, wherein A, A¹, A², A³, Z, Z¹ and R³ are as defined in connection with Formula I.

In some embodiments, the compound is of Formula IV:

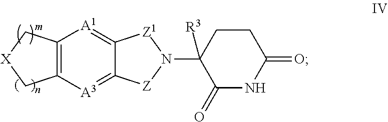

or a pharmaceutically acceptable salt or solvate thereof, wherein A¹, A³, X, m, n, Z, Z¹, and R³ are as defined in connection with Formula I.

In some embodiments, the compound is of Formula V:

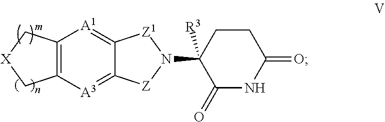

or a pharmaceutically acceptable salt or solvate thereof, wherein A¹, A³, X, m, n, Z, Z¹, and R³ are as defined in connection with Formula I.

In some embodiments, the compound is of Formula VI:

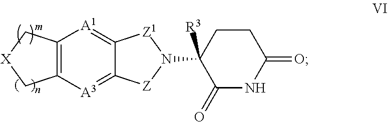

or a pharmaceutically acceptable salt or solvate thereof, wherein A¹, A³, X, m, n, Z, Z¹, and R³ are as defined in connection with Formula I.

In some embodiments, the compound is of any one of Formulae I-VI, or a pharmaceutically acceptable salt or solvate thereof, wherein A¹ is —CR^{2a}═ and A³ is —CR^{2d}—. In some embodiments, R^{2a} is selected from hydrogen, —NH₂, fluoro, and methoxy. In some embodiments, R^{2d} is selected from hydrogen, —NH₂, fluoro, and methoxy.

In some embodiments, the compound is of any one of Formulae I-VI, or a pharmaceutically acceptable salt or solvate thereof, wherein A¹ is —N═ and A³ is —CR^{2d}—. In some embodiments, wherein R^{2d} is selected from hydrogen, —NH₂, fluoro, and methoxy.

In some embodiments, the compound is of any one of Formulae I-VI, or a pharmaceutically acceptable salt or solvate thereof, wherein A¹ is —CR^{2a}═ and A³ is —N═. In some embodiments, R^{2a} is selected from hydrogen, —NH₂, fluoro, and methoxy.

In some embodiments, the compound is of Formula VII:

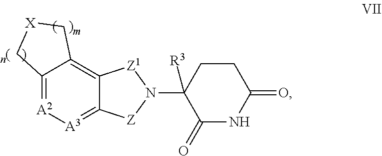

or a pharmaceutically acceptable salt or solvate thereof, wherein A², A³, X, m, n, Z, Z¹, and R³ are as defined in connection with Formula I.

In some embodiments, the compound is of Formula VIII:

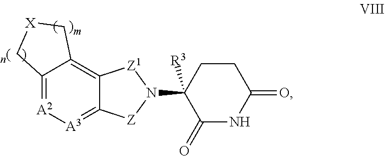

or a pharmaceutically acceptable salt or solvate thereof, wherein A², A³, X, m, n, Z, Z¹, and R³ are as defined in connection with Formula I.

In some embodiments, the compound is of Formula IX:

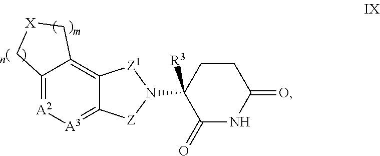

or a pharmaceutically acceptable salt or solvate thereof, wherein A², A³, X, m, n, Z, Z¹, and R³ are as defined in connection with Formula I.

In some embodiments, the compound is of any one of Formulae VII-IX, or a pharmaceutically acceptable salt or solvate thereof, wherein A² is —CR^{2c}═ and A³ is —CR^{2d}—. In some embodiments, R^{2c} is selected from hydrogen, —NH₂, fluoro, and methoxy. In some embodiments, R^{2d} is selected from hydrogen, —NH₂, fluoro, and methoxy.

In some embodiments, the compound is of any one of Formulae VII-IX, or a pharmaceutically acceptable salt or solvate thereof, wherein A² is —N═ and A³ is —CR^{2d}—. In some embodiments, R^{2d} is selected from hydrogen, —NH₂, fluoro, and methoxy.

In some embodiments, the compound is of any one of Formulae VII-IX, or a pharmaceutically acceptable salt or solvate thereof, wherein A² is —CR^{2c}═ and A³ is —N═. In some embodiments, R^{2c} is selected from hydrogen, —NH₂, fluoro, and methoxy.

In some embodiments, the compound is of any one of Formulae IV-IX, or a pharmaceutically acceptable salt or solvate thereof, wherein m is 1.

In some embodiments, the compound is of any one of Formulae IV-IX, or a pharmaceutically acceptable salt or solvate thereof, wherein m is 2.

In some embodiments, the compound is of any one of Formulae IV-IX, or a pharmaceutically acceptable salt or solvate thereof, wherein m is 3.

In some embodiments, the compound is of any one of Formulae IV-IX, or a pharmaceutically acceptable salt or solvate thereof, wherein n is 1.

In some embodiments, the compound is of any one of Formulae IV-IX, or a pharmaceutically acceptable salt or solvate thereof, wherein n is 2.

In some embodiments, the compound is of any one of Formulae IV-IX, or a pharmaceutically acceptable salt or solvate thereof, wherein n is 3

In some embodiments, the compound is of any one of Formulae IV-IX, or a pharmaceutically acceptable salt or solvate thereof, wherein X is —CR^{1b}R^{1c}—. In some embodiments, R^{1c} is hydrogen. In some embodiments, R^{1b} is selected from hydroxy, —NH₂, —CHO, —C(═O)OH, and —CH₂OH. In some embodiments, R^{1b} is Q-L-. In some embodiments, R^{1b} and R^{1c} taken together with the carbon atom to which they are attached form —C(═O)—.

In some embodiments, the compound is of any one of Formulae IV-IX, or a pharmaceutically acceptable salt or solvate thereof, wherein X is —N(R^{1a})—. In some embodiments, R^{1a} is hydrogen. In some embodiments, R^{1a} is Q-L-.

In some embodiments, the compound is of Formula X:

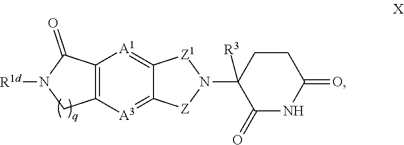

or a pharmaceutically acceptable salt or solvate thereof, wherein A¹, A³, q, Z, Z¹, R^{1d}, and R³ are as defined in connection with Formula I.

In some embodiments, the compound is of Formula XI:

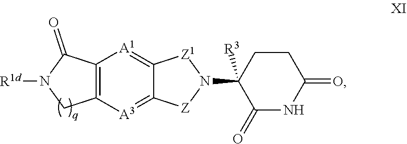

or a pharmaceutically acceptable salt or solvate thereof, wherein A¹, A³, q, Z, Z¹, R^{1d}, and R³ are as defined in connection with Formula I.

In some embodiments, the compound is of Formula XII:

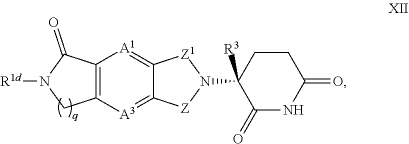

or a pharmaceutically acceptable salt or solvate thereof, wherein A¹, A³, q, Z, Z¹, R^{1d}, and R³ are as defined in connection with Formula I.

In some embodiments, the compound is of any one of Formulae X-XII, or a pharmaceutically acceptable salt or solvate thereof, wherein A1 is —CR^{2a}═ and A³ is —CR^{2d}═. In some embodiments, R^{2a} is selected from hydrogen, —NH₂, fluoro, and methoxy. In some embodiments, R^{2d} is selected from hydrogen, —NH₂, fluoro, and methoxy.

In some embodiments, the compound is of any one of Formulae X-XII, or a pharmaceutically acceptable salt or solvate thereof, wherein A¹ is —N═ and A³ is —CR^{2d}—. In some embodiments, R^{2d} is selected from hydrogen, —NH₂, fluoro, and methoxy.

In some embodiments, the compound is of any one of Formulae X-XII, or a pharmaceutically acceptable salt or solvate thereof, wherein A¹ is —CR^{2a}═ and A³ is —N═. In some embodiments, R^{2a} is selected from hydrogen, —NH₂, fluoro, and methoxy.

In some embodiments, the compound is of any one of Formulae X-XII, or a pharmaceutically acceptable salt or solvate thereof, wherein q is 2. In some embodiments, q is 1.

In some embodiments, the compound is of any one of Formulae X-XII, or a pharmaceutically acceptable salt or solvate thereof, wherein R^{1d} is hydrogen. In some embodiments, R^{1d} is Q-L-.

In some embodiments, the compound is of Formula XIII:

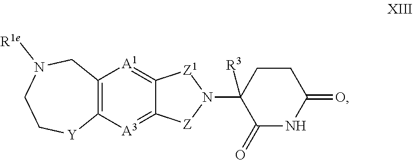

or a pharmaceutically acceptable salt or solvate thereof, wherein A¹, A³, Y, Z, Z¹, R^{1e}, and R³ are as defined in connection with Formula I.

In some embodiments, the compound is of Formula XIV:

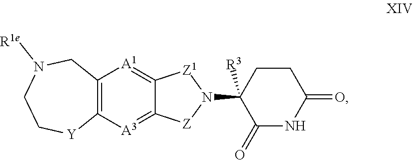

or a pharmaceutically acceptable salt or solvate thereof, wherein A¹, A³, Y, Z, Z¹, R^{1e}, and R³ are as defined in connection with Formula I.

In some embodiments, the compound is of Formula XV:

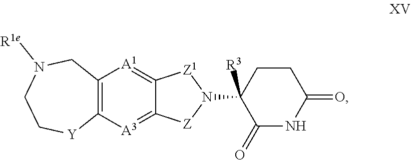

or a pharmaceutically acceptable salt or solvate thereof, wherein A¹, A³, Y, Z, Z¹, R^{1e}, and R³ are as defined in connection with Formula I.

In some embodiments, the compound is of any one of Formulae XIII-XV, or a pharmaceutically acceptable salt or solvate thereof, wherein A¹ is —CR^{2a}═ and A³ is —CR^{2d}═. In some embodiments, R^{2a} is selected from hydrogen, —NH₂, fluoro, and methoxy. In some embodiments, R^{2d} is selected from hydrogen, —NH₂, fluoro, and methoxy.

In some embodiments, the compound is of any one of Formulae XIII-XV, or a pharmaceutically acceptable salt or solvate thereof, wherein A¹ is —N═ and A³ is —CR^{2d}═. In some embodiments, R^{2d} is selected from hydrogen, —NH₂, fluoro, and methoxy.

In some embodiments, the compound is of any one of Formulae XIII-XV, or a pharmaceutically acceptable salt or solvate thereof, wherein A¹ is —CR^{2a}═ and A³ is —N═. In some embodiments, R^{2a} is selected from hydrogen, —NH₂, fluoro, and methoxy.

In some embodiments, the compound is of Formula XVI:

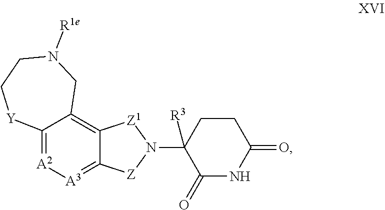

or a pharmaceutically acceptable salt or solvate thereof, wherein A, A¹, Y, Z, Z¹, R^{1e}, and R³ are as defined in connection with Formula I.

In some embodiments, the compound is of Formula XVII:

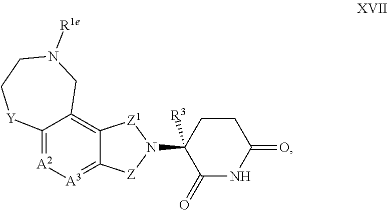

or a pharmaceutically acceptable salt or solvate thereof, wherein A, A¹, Y, Z, Z¹, R^{1e}, and R³ are as defined in connection with Formula I.

In some embodiments, the compound is of Formula XVIII:

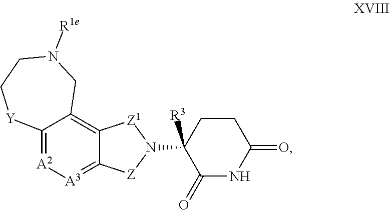

or a pharmaceutically acceptable salt or solvate thereof, wherein A, A¹, Y, Z, Z¹, R^{1e}, and R³ are as defined in connection with Formula I.

In some embodiments, the compound is of Formula XIX:

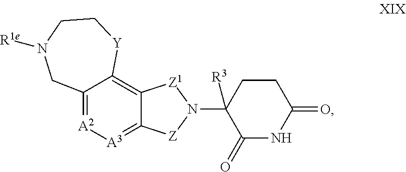

or a pharmaceutically acceptable salt or solvate thereof, wherein A, A¹, Y, Z, Z¹, R^{1e}, and R³ are as defined in connection with Formula I.

In some embodiments, the compound is of Formula XX:

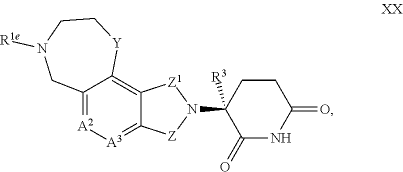

or a pharmaceutically acceptable salt or solvate thereof, wherein A, A¹, Y, Z, Z¹, R^{1e}, and R³ are as defined in connection with Formula I.

In some embodiments, the compound is of Formula XXI:

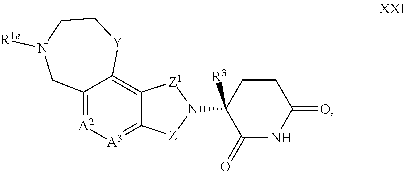

or a pharmaceutically acceptable salt or solvate thereof, wherein A, A¹, Y, Z, Z¹, R^{1e}, and R³ are as defined in connection with Formula I.

In some embodiments, the compound is of any one of Formulae XVI-XXI, or a pharmaceutically acceptable salt or solvate thereof, wherein A² is —CR^{2c}═ and A³ is —CR^{2d}═. In some embodiments, R^{2c} is selected from hydrogen, —NH₂, fluoro, and methoxy. In some embodiments, R^{2d} is selected from hydrogen, —NH₂, fluoro, and methoxy.

In some embodiments, the compound is of any one of Formulae XVI-XXI, or a pharmaceutically acceptable salt or solvate thereof, wherein A² is —N═ and A³ is —CR^{2d}═. In some embodiments, R^{2d} is selected from hydrogen, —NH₂, fluoro, and methoxy.

In some embodiments, the compound is of any one of Formulae XVI-XXI, or a pharmaceutically acceptable salt or solvate thereof, wherein A² is —CR^{2c}═ and A is —N═. In some embodiments, R^{2c} is selected from hydrogen, —NH₂, fluoro, and methoxy.

In some embodiments, the compound is of any one of Formulae XIII-XXI, or a pharmaceutically acceptable salt or solvate thereof, wherein R^{1e} is hydrogen. In some embodiments, R^{1e} is Q-L-.

In some embodiments, the compound is of Formula XXII:

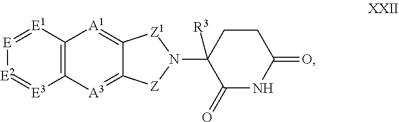

or a pharmaceutically acceptable salt or solvate thereof, wherein A, A¹, E, E¹, E², E³, Z, Z¹, and R³ are as defined in connection with Formula I.

In some embodiments, the compound is of Formula XXIII:

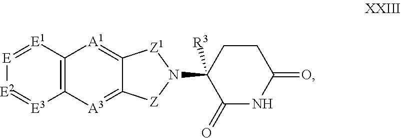

or a pharmaceutically acceptable salt or solvate thereof, wherein A, A¹, E, E¹, E², E³, Z, Z¹, and R³ are as defined in connection with Formula I.

In some embodiments, the compound is of Formula XXIV:

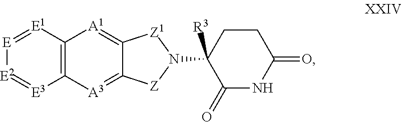

or a pharmaceutically acceptable salt or solvate thereof, wherein A, A¹, E, E¹, E², E³, Z, Z¹, and R³ are as defined in connection with Formula I.

In some embodiments, the compound is of any one of Formulae XXII-XXIV, or a pharmaceutically acceptable salt or solvate thereof, wherein A¹ is —CR^{2a}═ and A³ is —CR^{2d}═. In some embodiments, R^{2a} is selected from hydrogen, —NH₂, fluoro, and methoxy. In some embodiments, R^{2d} is selected from hydrogen, —NH₂, fluoro, and methoxy.

In some embodiments, the compound is of any one of Formulae XXII-XXIV, or a pharmaceutically acceptable salt or solvate thereof, wherein A¹ is —N═ and A³ is —CR^{2d}═. In some embodiments, R^{2d} is selected from hydrogen, —NH₂, fluoro, and methoxy.

In some embodiments, the compound is of any one of Formulae XXII-XXIV, or a pharmaceutically acceptable salt or solvate thereof, wherein A¹ is —CR^{2a}═ and A³ is —N═. In some embodiments, R^{2a} is selected from hydrogen, —NH₂, fluoro, and methoxy.

In some embodiments, the compound is of any one of Formulae XXII-XXIV, or a pharmaceutically acceptable salt or solvate thereof, wherein E¹ is —N═. In some embodiments, E is —N═. In some embodiments, E is —CR^{2f}═. In some embodiments, E² is —N═. In some embodiments, E² is —CR^{2g}═. In some embodiments, E³ is —N═. In some embodiments, E³ is —CR^{2h}═.

In some embodiments, the compound is of any one of Formulae XXII-XXIV, or a pharmaceutically acceptable salt or solvate thereof, wherein E¹ is —CR^{2e}═ and R^{2e} is Q-L-. In some embodiments, E is —N═. In some embodiments, E is —CH═. In some embodiments, E² is —N═. In some embodiments, E² is —CH═. In some embodiments, E³ is —N═. In some embodiments, E³ is —CH═.

In some embodiments, the compound is of any one of Formulae XXII-XXIV, or a pharmaceutically acceptable salt or solvate thereof, wherein E is —N═. In some embodiments, E¹ is —N═. In some embodiments, E¹ is —CR^{2e}═. In some embodiments, E² is —N═. In some embodiments, E² is —CR^{2g}═. In some embodiments, E³ is —N═. In some embodiments, E³ is —CR^{2h}═.

In some embodiments, the compound is of any one of Formulae XXII-XXIV, or a pharmaceutically acceptable salt or solvate thereof, wherein E is —CR^{2f}═ and R^{2f} is Q-L-. In some embodiments, E¹ is —N═. In some embodiments, E¹ is —CH═. In some embodiments, E² is —N═. In some embodiments, E² is —CH═. In some embodiments, E³ is —N═. In some embodiments, E³ is —CH=

In some embodiments, the compound is of any one of Formulae XXII-XXIV, or a pharmaceutically acceptable salt or solvate thereof, wherein E² is —N═. In some embodiments, E¹ is —N═. In some embodiments, E¹ is —CR^{2e}═. In some embodiments, E is —N═. In some embodiments, E is —CR^{2f}═. In some embodiments, E³ is —N═. In some embodiments, E³ is —CR^{2h}═.

In some embodiments, the compound is of any one of Formulae XXII-XXIV, or a pharmaceutically acceptable salt or solvate thereof, wherein E² is —CR^{2g}═ and R^{2g} is Q-L-. In some embodiments, E¹ is —N═. In some embodiments, E¹ is —CH═. In some embodiments, E is —N═. In some embodiments, E is —CH═. In some embodiments, E³ is —N═. In some embodiments, E³ is —CH═.

In some embodiments, the compound is of any one of Formulae XXII-XXIV, or a pharmaceutically acceptable salt or solvate thereof, wherein E³ is —N═. In some embodiments, E¹ is —N═. In some embodiments, E¹ is —CR^{2e}═. In some embodiments, E is —N═. In some embodiments, E is —CR^{2f}═. In some embodiments, E² is —N═. In some embodiments, E² is —CR^{2g}═.

In some embodiments, the compound is of any one of Formulae XXII-XXIV, or a pharmaceutically acceptable salt or solvate thereof, wherein E³ is —CR^{2h}═ and R^{2h} is Q-L-. In some embodiments, E¹ is —N═. In some embodiments, E¹ is —CH═. In some embodiments, E is —N═. In some embodiments, E is —CH═. In some embodiments, E² is —N═. In some embodiments, E² is —CH═.

In some embodiments, the compound is of Formula XXV:

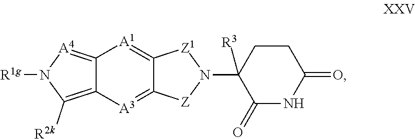

or a pharmaceutically acceptable salt or solvate thereof, wherein A¹, A³, A⁴, Z, Z¹, R^{1g}, R^{2k}, and R³ are as defined in connection with Formula I.

In some embodiments, the compound is of Formula XXVI:

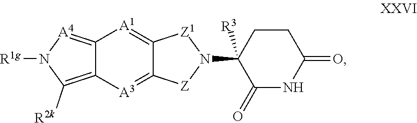

or a pharmaceutically acceptable salt or solvate thereof, wherein A¹, A³, A⁴, Z, Z¹, R^{1g}, R^{2k}, and R³ are as defined in connection with Formula I.

In some embodiments, the compound is of Formula XXVII:

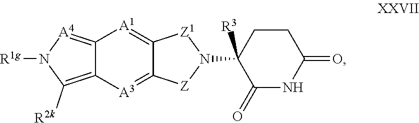

or a pharmaceutically acceptable salt or solvate thereof, wherein A¹, A³, A⁴, Z, Z¹, R^{1g}, R^{2k}, and R³ are as defined in connection with Formula I.

In some embodiments, the compound is of any one of Formulae XXV-XXVII, or a pharmaceutically acceptable salt or solvate thereof, wherein A¹ is —CR^{2a}═ and A³ is —CR^{2d}═. In some embodiments, R^{2a} is selected from hydrogen, —NH₂, fluoro, and methoxy. In some embodiments, R^{2d} is selected from hydrogen, —NH₂, fluoro, and methoxy.

In some embodiments, the compound is of any one of Formulae XXV-XXVII, or a pharmaceutically acceptable salt or solvate thereof, wherein A¹ is —N═ and A³ is —CR^{2d}═. In some embodiments, R^{2d} is selected from hydrogen, —NH₂, fluoro, and methoxy.

In some embodiments, the compound is of any one of Formulae XXV-XXVII, or a pharmaceutically acceptable salt or solvate thereof, wherein A¹ is —CR^{2a}═ and A³ is —N═. In some embodiments, R^{2a} is selected from hydrogen, —NH₂, fluoro, and methoxy.

In some embodiments, the compound is of any one of Formulae XXV-XXVII, or a pharmaceutically acceptable salt or solvate thereof, wherein A⁴ is —N═. In some embodiments, A⁴ is —CH═.

In some embodiments, the compound is of any one of Formulae XXV-XXVII, a pharmaceutically acceptable salt or solvate thereof, wherein R^{2k} is hydrogen.

In some embodiments, the compound is of any one of Formulae XXV-XXVII, a pharmaceutically acceptable salt or solvate thereof, wherein R¹⁹ is hydrogen. In some embodiments, R^{1g} is Q-L.

In some embodiments, the compound is of Formula XXVIII:

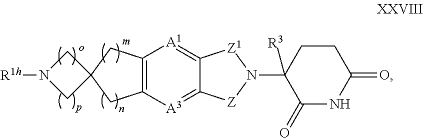

or a pharmaceutically acceptable salt or solvate thereof, wherein A¹, A³, m, n, o, p, Z, Z¹, R^{1h} and R³ are as defined in connection with Formula I.

In some embodiments, the compound is of Formula XXIX:

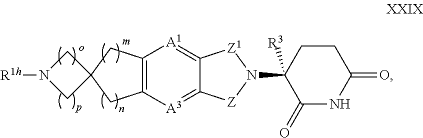

or a pharmaceutically acceptable salt or solvate thereof, wherein A¹, A³, m, n, o, p, Z, Z¹, R^{1h} and R³ are as defined in connection with Formula I.

In some embodiments, the compound is of Formula XXX:

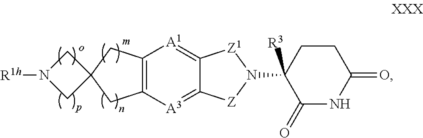

or a pharmaceutically acceptable salt or solvate thereof, wherein A¹, A³, m, n, o, p, Z, Z¹, R^{1h} and R³ are as defined in connection with Formula I.

In some embodiments, the compound is of any one of Formulae XXVIII-XXX, or a pharmaceutically acceptable salt or solvate thereof, wherein A¹ is —CR^{2a}═ and A³ is —CR^{2d}═. In some embodiments, R^{2a} is selected from hydrogen, —NH₂, fluoro, and methoxy. In some embodiments, R^{2d} is selected from hydrogen, —NH₂, fluoro, and methoxy.

In some embodiments, the compound is of any one of Formulae XXVIII-XXX, or a pharmaceutically acceptable salt or solvate thereof, wherein A¹ is —N═ and A³ is —CR^{2d}═. In some embodiments, R^{2d} is selected from hydrogen, —NH₂, fluoro, and methoxy.

In some embodiments, the compound is of any one of Formulae XXVIII-XXX, or a pharmaceutically acceptable salt or solvate thereof, wherein A¹ is —CR^{2a}═ and A³ is —N═. In some embodiments, R^{2a} is selected from hydrogen, —NH₂, fluoro, and methoxy.

In some embodiments, the compound is of any one of Formulae XXVIII-XXX, or a pharmaceutically acceptable salt or solvate thereof, wherein m is 1. In some embodiments, m is 2.

In some embodiments, the compound is of any one of Formulae XXVIII-XXX, or a pharmaceutically acceptable salt or solvate thereof, wherein n is 1. In some embodiments, n is 2.

In some embodiments, the compound is of any one of Formulae XXVIII-XXX, or a pharmaceutically acceptable salt or solvate thereof, wherein o is 1. In some embodiments, o is 2.

In some embodiments, the compound is of any one of Formulae XXVIII-XXX, or a pharmaceutically acceptable salt or solvate thereof, wherein p is 1. In some embodiments, p is 2.

In some embodiments, the compound is of any one of Formulae XXVIII-XXX, or a pharmaceutically acceptable salt or solvate thereof, wherein R^{1h} is hydrogen. In some embodiments, R^{1h} is Q-L-.

In some embodiments, the compound is of Formula XXXI:

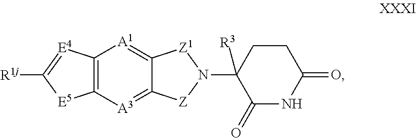

or a pharmaceutically acceptable salt or solvate thereof, wherein A¹, A³, E⁴, E⁵, Z, Z¹, R^{1j}, and R³ are as defined in connection with Formula I.

In some embodiments, the compound is of Formula XXXII:

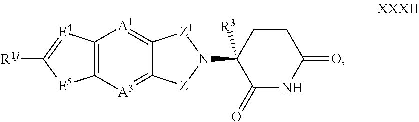

or a pharmaceutically acceptable salt or solvate thereof, wherein A¹, A³, E⁴, E⁵, Z, Z¹, R^{1j}, and R³ are as defined in connection with Formula I.

In some embodiments, the compound is of Formula XXXIII:

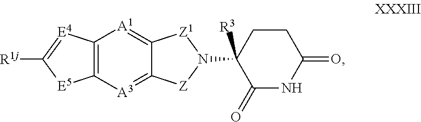

or a pharmaceutically acceptable salt or solvate thereof, wherein A¹, A³, E⁴, E⁵, Z, Z¹, R^{1j}, and R³ are as defined in connection with Formula I.

In some embodiments, the compound is of any one of Formulae XXXI-XXXIII, or a pharmaceutically acceptable salt or solvate thereof, wherein A¹ is —CR^{2a}═ and A³ is —CR^{2d}═. In some embodiments, R^{2a} is selected from hydrogen, —NH₂, fluoro, and methoxy. In some embodiments, R^{2d} is selected from hydrogen, —NH₂, fluoro, and methoxy.

In some embodiments, the compound is of any one of Formulae XXXI-XXXIII, or a pharmaceutically acceptable salt or solvate thereof, wherein A¹ is —N═ and A³ is —CR^{2d}═. In some embodiments, R^{2d} is selected from hydrogen, —NH₂, fluoro, and methoxy.

In some embodiments, the compound is of any one of Formulae XXXI-XXXIII, or a pharmaceutically acceptable salt or solvate thereof, wherein A¹ is —CR^{2a}═ and A³ is —N═. In some embodiments, R^{2a} is selected from hydrogen, —NH₂, fluoro, and methoxy.

In some embodiments, the compound is of Formula XXXIV:

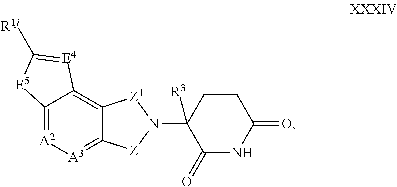

or a pharmaceutically acceptable salt or solvate thereof, wherein A², A³, E⁴, E⁵, Z, Z¹, R¹, and R³ are as defined in connection with Formula I.

In some embodiments, the compound is of Formula XXXV:

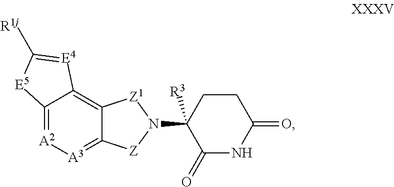

or a pharmaceutically acceptable salt or solvate thereof, wherein A¹, A³, E⁴, E⁵, Z, Z¹, R^{1j}, and R³ are as defined in connection with Formula I.

In some embodiments, the compound is of Formula XXXVI:

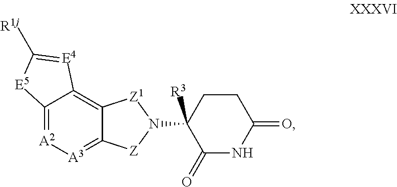

or a pharmaceutically acceptable salt or solvate thereof, wherein A¹, A³, E⁴, E⁵, Z, Z¹, R^{1j}, and R³ are as defined in connection with Formula I.

In some embodiments, the compound is of Formula XXXVII:

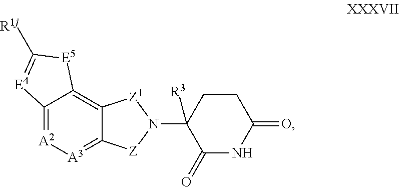

or a pharmaceutically acceptable salt or solvate thereof, wherein A¹, A³, E⁴, E⁵, Z, Z¹, R^{1j}, and R³ are as defined in connection with Formula I.

In some embodiments, the compound is of Formula XXXVIII:

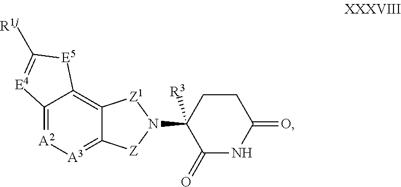

or a pharmaceutically acceptable salt or solvate thereof, wherein A¹, A³, E⁴, E⁵, Z, Z¹, R¹, and R³ are as defined in connection with Formula I.

In some embodiments, the compound is of Formula XXXIX:

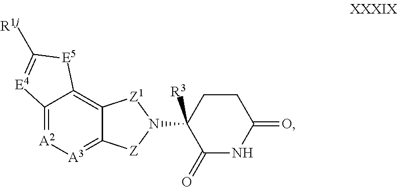

or a pharmaceutically acceptable salt or solvate thereof, wherein A¹, A³, E⁴, E⁵, Z, Z¹, R¹, and R³ are as defined in connection with Formula I.

In some embodiments, the compound is of any one of Formulae XXXIV-XXXIX, or a pharmaceutically acceptable salt or solvate thereof, wherein A² is —CR^{2c}═ and A³ is —CR^{2d}═. In some embodiments, R^{2c} is selected from hydrogen, —NH₂, fluoro, and methoxy. In some embodiments, R^{2d} is selected from hydrogen, —NH₂, fluoro, and methoxy.

In some embodiments, the compound is of any one of Formulae XXXIV-XXXIX, or a pharmaceutically acceptable salt or solvate thereof, wherein A² is —N═ and A³ is —CR^{2d}═. In some embodiments, R^{2d} is selected from hydrogen, —NH₂, fluoro, and methoxy.

In some embodiments, the compound is of any one of Formulae XXXIV-XXXIX, or a pharmaceutically acceptable salt or solvate thereof, wherein A² is —CR^{2c}═ and A is —N═. In some embodiments, R^{2c} is selected from hydrogen, —NH₂, fluoro, and methoxy.

In some embodiments, the compound is of any one of Formulae XXXIV-XXXIX, or a pharmaceutically acceptable salt or solvate thereof, wherein E⁴ is ═C(H)—.

In some embodiments, the compound is of any one of Formulae XXXIV-XXXIX, or a pharmaceutically acceptable salt or solvate thereof, wherein E⁴ is ═N—.

In some embodiments, the compound is of any one of Formulae XXXIV-XXXIX, or a pharmaceutically acceptable salt or solvate thereof, wherein E⁵ is —O—.

In some embodiments, the compound is of any one of Formulae XXXIV-XXXIX, or a pharmaceutically acceptable salt or solvate thereof, wherein E⁵ is —S—.

In some embodiments, the compound is of any one of Formulae XXXIV-XXXIX, or a pharmaceutically acceptable salt or solvate thereof, wherein E⁵ is selected from —NH— and —N(CH₃)—.

In some embodiments, the compound is of any one of Formulae XXXIV-XXXIX, or a pharmaceutically acceptable salt or solvate thereof, wherein R^{1j} is selected from hydrogen, C₁-C₄ alkyl, (hydroxy)alkyl, and (heterocyclo)alkyl.

In some embodiments, the compound is of any one of Formulae XXXIV-XXXIX, or a pharmaceutically acceptable salt or solvate thereof, wherein R^{1j} is Q-L-.

In some embodiments, the compound is of any one of Formulae I-XXXIX, or a pharmaceutically acceptable salt or solvate thereof, wherein R³ is hydrogen.

In some embodiments, the compound is of any one of Formulae I-XXXIX, or a pharmaceutically acceptable salt or solvate thereof, wherein Z and Z¹ are —C(═O)—.

In some embodiments, the compound is of any one of Formulae I-XXXIX, or a pharmaceutically acceptable salt or solvate thereof, wherein Z is —C(═O)— and Z¹ is —CR^{4a}R^{4b}—.

In some embodiments, R^{4a} and R^{4b} are hydrogen.

In some embodiments, the compound is of any one of Formulae I-XXXIX, or a pharmaceutically acceptable salt or solvate thereof, wherein Z is —CR^{4a}R^{4b} and Z¹ is —C(═O)—.

In some embodiments, R^{4a} and R^{4b} are hydrogen.

In some embodiments, the compound is of any one of Formulae I-XXXIX, or a pharmaceutically acceptable salt or solvate thereof, wherein Z is —N═C(CH₃)— and Z¹ is —C(═O)—e.g., a compound of Formula I-A:

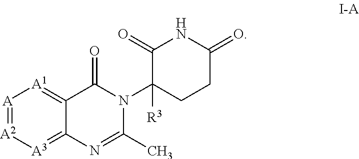

In some embodiments, the compound is of any one of Formulae I-XXXIX, or a pharmaceutically acceptable salt or solvate thereof, wherein Z is —C(═O)— and Z¹ is —N═C(CH₃)—, e.g., a compound of Formula I-B:

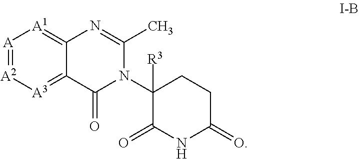

In some embodiments, the compound is of any one of Formulae I-XXXIX, or a pharmaceutically acceptable salt or solvate thereof, wherein Z is absent, i.e., Z is a bond, and Z¹ is —N(R²ⁿ)C(═O)—, e.g., a compound of Formula I-C:

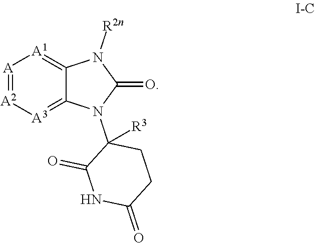

In some embodiments, the compound is of any one of Formulae I-XXXIX, or a pharmaceutically acceptable salt or solvate thereof, wherein Z is —N(R²ⁿ)C(═O)— and Z¹ is absent, i.e., Z¹ is a bond, e.g., a compound of Formula I-D:

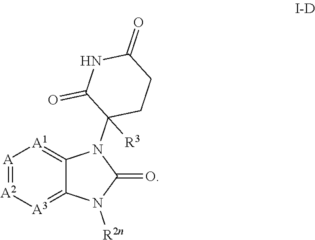

I. Cereblon Ligands

Without wishing to be bound by any particular theory, Cereblon Ligands may inhibit the ubiquitination of CRBN. Without wishing to be bound by any particular theory, inhibition of CRBN ubiquitination may allow CRBN to accumulate, leading to the increased cullin-4 RING E3 ligase-mediated degradation of target proteins. See Liu et al., FASEB J 29:4829-4839 (2015).

In some embodiments, Cereblon Ligands are compounds of any one of Formulae IV-IX, or a pharmaceutically acceptable salt or solvate thereof, wherein X is —N(R^{1a})— and R^{1a} is hydrogen.

In some embodiments, Cereblon Ligands are compounds of any one of Formulae IV-IX, or a pharmaceutically acceptable salt or solvate thereof, wherein X is —CR^{1b}R^{1c}—; R^{1b} is selected from hydrogen, —CHO, —C(═O)OH, hydroxy, (hydroxy)C₁-C₃ alkyl, amino, and (amino)alkyl; and R^{1c} is hydrogen.

In some embodiments, Cereblon Ligands are compounds of any one of Formulae IV-IX, or a pharmaceutically acceptable salt or solvate thereof, wherein X is —CR^{1b}R^{1c}—; and R^{1b} and R^{1c} taken together with the carbon atom to which they are attached form a —C(═O)—.

In some embodiments, Cereblon Ligands are compounds of any one of Formulae X-XII, or a pharmaceutically acceptable salt or solvate thereof, wherein R^{1d} is hydrogen.

In some embodiments, Cereblon Ligands are compounds of any one of Formulae XIII-XXI, or a pharmaceutically acceptable salt or solvate thereof, wherein R^{1e} is hydrogen.

In some embodiments, Cereblon Ligands are compounds of any one of Formulae XXII-XXIV, or a pharmaceutically acceptable salt or solvate thereof, wherein E¹ is —CR^{2e}═ or —N═; and R^{2e} is hydrogen, halo, hydroxy, amino, —CHO, C₁-C₃ alkyl, or C₁-C₃ alkoxy.

In some embodiments, Cereblon Ligands are compounds of any one of Formulae XXII-XXIV, or a pharmaceutically acceptable salt or solvate thereof, wherein E is —CR^{2f}═ or —N═; and R^{2f} is hydrogen, halo, hydroxy, amino, —CHO, C₁-C₃ alkyl, or C₁-C₃ alkoxy.

In some embodiments, Cereblon Ligands are compounds of any one of Formulae XXII-XXIV, or a pharmaceutically acceptable salt or solvate thereof, wherein E² is —CR^{2g}═ or —N═; and R^{2g} is hydrogen, halo, hydroxy, amino, —CHO, C₁-C₃ alkyl, or C₁-C₃ alkoxy.

In some embodiments, Cereblon Ligands are compounds of any one of Formulae XXII-XXIV, or a pharmaceutically acceptable salt or solvate thereof, wherein E³ is —CR^{2h}═ or —N═; and R^{2e} is hydrogen, halo, hydroxy, amino, —CHO, C₁-C₃ alkyl, or C₁-C₃ alkoxy.

In some embodiments, Cereblon Ligands are compounds of any one of Formulae XXV-XXVII, or a pharmaceutically acceptable salt or solvate thereof, wherein R¹⁹ is hydrogen.

In some embodiments, Cereblon Ligands are compounds of any one of Formulae XXVIII-XXX, or a pharmaceutically acceptable salt or solvate thereof, wherein R^{1h} is hydrogen.

In some embodiments, Cereblon Ligands are compounds of any one of Formulae XXXI-XXXIX, or a pharmaceutically acceptable salt or solvate thereof, wherein R^{1j} is selected from hydrogen, C₁-C₄ alkyl, (hydroxy)alkyl, and (heterocyclo)alkyl.

In some embodiments, Cereblon Ligands are any one or more of the compounds of Table 1, or a pharmaceutically acceptable salt or solvate thereof.

In some embodiments, Cereblon Ligands are any one or more of the compounds of Table 1, or a pharmaceutically acceptable salt thereof.

In some embodiments, Cereblon Ligands are any one or more of the compounds of Table 1.

TABLE 1

| Cpd. | |
|---|---|
| No. | Structure |
| 1 | 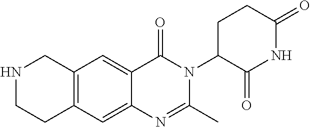 [SMILES: Cc1nc2cc3c(cc2c(=O)n1C1CCC(=O)NC1=O)CNCC3] |
| 2 | 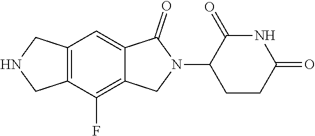 [SMILES: O=C1CCC(N2Cc3c(cc4c(c3F)CNC4)C2=O)C(=O)N1] |
| 3 | 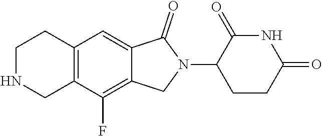 [SMILES: O=C1CCC(N2Cc3c(cc4c(c3F)CNCC4)C2=O)C(=O)N1] |
| 4 | 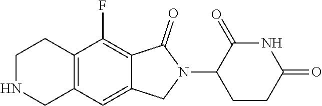 [SMILES: O=C1CCC(N2Cc3cc4c(c(F)c3C2=O)CCNC4)C(=O)N1] |
| 5 | 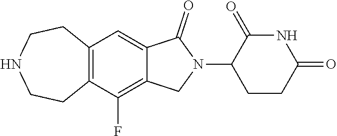 [SMILES: O=C1CCC(N2Cc3c(cc4c(c3F)CCNCC4)C2=O)C(=O)N1] |
| 6 | 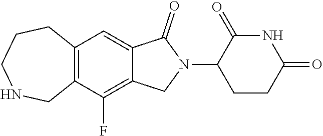 [SMILES: O=C1CCC(N2Cc3c(cc4c(c3F)CNCCC4)C2=O)C(=O)N1] |
| 7 | 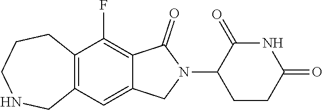 [SMILES: O=C1CCC(N2Cc3cc4c(c(F)c3C2=O)CCCNC4)C(=O)N1] |
| 8 | 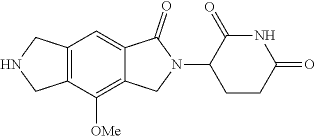 [SMILES: COc1c2c(cc3c1CN(C1CCC(=O)NC1=O)C3=O)CNC2] |
| 9 | 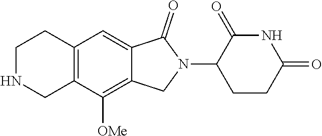 [SMILES: COc1c2c(cc3c1CN(C1CCC(=O)NC1=O)C3=O)CCNC2] |
| 10 | 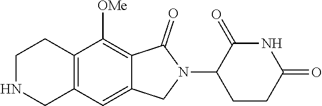 [SMILES: COc1c2c(cc3c1C(=O)N(C1CCC(=O)NC1=O)C3)CNCC2] |
| 11 | 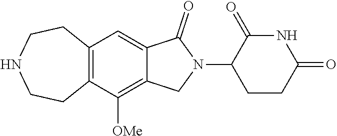 [SMILES: COc1c2c(cc3c1CN(C1CCC(=O)NC1=O)C3=O)CCNCC2] |
| 12 | 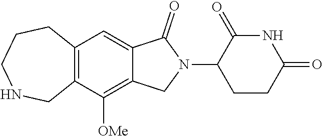 [SMILES: COc1c2c(cc3c1CN(C1CCC(=O)NC1=O)C3=O)CCCNC2] |
| 13 | 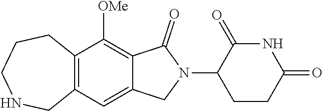 [SMILES: COc1c2c(cc3c1C(=O)N(C1CCC(=O)NC1=O)C3)CNCCC2] |
| 14 | 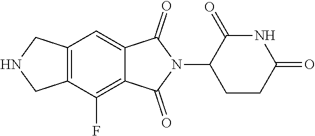 [SMILES: O=C1CCC(N2C(=O)c3cc4c(c(F)c3C2=O)CNC4)C(=O)N1] |
| 15 | 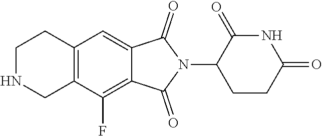 [SMILES: O=C1CCC(N2C(=O)c3cc4c(c(F)c3C2=O)CNCC4)C(=O)N1] |
| 16 | 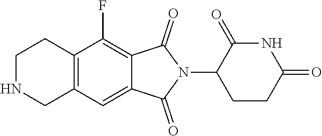 [SMILES: O=C1CCC(N2C(=O)c3cc4c(c(F)c3C2=O)CCNC4)C(=O)N1] |
| 17 | 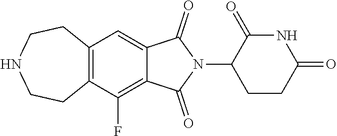 [SMILES: O=C1CCC(N2C(=O)c3cc4c(c(F)c3C2=O)CCNCC4)C(=O)N1] |
| 18 | 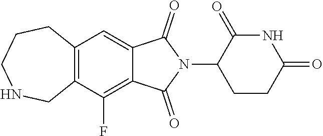 [SMILES: O=C1CCC(N2C(=O)c3cc4c(c(F)c3C2=O)CNCCC4)C(=O)N1] |
| 19 | 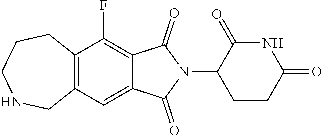 [SMILES: O=C1CCC(N2C(=O)c3cc4c(c(F)c3C2=O)CCCNC4)C(=O)N1] |
| 20 | 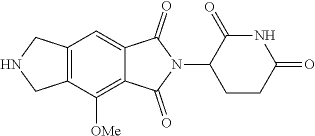 [SMILES: COc1c2c(cc3c1C(=O)N(C1CCC(=O)NC1=O)C3=O)CNC2] |
| 21 | 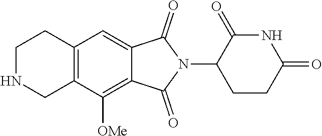 [SMILES: COc1c2c(cc3c1C(=O)N(C1CCC(=O)NC1=O)C3=O)CCNC2] |
| 22 | 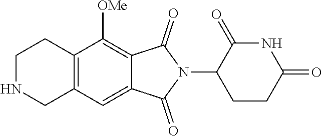 [SMILES: COc1c2c(cc3c1C(=O)N(C1CCC(=O)NC1=O)C3=O)CNCC2] |
| 23 | 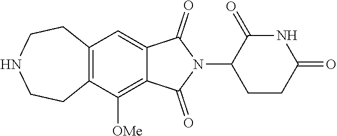 [SMILES: COc1c2c(cc3c1C(=O)N(C1CCC(=O)NC1=O)C3=O)CCNCC2] |
| 24 | 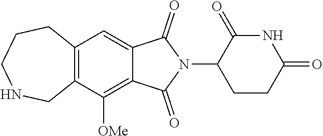 [SMILES: COc1c2c(cc3c1C(=O)N(C1CCC(=O)NC1=O)C3=O)CCCNC2] |
| 25 | 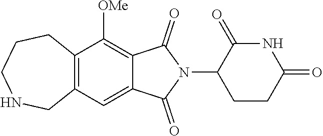 [SMILES: COc1c2c(cc3c1C(=O)N(C1CCC(=O)NC1=O)C3=O)CNCCC2] |
| 26 | 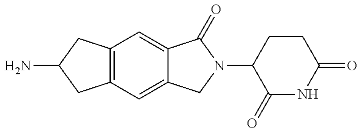 [SMILES: NC1Cc2cc3c(cc2C1)C(=O)N(C1CCC(=O)NC1=O)C3] |
| 27 | 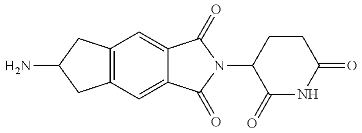 [SMILES: NC1Cc2cc3c(cc2C1)C(=O)N(C1CCC(=O)NC1=O)C3=O] |
| 28 | 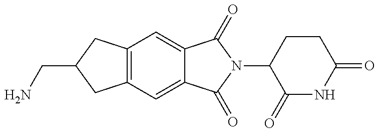 [SMILES: NCC1Cc2cc3c(cc2C1)C(=O)N(C1CCC(=O)NC1=O)C3=O] |
| 29 | 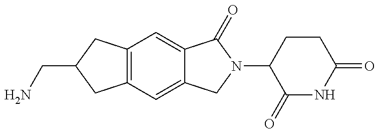 [SMILES: NCC1Cc2cc3c(cc2C1)C(=O)N(C1CCC(=O)NC1=O)C3] |
| 30 | 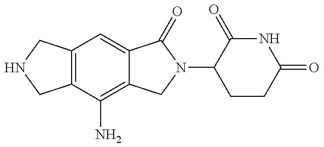 [SMILES: Nc1c2c(cc3c1CN(C1CCC(=O)NC1=O)C3=O)CNC2] |
| 31 | 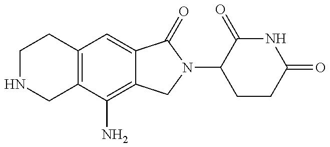 [SMILES: Nc1c2c(cc3c1CN(C1CCC(=O)NC1=O)C3=O)CCNC2] |
| 32 | 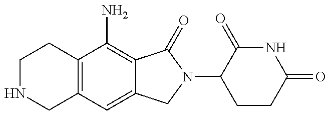 [SMILES: Nc1c2c(cc3c1C(=O)N(C1CCC(=O)NC1=O)C3)CNCC2] |
| 33 | 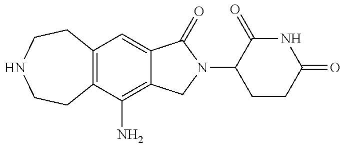 [SMILES: Nc1c2c(cc3c1CN(C1CCC(=O)NC1=O)C3=O)CCNCC2] |
| 34 | 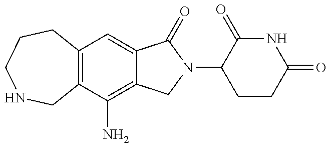 [SMILES: Nc1c2c(cc3c1CN(C1CCC(=O)NC1=O)C3=O)CCCNC2] |
| 35 | 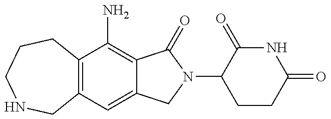 [SMILES: Nc1c2c(cc3c1C(=O)N(C1CCC(=O)NC1=O)C3)CNCCC2] |
| 36 | 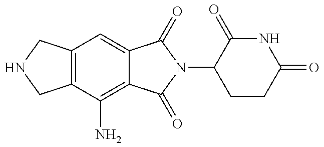 [SMILES: Nc1c2c(cc3c1C(=O)N(C1CCC(=O)NC1=O)C3=O)CNC2] |
| 37 | 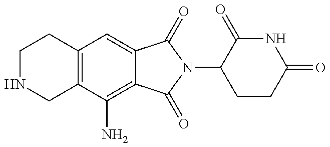 [SMILES: Nc1c2c(cc3c1C(=O)N(C1CCC(=O)NC1=O)C3=O)CCNC2] |
| 38 | 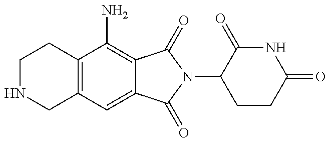 [SMILES: Nc1c2c(cc3c1C(=O)N(C1CCC(=O)NC1=O)C3=O)CNCC2] |
| 39 | 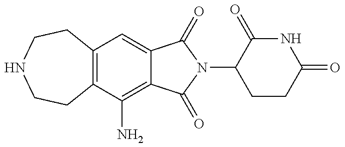 [SMILES: Nc1c2c(cc3c1C(=O)N(C1CCC(=O)NC1=O)C3=O)CCNCC2] |
| 40 | 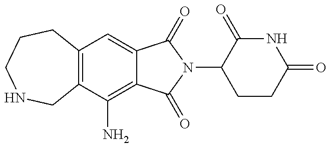 [SMILES: Nc1c2c(cc3c1C(=O)N(C1CCC(=O)NC1=O)C3=O)CCCNC2] |
| 41 | 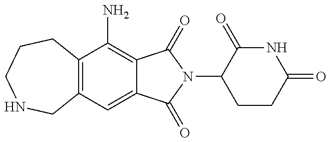 [SMILES: Nc1c2c(cc3c1C(=O)N(C1CCC(=O)NC1=O)C3=O)CNCCC2] |
| 42 | 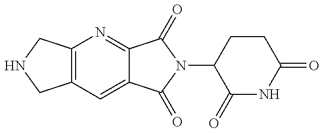 [SMILES: O=C1CCC(N2C(=O)c3cc4c(nc3C2=O)CNC4)C(=O)N1] |
| 43 | 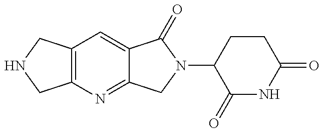 [SMILES: O=C1CCC(N2Cc3nc4c(cc3C2=O)CNC4)C(=O)N1] |
| 44 | 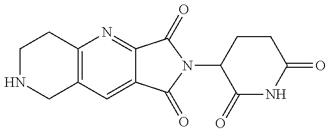 [SMILES: O=C1CCC(N2C(=O)c3cc4c(nc3C2=O)CCNC4)C(=O)N1] |
| 45 | 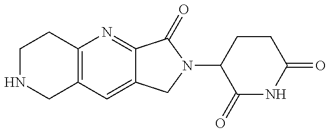 [SMILES: O=C1CCC(N2Cc3cc4c(nc3C2=O)CCNC4)C(=O)N1] |
| 46 | 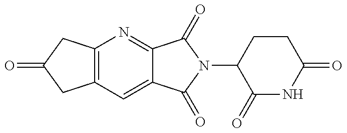 [SMILES: O=C1Cc2cc3c(nc2C1)C(=O)N(C1CCC(=O)NC1=O)C3=O] |
| 47 | 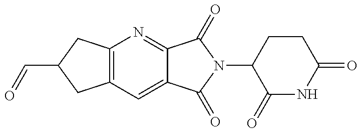 [SMILES: O=CC1Cc2cc3c(nc2C1)C(=O)N(C1CCC(=O)NC1=O)C3=O] |
| 48 | 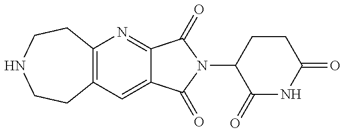 [SMILES: O=C1CCC(N2C(=O)c3cc4c(nc3C2=O)CCNCC4)C(=O)N1] |
| 49 | 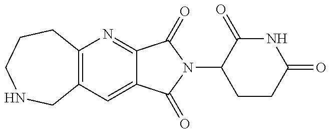 [SMILES: O=C1CCC(N2C(=O)c3cc4c(nc3C2=O)CCCNC4)C(=O)N1] |
| 50 | 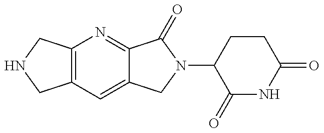 [SMILES: O=C1CCC(N2Cc3cc4c(nc3C2=O)CNC4)C(=O)N1] |
| 51 | 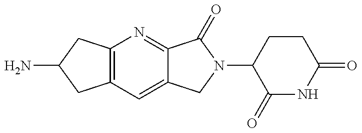 [SMILES: NC1Cc2cc3c(nc2C1)C(=O)N(C1CCC(=O)NC1=O)C3] |
| 52 | 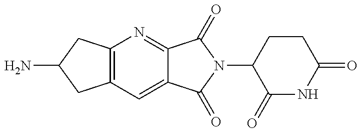 [SMILES: NC1Cc2cc3c(nc2C1)C(=O)N(C1CCC(=O)NC1=O)C3=O] |
| 53 | 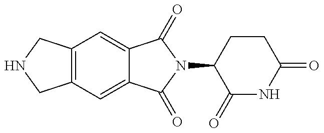 [SMILES: O=C1CC[C@H](N2C(=O)c3cc4c(cc3C2=O)CNC4)C(=O)N1] |
| 54 | 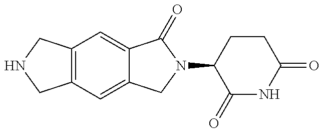 [SMILES: O=C1CC[C@H](N2Cc3cc4c(cc3C2=O)CNC4)C(=O)N1] |
| 55 | 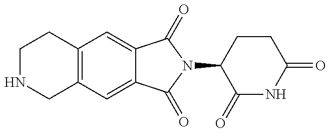 [SMILES: O=C1CC[C@H](N2C(=O)c3cc4c(cc3C2=O)CNCC4)C(=O)N1] |
| 56 | 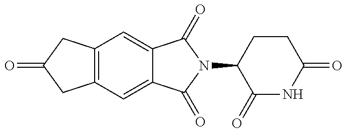 [SMILES: O=C1Cc2cc3c(cc2C1)C(=O)N([C@H]1CCC(=O)NC1=O)C3=O] |
| 57 | 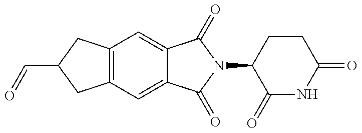 [SMILES: O=CC1Cc2cc3c(cc2C1)C(=O)N([C@H]1CCC(=O)NC1=O)C3=O] |
| 58 | 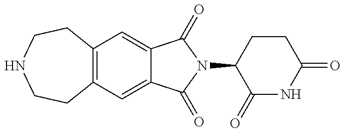 [SMILES: O=C1CC[C@H](N2C(=O)c3cc4c(cc3C2=O)CCNCC4)C(=O)N1] |
| 59 | 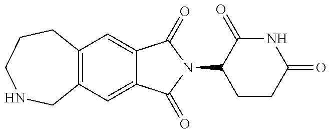 |
| 60 | 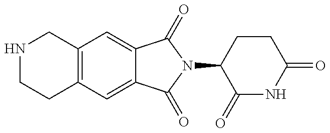 [SMILES: O=C1CC[C@H](N2C(=O)c3cc4c(cc3C2=O)CNCC4)C(=O)N1] |
| 61 | 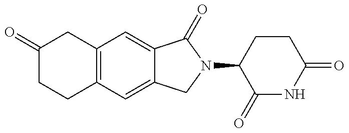 [SMILES: O=C1CCc2cc3c(cc2C1)C(=O)N([C@H]1CCC(=O)NC1=O)C3] |
| 62 | 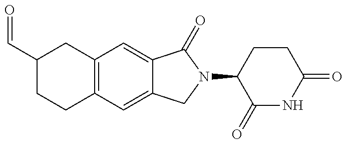 [SMILES: O=CC1CCc2cc3c(cc2C1)C(=O)N([C@H]1CCC(=O)NC1=O)C3] |
| 63 | 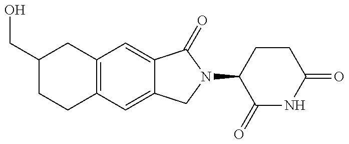 [SMILES: O=C1CC[C@H](N2Cc3cc4c(cc3C2=O)CC(CO)CC4)C(=O)N1] |
| 64 | 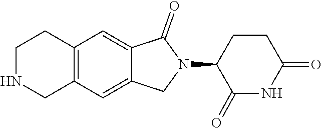 [SMILES: O=C1CC[C@H](N2Cc3cc4c(cc3C2=O)CCNC4)C(=O)N1] |
| 65 | 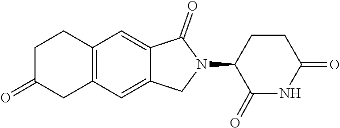 [SMILES: O=C1CCc2cc3c(cc2C1)CN([C@H]1CCC(=O)NC1=O)C3=O] |
| 66 | 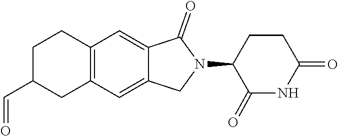 [SMILES: O=CC1CCc2cc3c(cc2C1)CN([C@H]1CCC(=O)NC1=O)C3=O] |
| 67 | 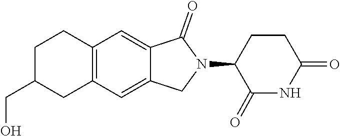 [SMILES: O=C1CC[C@H](N2Cc3cc4c(cc3C2=O)CCC(CO)C4)C(=O)N1] |
| 68 | 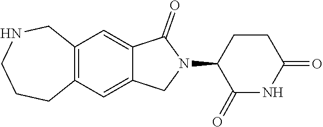 [SMILES: O=C1CC[C@H](N2Cc3cc4c(cc3C2=O)CNCCC4)C(=O)N1] |
| 69 | 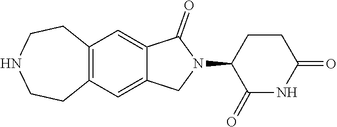 [SMILES: O=C1CC[C@H](N2Cc3cc4c(cc3C2=O)CCNCC4)C(=O)N1] |
| 70 | 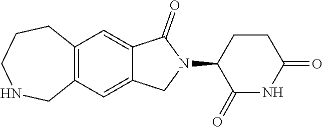 [SMILES: O=C1CC[C@H](N2Cc3cc4c(cc3C2=O)CCCNC4)C(=O)N1] |
| 71 | 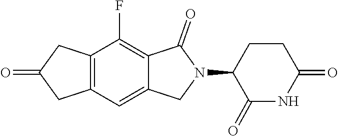 [SMILES: O=C1Cc2cc3c(c(F)c2C1)C(=O)N([C@H]1CCC(=O)NC1=O)C3] |
| 72 | 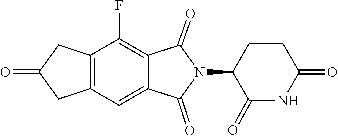 [SMILES: O=C1Cc2cc3c(c(F)c2C1)C(=O)N([C@H]1CCC(=O)NC1=O)C3=O] |
| 73 | 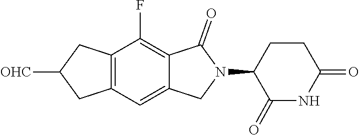 [SMILES: O=CC1Cc2cc3c(c(F)c2C1)C(=O)N([C@H]1CCC(=O)NC1=O)C3] |
| 74 | 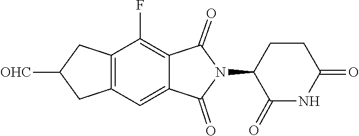 [SMILES: O=CC1Cc2cc3c(c(F)c2C1)C(=O)N([C@H]1CCC(=O)NC1=O)C3=O] |
| 75 | 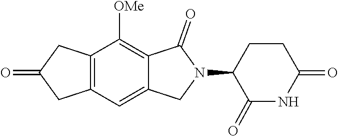 [SMILES: COc1c2c(cc3c1C(=O)N([C@H]1CCC(=O)NC1=O)C3)CC(=O)C2] |
| 76 | 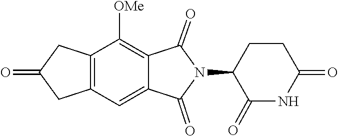 [SMILES: COc1c2c(cc3c1C(=O)N([C@H]1CCC(=O)NC1=O)C3=O)CC(=O)C2] |
| 77 | 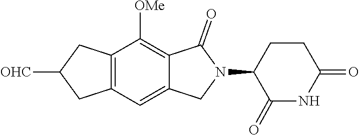 [SMILES: COc1c2c(cc3c1C(=O)N([C@H]1CCC(=O)NC1=O)C3)CC(C=O)C2] |
| 78 | 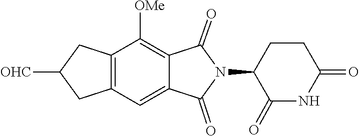 [SMILES: COc1c2c(cc3c1C(=O)N([C@H]1CCC(=O)NC1=O)C3=O)CC(C=O)C2] |
| 79 | 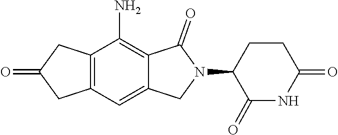 [SMILES: Nc1c2c(cc3c1C(=O)N([C@H]1CCC(=O)NC1=O)C3)CC(=O)C2] |
| 80 | 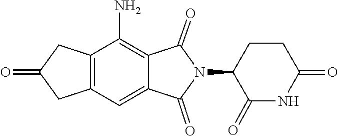 [SMILES: Nc1c2c(cc3c1C(=O)N([C@H]1CCC(=O)NC1=O)C3=O)CC(=O)C2] |
| 81 | 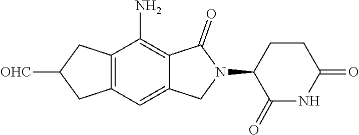 [SMILES: Nc1c2c(cc3c1C(=O)N([C@H]1CCC(=O)NC1=O)C3)CC(C=O)C2] |
| 82 | 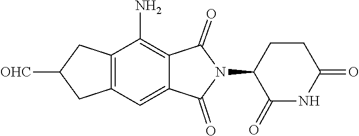 [SMILES: Nc1c2c(cc3c1C(=O)N([C@H]1CCC(=O)NC1=O)C3=O)CC(C=O)C2] |
| 83 | 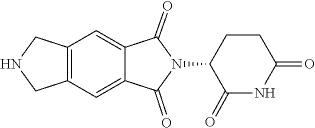 [SMILES: O=C1CC[C@@H](N2C(=O)c3cc4c(cc3C2=O)CNC4)C(=O)N1] |
| 84 | 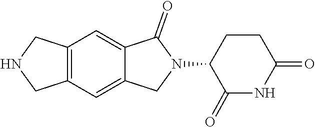 [SMILES: O=C1CC[C@@H](N2Cc3cc4c(cc3C2=O)CNC4)C(=O)N1] |
| 85 | 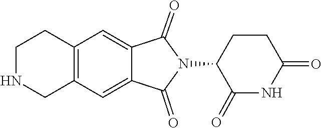 [SMILES: O=C1CC[C@@H](N2C(=O)c3cc4c(cc3C2=O)CNCC4)C(=O)N1] |
| 86 | 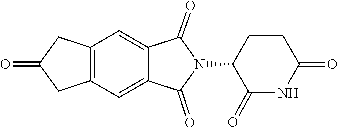 [SMILES: O=C1Cc2cc3c(cc2C1)C(=O)N([C@@H]1CCC(=O)NC1=O)C3=O] |
| 87 | 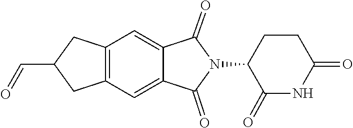 [SMILES: O=CC1Cc2cc3c(cc2C1)C(=O)N([C@@H]1CCC(=O)NC1=O)C3=O] |
| 88 | 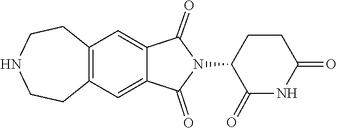 [SMILES: O=C1CC[C@@H](N2C(=O)c3cc4c(cc3C2=O)CCNCC4)C(=O)N1] |
| 89 | 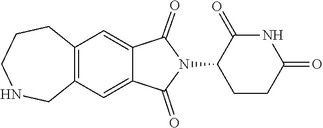 [SMILES: O=C1CC[C@H](N2C(=O)c3cc4c(cc3C2=O)CNCCC4)C(=O)N1] |
| 90 | 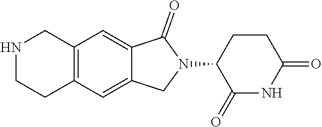 [SMILES: O=C1CC[C@@H](N2Cc3cc4c(cc3C2=O)CNCC4)C(=O)N1] |
| 91 | 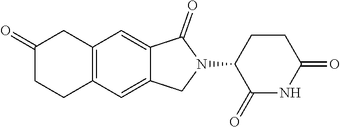 [SMILES: O=C1CCc2cc3c(cc2C1)C(=O)N([C@@H]1CCC(=O)NC1=O)C3] |
| 92 | 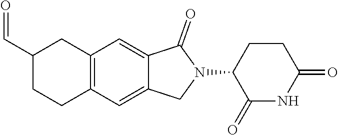 [SMILES: O=CC1CCc2cc3c(cc2C1)C(=O)N([C@@H]1CCC(=O)NC1=O)C3] |
| 93 | 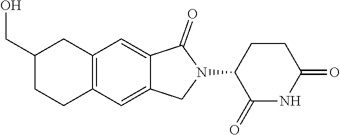 [SMILES: O=C1CC[C@@H](N2Cc3cc4c(cc3C2=O)CC(CO)CC4)C(=O)N1] |
| 94 | 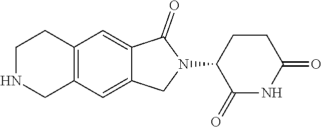 [SMILES: O=C1CC[C@@H](N2Cc3cc4c(cc3C2=O)CCNC4)C(=O)N1] |
| 95 | 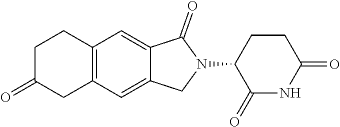 [SMILES: O=C1CCc2cc3c(cc2C1)CN([C@@H]1CCC(=O)NC1=O)C3=O] |
| 96 | 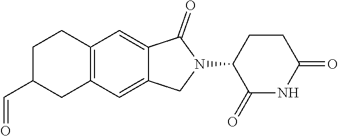 [SMILES: O=CC1CCc2cc3c(cc2C1)CN([C@@H]1CCC(=O)NC1=O)C3=O] |
| 97 | 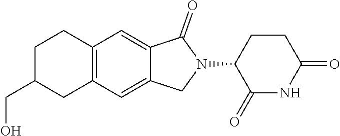 [SMILES: O=C1CC[C@@H](N2Cc3cc4c(cc3C2=O)CCC(CO)C4)C(=O)N1] |
| 98 | 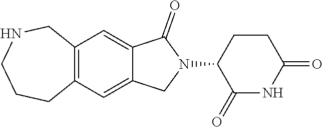 [SMILES: O=C1CC[C@@H](N2Cc3cc4c(cc3C2=O)CNCCC4)C(=O)N1] |
| 99 | 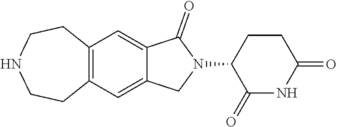 [SMILES: O=C1CC[C@@H](N2Cc3cc4c(cc3C2=O)CCNCC4)C(=O)N1] |
| 100 | 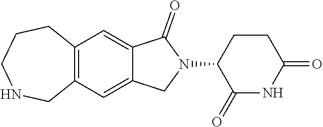 [SMILES: O=C1CC[C@@H](N2Cc3cc4c(cc3C2=O)CCCNC4)C(=O)N1] |
| 101 | 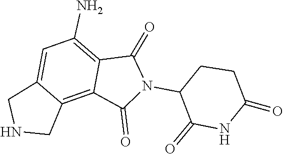 [SMILES: Nc1cc2c(c3c1C(=O)N(C1CCC(=O)NC1=O)C3=O)CNC2] |
| 102 | 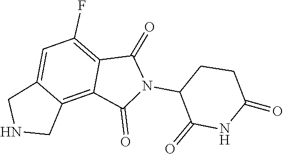 [SMILES: O=C1CCC(N2C(=O)c3c(F)cc4c(c3C2=O)CNC4)C(=O)N1] |
| 103 | 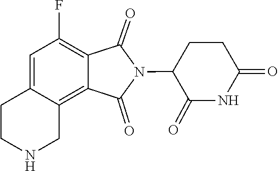 [SMILES: O=C1CCC(N2C(=O)c3c(F)cc4c(c3C2=O)CNCC4)C(=O)N1] |
| 104 | 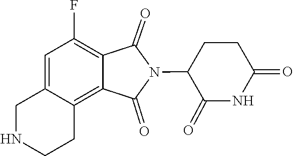 [SMILES: O=C1CCC(N2C(=O)c3c(F)cc4c(c3C2=O)CCNC4)C(=O)N1] |
| 105 | 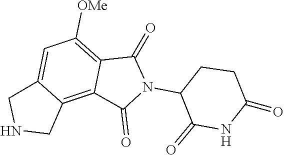 [SMILES: COc1cc2c(c3c1C(=O)N(C1CCC(=O)NC1=O)C3=O)CNC2] |
| 106 | 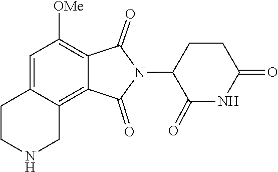 [SMILES: COc1cc2c(c3c1C(=O)N(C1CCC(=O)NC1=O)C3=O)CNCC2] |
| 107 | 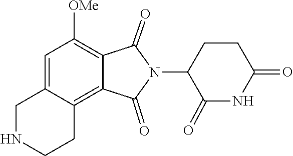 [SMILES: COc1cc2c(c3c1C(=O)N(C1CCC(=O)NC1=O)C3=O)CCNC2] |
| 108 | 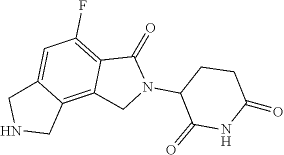 [SMILES: O=C1CCC(N2Cc3c4c(cc(F)c3C2=O)CNC4)C(=O)N1] |
| 109 | 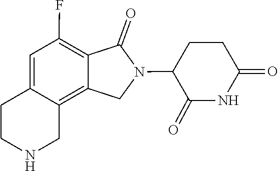 [SMILES: O=C1CCC(N2Cc3c4c(cc(F)c3C2=O)CCNC4)C(=O)N1] |
| 110 | 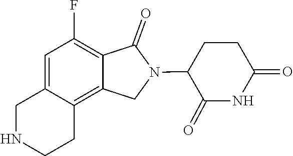 [SMILES: O=C1CCC(N2Cc3c4c(cc(F)c3C2=O)CNCC4)C(=O)N1] |
| 111 | 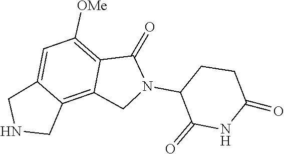 [SMILES: COc1cc2c(c3c1C(=O)N(C1CCC(=O)NC1=O)C3)CNC2] |
| 112 | 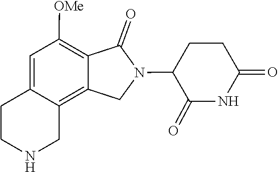 [SMILES: COc1cc2c(c3c1C(=O)N(C1CCC(=O)NC1=O)C3)CNCC2] |
| 113 | 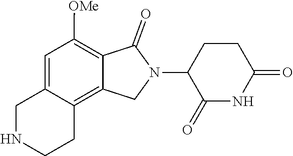 [SMILES: COc1cc2c(c3c1C(=O)N(C1CCC(=O)NC1=O)C3)CCNC2] |
| 114 | 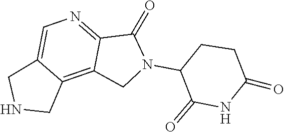 [SMILES: O=C1CCC(N2Cc3c(ncc4c3CNC4)C2=O)C(=O)N1] |
| 115 | 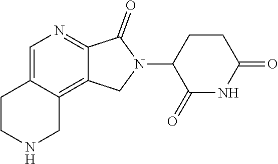 [SMILES: O=C1CCC(N2Cc3c(ncc4c3CNCC4)C2=O)C(=O)N1] |
| 116 | 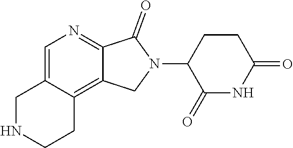 [SMILES: O=C1CCC(N2Cc3c(ncc4c3CCNC4)C2=O)C(=O)N1] |
| 117 | 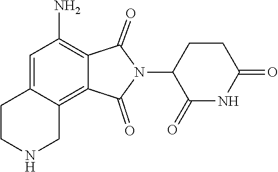 [SMILES: Nc1cc2c(c3c1C(=O)N(C1CCC(=O)NC1=O)C3=O)CNCC2] |
| 118 | 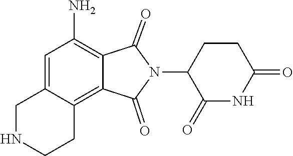 [SMILES: Nc1cc2c(c3c1C(=O)N(C1CCC(=O)NC1=O)C3=O)CCNC2] |
| 119 | 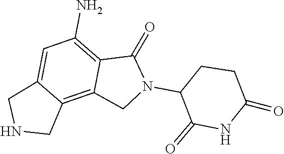 [SMILES: Nc1cc2c(c3c1C(=O)N(C1CCC(=O)NC1=O)C3)CNC2] |
| 120 | 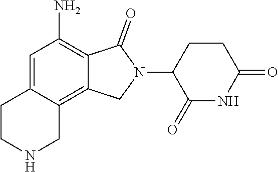 [SMILES: Nc1cc2c(c3c1C(=O)N(C1CCC(=O)NC1=O)C3)CNCC2] |
| 121 | 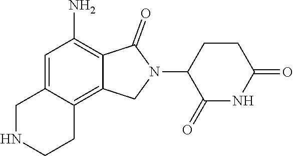 [SMILES: Nc1cc2c(c3c1C(=O)N(C1CCC(=O)NC1=O)C3)CCNC2] |
| 122 | 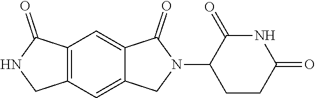 [SMILES: O=C1CCC(N2Cc3cc4c(cc3C2=O)C(=O)NC4)C(=O)N1] |
| 123 | 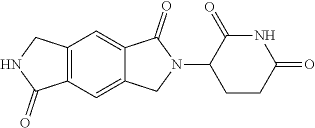 [SMILES: O=C1CCC(N2Cc3cc4c(cc3C2=O)CNC4=O)C(=O)N1] |
| 124 | 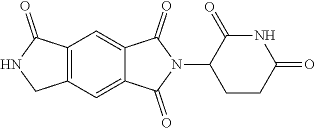 [SMILES: O=C1CCC(N2C(=O)c3cc4c(cc3C2=O)C(=O)NC4)C(=O)N1] |
| 125 | 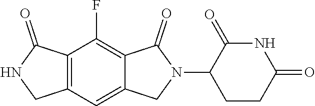 [SMILES: O=C1CCC(N2Cc3cc4c(c(F)c3C2=O)C(=O)NC4)C(=O)N1] |
| 126 | 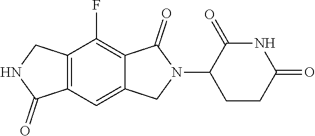 [SMILES: O=C1CCC(N2Cc3cc4c(c(F)c3C2=O)CNC4=O)C(=O)N1] |
| 127 | 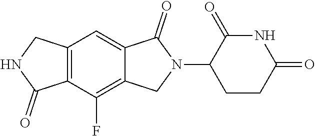 [SMILES: O=C1CCC(N2Cc3c(cc4c(c3F)C(=O)NC4)C2=O)C(=O)N1] |
| 128 | 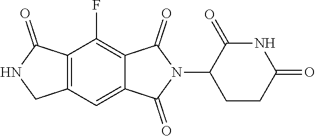 [SMILES: O=C1CCC(N2C(=O)c3cc4c(c(F)c3C2=O)C(=O)NC4)C(=O)N1] |
| 129 | 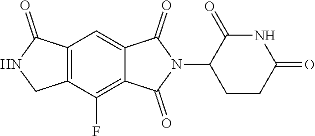 [SMILES: O=C1CCC(N2C(=O)c3cc4c(c(F)c3C2=O)CNC4=O)C(=O)N1] |
| 130 | 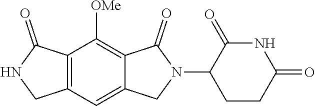 [SMILES: COc1c2c(cc3c1C(=O)N(C1CCC(=O)NC1=O)C3)CNC2=O] |
| 131 | 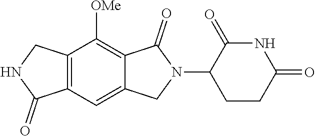 [SMILES: COc1c2c(cc3c1C(=O)N(C1CCC(=O)NC1=O)C3)C(=O)NC2] |
| 132 | 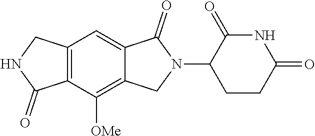 [SMILES: COc1c2c(cc3c1C(=O)NC3)C(=O)N(C1CCC(=O)NC1=O)C2] |
| 133 | 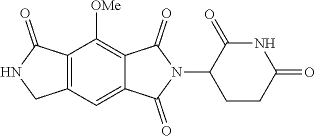 [SMILES: COc1c2c(cc3c1C(=O)N(C1CCC(=O)NC1=O)C3=O)CNC2=O] |
| 134 | 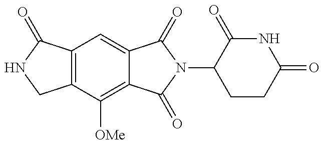 [SMILES: COc1c2c(cc3c1C(=O)N(C1CCC(=O)NC1=O)C3=O)C(=O)NC2] |
| 135 | 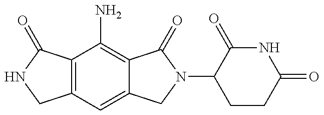 [SMILES: Nc1c2c(cc3c1C(=O)N(C1CCC(=O)NC1=O)C3)CNC2=O] |
| 136 | 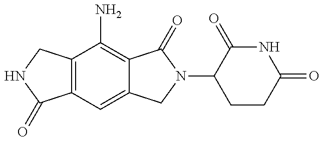 [SMILES: Nc1c2c(cc3c1C(=O)N(C1CCC(=O)NC1=O)C3)C(=O)NC2] |
| 137 | 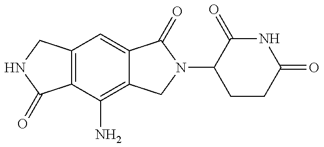 [SMILES: Nc1c2c(cc3c1C(=O)NC3)C(=O)N(C1CCC(=O)NC1=O)C2] |
| 138 | 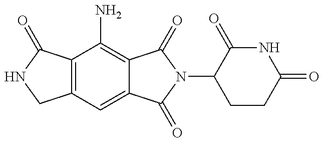 [SMILES: Nc1c2c(cc3c1C(=O)N(C1CCC(=O)NC1=O)C3=O)CNC2=O] |
| 139 | 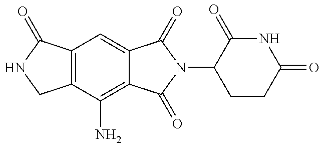 [SMILES: Nc1c2c(cc3c1C(=O)N(C1CCC(=O)NC1=O)C3=O)C(=O)NC2] |
| 140 | 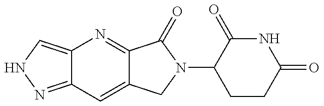 [SMILES: O=C1CCC(N2Cc3cc4n[nH]cc4nc3C2=O)C(=O)N1] |
| 141 | 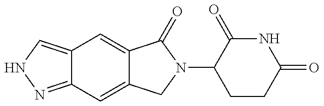 [SMILES: O=C1CCC(N2Cc3cc4n[nH]cc4cc3C2=O)C(=O)N1] |
| 142 | 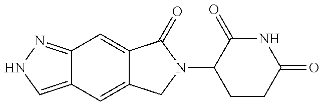 [SMILES: O=C1CCC(N2Cc3cc4c[nH]nc4cc3C2=O)C(=O)N1] |
| 143 | 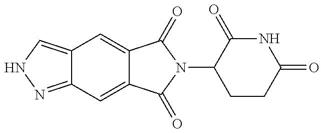 [SMILES: O=C1CCC(N2C(=O)c3cc4c[nH]nc4cc3C2=O)C(=O)N1] |
| 144 | 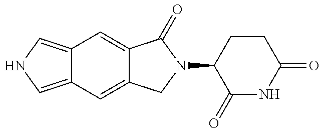 [SMILES: O=C1CC[C@H](N2Cc3cc4c[nH]cc4cc3C2=O)C(=O)N1] |
| 145 | 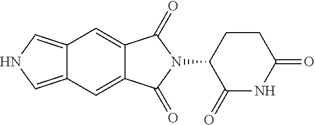 [SMILES: O=C1CC[C@@H](N2C(=O)c3cc4c[nH]cc4cc3C2=O)C(=O)N1] |
| 146 | 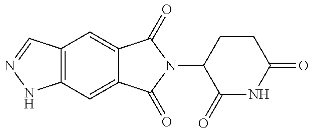 [SMILES: O=C1CCC(N2C(=O)c3cc4cn[nH]c4cc3C2=O)C(=O)N1] |
| 147 | 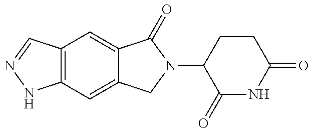 [SMILES: O=C1CCC(N2Cc3cc4[nH]ncc4cc3C2=O)C(=O)N1] |
| 148 | 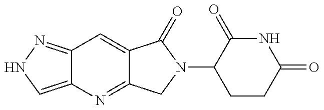 [SMILES: O=C1CCC(N2Cc3nc4c[nH]nc4cc3C2=O)C(=O)N1] |
| 149 | 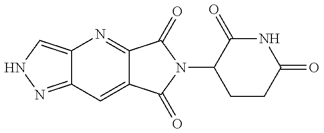 [SMILES: O=C1CCC(N2C(=O)c3cc4n[nH]cc4nc3C2=O)C(=O)N1] |
| 150 | 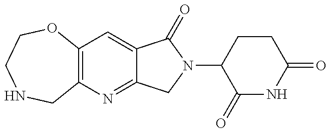 [SMILES: O=C1CCC(N2Cc3nc4c(cc3C2=O)OCCNC4)C(=O)N1] |
| 151 | 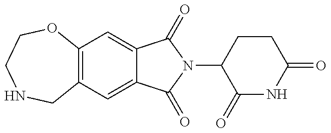 [SMILES: O=C1CCC(N2C(=O)c3cc4c(cc3C2=O)OCCNC4)C(=O)N1] |
| 152 | 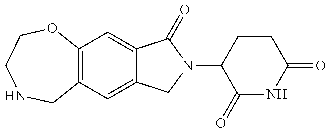 [SMILES: O=C1CCC(N2Cc3cc4c(cc3C2=O)OCCNC4)C(=O)N1] |
| 153 | 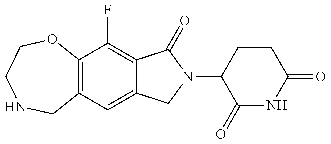 [SMILES: O=C1CCC(N2Cc3cc4c(c(F)c3C2=O)OCCNC4)C(=O)N1] |
| 154 | 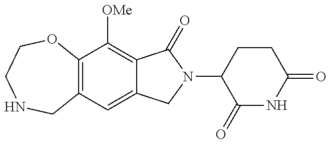 [SMILES: COc1c2c(cc3c1C(=O)N(C1CCC(=O)NC1=O)C3)CNCCO2] |
| 155 | 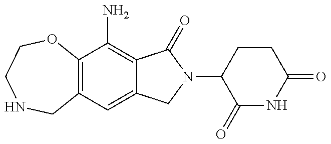 [SMILES: Nc1c2c(cc3c1C(=O)N(C1CCC(=O)NC1=O)C3)CNCCO2] |
| 156 | 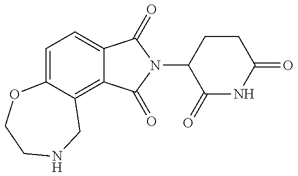 [SMILES: O=C1CCC(N2C(=O)c3ccc4c(c3C2=O)CNCCO4)C(=O)N1] |
| 157 | 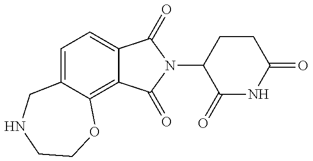 [SMILES: O=C1CCC(N2C(=O)c3ccc4c(c3C2=O)OCCNC4)C(=O)N1] |
| 158 | 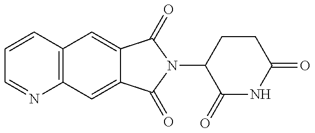 [SMILES: O=C1CCC(N2C(=O)c3cc4cccnc4cc3C2=O)C(=O)N1] |
| 159 | 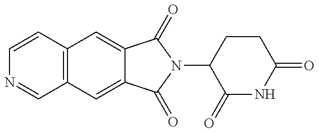 [SMILES: O=C1CCC(N2C(=O)c3cc4ccncc4cc3C2=O)C(=O)N1] |
| 160 | 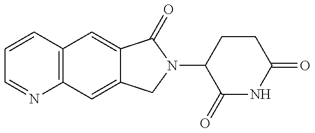 [SMILES: O=C1CCC(N2Cc3cc4ncccc4cc3C2=O)C(=O)N1] |
| 161 | 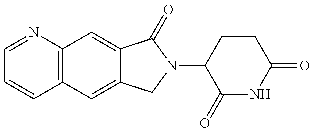 [SMILES: O=C1CCC(N2Cc3cc4cccnc4cc3C2=O)C(=O)N1] |
| 162 | 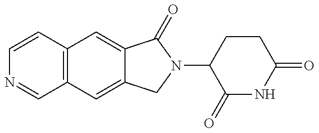 [SMILES: O=C1CCC(N2Cc3cc4cnccc4cc3C2=O)C(=O)N1] |
| 163 | 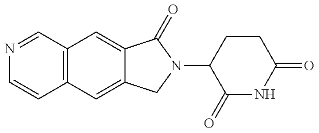 [SMILES: O=C1CCC(N2Cc3cc4ccncc4cc3C2=O)C(=O)N1] |
| 164 | 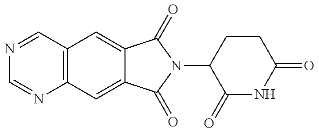 [SMILES: O=C1CCC(N2C(=O)c3cc4cncnc4cc3C2=O)C(=O)N1] |
| 165 | 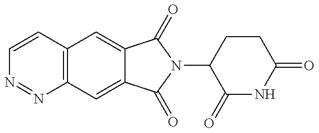 [SMILES: O=C1CCC(N2C(=O)c3cc4ccnnc4cc3C2=O)C(=O)N1] |
| 166 | 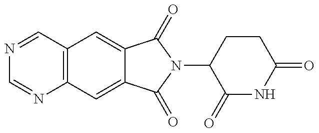 [SMILES: O=C1CCC(N2C(=O)c3cc4cncnc4cc3C2=O)C(=O)N1] |
| 167 | 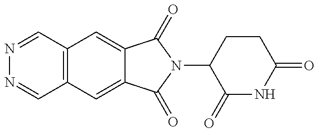 [SMILES: O=C1CCC(N2C(=O)c3cc4cnncc4cc3C2=O)C(=O)N1] |
| 168 | 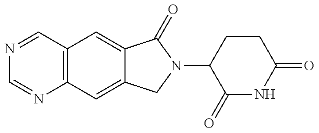 [SMILES: O=C1CCC(N2Cc3cc4ncncc4cc3C2=O)C(=O)N1] |
| 169 | 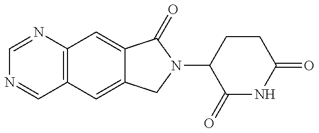 [SMILES: O=C1CCC(N2Cc3cc4cncnc4cc3C2=O)C(=O)N1] |
| 170 | 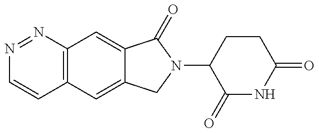 [SMILES: O=C1CCC(N2Cc3cc4ccnnc4cc3C2=O)C(=O)N1] |
| 171 | 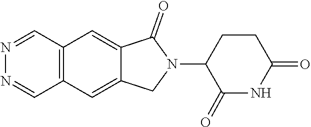 [SMILES: O=C1CCC(N2Cc3cc4cnncc4cc3C2=O)C(=O)N1] |
| 172 | 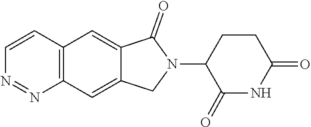 [SMILES: O=C1CCC(N2Cc3cc4nnccc4cc3C2=O)C(=O)N1] |
| 173 | 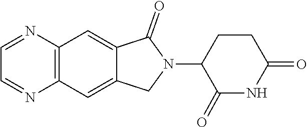 [SMILES: O=C1CCC(N2Cc3cc4nccnc4cc3C2=O)C(=O)N1] |
| 174 | 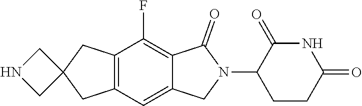 [SMILES: O=C1CCC(N2Cc3cc4c(c(F)c3C2=O)CC2(CNC2)C4)C(=O)N1] |
| 175 | 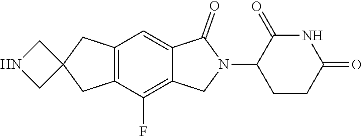 [SMILES: O=C1CCC(N2Cc3c(cc4c(c3F)CC3(CNC3)C4)C2=O)C(=O)N1] |
| 176 | 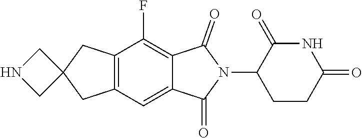 [SMILES: O=C1CCC(N2C(=O)c3cc4c(c(F)c3C2=O)CC2(CNC2)C4)C(=O)N1] |
| 177 | 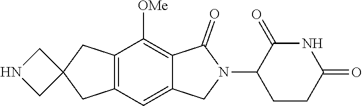 [SMILES: COc1c2c(cc3c1C(=O)N(C1CCC(=O)NC1=O)C3)CC1(CNC1)C2] |
| 178 | 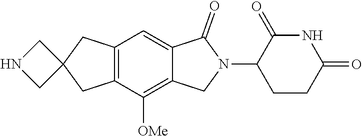 [SMILES: COc1c2c(cc3c1CN(C1CCC(=O)NC1=O)C3=O)CC1(CNC1)C2] |
| 179 | 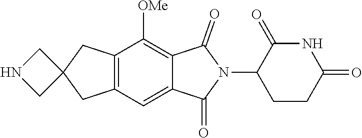 [SMILES: COc1c2c(cc3c1C(=O)N(C1CCC(=O)NC1=O)C3=O)CC1(CNC1)C2] |
| 180 | 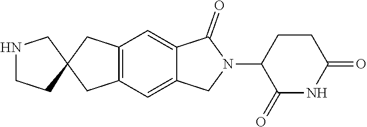 [SMILES: O=C1CCC(N2Cc3cc4c(cc3C2=O)C[C@]2(CCNC2)C4)C(=O)N1] |
| 181 | 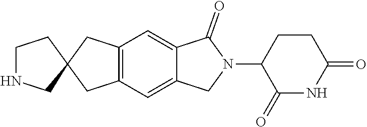 [SMILES: O=C1CCC(N2Cc3cc4c(cc3C2=O)C[C@@]2(CCNC2)C4)C(=O)N1] |
| 182 | 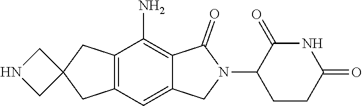 [SMILES: Nc1c2c(cc3c1C(=O)N(C1CCC(=O)NC1=O)C3)CC1(CNC1)C2] |
| 183 | 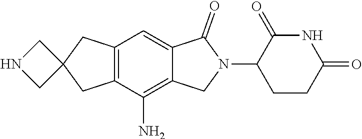 [SMILES: Nc1c2c(cc3c1CN(C1CCC(=O)NC1=O)C3=O)CC1(CNC1)C2] |
| 184 | 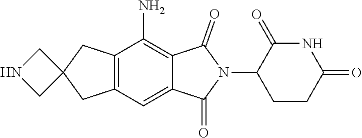 [SMILES: Nc1c2c(cc3c1C(=O)N(C1CCC(=O)NC1=O)C3=O)CC1(CNC1)C2] |
| 185 | 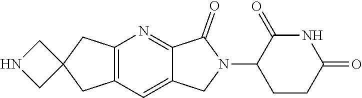 [SMILES: O=C1CCC(N2Cc3cc4c(nc3C2=O)CC2(CNC2)C4)C(=O)N1] |
| 186 | 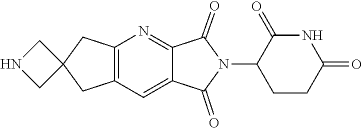 [SMILES: O=C1CCC(N2C(=O)c3cc4c(nc3C2=O)CC2(CNC2)C4)C(=O)N1] |
| 187 | 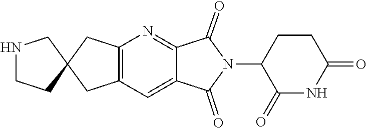 [SMILES: O=C1CCC(N2C(=O)c3cc4c(nc3C2=O)C[C@]2(CCNC2)C4)C(=O)N1] |
| 188 | 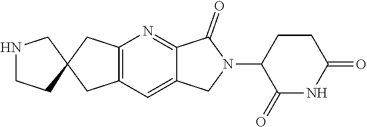 [SMILES: O=C1CCC(N2Cc3cc4c(nc3C2=O)C[C@]2(CCNC2)C4)C(=O)N1] |
| 189 | 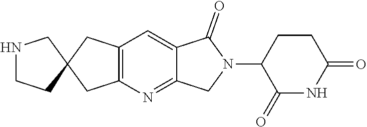 [SMILES: O=C1CCC(N2Cc3nc4c(cc3C2=O)C[C@]2(CCNC2)C4)C(=O)N1] |
| 190 | 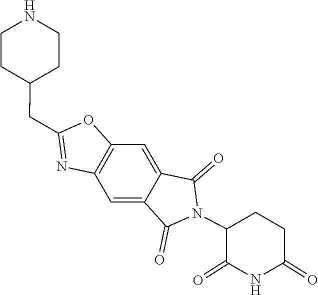 [SMILES: O=C1CCC(N2C(=O)c3cc4nc(CC5CCNCC5)oc4cc3C2=O)C(=O)N1] |
| 191 | 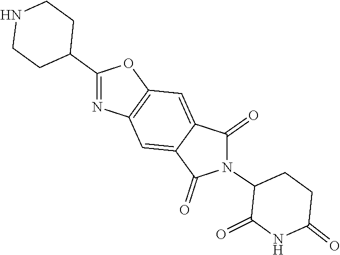 [SMILES: O=C1CCC(N2C(=O)c3cc4nc(C5CCNCC5)oc4cc3C2=O)C(=O)N1] |
| 192 | 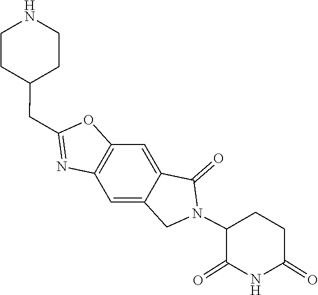 [SMILES: O=C1CCC(N2Cc3cc4nc(CC5CCNCC5)oc4cc3C2=O)C(=O)N1] |
| 193 | 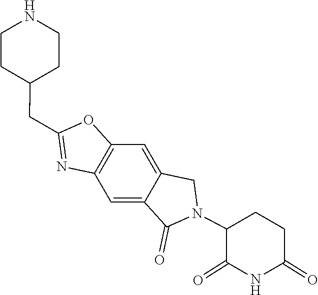 [SMILES: O=C1CCC(N2Cc3cc4oc(CC5CCNCC5)nc4cc3C2=O)C(=O)N1] |
| 194 | 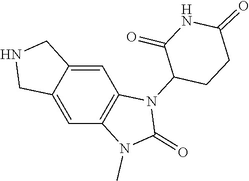 [SMILES: Cn1c(=O)n(C2CCC(=O)NC2=O)c2cc3c(cc21)CNC3] |
| 195 | 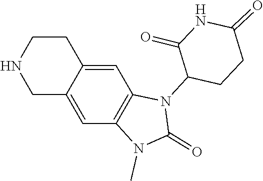 [SMILES: Cn1c(=O)n(C2CCC(=O)NC2=O)c2cc3c(cc21)CNCC3] |
| 196 | 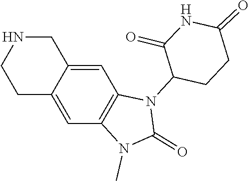 [SMILES: Cn1c(=O)n(C2CCC(=O)NC2=O)c2cc3c(cc21)CCNC3] |
| 197 | 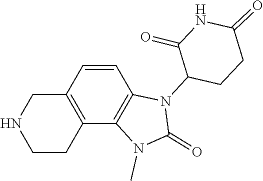 [SMILES: Cn1c(=O)n(C2CCC(=O)NC2=O)c2ccc3c(c21)CCNC3] |
| 198 | 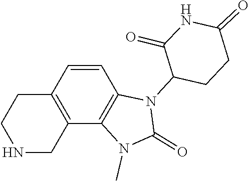 [SMILES: Cn1c(=O)n(C2CCC(=O)NC2=O)c2ccc3c(c21)CNCC3] |
| 199 | 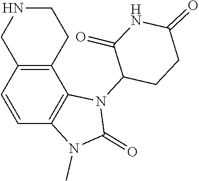 [SMILES: Cn1c(=O)n(C2CCC(=O)NC2=O)c2c3c(ccc21)CNCC3] |
| 200 | 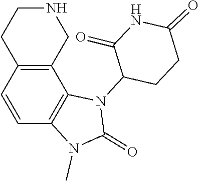 [SMILES: Cn1c(=O)n(C2CCC(=O)NC2=O)c2c3c(ccc21)CCNC3] |
| 201 | 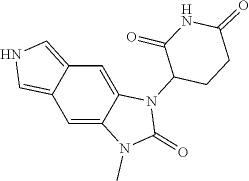 [SMILES: Cn1c(=O)n(C2CCC(=O)NC2=O)c2cc3c[nH]cc3cc21] |
| 202 | 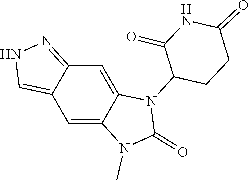 [SMILES: Cn1c(=O)n(C2CCC(=O)NC2=O)c2cc3n[nH]cc3cc21] |
| 203 | 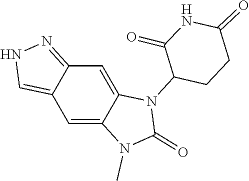 [SMILES: Cn1c(=O)n(C2CCC(=O)NC2=O)c2cc3n[nH]cc3cc21] |
| 204 | 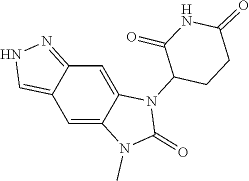 [SMILES: Cn1c(=O)n(C2CCC(=O)NC2=O)c2cc3n[nH]cc3cc21] |
| 205 | 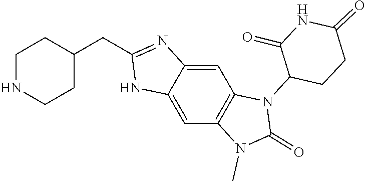 [SMILES: Cn1c(=O)n(C2CCC(=O)NC2=O)c2cc3nc(CC4CCNCC4)[nH]c3cc21] |
| 206 | 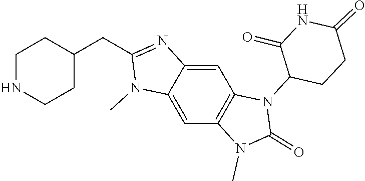 [SMILES: Cn1c(CC2CCNCC2)nc2cc3c(cc21)n(C)c(=O)n3C1CCC(=O)NC1=O] |
| 207 | 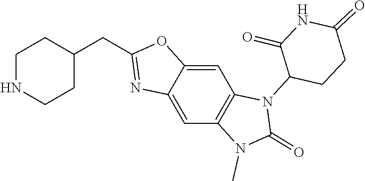 [SMILES: Cn1c(=O)n(C2CCC(=O)NC2=O)c2cc3oc(CC4CCNCC4)nc3cc21] |
| 208 | 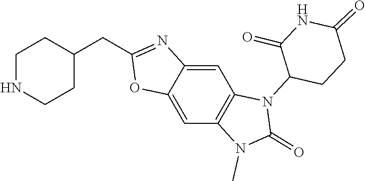 [SMILES: Cn1c(=O)n(C2CCC(=O)NC2=O)c2cc3nc(CC4CCNCC4)oc3cc21] |
| 209 | 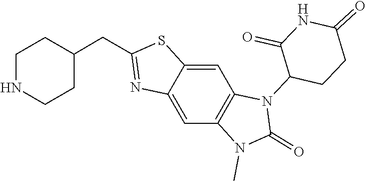 [SMILES: Cn1c(=O)n(C2CCC(=O)NC2=O)c2cc3sc(CC4CCNCC4)nc3cc21] |
| 210 | 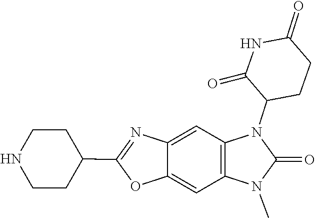 [SMILES: Cn1c(=O)n(C2CCC(=O)NC2=O)c2cc3nc(C4CCNCC4)oc3cc21] |
| 211 | 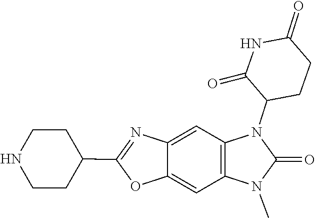 [SMILES: Cn1c(=O)n(C2CCC(=O)NC2=O)c2cc3nc(C4CCNCC4)oc3cc21] |
| 212 | 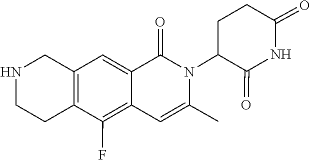 [SMILES: Cc1cc2c(F)c3c(cc2c(=O)n1C1CCC(=O)NC1=O)CNCC3] |
| 213 | 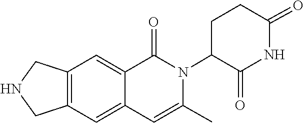 [SMILES: Cc1cc2cc3c(cc2c(=O)n1C1CCC(=O)NC1=O)CNC3] |
| 214 | 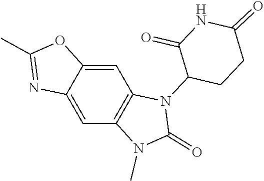 [SMILES: Cc1nc2cc3c(cc2o1)n(C1CCC(=O)NC1=O)c(=O)n3C] |
| 215 | 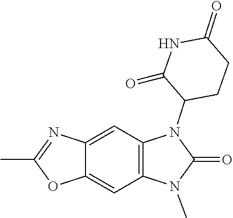 [SMILES: Cc1nc2cc3c(cc2o1)n(C)c(=O)n3C1CCC(=O)NC1=O] |
| 216 | 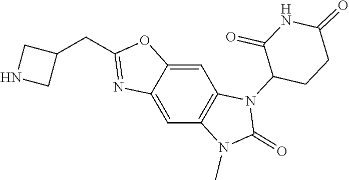 [SMILES: Cn1c(=O)n(C2CCC(=O)NC2=O)c2cc3oc(CC4CNC4)nc3cc21] |

In some embodiments, Cereblon Ligands are any one or more of the compounds of Table 2, or a pharmaceutically acceptable salt or solvate thereof.

In some embodiments, Cereblon Ligands are any one or more of the compounds of Table 2, or a pharmaceutically acceptable salt thereof.

In some embodiments, Cereblon Ligands are any one or more of the compounds of Table 2.

TABLE 2

| Cpd. No. | Structure |
|---|---|
| 217 | 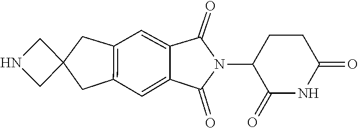 [SMILES: O=C1CCC(N2C(=O)c3cc4c(cc3C2=O)CC2(CNC2)C4)C(=O)N1] |
| 218 | 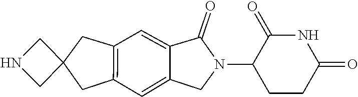 [SMILES: O=C1CCC(N2Cc3cc4c(cc3C2=O)CC2(CNC2)C4)C(=O)N1] |
| 219 | 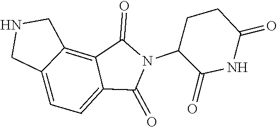 [SMILES: O=C1CCC(N2C(=O)c3ccc4c(c3C2=O)CNC4)C(=O)N1] |
| 220 | 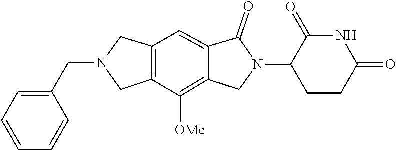 [SMILES: COc1c2c(cc3c1CN(C1CCC(=O)NC1=O)C3=O)CN(Cc1ccccc1)C2] |
| 221 | 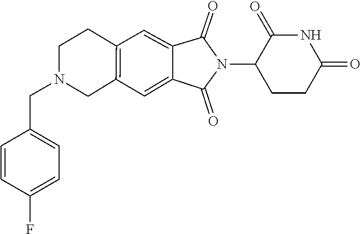 [SMILES: O=C1CCC(N2C(=O)c3cc4c(cc3C2=O)CN(Cc2ccc(F)cc2)CC4)C(=O)N1] |
| 222 | 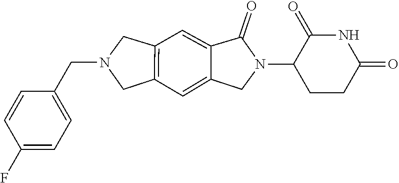 [SMILES: O=C1CCC(N2Cc3cc4c(cc3C2=O)CN(Cc2ccc(F)cc2)C4)C(=O)N1] |
| 223 | 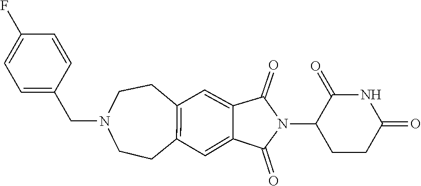 [SMILES: O=C1CCC(N2C(=O)c3cc4c(cc3C2=O)CCN(Cc2ccc(F)cc2)CC4)C(=O)N1] |
| 224 | 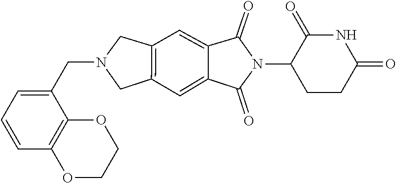 [SMILES: O=C1CCC(N2C(=O)c3cc4c(cc3C2=O)CN(Cc2cccc3c2OCCO3)C4)C(=O)N1] |
| 225 | 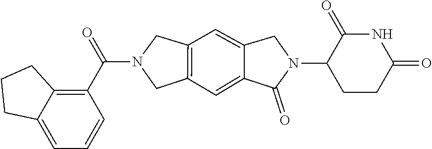 [SMILES: O=C1CCC(N2Cc3cc4c(cc3C2=O)CN(C(=O)c2cccc3c2CCC3)C4)C(=O)N1] |
| 226 | 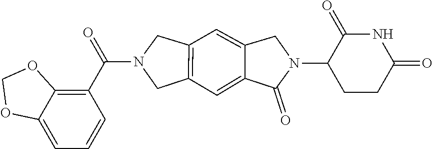 [SMILES: O=C1CCC(N2Cc3cc4c(cc3C2=O)CN(C(=O)c2cccc3c2OCO3)C4)C(=O)N1] |
| 227 | 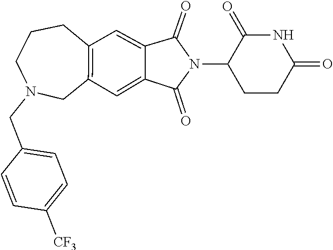 [SMILES: O=C1CCC(N2C(=O)c3cc4c(cc3C2=O)CN(Cc2ccc(C(F)(F)F)cc2)CCC4)C(=O)N1] |
| 228 | 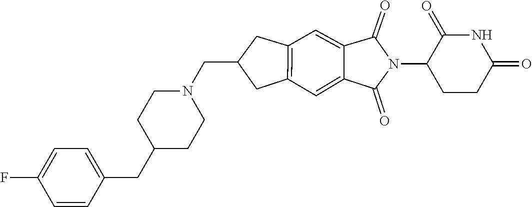 [SMILES: O=C1CCC(N2C(=O)c3cc4c(cc3C2=O)CC(CN2CCC(Cc3ccc(F)cc3)CC2)C4)C(=O)N1] |
| 229 | 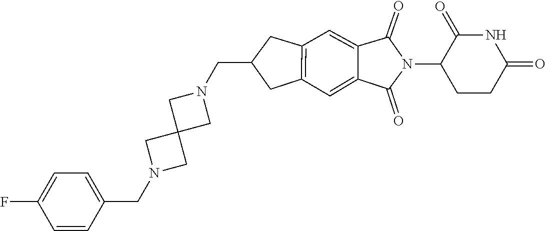 [SMILES: O=C1CCC(N2C(=O)c3cc4c(cc3C2=O)CC(CN2CC3(CN(Cc5ccc(F)cc5)C3)C2)C4)C(=O)N1] |
| 230 | 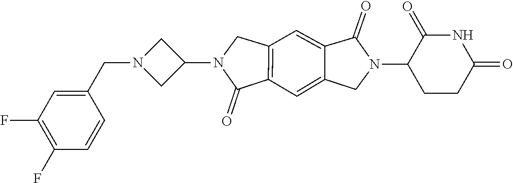 [SMILES: O=C1CCC(N2Cc3cc4c(cc3C2=O)CN(C2CN(Cc3ccc(F)c(F)c3)C2)C4=O)C(=O)N1] |
| 231 | 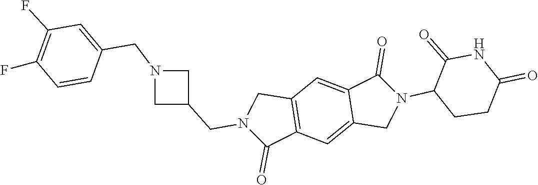 [SMILES: O=C1CCC(N2Cc3cc4c(cc3C2=O)CN(CC2CN(Cc3ccc(F)c(F)c3)C2)C4=O)C(=O)N1] |
| 232 | 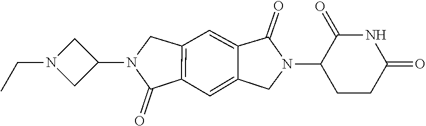 [SMILES: CCN1CC(N2Cc3cc4c(cc3C2=O)CN(C2CCC(=O)NC2=O)C4=O)C1] |
| 233 | 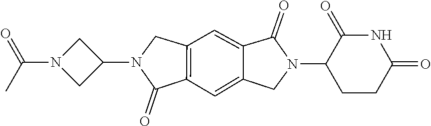 [SMILES: CC(=O)N1CC(N2Cc3cc4c(cc3C2=O)CN(C2CCC(=O)NC2=O)C4=O)C1] |
| 234 | 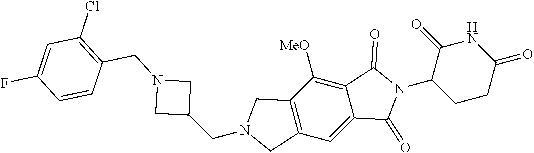 [SMILES: COc1c2c(cc3c1C(=O)N(C1CCC(=O)NC1=O)C3=O)CN(CC1CN(Cc3ccc(F)cc3Cl)C1)C2] |
| 235 | 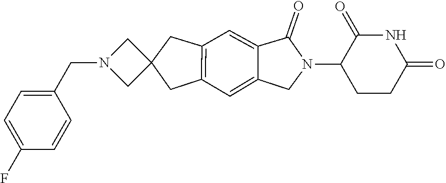 [SMILES: O=C1CCC(N2Cc3cc4c(cc3C2=O)CC2(C4)CN(Cc3ccc(F)cc3)C2)C(=O)N1] |
| 236 | 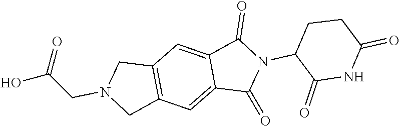 [SMILES: O=C(O)CN1Cc2cc3c(cc2C1)C(=O)N(C1CCC(=O)NC1=O)C3=O] |
| 237 | 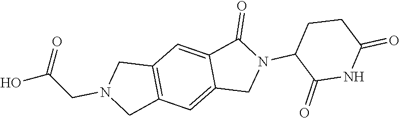 [SMILES: O=C(O)CN1Cc2cc3c(cc2C1)C(=O)N(C1CCC(=O)NC1=O)C3] |
| 238 | 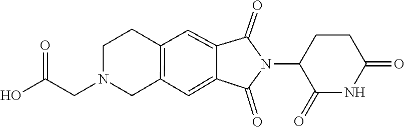 [SMILES: O=C(O)CN1CCc2cc3c(cc2C1)C(=O)N(C1CCC(=O)NC1=O)C3=O] |
| 239 | 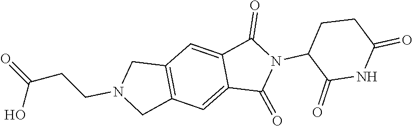 [SMILES: O=C(O)CCN1Cc2cc3c(cc2C1)C(=O)N(C1CCC(=O)NC1=O)C3=O] |
| 240 | 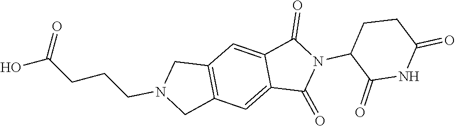 [SMILES: O=C(O)CCCN1Cc2cc3c(cc2C1)C(=O)N(C1CCC(=O)NC1=O)C3=O] |
| 241 | 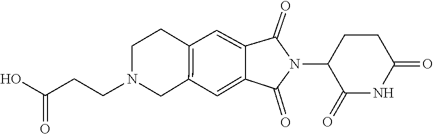 [SMILES: O=C(O)CCN1CCc2cc3c(cc2C1)C(=O)N(C1CCC(=O)NC1=O)C3=O] |
| 242 | 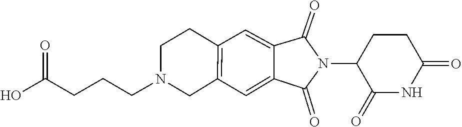 [SMILES: O=C(O)CCCN1CCc2cc3c(cc2C1)C(=O)N(C1CCC(=O)NC1=O)C3=O] |
| 243 | 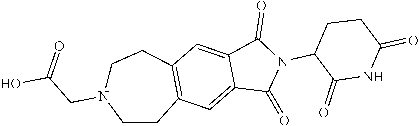 [SMILES: O=C(O)CN1CCc2cc3c(cc2CC1)C(=O)N(C1CCC(=O)NC1=O)C3=O] |
| 244 | 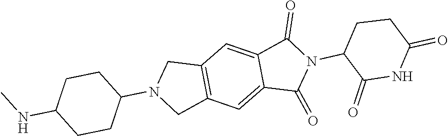 [SMILES: CNC1CCC(N2Cc3cc4c(cc3C2)C(=O)N(C2CCC(=O)NC2=O)C4=O)CC1] |
| 245 | 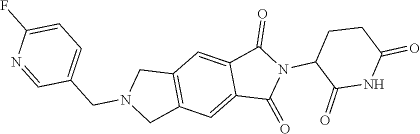 [SMILES: O=C1CCC(N2C(=O)c3cc4c(cc3C2=O)CN(Cc2ccc(F)nc2)C4)C(=O)N1] |
| 246 | 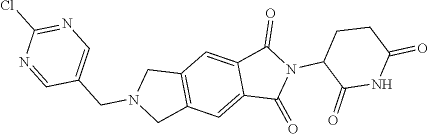 [SMILES: O=C1CCC(N2C(=O)c3cc4c(cc3C2=O)CN(Cc2cnc(Cl)nc2)C4)C(=O)N1] |
| 247 | 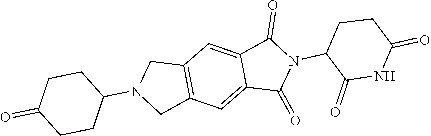 [SMILES: O=C1CCC(N2Cc3cc4c(cc3C2)C(=O)N(C2CCC(=O)NC2=O)C4=O)CC1] |
| 248 | 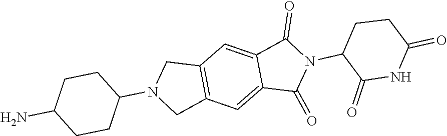 [SMILES: NC1CCC(N2Cc3cc4c(cc3C2)C(=O)N(C2CCC(=O)NC2=O)C4=O)CC1] |
| 249 | 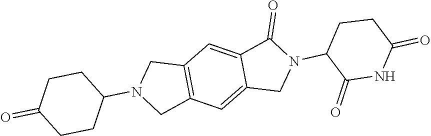 [SMILES: O=C1CCC(N2Cc3cc4c(cc3C2)C(=O)N(C2CCC(=O)NC2=O)C4)CC1] |
| 250 | 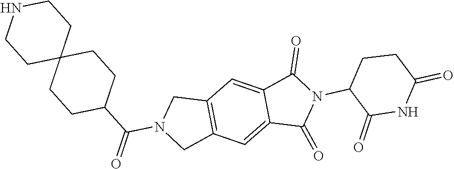 [SMILES: O=C1CCC(N2C(=O)c3cc4c(cc3C2=O)CN(C(=O)C2CCC3(CCNCC3)CC2)C4)C(=O)N1] |
| 251 | 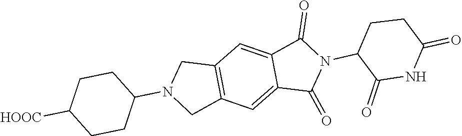 [SMILES: O=C1CCC(N2C(=O)c3cc4c(cc3C2=O)CN(C2CCC(C(=O)O)CC2)C4)C(=O)N1] |
| 252 | 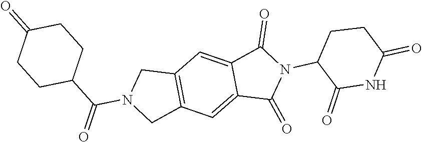 [SMILES: O=C1CCC(C(=O)N2Cc3cc4c(cc3C2)C(=O)N(C2CCC(=O)NC2=O)C4=O)CC1] |
| 253 | 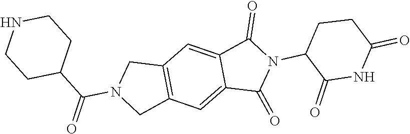 [SMILES: O=C1CCC(N2C(=O)c3cc4c(cc3C2=O)CN(C(=O)C2CCNCC2)C4)C(=O)N1] |
| 254 | 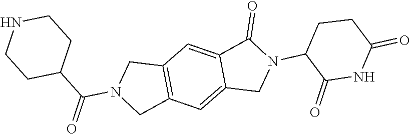 [SMILES: O=C1CCC(N2Cc3cc4c(cc3C2=O)CN(C(=O)C2CCNCC2)C4)C(=O)N1] |
| 255 | 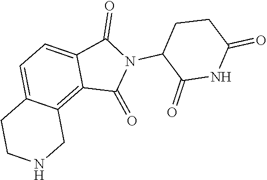 [SMILES: O=C1CCC(N2C(=O)c3ccc4c(c3C2=O)CNCC4)C(=O)N1] |
| 256 | 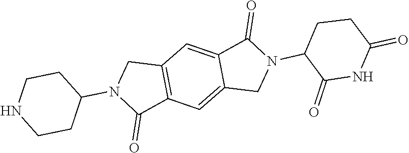 [SMILES: O=C1CCC(N2Cc3cc4c(cc3C2=O)CN(C2CCNCC2)C4=O)C(=O)N1] |
| 257 | 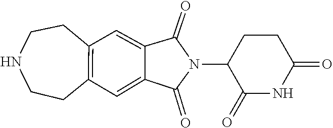 [SMILES: O=C1CCC(N2C(=O)c3cc4c(cc3C2=O)CCNCC4)C(=O)N1] |
| 258 | 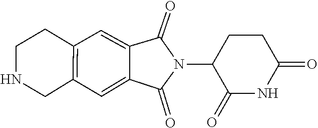 [SMILES: O=C1CCC(N2C(=O)c3cc4c(cc3C2=O)CNCC4)C(=O)N1] |
| 259 | 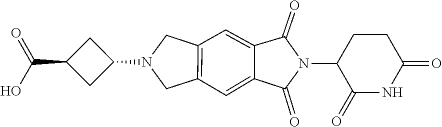 [SMILES: O=C1CCC(N2C(=O)c3cc4c(cc3C2=O)CN([C@H]2C[C@H](C(=O)O)C2)C4)C(=O)N1] |
| 260 | 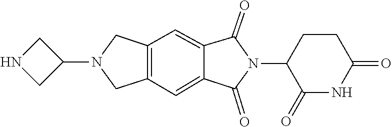 [SMILES: O=C1CCC(N2C(=O)c3cc4c(cc3C2=O)CN(C2CNC2)C4)C(=O)N1] |
| 261 | 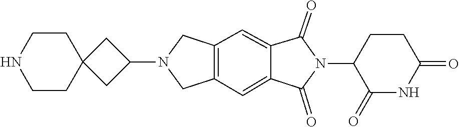 [SMILES: O=C1CCC(N2C(=O)c3cc4c(cc3C2=O)CN(C2CC3(CCNCC3)C2)C4)C(=O)N1] |
| 262 | 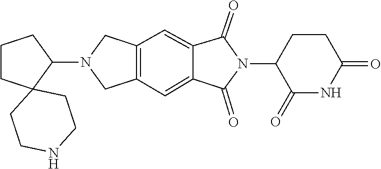 [SMILES: O=C1CCC(N2C(=O)c3cc4c(cc3C2=O)CN(C2CCCC23CCNCC3)C4)C(=O)N1] |
| 263 | 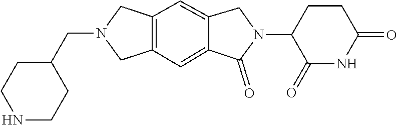 [SMILES: O=C1CCC(N2Cc3cc4c(cc3C2=O)CN(CC2CCNCC2)C4)C(=O)N1] |
| 264 | 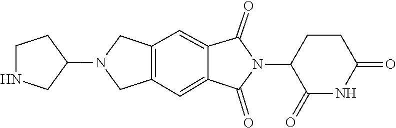 [SMILES: O=C1CCC(N2C(=O)c3cc4c(cc3C2=O)CN(C2CCNC2)C4)C(=O)N1] |
| 265 | 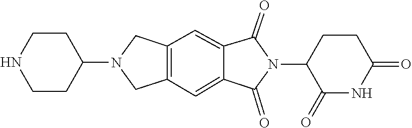 [SMILES: O=C1CCC(N2C(=O)c3cc4c(cc3C2=O)CN(C2CCNCC2)C4)C(=O)N1] |
| 266 | 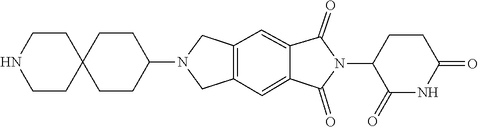 [SMILES: O=C1CCC(N2C(=O)c3cc4c(cc3C2=O)CN(C2CCC3(CCNCC3)CC2)C4)C(=O)N1] |
| 267 | 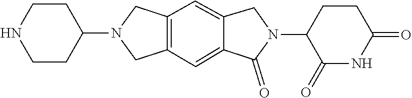 [SMILES: O=C1CCC(N2Cc3cc4c(cc3C2=O)CN(C2CCNCC2)C4)C(=O)N1] |
| 268 | 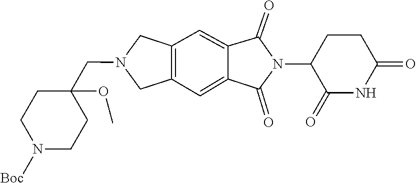 [SMILES: COC1(CN2Cc3cc4c(cc3C2)C(=O)N(C2CCC(=O)NC2=O)C4=O)CCN(C(=O)OC(C)(C)C)CC1] |
| 269 | 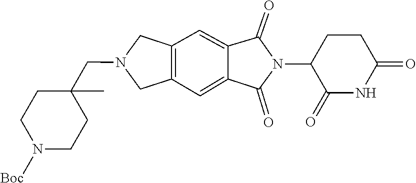 [SMILES: CC1(CN2Cc3cc4c(cc3C2)C(=O)N(C2CCC(=O)NC2=O)C4=O)CCN(C(=O)OC(C)(C)C)CC1] |
| 270 | 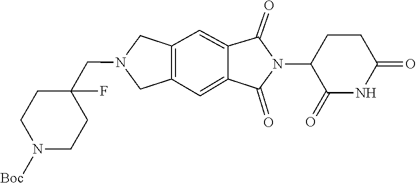 [SMILES: CC(C)(C)OC(=O)N1CCC(F)(CN2Cc3cc4c(cc3C2)C(=O)N(C2CCC(=O)NC2=O)C4=O)CC1] |
| 271 | 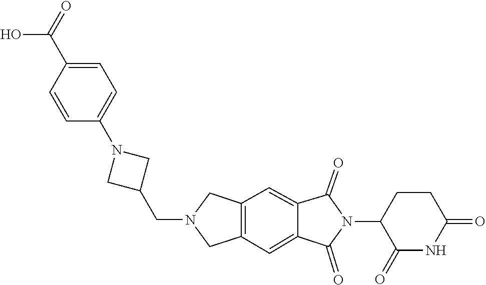 [SMILES: O=C1CCC(N2C(=O)c3cc4c(cc3C2=O)CN(CC2CN(c3ccc(C(=O)O)cc3)C2)C4)C(=O)N1] |
| 272 | 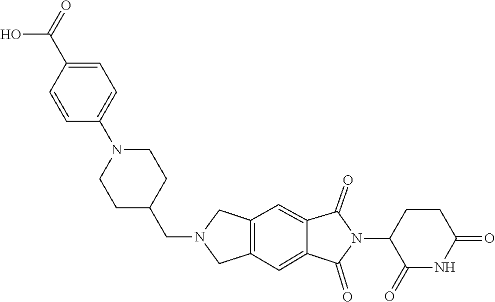 [SMILES: O=C1CCC(N2C(=O)c3cc4c(cc3C2=O)CN(CC2CCN(c3ccc(C(=O)O)cc3)CC2)C4)C(=O)N1] |
| 273 | 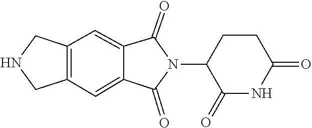 [SMILES: O=C1CCC(N2C(=O)c3cc4c(cc3C2=O)CNC4)C(=O)N1] |
| 274 | 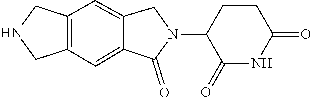 [SMILES: O=C1CCC(N2Cc3cc4c(cc3C2=O)CNC4)C(=O)N1] |
| 275 | 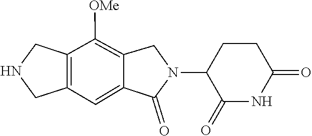 [SMILES: COc1c2c(cc3c1CN(C1CCC(=O)NC1=O)C3=O)CNC2] |

Without wishing to be bound by any particular theory, the Cereblon Ligands of the present disclosure can be used to inhibit the ubiquitination of CRBN and/or used as synthetic intermediates to prepare PROTAC Molecules.

### II. PROTAC Molecules

Proteolysis-targeting chimera (PROTAC) is a useful technology for targeted protein degradation. A bifunctional PROTAC molecule consists of a ligand (usually a small-molecule inhibitor) of the protein of interest and a covalently linked ligand of an E3 ubiquitin ligase. Upon binding to the protein of interest, the PROTAC can recruit E3 ubiquitin ligase for ubiquitination of the protein of interest, which is subjected to proteasome-mediated degradation. See, e.g., Bondeson and Crews, Annu Rev Pharmacol Toxicol. 57:107-123 (2017); Sun et al., Sig Transduct Target Ther 4:64 (2019) https://doi.org/10.1038/s41392-019-0101-6; Li and Song, J Hematol Oncol 13: 50 (2020) https://doi.org/10.1186/s13045-020-00885-3; Wang et al., Acta Pharmaceutica Sinica B 10:207-238 (2020). Without wishing to be bound by any particular theory, Cereblon Ligands can be tethered to a moiety of interest, e.g., ligand that binds to a protein, e.g., small molecule inhibitor of a protein, to give a PROTAC.

In some embodiments, PROTAC Molecules are compounds of any one of Formulae IV-IX, or a pharmaceutically acceptable salt or solvate thereof, wherein X is —N(R^{1a})— and R^{1a} is Q-L-.

In some embodiments, PROTAC Molecules are compounds of any one of Formulae IV-IX, or a pharmaceutically acceptable salt or solvate thereof, wherein X is —CR^{1b}R^{1c}—; R^{1b} is Q-L-; and R^{1c} is hydrogen.

In some embodiments, PROTAC Molecules are compounds of any one of Formulae X-XII, or a pharmaceutically acceptable salt or solvate thereof, wherein R^{1d} is Q-L-.

In some embodiments, PROTAC Molecules are compounds of any one of Formulae XIII-XXI, or a pharmaceutically acceptable salt or solvate thereof, wherein R^{1e} is Q-L-.

In some embodiments, PROTAC Molecules are compounds of any one of Formulae XXII-XXIV, or a pharmaceutically acceptable salt or solvate thereof, wherein E¹ is —CR^{2e}═; and R^{2e} is Q-L-.

In some embodiments, PROTAC Molecules are compounds of any one of Formulae XXII-XXIV, or a pharmaceutically acceptable salt or solvate thereof, wherein E is —CR^{2f}═; and R^{2f} is Q-L-.

In some embodiments, PROTAC Molecules are compounds of any one of Formulae XXII-XXIV, or a pharmaceutically acceptable salt or solvate thereof, wherein E² is —CR^{2g}═; and R^{2g} is Q-L-.

In some embodiments, PROTAC Molecules are compounds of any one of Formulae XXII-XXIV, or a pharmaceutically acceptable salt or solvate thereof, wherein E³ is —CR^{2h}═; and R^{2e} is Q-L-.

In some embodiments, PROTAC Molecules are compounds of any one of Formulae XXV-XXVII, or a pharmaceutically acceptable salt or solvate thereof, wherein R^{1g} is Q-L-.

In some embodiments, PROTAC Molecules are compounds of any one of Formulae XXVIII-XXX, or a pharmaceutically acceptable salt or solvate thereof, wherein R^{1h} is Q-L-.

In some embodiments, PROTAC Molecules are compounds of any one of Formulae XXXI-XXXIX, or a pharmaceutically acceptable salt or solvate thereof, wherein R^{1j} is Q-L-.

In some embodiments, PROTAC Molecules are compounds of any one of Formulae IV-XXXIX, or a pharmaceutically acceptable salt or solvate thereof, wherein L is any one or more of the -J¹-, -J¹-J²-, -J¹-J²-J³-, -J¹-J²-J³-J⁴-, or -J¹-J²-J³-J⁴-J⁵- groups listed in Table 3.

TABLE 3

| No. | J¹ | J² | J³ | J⁴ | J⁵ |
|---|---|---|---|---|---|
| 1 | alkylenyl | — | — | — | — |
| 2 | cycloalkylenyl | — | — | — | — |
| 3 | heterocyclenyl | — | — | — | — |
| 4 | — | —C(═O)— | — | — | — |
| 5 | alkylenyl | —C(═O)— | — | — | — |
| 6 | cycloalkylenyl | —C(═O)— | — | — | — |
| 7 | heterocyclenyl | —C(═O)— | — | — | — |
| 8 | — | —C(═O)NH— | — | — | — |
| 9 | alkylenyl | —C(═O)NH— | — | — | — |
| 10 | cycloalkylenyl | —C(═O)NH— | — | — | — |
| 11 | heterocyclenyl | —C(═O)NH— | — | — | — |
| 12 | — | —C≡C— | — | — | — |
| 13 | alkylenyl | —C≡C— | — | — | — |
| 14 | cycloalkylenyl | —C≡C— | — | — | — |
| 15 | heterocyclenyl | —C≡C— | — | — | — |
| 16 | alkylenyl | — | heterocyclenyl | — | — |
| 17 | cycloalkylenyl | — | heterocyclenyl | — | — |
| 18 | heterocyclenyl | — | heterocyclenyl | — | — |
| 19 | — | —C(═O)— | heterocyclenyl | — | — |
| 20 | alkylenyl | —C(═O)— | heterocyclenyl | — | — |
| 21 | cycloalkylenyl | —C(═O)— | heterocyclenyl | — | — |
| 22 | heterocyclenyl | —C(═O)— | heterocyclenyl | — | — |
| 23 | — | —C(═O)NH— | heterocyclenyl | — | — |
| 24 | alkylenyl | —C(═O)NH— | heterocyclenyl | — | — |
| 25 | cycloalkylenyl | —C(═O)NH— | heterocyclenyl | — | — |
| 26 | heterocyclenyl | —C(═O)NH— | heterocyclenyl | — | — |
| 27 | — | —C≡C— | heterocyclenyl | — | — |
| 28 | alkylenyl | —C≡C— | heterocyclenyl | — | — |
| 29 | cycloalkylenyl | —C≡C— | heterocyclenyl | — | — |
| 30 | heterocyclenyl | —C≡C— | heterocyclenyl | — | — |
| 31 | cycloalkylenyl | — | alkylenyl | heterocyclenyl | — |
| 32 | heterocyclenyl | — | alkylenyl | heterocyclenyl | — |
| 33 | — | —C(═O)— | alkylenyl | heterocyclenyl | — |
| 34 | alkylenyl | —C(═O)— | alkylenyl | heterocyclenyl | — |
| 35 | cycloalkylenyl | —C(═O)— | alkylenyl | heterocyclenyl | — |
| 36 | heterocyclenyl | —C(═O)— | alkylenyl | heterocyclenyl | — |
| 37 | — | —C(═O)NH— | alkylenyl | heterocyclenyl | — |
| 38 | alkylenyl | —C(═O)NH— | alkylenyl | heterocyclenyl | — |
| 39 | cycloalkylenyl | —C(═O)NH— | alkylenyl | heterocyclenyl | — |
| 40 | heterocyclenyl | —C(═O)NH— | alkylenyl | heterocyclenyl | — |
| 41 | — | —C≡C— | alkylenyl | heterocyclenyl | — |
| 42 | alkylenyl | —C≡C— | alkylenyl | heterocyclenyl | — |
| 43 | cycloalkylenyl | —C≡C— | alkylenyl | heterocyclenyl | — |
| 44 | heterocyclenyl | —C≡C— | alkylenyl | heterocyclenyl | — |
| 45 | alkylenyl | — | cycloalkylenyl | heterocyclenyl | — |
| 46 | cycloalkylenyl | — | cycloalkylenyl | heterocyclenyl | — |
| 47 | heterocyclenyl | — | cycloalkylenyl | heterocyclenyl | — |
| 48 | — | —C(═O)— | cycloalkylenyl | heterocyclenyl | — |
| 49 | alkylenyl | —C(═O)— | cycloalkylenyl | heterocyclenyl | — |
| 50 | cycloalkylenyl | —C(═O)— | cycloalkylenyl | heterocyclenyl | — |
| 51 | heterocyclenyl | —C(═O)— | cycloalkylenyl | heterocyclenyl | — |
| 52 | — | —C(═O)NH— | cycloalkylenyl | heterocyclenyl | — |
| 53 | alkylenyl | —C(═O)NH— | cycloalkylenyl | heterocyclenyl | — |
| 54 | cycloalkylenyl | —C(═O)NH— | cycloalkylenyl | heterocyclenyl | — |
| 55 | heterocyclenyl | —C(═O)NH— | cycloalkylenyl | heterocyclenyl | — |
| 56 | — | —C≡C— | cycloalkylenyl | heterocyclenyl | — |
| 57 | alkylenyl | —C≡C— | cycloalkylenyl | heterocyclenyl | — |
| 58 | cycloalkylenyl | —C≡C— | cycloalkylenyl | heterocyclenyl | — |
| 59 | heterocyclenyl | —C≡C— | cycloalkylenyl | heterocyclenyl | — |
| 60 | alkylenyl | — | phenylenyl | heterocyclenyl | — |
| 61 | cycloalkylenyl | — | phenylenyl | heterocyclenyl | — |
| 62 | heterocyclenyl | — | phenylenyl | heterocyclenyl | — |
| 63 | — | —C(═O)— | phenylenyl | heterocyclenyl | — |
| 64 | alkylenyl | —C(═O)— | phenylenyl | heterocyclenyl | — |
| 65 | cycloalkylenyl | —C(═O)— | phenylenyl | heterocyclenyl | — |
| 66 | heterocyclenyl | —C(═O)— | phenylenyl | heterocyclenyl | — |
| 67 | — | —C(═O)NH— | phenylenyl | heterocyclenyl | — |
| 68 | alkylenyl | —C(═O)NH— | phenylenyl | heterocyclenyl | — |
| 69 | cycloalkylenyl | —C(═O)NH— | phenylenyl | heterocyclenyl | — |
| 70 | heterocyclenyl | —C(═O)NH— | phenylenyl | heterocyclenyl | — |
| 71 | — | —C≡C— | phenylenyl | heterocyclenyl | — |
| 72 | alkylenyl | —C≡C— | phenylenyl | heterocyclenyl | — |
| 73 | cycloalkylenyl | —C≡C— | phenylenyl | heterocyclenyl | — |
| 74 | heterocyclenyl | —C≡C— | phenylenyl | heterocyclenyl | — |
| 75 | cycloalkylenyl | — | alkylenyl | — | —C(═O)— |
| 76 | heterocyclenyl | — | alkylenyl | — | —C(═O)— |
| 77 | — | —C(═O)— | alkylenyl | — | —C(═O)— |
| 78 | alkylenyl | —C(═O)— | alkylenyl | — | —C(═O)— |
| 79 | cycloalkylenyl | —C(═O)— | alkylenyl | — | —C(═O)— |
| 80 | heterocyclenyl | —C(═O)— | alkylenyl | — | —C(═O)— |
| 81 | — | —C(═O)NH— | alkylenyl | — | —C(═O)— |
| 82 | alkylenyl | —C(═O)NH— | alkylenyl | — | —C(═O)— |
| 83 | cycloalkylenyl | —C(═O)NH— | alkylenyl | — | —C(═O)— |
| 84 | heterocyclenyl | —C(═O)NH— | alkylenyl | — | —C(═O)— |
| 85 | — | —C≡C— | alkylenyl | — | —C(═O)— |
| 86 | alkylenyl | —C≡C— | alkylenyl | — | —C(═O)— |
| 87 | cycloalkylenyl | —C≡C— | alkylenyl | — | —C(═O)— |
| 88 | heterocyclenyl | —C≡C— | alkylenyl | — | —C(═O)— |
| 89 | — | — | heteroalkylenyl | — | —C(═O)— |
| 90 | alkylenyl | — | heteroalkylenyl | — | —C(═O)— |
| 91 | cycloalkylenyl | — | heteroalkylenyl | — | —C(═O)— |
| 92 | heterocyclenyl | — | heteroalkylenyl | — | —C(═O)— |
| 93 | — | —C(═O)— | heteroalkylenyl | — | —C(═O)— |
| 94 | alkylenyl | —C(═O)— | heteroalkylenyl | — | —C(═O)— |
| 95 | cycloalkylenyl | —C(═O)— | heteroalkylenyl | — | —C(═O)— |
| 96 | heterocyclenyl | —C(═O)— | heteroalkylenyl | — | —C(═O)— |
| 97 | — | —C(═O)NH— | heteroalkylenyl | — | —C(═O)— |
| 98 | alkylenyl | —C(═O)NH— | heteroalkylenyl | — | —C(═O)— |
| 99 | cycloalkylenyl | —C(═O)NH— | heteroalkylenyl | — | —C(═O)— |
| 100 | heterocyclenyl | —C(═O)NH— | heteroalkylenyl | — | —C(═O)— |
| 101 | — | —C≡C— | heteroalkylenyl | — | —C(═O)— |
| 102 | alkylenyl | —C≡C— | heteroalkylenyl | — | —C(═O)— |
| 103 | cycloalkylenyl | —C≡C— | heteroalkylenyl | — | —C(═O)— |
| 104 | heterocyclenyl | —C≡C— | heteroalkylenyl | — | —C(═O)— |
| 105 | alkylenyl | — | heterocyclenyl | — | —C(═O)— |
| 106 | cycloalkylenyl | — | heterocyclenyl | — | —C(═O)— |
| 107 | heterocyclenyl | — | heterocyclenyl | — | —C(═O)— |
| 108 | — | —C(═O)— | heterocyclenyl | — | —C(═O)— |
| 109 | alkylenyl | —C(═O)— | heterocyclenyl | — | —C(═O)— |
| 110 | cycloalkylenyl | —C(═O)— | heterocyclenyl | — | —C(═O)— |
| 111 | heterocyclenyl | —C(═O)— | heterocyclenyl | — | —C(═O)— |
| 112 | — | —C(═O)NH— | heterocyclenyl | — | —C(═O)— |
| 113 | alkylenyl | —C(═O)NH— | heterocyclenyl | — | —C(═O)— |
| 114 | cycloalkylenyl | —C(═O)NH— | heterocyclenyl | — | —C(═O)— |
| 115 | heterocyclenyl | —C(═O)NH— | heterocyclenyl | — | —C(═O)— |
| 116 | — | —C≡C— | heterocyclenyl | — | —C(═O)— |
| 117 | alkylenyl | —C≡C— | heterocyclenyl | — | —C(═O)— |
| 118 | cycloalkylenyl | —C≡C— | heterocyclenyl | — | —C(═O)— |
| 119 | heterocyclenyl | —C≡C— | heterocyclenyl | — | —C(═O)— |
| 120 | alkylenyl | — | alkylenyl | heterocyclenyl | —C(═O)— |
| 121 | cycloalkylenyl | — | alkylenyl | heterocyclenyl | —C(═O)— |
| 122 | heterocyclenyl | — | alkylenyl | heterocyclenyl | —C(═O)— |
| 123 | — | —C(═O)— | alkylenyl | heterocyclenyl | —C(═O)— |
| 124 | alkylenyl | —C(═O)— | alkylenyl | heterocyclenyl | —C(═O)— |
| 125 | cycloalkylenyl | —C(═O)— | alkylenyl | heterocyclenyl | —C(═O)— |
| 126 | heterocyclenyl | —C(═O)— | alkylenyl | heterocyclenyl | —C(═O)— |
| 127 | — | —C(═O)NH— | alkylenyl | heterocyclenyl | —C(═O)— |
| 128 | alkylenyl | —C(═O)NH— | alkylenyl | heterocyclenyl | —C(═O)— |
| 129 | cycloalkylenyl | —C(═O)NH— | alkylenyl | heterocyclenyl | —C(═O)— |
| 130 | heterocyclenyl | —C(═O)NH— | alkylenyl | heterocyclenyl | —C(═O)— |
| 131 | — | —C≡C— | alkylenyl | heterocyclenyl | —C(═O)— |
| 132 | alkylenyl | —C≡C— | alkylenyl | heterocyclenyl | —C(═O)— |
| 133 | cycloalkylenyl | —C≡C— | alkylenyl | heterocyclenyl | —C(═O)— |
| 134 | heterocyclenyl | —C≡C— | alkylenyl | heterocyclenyl | —C(═O)— |
| 135 | alkylenyl | — | heterocyclenyl | alkylenyl | —C(═O)— |
| 136 | cycloalkylenyl | — | heterocyclenyl | alkylenyl | —C(═O)— |
| 137 | heterocyclenyl | — | heterocyclenyl | alkylenyl | —C(═O)— |
| 138 | — | —C(═O)— | heterocyclenyl | alkylenyl | —C(═O)— |
| 139 | alkylenyl | —C(═O)— | heterocyclenyl | alkylenyl | —C(═O)— |
| 140 | cycloalkylenyl | —C(═O)— | heterocyclenyl | alkylenyl | —C(═O)— |
| 141 | heterocyclenyl | —C(═O)— | heterocyclenyl | alkylenyl | —C(═O)— |
| 142 | — | —C(═O)NH— | heterocyclenyl | alkylenyl | —C(═O)— |
| 143 | alkylenyl | —C(═O)NH— | heterocyclenyl | alkylenyl | —C(═O)— |
| 144 | cycloalkylenyl | —C(═O)NH— | heterocyclenyl | alkylenyl | —C(═O)— |
| 145 | heterocyclenyl | —C(═O)NH— | heterocyclenyl | alkylenyl | —C(═O)— |
| 146 | — | —C≡C— | heterocyclenyl | alkylenyl | —C(═O)— |
| 147 | alkylenyl | —C≡C— | heterocyclenyl | alkylenyl | —C(═O)— |
| 148 | cycloalkylenyl | —C≡C— | heterocyclenyl | alkylenyl | —C(═O)— |
| 149 | heterocyclenyl | —C≡C— | heterocyclenyl | alkylenyl | —C(═O)— |
| 150 | alkylenyl | — | — | cycloalkylenyl | —C(═O)— |
| 151 | cycloalkylenyl | — | — | cycloalkylenyl | —C(═O)— |
| 152 | heterocyclenyl | — | — | cycloalkylenyl | —C(═O)— |
| 153 | — | —C(═O)— | — | cycloalkylenyl | —C(═O)— |
| 154 | alkylenyl | —C(═O)— | — | cycloalkylenyl | —C(═O)— |
| 155 | cycloalkylenyl | —C(═O)— | — | cycloalkylenyl | —C(═O)— |
| 156 | heterocyclenyl | —C(═O)— | — | cycloalkylenyl | —C(═O)— |
| 157 | — | —C(═O)NH— | — | cycloalkylenyl | —C(═O)— |
| 158 | alkylenyl | —C(═O)NH— | — | cycloalkylenyl | —C(═O)— |
| 159 | cycloalkylenyl | —C(═O)NH— | — | cycloalkylenyl | —C(═O)— |
| 160 | heterocyclenyl | —C(═O)NH— | — | cycloalkylenyl | —C(═O)— |
| 161 | — | —C≡C— | — | cycloalkylenyl | —C(═O)— |
| 162 | alkylenyl | —C≡C— | — | cycloalkylenyl | —C(═O)— |
| 163 | cycloalkylenyl | —C≡C— | — | cycloalkylenyl | —C(═O)— |
| 164 | heterocyclenyl | —C≡C— | — | cycloalkylenyl | —C(═O)— |
| 165 | alkylenyl | — | alkylenyl | cycloalkylenyl | —C(═O)— |
| 166 | cycloalkylenyl | — | alkylenyl | cycloalkylenyl | —C(═O)— |
| 167 | heterocyclenyl | — | alkylenyl | cycloalkylenyl | —C(═O)— |
| 168 | — | —C(═O)— | alkylenyl | cycloalkylenyl | —C(═O)— |
| 169 | alkylenyl | —C(═O)— | alkylenyl | cycloalkylenyl | —C(═O)— |
| 170 | cycloalkylenyl | —C(═O)— | alkylenyl | cycloalkylenyl | —C(═O)— |
| 171 | heterocyclenyl | —C(═O)— | alkylenyl | cycloalkylenyl | —C(═O)— |
| 172 | — | —C(═O)NH— | alkylenyl | cycloalkylenyl | —C(═O)— |
| 173 | alkylenyl | —C(═O)NH— | alkylenyl | cycloalkylenyl | —C(═O)— |
| 174 | cycloalkylenyl | —C(═O)NH— | alkylenyl | cycloalkylenyl | —C(═O)— |
| 175 | heterocyclenyl | —C(═O)NH— | alkylenyl | cycloalkylenyl | —C(═O)— |
| 176 | — | —C≡C— | alkylenyl | cycloalkylenyl | —C(═O)— |
| 177 | alkylenyl | —C≡C— | alkylenyl | cycloalkylenyl | —C(═O)— |
| 178 | cycloalkylenyl | —C≡C— | alkylenyl | cycloalkylenyl | —C(═O)— |
| 179 | heterocyclenyl | —C≡C— | alkylenyl | cycloalkylenyl | —C(═O)— |
| 180 | alkylenyl | — | heterocyclenyl | cycloalkylenyl | —C(═O)— |
| 181 | cycloalkylenyl | — | heterocyclenyl | cycloalkylenyl | —C(═O)— |
| 182 | heterocyclenyl | — | heterocyclenyl | cycloalkylenyl | —C(═O)— |
| 183 | — | —C(═O)— | heterocyclenyl | cycloalkylenyl | —C(═O)— |
| 184 | alkylenyl | —C(═O)— | heterocyclenyl | cycloalkylenyl | —C(═O)— |
| 185 | cycloalkylenyl | —C(═O)— | heterocyclenyl | cycloalkylenyl | —C(═O)— |
| 186 | heterocyclenyl | —C(═O)— | heterocyclenyl | cycloalkylenyl | —C(═O)— |
| 187 | — | —C(═O)NH— | heterocyclenyl | cycloalkylenyl | —C(═O)— |
| 188 | alkylenyl | —C(═O)NH— | heterocyclenyl | cycloalkylenyl | —C(═O)— |
| 189 | cycloalkylenyl | —C(═O)NH— | heterocyclenyl | cycloalkylenyl | —C(═O)— |
| 190 | heterocyclenyl | —C(═O)NH— | heterocyclenyl | cycloalkylenyl | —C(═O)— |
| 191 | — | —C≡C— | heterocyclenyl | cycloalkylenyl | —C(═O)— |
| 192 | alkylenyl | —C≡C— | heterocyclenyl | cycloalkylenyl | —C(═O)— |
| 193 | cycloalkylenyl | —C≡C— | heterocyclenyl | cycloalkylenyl | —C(═O)— |
| 194 | heterocyclenyl | —C≡C— | heterocyclenyl | cycloalkylenyl | —C(═O)— |
| 195 | alkylenyl | — | — | — | —O— |
| 196 | cycloalkylenyl | — | — | — | —O— |
| 197 | heterocyclenyl | — | — | — | —O— |
| 198 | cycloalkylenyl | — | alkylenyl | — | —O— |
| 199 | heterocyclenyl | — | alkylenyl | — | —O— |
| 200 | — | —C(═O)— | alkylenyl | — | —O— |
| 201 | alkylenyl | —C(═O)— | alkylenyl | — | —O— |
| 202 | cycloalkylenyl | —C(═O)— | alkylenyl | — | —O— |
| 203 | heterocyclenyl | —C(═O)— | alkylenyl | — | —O— |
| 204 | — | —C(═O)NH— | alkylenyl | — | —O— |
| 205 | alkylenyl | —C(═O)NH— | alkylenyl | — | —O— |
| 206 | cycloalkylenyl | —C(═O)NH— | alkylenyl | — | —O— |
| 207 | heterocyclenyl | —C(═O)NH— | alkylenyl | — | —O— |
| 208 | — | —C≡C— | alkylenyl | — | —O— |
| 209 | alkylenyl | —C≡C— | alkylenyl | — | —O— |
| 210 | cycloalkylenyl | —C≡C— | alkylenyl | — | —O— |
| 211 | heterocyclenyl | —C≡C— | alkylenyl | — | —O— |
| 212 | alkylenyl | — | heterocyclenyl | — | —O— |
| 213 | cycloalkylenyl | — | heterocyclenyl | — | —O— |
| 214 | heterocyclenyl | — | heterocyclenyl | — | —O— |
| 215 | — | —C(═O)— | heterocyclenyl | — | —O— |
| 216 | alkylenyl | —C(═O)— | heterocyclenyl | — | —O— |
| 217 | cycloalkylenyl | —C(═O)— | heterocyclenyl | — | —O— |
| 218 | heterocyclenyl | —C(═O)— | heterocyclenyl | — | —O— |
| 219 | — | —C(═O)NH— | heterocyclenyl | — | —O— |
| 220 | alkylenyl | —C(═O)NH— | heterocyclenyl | — | —O— |
| 221 | cycloalkylenyl | —C(═O)NH— | heterocyclenyl | — | —O— |
| 222 | heterocyclenyl | —C(═O)NH— | heterocyclenyl | — | —O— |
| 223 | — | —C≡C— | heterocyclenyl | — | —O— |
| 224 | alkylenyl | —C≡C— | heterocyclenyl | — | —O— |
| 225 | cycloalkylenyl | —C≡C— | heterocyclenyl | — | —O— |
| 226 | heterocyclenyl | —C≡C— | heterocyclenyl | — | —O— |
| 227 | alkylenyl | — | alkylenyl | heterocyclenyl | —O— |
| 228 | cycloalkylenyl | — | alkylenyl | heterocyclenyl | —O— |
| 229 | heterocyclenyl | — | alkylenyl | heterocyclenyl | —O— |
| 230 | — | —C(═O)— | alkylenyl | heterocyclenyl | —O— |
| 231 | alkylenyl | —C(═O)— | alkylenyl | heterocyclenyl | —O— |
| 232 | cycloalkylenyl | —C(═O)— | alkylenyl | heterocyclenyl | —O— |
| 233 | heterocyclenyl | —C(═O)— | alkylenyl | heterocyclenyl | —O— |
| 234 | — | —C(═O)NH— | alkylenyl | heterocyclenyl | —O— |
| 235 | alkylenyl | —C(═O)NH— | alkylenyl | heterocyclenyl | —O— |
| 236 | cycloalkylenyl | —C(═O)NH— | alkylenyl | heterocyclenyl | —O— |
| 237 | heterocyclenyl | —C(═O)NH— | alkylenyl | heterocyclenyl | —O— |
| 238 | — | —C≡C— | alkylenyl | heterocyclenyl | —O— |
| 239 | alkylenyl | —C≡C— | alkylenyl | heterocyclenyl | —O— |
| 240 | cycloalkylenyl | —C≡C— | alkylenyl | heterocyclenyl | —O— |
| 241 | heterocyclenyl | —C≡C— | alkylenyl | heterocyclenyl | —O— |
| 242 | alkylenyl | — | heterocyclenyl | alkylenyl | —O— |
| 243 | cycloalkylenyl | — | heterocyclenyl | alkylenyl | —O— |
| 244 | heterocyclenyl | — | heterocyclenyl | alkylenyl | —O— |
| 245 | — | —C(═O)— | heterocyclenyl | alkylenyl | —O— |
| 246 | alkylenyl | —C(═O)— | heterocyclenyl | alkylenyl | —O— |
| 247 | cycloalkylenyl | —C(═O)— | heterocyclenyl | alkylenyl | —O— |
| 248 | heterocyclenyl | —C(═O)— | heterocyclenyl | alkylenyl | —O— |
| 249 | — | —C(═O)NH— | heterocyclenyl | alkylenyl | —O— |
| 250 | alkylenyl | —C(═O)NH— | heterocyclenyl | alkylenyl | —O— |
| 251 | cycloalkylenyl | —C(═O)NH— | heterocyclenyl | alkylenyl | —O— |
| 252 | heterocyclenyl | —C(═O)NH— | heterocyclenyl | alkylenyl | —O— |
| 253 | — | —C≡C— | heterocyclenyl | alkylenyl | —O— |
| 254 | alkylenyl | —C≡C— | heterocyclenyl | alkylenyl | —O— |
| 255 | cycloalkylenyl | —C≡C— | heterocyclenyl | alkylenyl | —O— |
| 256 | heterocyclenyl | —C≡C— | heterocyclenyl | alkylenyl | —O— |
| 257 | alkylenyl | — | — | cycloalkylenyl | —O— |
| 258 | cycloalkylenyl | — | — | cycloalkylenyl | —O— |
| 259 | heterocyclenyl | — | — | cycloalkylenyl | —O— |
| 260 | — | —C(═O)— | — | cycloalkylenyl | —O— |
| 261 | alkylenyl | —C(═O)— | — | cycloalkylenyl | —O— |
| 262 | cycloalkylenyl | —C(═O)— | — | cycloalkylenyl | —O— |
| 263 | heterocyclenyl | —C(═O)— | — | cycloalkylenyl | —O— |
| 264 | — | —C(═O)NH— | — | cycloalkylenyl | —O— |
| 265 | alkylenyl | —C(═O)NH— | — | cycloalkylenyl | —O— |
| 266 | cycloalkylenyl | —C(═O)NH— | — | cycloalkylenyl | —O— |
| 267 | heterocyclenyl | —C(═O)NH— | — | cycloalkylenyl | —O— |
| 268 | — | —C≡C— | — | cycloalkylenyl | —O— |
| 269 | alkylenyl | —C≡C— | — | cycloalkylenyl | —O— |
| 270 | cycloalkylenyl | —C≡C— | — | cycloalkylenyl | —O— |
| 271 | heterocyclenyl | —C≡C— | — | cycloalkylenyl | —O— |
| 272 | alkylenyl | — | alkylenyl | cycloalkylenyl | —O— |
| 273 | cycloalkylenyl | — | alkylenyl | cycloalkylenyl | —O— |
| 274 | heterocyclenyl | — | alkylenyl | cycloalkylenyl | —O— |
| 275 | — | —C(═O)— | alkylenyl | cycloalkylenyl | —O— |
| 276 | alkylenyl | —C(═O)— | alkylenyl | cycloalkylenyl | —O— |
| 277 | cycloalkylenyl | —C(═O)— | alkylenyl | cycloalkylenyl | —O— |
| 278 | heterocyclenyl | —C(═O)— | alkylenyl | cycloalkylenyl | —O— |
| 279 | — | —C(═O)NH— | alkylenyl | cycloalkylenyl | —O— |
| 280 | alkylenyl | —C(═O)NH— | alkylenyl | cycloalkylenyl | —O— |
| 281 | cycloalkylenyl | —C(═O)NH— | alkylenyl | cycloalkylenyl | —O— |
| 282 | heterocyclenyl | —C(═O)NH— | alkylenyl | cycloalkylenyl | —O— |
| 283 | — | —C≡C— | alkylenyl | cycloalkylenyl | —O— |
| 284 | alkylenyl | —C≡C— | alkylenyl | cycloalkylenyl | —O— |
| 285 | cycloalkylenyl | —C≡C— | alkylenyl | cycloalkylenyl | —O— |
| 286 | heterocyclenyl | —C≡C— | alkylenyl | cycloalkylenyl | —O— |
| 287 | alkylenyl | — | heterocyclenyl | cycloalkylenyl | —O— |
| 288 | cycloalkylenyl | — | heterocyclenyl | cycloalkylenyl | —O— |
| 289 | heterocyclenyl | — | heterocyclenyl | cycloalkylenyl | —O— |
| 290 | — | —C(═O)— | heterocyclenyl | cycloalkylenyl | —O— |
| 291 | alkylenyl | —C(═O)— | heterocyclenyl | cycloalkylenyl | —O— |
| 292 | cycloalkylenyl | —C(═O)— | heterocyclenyl | cycloalkylenyl | —O— |
| 293 | heterocyclenyl | —C(═O)— | heterocyclenyl | cycloalkylenyl | —O— |
| 294 | — | —C(═O)NH— | heterocyclenyl | cycloalkylenyl | —O— |
| 295 | alkylenyl | —C(═O)NH— | heterocyclenyl | cycloalkylenyl | —O— |
| 296 | cycloalkylenyl | —C(═O)NH— | heterocyclenyl | cycloalkylenyl | —O— |
| 297 | heterocyclenyl | —C(═O)NH— | heterocyclenyl | cycloalkylenyl | —O— |
| 298 | — | —C≡C— | heterocyclenyl | cycloalkylenyl | —O— |
| 299 | alkylenyl | —C≡C— | heterocyclenyl | cycloalkylenyl | —O— |
| 300 | cycloalkylenyl | —C≡C— | heterocyclenyl | cycloalkylenyl | —O— |
| 301 | heterocyclenyl | —C≡C— | heterocyclenyl | cycloalkylenyl | —O— |
| 302 | alkylenyl | — | — | — | —NH— |
| 303 | cycloalkylenyl | — | — | — | —NH— |
| 304 | heterocyclenyl | — | — | — | —NH— |
| 305 | cycloalkylenyl | — | alkylenyl | — | —NH— |
| 306 | heterocyclenyl | — | alkylenyl | — | —NH— |
| 307 | — | —C(═O)— | alkylenyl | — | —NH— |
| 308 | alkylenyl | —C(═O)— | alkylenyl | — | —NH— |
| 309 | cycloalkylenyl | —C(═O)— | alkylenyl | — | —NH— |
| 310 | heterocyclenyl | —C(═O)— | alkylenyl | — | —NH— |
| 311 | — | —C(═O)NH— | alkylenyl | — | —NH— |
| 312 | alkylenyl | —C(═O)NH— | alkylenyl | — | —NH— |
| 313 | cycloalkylenyl | —C(═O)NH— | alkylenyl | — | —NH— |
| 314 | heterocyclenyl | —C(═O)NH— | alkylenyl | — | —NH— |
| 315 | — | —C≡C— | alkylenyl | — | —NH— |
| 316 | alkylenyl | —C≡C— | alkylenyl | — | —NH— |
| 317 | cycloalkylenyl | —C≡C— | alkylenyl | — | —NH— |
| 318 | heterocyclenyl | —C≡C— | alkylenyl | — | —NH— |
| 319 | alkylenyl | — | heterocyclenyl | — | —NH— |
| 320 | cycloalkylenyl | — | heterocyclenyl | — | —NH— |
| 321 | heterocyclenyl | — | heterocyclenyl | — | —NH— |
| 322 | — | —C(═O)— | heterocyclenyl | — | —NH— |
| 323 | alkylenyl | —C(═O)— | heterocyclenyl | — | —NH— |
| 324 | cycloalkylenyl | —C(═O)— | heterocyclenyl | — | —NH— |
| 325 | heterocyclenyl | —C(═O)— | heterocyclenyl | — | —NH— |
| 326 | — | —C(═O)NH— | heterocyclenyl | — | —NH— |
| 327 | alkylenyl | —C(═O)NH— | heterocyclenyl | — | —NH— |
| 328 | cycloalkylenyl | —C(═O)NH— | heterocyclenyl | — | —NH— |
| 329 | heterocyclenyl | —C(═O)NH— | heterocyclenyl | — | —NH— |
| 330 | — | —C≡C— | heterocyclenyl | — | —NH— |
| 331 | alkylenyl | —C≡C— | heterocyclenyl | — | —NH— |
| 332 | cycloalkylenyl | —C≡C— | heterocyclenyl | — | —NH— |
| 333 | heterocyclenyl | —C≡C— | heterocyclenyl | — | —NH— |
| 334 | alkylenyl | — | alkylenyl | heterocyclenyl | —NH— |
| 335 | cycloalkylenyl | — | alkylenyl | heterocyclenyl | —NH— |
| 336 | heterocyclenyl | — | alkylenyl | heterocyclenyl | —NH— |
| 337 | — | —C(═O)— | alkylenyl | heterocyclenyl | —NH— |
| 338 | alkylenyl | —C(═O)— | alkylenyl | heterocyclenyl | —NH— |
| 339 | cycloalkylenyl | —C(═O)— | alkylenyl | heterocyclenyl | —NH— |
| 340 | heterocyclenyl | —C(═O)— | alkylenyl | heterocyclenyl | —NH— |
| 341 | — | —C(═O)NH— | alkylenyl | heterocyclenyl | —NH— |
| 342 | alkylenyl | —C(═O)NH— | alkylenyl | heterocyclenyl | —NH— |
| 343 | cycloalkylenyl | —C(═O)NH— | alkylenyl | heterocyclenyl | —NH— |
| 344 | heterocyclenyl | —C(═O)NH— | alkylenyl | heterocyclenyl | —NH— |
| 345 | — | —C≡C— | alkylenyl | heterocyclenyl | —NH— |
| 346 | alkylenyl | —C≡C— | alkylenyl | heterocyclenyl | —NH— |
| 347 | cycloalkylenyl | —C≡C— | alkylenyl | heterocyclenyl | —NH— |
| 348 | heterocyclenyl | —C≡C— | alkylenyl | heterocyclenyl | —NH— |
| 349 | alkylenyl | — | heterocyclenyl | alkylenyl | —NH— |
| 350 | cycloalkylenyl | — | heterocyclenyl | alkylenyl | —NH— |
| 351 | heterocyclenyl | — | heterocyclenyl | alkylenyl | —NH— |
| 352 | — | —C(═O)— | heterocyclenyl | alkylenyl | —NH— |
| 353 | alkylenyl | —C(═O)— | heterocyclenyl | alkylenyl | —NH— |
| 354 | cycloalkylenyl | —C(═O)— | heterocyclenyl | alkylenyl | —NH— |
| 355 | heterocyclenyl | —C(═O)— | heterocyclenyl | alkylenyl | —NH— |
| 356 | — | —C(═O)NH— | heterocyclenyl | alkylenyl | —NH— |
| 357 | alkylenyl | —C(═O)NH— | heterocyclenyl | alkylenyl | —NH— |
| 358 | cycloalkylenyl | —C(═O)NH— | heterocyclenyl | alkylenyl | —NH— |
| 359 | heterocyclenyl | —C(═O)NH— | heterocyclenyl | alkylenyl | —NH— |
| 360 | — | —C≡C— | heterocyclenyl | alkylenyl | —NH— |
| 361 | alkylenyl | —C≡C— | heterocyclenyl | alkylenyl | —NH— |
| 362 | cycloalkylenyl | —C≡C— | heterocyclenyl | alkylenyl | —NH— |
| 363 | heterocyclenyl | —C≡C— | heterocyclenyl | alkylenyl | —NH— |
| 364 | alkylenyl | — | — | cycloalkylenyl | —NH— |
| 365 | cycloalkylenyl | — | — | cycloalkylenyl | —NH— |
| 366 | heterocyclenyl | — | — | cycloalkylenyl | —NH— |
| 367 | — | —C(═O)— | — | cycloalkylenyl | —NH— |
| 368 | alkylenyl | —C(═O)— | — | cycloalkylenyl | —NH— |
| 369 | cycloalkylenyl | —C(═O)— | — | cycloalkylenyl | —NH— |
| 370 | heterocyclenyl | —C(═O)— | — | cycloalkylenyl | —NH— |
| 371 | — | —C(═O)NH— | — | cycloalkylenyl | —NH— |
| 372 | alkylenyl | —C(═O)NH— | — | cycloalkylenyl | —NH— |
| 373 | cycloalkylenyl | —C(═O)NH— | — | cycloalkylenyl | —NH— |
| 374 | heterocyclenyl | —C(═O)NH— | — | cycloalkylenyl | —NH— |
| 375 | — | —C≡C— | — | cycloalkylenyl | —NH— |
| 376 | alkylenyl | —C≡C— | — | cycloalkylenyl | —NH— |
| 377 | cycloalkylenyl | —C≡C— | — | cycloalkylenyl | —NH— |
| 378 | heterocyclenyl | —C≡C— | — | cycloalkylenyl | —NH— |
| 379 | alkylenyl | — | alkylenyl | cycloalkylenyl | —NH— |
| 380 | cycloalkylenyl | — | alkylenyl | cycloalkylenyl | —NH— |
| 381 | heterocyclenyl | — | alkylenyl | cycloalkylenyl | —NH— |
| 382 | — | —C(═O)— | alkylenyl | cycloalkylenyl | —NH— |
| 383 | alkylenyl | —C(═O)— | alkylenyl | cycloalkylenyl | —NH— |
| 384 | cycloalkylenyl | —C(═O)— | alkylenyl | cycloalkylenyl | —NH— |
| 385 | heterocyclenyl | —C(═O)— | alkylenyl | cycloalkylenyl | —NH— |
| 386 | — | —C(═O)NH— | alkylenyl | cycloalkylenyl | —NH— |
| 387 | alkylenyl | —C(═O)NH— | alkylenyl | cycloalkylenyl | —NH— |
| 388 | cycloalkylenyl | —C(═O)NH— | alkylenyl | cycloalkylenyl | —NH— |
| 389 | heterocyclenyl | —C(═O)NH— | alkylenyl | cycloalkylenyl | —NH— |
| 390 | — | —C≡C— | alkylenyl | cycloalkylenyl | —NH— |
| 391 | alkylenyl | —C≡C— | alkylenyl | cycloalkylenyl | —NH— |
| 392 | cycloalkylenyl | —C≡C— | alkylenyl | cycloalkylenyl | —NH— |
| 393 | heterocyclenyl | —C≡C— | alkylenyl | cycloalkylenyl | —NH— |
| 394 | alkylenyl | — | heterocyclenyl | cycloalkylenyl | —NH— |
| 395 | cycloalkylenyl | — | heterocyclenyl | cycloalkylenyl | —NH— |
| 396 | heterocyclenyl | — | heterocyclenyl | cycloalkylenyl | —NH— |
| 397 | — | —C(═O)— | heterocyclenyl | cycloalkylenyl | —NH— |
| 398 | alkylenyl | —C(═O)— | heterocyclenyl | cycloalkylenyl | —NH— |
| 399 | cycloalkylenyl | —C(═O)— | heterocyclenyl | cycloalkylenyl | —NH— |
| 400 | heterocyclenyl | —C(═O)— | heterocyclenyl | cycloalkylenyl | —NH— |
| 401 | — | —C(═O)NH— | heterocyclenyl | cycloalkylenyl | —NH— |
| 402 | alkylenyl | —C(═O)NH— | heterocyclenyl | cycloalkylenyl | —NH— |
| 403 | cycloalkylenyl | —C(═O)NH— | heterocyclenyl | cycloalkylenyl | —NH— |
| 404 | heterocyclenyl | —C(═O)NH— | heterocyclenyl | cycloalkylenyl | —NH— |
| 405 | — | —C≡C— | heterocyclenyl | cycloalkylenyl | —NH— |
| 406 | alkylenyl | —C≡C— | heterocyclenyl | cycloalkylenyl | —NH— |
| 407 | cycloalkylenyl | —C≡C— | heterocyclenyl | cycloalkylenyl | —NH— |
| 408 | heterocyclenyl | —C≡C— | heterocyclenyl | cycloalkylenyl | —NH— |

In some embodiments, PROTAC Molecules are compounds of any one of Formulae IV-XXX, or a pharmaceutically acceptable salt or solvate thereof, wherein Q is a small molecule that binds to a target protein of interest.

In some embodiments, PROTAC Molecules are compounds of any one of Formulae IV-XXX, or a pharmaceutically acceptable salt or solvate thereof, wherein Q is a Hsp90 inhibitor, a kinase inhibitor, a MDM2 inhibitor, a compound targeting cytosolic signaling protein, a HDAC inhibitor, a human lysine methyltransferase inhibitor, an angiogenesis inhibitor, an immunosuppressive compound, or compound a targeting the aryl hydrocarbon receptor (AHR).

In some embodiments, PROTAC Molecules are compounds of any one of Formulae IV-XXX, or a pharmaceutically acceptable salt or solvate thereof, wherein Q binds to a kinase, a cytosolic signaling protein, e.g., FKBP12, a nuclear protein, a histone deacetylase, a lysine methyltransferase, a protein regulating angiogenesis, a protein regulating immune response, an aryl hydrocarbon receptor (AHR), a glucocorticoid receptor, or a transcription factor, e.g., SMARCA4, SMARCA2, TRTM24.

In some embodiments, PROTAC Molecules are compounds of any one of Formulae IV-XXX, or a pharmaceutically acceptable salt or solvate thereof, wherein Q binds to a kinase, e.g., a tyrosine kinase, e.g., AATK, ABL, ABL2, ALK, AXL, BLK, BMX, BTK, CSF1R, CSK, DDR1, DDR2, EGFR, EPHA1, EPHA2, EPHA3, EPHA4, EPHA5, EPHA6, EPHA7, EPHA8, EPHA10, EPHB1, EPHB2, EPHB3, EPHB4, EPHB6, ERBB2, ERBB3, ERBB4, FER, FES, FGFR1, FGFR2, FGFR3, FGFR4, FGR, FLT1, FLT3, FLT4, FRK, FYN, GSG2, HCK, IGF1R, ILK, INSR, INSRR, IRAK4, ITK, JAK1, JAK2, JAK3, KDR, KIT, KSR1, LCK, LMTK2, LMTK3, LTK, LYN, MATK, MERTK, MET, MLTK, MST1R, MUSK, NPR1, NTRK1, NTRK2, NTRK3, PDGFRA, PDGFRB, PLK4, PTK2, PTK2B, PTK6, PTK7, RET, ROR1, ROR2, ROS1, RYK, SGK493, SRC, SRMS, STYK1, SYK, TEC, TEK, TEX14, TIEl, TNK1, TNK2, TNNI3K, TXK, TYK2, TYRO3, YES1, or ZAP70; a aserine/threonine kinase, e.g., casein kinase 2, protein kinase A, protein kinase B, protein kinase C, Raf kinases, CaM kinases, AKT1, AKT2, AKT3, ALK1, ALK2, ALK3, ALK4, Aurora A, Aurora B, Aurora C, CHK1, CHK2, CLK1, CLK2, CLK3, DAPK1, DAPK2, DAPK3, DMPK, ERK1, ERK2, ERK5, GCK, GSK3, HIPK, KHS1, LKB1, LOK, MAPKAPK2, MAPKAPK, MNK1, MSSK1, MST1, MST2, MST4, NDR, NEK2, NEK3, NEK6, NEK7, NEK9, NEK11, PAK1, PAK2, PAK3, PAK4, PAK5, PAK6, PIM1, PIM2, PLK1, RIP2, RIP5, RSK1, RSK2, SGK2, SGK3, SIK1, STK33, TAO1, TAO2, TGF-beta, TLK2, TSSK1, TSSK2, ULK1, or ULK2; a cyclin dependent kinase, e.g., Cdk1-Cdk11, or a leucine-rich repeat kinase, e.g., LRRK2.

In some embodiments, PROTAC Molecules are compounds of any one of Formulae IV-XXX, or a pharmaceutically acceptable salt or solvate thereof, wherein Q binds to antennapedia homeodomain protein, BRCA1, BRCA2, CCAAT-enhanced-binding proteins, histones, polycomb-group proteins, high mobility group proteins, telomere binding proteins, FANCA, FANCD2, FANCE, FANCF, hepatocyte nuclear factors, Mad2, NF-kappa B, nuclear receptor coactivators, CREB-binding protein, p55, p107, p130, Rb proteins, p53, c-fos, c-jun, c-mdm2, c-myc, or c-rel.

In some embodiments, PROTAC Molecules are compounds of any one of Formulae IV-XXX, or a pharmaceutically acceptable salt or solvate thereof, wherein Q is a half-life extending moiety, see, e.g., Bech et al., ASC Med. Chem. Lett. 9:577-580 (2018), a fluorophore, or a dye.

The present disclosure encompasses the preparation and use of salts of Cereblon Ligands and PROTAC Molecules. As used herein, the pharmaceutical “pharmaceutically acceptable salt” refers to salts or zwitterionic forms of Cereblon Ligands and PROTAC Molecules. Salts of Cereblon Ligands and PROTAC Molecules can be prepared during the final isolation and purification of the compounds or separately by reacting the compound with a suitable acid. The pharmaceutically acceptable salts of Cereblon Ligands and PROTAC Molecules can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Non-limiting examples of salts of Cereblon Ligands and PROTAC Molecules include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and p-toluenesulfonate salts. In addition, available amino groups present in the Cereblon Ligands and PROTAC Molecules can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference Cereblon Ligands and PROTAC Molecules appearing herein is intended to include the compounds as well as pharmaceutically acceptable salts, hydrates, or solvates thereof.

The present disclosure encompasses the preparation and use of solvates of Cereblon Ligands and PROTAC Molecules. Solvates typically do not significantly alter the physiological activity or toxicity of the compounds, and as such may function as pharmacological equivalents. The term “solvate” as used herein is a combination, physical association and/or solvation of a compound of the present disclosure with a solvent molecule such as, e.g. a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Cereblon Ligands and PROTAC Molecules can be present as solvated forms with a pharmaceutically acceptable solvent, such as water, methanol, and ethanol, and it is intended that the disclosure includes both solvated and unsolvated forms of Cereblon Ligands and PROTAC Molecules. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5(1): Article 12 (2004), and A. L. Bingham et al., Chem. Commun. 603-604 (2001). A typical, non-limiting, process of preparing a solvate would involve dissolving a Cereblon Ligand and PROTAC Molecule in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvate in a crystal of the solvate.

Methods of Use

In some aspects, the present disclosure provides methods of inhibiting CRBN ubiquitination in a subject, comprising administering to the subject a Compound of the Disclosure (e.g., a Cereblon Ligand).

In some aspects, the present disclosure provides uses of a Compound of the Disclosure (e.g., a Cereblon Ligand) in the manufacture of a medicament for inhibiting CRBN ubiquitination in a subject.

In some aspects, the present disclosure provides Compounds of the Disclosure (e.g., a Cereblon Ligand) for use in inhibiting CRBN ubiquitination in a subject.

In some aspects, the present disclosure provides methods of degrading a protein in a subject, comprising administering to the subject a Compound of the Disclosure (e.g., a PROTAC Molecule).

In some aspects, the present disclosure provides uses of a Compound of the Disclosure (e.g., a PROTAC Molecule) in the manufacture of a medicament for degrading a protein in a subject.

In some aspects, the present disclosure provides Compounds of the Disclosure (e.g., a PROTAC Molecule) for use in degrading a protein in a subject.

In some aspects, the present disclosure provides methods of treating or preventing a disease (e.g., a disease associated with degradation of a protein) in a subject in need thereof, comprising administering to the subject a Compound of the Disclosure (e.g., a PROTAC Molecule), e.g., in a therapeutically effective amount.

In some aspects, the present disclosure provides methods of treating a disease (e.g., a disease associated with degradation of a protein) in a subject in need thereof, comprising administering to the subject a Compound of the Disclosure (e.g., a PROTAC Molecule), e.g., in a therapeutically effective amount.

In some aspects, the present disclosure provides uses of a Compound of the Disclosure (e.g., a PROTAC Molecule) in the manufacture of a medicament for treating or preventing a disease (e.g., a disease associated with degradation of a protein) in a subject in need thereof.

In some aspects, the present disclosure provides uses of a Compound of the Disclosure (e.g., a PROTAC Molecule) in the manufacture of a medicament for treating a disease (e.g., a disease associated with degradation of a protein) in a subject in need thereof.

In some aspects, the present disclosure provides Compounds of the Disclosure (e.g., a PROTAC Molecule) for use in treating or preventing a disease (e.g., a disease associated with degradation of a protein) in a subject in need thereof.

In some aspects, the present disclosure provides Compounds of the Disclosure (e.g., a PROTAC Molecule) for use in treating a disease (e.g., a disease associated with degradation of a protein) in a subject in need thereof.

In some embodiments, the subject is a mammal.

In some embodiments, the subject is a human.

In some embodiments, the subject is a biological sample (e.g., a cell population).

In some embodiments, the disease is a cancer.

Cereblon Ligands inhibit CRBN ubiquitination and are thus useful in the treatment or prevention of a variety of diseases and conditions. In particular, Cereblon Ligands are useful in methods of treating or preventing a disease or condition wherein inhibition of CRBN ubiquitination provides a benefit. Foremost among these diseases and conditions are cancers and proliferative diseases. In some embodiments, such a cancer is referred to as a “CRBN-mediated cancer.” CRBN-mediated cancers are known in the art. The therapeutic methods of this disclosure comprise administering a therapeutically effective amount of a Cereblon Ligand to a subject, e.g., human, in need thereof. The present methods also encompass optionally administering an optional therapeutic agent to the subject in addition to the Cereblon Ligand.

The optional therapeutic agent is selected from drugs known as useful in treating the disease or condition afflicting the subject in need thereof, e.g., a chemotherapeutic agent and/or radiation known as useful in treating a particular cancer.

In some embodiments, the present disclosure is directed to a method of inhibiting CRBN ubiquitination in a subject in need thereof, said method comprising administering to the subject an effective amount of a Cereblon Ligand.

PROTAC Molecules are protein degraders. Protein degraders are useful in methods of treating or preventing a disease or condition wherein degradation of one or more proteins provides a benefit, for example, cancers and proliferative diseases. The therapeutic methods of this disclosure comprise administering a therapeutically effective amount of a PROTAC Molecule to a subject in need thereof. The present methods also encompass administering a second therapeutic agent to the subject in addition to the PROTAC Molecule. The second therapeutic agent is selected from drugs known as useful in treating the disease or condition afflicting the subject in need thereof, e.g., a chemotherapeutic agent and/or radiation known as useful in treating a particular cancer.

PROTAC Molecules typically have DC₅₀ (the drug concentration that results in 50% target protein degradation) values of less than 100 μM, e.g., less than 50 μM, less than 25 μM, and less than 5 μM, less than about 1 μM, less than about 0.5 μM, or less than about 0.1 μM.

In some embodiments, PROTAC Molecules have DC₅₀ values of less than about 0.05 μM. In some embodiments, PROTAC Molecule have DC₅₀ values of less than about 0.01 μM. In some embodiments, the present disclosure relates to a method of treating a subject suffering from a disease or condition wherein the degradation of one or more proteins provides a benefit comprising administering a therapeutically effective amount of a PROTAC Molecule to a subject in need thereof.

Since Cereblon Ligands inhibit CRBN ubiquitination, a number of diseases and conditions mediated by CRBN ubiquitination can be treated by employing these compounds.

The present disclosure is thus directed generally to a method for treating a condition or disorder responsive to inhibition of CRBN ubiquitination in a subject, e.g., a human subject, suffering from, or at risk of suffering from, a condition or disorder, e.g., cancer or inflammatory disease, the method comprising administering to the subject an effective amount of a Cereblon Ligand.

The methods of the present disclosure can be accomplished by administering a Cereblon Ligand or PROTAC Molecule as the neat compound or as a pharmaceutical composition. Administration of a pharmaceutical composition, or neat compound of a Cereblon Ligand or PROTAC Molecule, can be performed during or after the onset of the disease or condition of interest. Typically, the pharmaceutical compositions are sterile, and contain no toxic, carcinogenic, or mutagenic compounds that would cause an adverse reaction when administered. Further provided are kits comprising a Cereblon Ligand or PROTAC Molecule and, optionally, an optional therapeutic agent, packaged separately or together, and an insert having instructions for using these active agents.

In some embodiments, a Cereblon Ligand is administered in conjunction with an optional therapeutic agent useful in the treatment of a disease or condition wherein inhibition of CRBN ubiquitination provides a benefit. The optional therapeutic agent is different from the Cereblon Ligand. A Cereblon Ligand and the optional therapeutic agent can be administered simultaneously or sequentially to achieve the desired effect. In addition, the Cereblon Ligand and optional therapeutic agent can be administered from a single composition or two separate compositions. Likewise, in some embodiments, a PROTAC Molecule is administered in conjunction with an optional therapeutic agent.

The optional therapeutic agent is administered in an amount to provide its desired therapeutic effect. The effective dosage range for each optional therapeutic agent is known in the art, and the optional therapeutic agent is administered to an individual in need thereof within such established ranges.

A Cereblon Ligand or PROTAC Molecule and the optional therapeutic agent can be administered together as a single-unit dose or separately as multi-unit doses, wherein the Cereblon Ligand or PROTAC Molecule is administered before the optional therapeutic agent or vice versa. One or more doses of the Cereblon Ligand and/or one or more dose of the optional therapeutic agent can be administered. The Cereblon Ligand or PROTAC Molecule therefore can be used in conjunction with one or more optional therapeutic agents, for example, but not limited to, anticancer agents.

Diseases and conditions treatable by the methods of the present disclosure include, but are not limited to, cancer and other proliferative disorders, or an inflammatory disease. In some embodiments, a human subject is treated with a Cereblon Ligand, or a pharmaceutical composition comprising a Cereblon Ligand, wherein the compound is administered in an amount sufficient to inhibit CRBN ubiquitination in the subject.

In some aspects, the present disclosure provides a method of treating cancer in a subject comprising administering a therapeutically effective amount of a Cereblon Ligand. While not being limited to a specific mechanism, in some embodiments, Cereblon Ligands treat cancer by inhibiting CRBN ubiquitination.

In some aspects, the present disclosure provides a method of treating cancer in a subject comprising administering a therapeutically effective amount of a PROTAC Molecule to the subject.

Examples of treatable cancers include, but are not limited to, any one or more of the cancers of Table I.

TABLE I

| | | | |
|---|---|---|---|
| adrenal cancer | acinic cell | acoustic neuroma | acral lentigious |
| | carcinoma | | melanoma |
| acrospiroma | acute | acute erythroid | acute |
| | eosinophilic | leukemia | lymphoblastic |
| | leukemia | | leukemia |
| acute | acute monocytic | acute | adenocarcinoma |
| megakaryoblastic | leukemia | promyelocytic | |
| leukemia | | leukemia | |
| adenoid cystic | adenoma | adenomatoid | adenosquamous |
| carcinoma | | odontogenic | carcinoma |
| | | tumor | |
| adipose tissue | adrenocortical | adult T-cell | aggressive NK- |
| neoplasm | carcinoma | leukemia/ | cell leukemia |
| | | lymphoma | |
| AIDS-related | alveolar | alveolar soft part | ameloblastic |
| lymphoma | rhabdo- | sarcoma | fibroma |
| | myosarcoma | | |
| anaplastic | anaplastic | angioimmuno- | angio- |
| large cell | thyroid | blastic | myolipoma |
| lymphoma | cancer | T-cell lymphoma | |
| angiosarcoma | astrocytoma | atypical teratoid | B-cell chronic |
| | | rhabdoid tumor | lymphocytic |
| | | | leukemia |
| B-cell | B-cell | basal cell | biliary tract |
| prolymphocytic | lymphoma | carcinoma | cancer |
| leukemia | | | |
| bladder cancer | blastoma | bone cancer | Brenner tumor |
| Brown tumor | Burkitt's | breast cancer | brain cancer |
| | lymphoma | | |
| carcinoma | carcinoma in situ | carcinosarcoma | cartilage tumor |
| cementoma | myeloid sarcoma | chondroma | chordoma |
| choriocarcinoma | choroid plexus | clear-cell sarcoma | cranio- |
| | papilloma | of the kidney | pharyngioma |
| cutaneous T-cell | cervical cancer | colorectal cancer | Degos disease |
| lymphoma | | | |
| desmoplastic | diffuse large | dysembryoplastic | dysgerminoma |
| small round | B-cell | neuroepithelial | |
| cell tumor | lymphoma | tumor | |
| embryonal | endocrine gland | endodermal sinus | enteropathy- |
| carcinoma | neoplasm | tumor | associated |
| | | | T-cell |
| | | | lymphoma |
| esophageal | fetus in fetu | fibroma | fibrosarcoma |
| cancer | | | |
| follicular | follicular thyroid | ganglioneuroma | gastrointestinal |
| lymphoma | cancer | | cancer |
| germ cell tumor | gestational | giant cell | giant cell tumor |
| | choriocarcinoma | fibroblastoma | of the bone |
| glial tumor | glioblastoma | glioma | gliomatosis |
| | multiforme | | cerebri |
| glucagonoma | gonadoblastoma | granulosa | gynandro- |
| | | cell tumor | blastoma |
| gallbladder | gastric cancer | hairy cell | hemangio- |
| cancer | | leukemia | blastoma |
| head and neck | hemangio- | hematological | hepatoblastoma |
| cancer | pericytoma | cancer | |
| hepatosplenic | Hodgkin's | non-Hodgkin's | invasive lobular |
| T-cell | lymphoma | lymphoma | carcinoma |
| lymphoma | | | |
| intestinal cancer | kidney cancer | laryngeal cancer | lentigo maligna |
| lethal midline | leukemia | leydig cell tumor | liposarcoma |
| carcinoma | | | |
| lung cancer | lymphangioma | lymphangio- | lympho- |
| | | sarcoma | epithelioma |
| lymphoma | acute | acute | chronic |
| | lymphocytic | myelogeous | lymphocytic |
| | leukemia | leukemia | leukemia |
| liver cancer | small cell lung | non-small cell | MALT |
| | cancer | lung cancer | lymphoma |
| malignant fibrous | malignant | malignant triton | mantle cell |
| histiocytoma | peripheral nerve | tumor | lymphoma |
| | sheath tumor | | |
| marginal zone B- | mast cell | mediastinal germ | medullary |
| cell lymphoma | leukemia | cell tumor | carcinoma of |
| | | | the breast |
| medullary | medulloblastoma | melanoma | meningioma |
| thyroid cancer | | | |
| merkel cell | mesothelioma | metastatic | mixed |
| cancer | | urothelial | Mullerian |
| | | carcinoma | tumor |
| mucinous tumor | multiple | muscle tissue | mycosis |
| | myeloma | neoplasm | fungoides |
| myxoid | myxoma | myxosarcoma | nasopharyngeal |
| liposarcoma | | | carcinoma |
| neurinoma | neuroblastoma | neurofibroma | neuroma |
| nodular | ocular cancer | oligoastro- | oligodendro- |
| melanoma | | cytoma | glioma |
| oncocytoma | optic nerve | optic nerve tumor | oral cancer |
| | sheath | | |
| | meningioma | | |
| osteosarcoma | ovarian cancer | Pancoast tumor | papillary |
| | | | thyroid cancer |
| paraganglioma | pinealoblastoma | pineocytoma | pituicytoma |
| pituitary adenoma | pituitary tumor | plasmacytoma | polyembryoma |
| precursor T- | primary central | primary effusion | preimary |
| lymphoblastic | nervous system | lymphoma | peritoneal |
| lymphoma | lymphoma | | cancer |
| prostate cancer | pancreatic | pharyngeal | pseudomyxoma |
| | cancer | cancer | periotonei |
| renal cell | renal medullary | retinoblastoma | rhabdomyoma |
| carcinoma | carcinoma | | |
| rhabdomyo- | Richter's | rectal cancer | sarcoma |
| sarcoma | transformation | | |
| Schwannomatosis | seminoma | Sertoli cell tumor | sex cord- |
| | | | gonadal |
| | | | stromal tumor |
| signet ring cell | skin cancer | small blue round | small cell |
| carcinoma | | cell tumors | carcinoma |
| soft tissue | somatostatinoma | soot wart | spinal tumor |
| sarcoma | | | |
| splenic marginal | squamous cell | synovial sarcoma | Sezary's disease |
| zone lymphoma | carcinoma | | |
| small intestine | squamous | stomach cancer | T-cell |
| cancer | carcinoma | | lymphoma |
| testicular cancer | thecoma | thyroid cancer | transitional cell |
| | | | carcinoma |
| throat cancer | urachal cancer | urogenital cancer | urothelial |
| | | | carcinoma |
| uveal melanoma | uterine cancer | verrucous | visual pathway |
| | | carcinoma | glioma |
| vulvar cancer | vaginal cancer | Waldenstrom's | Warthin's tumor |
| | | macro- | |
| | | globulinemia | |
| Wilms' tumor | | | |

In some embodiments, the cancer is a solid tumor. In some embodiments, the cancer a hematological cancer. Exemplary hematological cancers include, but are not limited to, the cancers listed in Table II. In some embodiments, the hematological cancer is acute lymphocytic leukemia, chronic lymphocytic leukemia (including B-cell chronic lymphocytic leukemia), or acute myeloid leukemia. In some embodiments, the hematological cancer is multiple myeloma.

TABLE II

| | |
|---|---|
| acute lymphocytic leukemia (ALL) | acute eosinophilic leukemia |
| acute myeloid leukemia (AML) | acute erythroid leukemia |
| chronic lymphocytic leukemia (CLL) | acute lymphoblastic leukemia |
| small lymphocytic lymphoma (SLL) | acute megakaryoblastic leukemia |
| multiple myeloma (MM) | acute monocytic leukemia |
| Hodgkins lymphoma (HL) | acute promyelocytic leukemia |
| non-Hodgkin's lymphoma (NHL) | acute myelogeous leukemia |
| mantle cell lymphoma (MCL) | B-cell prolymphocytic leukemia |
| marginal zone B-cell lymphoma | B-cell lymphoma |
| splenic marginal zone lymphoma | MALT lymphoma |
| follicular lymphoma (FL) | precursor T-lymphoblastic |
| | lymphoma |
| Waldenstrom's macroglobulinemia | T-cell lymphoma |
| (WM) | |
| diffuse large B-cell lymphoma | mast cell leukemia |
| (DLBCL) | |
| marginal zone lymphoma (MZL) | adult T cell leukemia/lymphoma |
| hairy cell leukemia (HCL) | aggressive NK-cell leukemia |
| Burkitt's lymphoma (BL) | angioimmunoblastic T-cell |
| | lymphoma |
| Richter's transformation | |

In some embodiments, the cancer is a leukemia, for example a leukemia selected from acute monocytic leukemia, acute myelogenous leukemia, chronic myelogenous leukemia, chronic lymphocytic leukemia and mixed lineage leukemia (MLL). In another embodiment the cancer is NUT-midline carcinoma. In another embodiment the cancer is multiple myeloma. In another embodiment the cancer is a lung cancer such as small cell lung cancer (SCLC). In another embodiment the cancer is a neuroblastoma. In another embodiment the cancer is Burkitt's lymphoma. In another embodiment the cancer is cervical cancer. In another embodiment the cancer is esophageal cancer. In another embodiment the cancer is ovarian cancer. In another embodiment the cancer is colorectal cancer. In some embodiments, the cancer is prostate cancer. In some embodiments, the cancer is breast cancer.

In some embodiments, the cancer is selected from acute monocytic leukemia, acute myelogenous leukemia, chronic myelogenous leukemia, chronic lymphocytic leukemia mixed lineage leukemia, NUT-midline carcinoma, multiple myeloma, small cell lung cancer, non-small cell lung cancer, neuroblastoma, Burkitt's lymphoma, cervical cancer, esophageal cancer, ovarian cancer, colorectal cancer, prostate cancer, breast cancer, bladder cancer, ovary cancer, glioma, sarcoma, esophageal squamous cell carcinoma, and papillary thyroid carcinoma.

In some embodiments, the present disclosure provides a method of treating a benign proliferative disorder, such as, but are not limited to, benign soft tissue tumors, bone tumors, brain and spinal tumors, eyelid and orbital tumors, granuloma, lipoma, meningioma, multiple endocrine neoplasia, nasal polyps, pituitary tumors, prolactinoma, pseudotumor cerebri, seborrheic keratoses, stomach polyps, thyroid nodules, cystic neoplasms of the pancreas, hemangiomas, vocal cord nodules, polyps, and cysts, Castleman disease, chronic pilonidal disease, dermatofibroma, pilar cyst, pyogenic granuloma, and juvenile polyposis syndrome.

In some embodiments, the present disclosure provides a method of treating an inflammatory disease. For example, Cereblon Ligands can be used to treat infectious and noninfectious inflammatory events and autoimmune and other inflammatory diseases by administration of a therapeutically effective amount to a subject, in particular a human in need of such treatment. Examples of autoimmune and inflammatory diseases, disorders, and syndromes treated using the compounds and methods described herein include inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, Type I diabetes, septic shock, lupus, e.g., cutaneous lupus, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituatarism, Guillain-Barre syndrome, Behcet's disease, scleracierma, mycosis fungoides, acute inflammatory responses (such as acute respiratory distress syndrome and ischemia/reperfusion injury), and Graves' disease.

In some embodiments, the present disclosure provides a therapeutic method of modulating CRBN ubiquitination in vivo in diseases mentioned above, in particular cancer, by administering a therapeutically effective amount of a Cereblon Ligand to a subject in need of such therapy.

In methods of the present disclosure, a therapeutically effective amount of a Cereblon Ligand or PROTAC Molecule, typically formulated in accordance with pharmaceutical practice, is administered to a human being in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

A Cereblon Ligand or PROTAC Molecule can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high pressure technique.

Pharmaceutical compositions include those wherein a Cereblon Ligand or PROTAC Molecule is administered in an effective amount to achieve its intended purpose. The exact formulation, route of administration, and dosage is determined by an individual physician in view of the diagnosed condition or disease. Dosage amount and interval can be adjusted individually to provide levels of a Cereblon Ligand or PROTAC Molecule that is sufficient to maintain therapeutic effects.

Toxicity and therapeutic efficacy of the Cereblon Ligands or the PROTAC Molecules can be determined by standard pharmaceutical procedures in cell cultures or experimental animals, e.g., for determining the maximum tolerated dose (MTD) of a compound, which defines as the highest dose that causes no toxicity in animals. The dose ratio between the maximum tolerated dose and therapeutic effects (e.g. inhibiting of tumor growth) is the therapeutic index. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of a Cereblon Ligand or PROTAC Molecule required for use in therapy varies with the nature of the condition being treated, the length of time that activity is desired, and the age and the condition of the subject, and ultimately is determined by the attendant physician. Dosage amounts and intervals can be adjusted individually to provide plasma levels of the Cereblon Ligand that are sufficient to maintain the desired therapeutic effects. The desired dose can be administered in a single dose, or as multiple doses administered at appropriate intervals, for example as one, two, three, four or more subdoses per day. Multiple doses often are desired, or required. For example, a Cereblon Ligand can be administered at a frequency of: four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

A Cereblon Ligand or PROTAC Molecule used in a method of the present disclosure can be administered in an amount of about 0.005 to about 500 milligrams per dose, about 0.05 to about 250 milligrams per dose, or about 0.5 to about 100 milligrams per dose. For example, a Cereblon Ligand or PROTAC Molecule can be administered, per dose, in an amount of about 0.005, about 0.05, about 0.5, about 5, about 10, about 20, about 30, about 40, about 50, about 100, about 150, about 200, about 250, about 300, about 350, about 400, about 450, or about 500 milligrams, including all doses between 0.005 and 500 milligrams.

The dosage of a composition containing a Cereblon Ligand or PROTAC Molecule, or a composition containing the same, can be from about 1 ng/kg to about 200 mg/kg, about 1 g/kg to about 100 mg/kg, or about 1 mg/kg to about 50 mg/kg. The above dosages are exemplary of the average case, but there can be individual instances in which higher or lower dosages are merited, and such are within the scope of this disclosure. In practice, the physician determines the actual dosing regimen that is most suitable for an individual subject, which can vary with the age, weight, and response of the particular subject.

Cereblon Ligands and PROTAC Molecules typically are administered in admixture with a pharmaceutical carrier to give a pharmaceutical composition selected with regard to the intended route of administration and standard pharmaceutical practice. Pharmaceutical compositions for use in accordance with the present disclosure are formulated in a conventional manner using one or more physiologically acceptable carriers comprising excipients and/or auxiliaries that facilitate processing of Cereblon Ligand or PROTAC Molecule.

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of the Cereblon Ligand is administered orally, the composition typically is in the form of a tablet, capsule, powder, solution, or elixir.

When administered in tablet form, the composition additionally can contain a solid carrier, such as a gelatin or an adjuvant. The tablet, capsule, and powder contain about 0.01% to about 95%, and preferably from about 1% to about 50%, of a Cereblon Ligand or PROTAC Molecule.

When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a Cereblon Ligand or PROTAC Molecule.

When a therapeutically effective amount of a Cereblon Ligand or PROTAC Molecule is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, an isotonic vehicle.

Cereblon Ligands or PROTAC Molecules can be readily combined with pharmaceutically acceptable carriers well-known in the art. Standard pharmaceutical carriers are described in Remington's Pharmaceutical Sciences, Mack Publishing Co., Easton, PA, 19th ed. 1995. Such carriers enable the active agents to be formulated as tablets, pills, dragees, capsules, liquids, gels, syrups, slurries, suspensions and the like, for oral ingestion by a subject to be treated. Pharmaceutical preparations for oral use can be obtained by adding the Cereblon Ligand to a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

Cereblon Ligands or PROTAC Molecules can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of a Cereblon Ligand or PROTAC Molecule can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

Cereblon Ligands or PROTAC Molecules also can be formulated in rectal compositions, such as suppositories or retention enemas, e.g., containing conventional suppository bases. In addition to the formulations described previously, the Cereblon Ligand also can be formulated as a depot preparation. Such long-acting formulations can be administered by implantation (for example, subcutaneously or intramuscularly) or by intramuscular injection. Thus, for example, the Cereblon Ligand can be formulated with suitable polymeric or hydrophobic materials (for example, as an emulsion in an acceptable oil) or ion exchange resins.

In particular, the Cereblon Ligands or PROTAC Molecules can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. Cereblon Ligands or PROTAC Molecules also can be injected parenterally, for example, intravenously, intramuscularly, subcutaneously, or intracoronarily. For parenteral administration, the Cereblon Ligands or PROTAC Molecules are typically used in the form of a sterile aqueous solution which can contain other substances, for example, salts or monosaccharides, such as mannitol or glucose, to make the solution isotonic with blood.

Optional Therapeutic Agents

In some therapeutic methods and uses of the disclosure, a Cereblon Ligand or PROTAC Molecule is administered to a subject having a disease, disorder, or condition, e.g., cancer, as a single agent. In other therapeutic methods and uses of the disclosure, a Cereblon Ligand or PROTAC Molecule is administered to a subject having a disease, disorder, or condition, e.g., cancer, in combination with one or more optional therapeutic agents. In some embodiments, a Cereblon Ligand or PROTAC Molecule is administered in combination with one optional therapeutic agent. In some embodiments, a Cereblon Ligand or PROTAC Molecule is administered in combination with two optional therapeutic agents. In some embodiments, a Cereblon Ligand or PROTAC Molecule is administered in combination with three optional therapeutic agents. Optional therapeutic agents useful in treating cancer patients include those known in the art as well as those developed in the future.

Optional therapeutic agents are administered in an amount to provide their desired therapeutic effect. The effective dosage range for each optional therapeutic agent is known in the art, and the optional therapeutic agent is administered to an individual in need thereof within such established ranges.

A Cereblon Ligand or PROTAC Molecule and the optional therapeutic agent(s) can be administered together as a single-unit dose or separately as multi-unit doses, and in any order, e.g., wherein a Cereblon Ligand is administered before the optional therapeutic agent(s), or vice versa. One or more doses of a Cereblon Ligand or PROTAC Molecule and the optional therapeutic agent(s) can be administered to the subject.

In some embodiments, the optional therapeutic agent is an immune checkpoint inhibitor. Immune checkpoint inhibitors are therapies that blockade immune system inhibitor checkpoints. Immune checkpoints can be stimulatory or inhibitory. Blockade of inhibitory immune checkpoint activates immune system function and can be used for cancer immunotherapy. Pardoll, Nature Reviews. Cancer 12:252-64 (2012). Tumor cells turn off activated T cells when they attach to specific T-cell receptors. Immune checkpoint inhibitors prevent tumor cells from attaching to T cells, which results in T cells remaining activated. In effect, the coordinated action by cellular and soluble components combats pathogens and injuries by cancers. The modulation of immune system pathways may involve changing the expression or the functional activity of at least one component of the pathway to then modulate the response by the immune system. U.S. 2015/0250853. Examples of immune checkpoint inhibitors include PD-1 inhibitors, PD-L1 inhibitors, CTLA-4 inhibitors, LAG3 inhibitors, TIM3 inhibitors, cd47 inhibitors, and B7-H1 inhibitors. Thus, in some embodiments, the immune checkpoint inhibitor is selected from a PD-1 inhibitor, a PD-L1 inhibitor, a CTLA-4 inhibitor, a LAG3 inhibitor, a TIM3 inhibitor, and a cd47 inhibitor.

In some embodiments, the immune checkpoint inhibitor is a programmed cell death (PD-1) inhibitor. PD-1 is a T-cell coinhibitory receptor that plays a pivotal role in the ability of tumor cells to evade the host's immune system. Blockage of interactions between PD-1 and PD-L1, a ligand of PD-1, enhances immune function and mediates antitumor activity. Examples of PD-1 inhibitors include antibodies that specifically bind to PD-1. Particular anti-PD-1 antibodies include, but are not limited to nivolumab, pembrolizumab, STI-A1014, pidilzumab, and cemiplimab-rwlc. For a general discussion of the availability, methods of production, mechanism of action, and clinical studies of anti-PD-1 antibodies, see U.S. 2013/0309250, U.S. Pat. Nos. 6,808,710, 7,595,048, 8,008,449, 8,728,474, 8,779,105, 8,952,136, 8,900,587, 9,073,994, 9,084,776, and Naido et al., British Journal of Cancer 111:2214-19 (2014).

In some embodiments, the immune checkpoint inhibitor is a PD-L1 (also known as B7-H1 or CD274) inhibitor. Examples of PD-L1 inhibitors include antibodies that specifically bind to PD-L1. Particular anti-PD-L1 antibodies include, but are not limited to, avelumab, atezolizumab, durvalumab, and BMS-936559. For a general discussion of the availability, methods of production, mechanism of action, and clinical studies, see U.S. Pat. No. 8,217,149, U.S. 2014/0341917, U.S. 2013/0071403, WO 2015036499, and Naido et al., British Journal of Cancer 111:2214-19 (2014).

In some embodiments, the immune checkpoint inhibitor is a CTLA-4 inhibitor. CTLA-4, also known as cytotoxic T-lymphocyte antigen 4, is a protein receptor that downregulates the immune system. CTLA-4 is characterized as a “brake” that binds costimulatory molecules on antigen-presenting cells, which prevents interaction with CD28 on T cells and also generates an overtly inhibitory signal that constrains T cell activation. Examples of CTLA-4 inhibitors include antibodies that specifically bind to CTLA-4. Particular anti-CTLA-4 antibodies include, but are not limited to, ipilimumab and tremelimumab. For a general discussion of the availability, methods of production, mechanism of action, and clinical studies, see U.S. Pat. Nos. 6,984,720, 6,207,156, and Naido et al., British Journal of Cancer 111:2214-19 (2014).

In some embodiments, the immune checkpoint inhibitor is a LAG3 inhibitor. LAG3, Lymphocyte Activation Gene 3, is a negative co-simulatory receptor that modulates T cell homeostatis, proliferation, and activation. In addition, LAG3 has been reported to participate in regulatory T cells (Tregs) suppressive function. A large proportion of LAG3 molecules are retained in the cell close to the microtubule-organizing center, and only induced following antigen specific T cell activation. U.S. 2014/0286935. Examples of LAG3 inhibitors include antibodies that specifically bind to LAG3. Particular anti-LAG3 antibodies include, but are not limited to, GSK2831781. For a general discussion of the availability, methods of production, mechanism of action, and studies, see, U.S. 2011/0150892, U.S. 2014/0093511, U.S. 20150259420, and Huang et al., Immunity 21:503-13 (2004).

In some embodiments, the immune checkpoint inhibitor is a TIM3 inhibitor. TIM3, T-cell immunoglobulin and mucin domain 3, is an immune checkpoint receptor that functions to limit the duration and magnitude of T_{H}1 and T_{C}1 T-cell responses. The TIM3 pathway is considered a target for anticancer immunotherapy due to its expression on dysfunctional CD8⁺ T cells and Tregs, which are two reported immune cell populations that constitute immunosuppression in tumor tissue. Anderson, Cancer Immunology Research 2:393-98 (2014). Examples of TIM3 inhibitors include antibodies that specifically bind to TIM3. For a general discussion of the availability, methods of production, mechanism of action, and studies of TIM3 inhibitors, see U.S. 20150225457, U.S. 20130022623, U.S. Pat. No. 8,522,156, Ngiow et al., Cancer Res 71: 6567-71 (2011), Ngiow, et al., Cancer Res 71:3540-51 (2011), and Anderson, Cancer Immunology Res 2:393-98 (2014).

In some embodiments, the immune checkpoint inhibitor is a cd47 inhibitor. See Unanue, E. R., PNAS 110:10886-87 (2013).

The term “antibody” is meant to include intact monoclonal antibodies, polyclonal antibodies, multispecific antibodies formed from at least two intact antibodies, and antibody fragments, so long as they exhibit the desired biological activity. In some embodiments, “antibody” is meant to include soluble receptors that do not possess the Fc portion of the antibody. In some embodiments, the antibodies are humanized monoclonal antibodies and fragments thereof made by means of recombinant genetic engineering.

Another class of immune checkpoint inhibitors include polypeptides that bind to and block PD-1 receptors on T-cells without triggering inhibitor signal transduction. Such peptides include B7-DC polypeptides, B7-H1 polypeptides, B7-1 polypeptides and B7-2 polypeptides, and soluble fragments thereof, as disclosed in U.S. Pat. No. 8,114,845.

Another class of immune checkpoint inhibitors include compounds with peptide moieties that inhibit PD-1 signaling. Examples of such compounds are disclosed in U.S. Pat. No. 8,907,053 and have the structure:

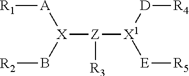

or a pharmaceutically acceptable salt thereof, wherein the compound comprises at least 5 amino acids useful as therapeutic agents capable of inhibiting the PD-1 signaling pathway.

Another class of immune checkpoint inhibitors include inhibitors of certain metabolic enzymes, such as indoleamine 2,3 dioxygenase (IDO), which is expressed by infiltrating myeloid cells and tumor cells, and isocitrate dehydrogenase (IDH), which is mutated in leukemia cells. Mutants of the IDH enzyme lead to increased levels of 2-hydroxyglutarate (2-HG), which prevent myeloid differentiation. Stein et al., Blood 130:722-31 (2017); Wouters, Blood 130:693-94 (2017). Particular mutant IDH blocking agents include, but are not limited to, ivosidenib and enasidenib mesylate. Dalle and DiNardo, Ther Adv Hematol 9(7):163-73 (2018); Nassereddine et al., Onco Targets Ther 12:303-08 (2018). The IDO enzyme inhibits immune responses by depleting amino acids that are necessary for anabolic functions in T cells or through the synthesis of particular natural ligands for cytosolic receptors that are able to alter lymphocyte functions. Pardoll, Nature Reviews. Cancer 12:252-64 (2012); Löb, Cancer Immunol Immunother 58:153-57 (2009). Particular IDO blocking agents include, but are not limited to, levo-1-methyl typtophan (L-1MT) and 1-methyl-tryptophan (1MT). Qian et al., Cancer Res 69:5498-504 (2009); and Lob et al., Cancer Immunol Immunother 58:153-7 (2009).

In some embodiments, the immune checkpoint inhibitor is nivolumab, pembrolizumab, pidilizumab, STI-A1110, avelumab, atezolizumab, durvalumab, STI-A1014, ipilimumab, tremelimumab, GSK2831781, BMS-936559 or MED14736.

In some embodiments, the optional therapeutic agent is an epigenetic drug. As used herein, the term “epigenetic drug” refers to a therapeutic agent that targets an epigenetic regulator. Examples of epigenetic regulators include the histone lysine methyltransferases, histone arginine methyl transferases, histone demethylases, histone deacetylases, histone acetylases, and DNA methyltransferases. Histone deacetylase inhibitors include, but are not limited to, vorinostat and panobinostat lactate.

In some embodiments, the optional therapeutic agent is a chemotherapeutic agent or other anti-proliferative agent that can be administered in combination with a Cereblon Ligand to treat cancer. Examples of conventional therapies and anticancer agents that can be used in combination with a Cereblon Ligand include surgery, radiotherapy (e.g., gamma-radiation, neutron beam radiotherapy, electron beam radiotherapy, proton therapy, brachytherapy, and systemic radioactive isotopes), endocrine therapy, a biologic response modifier (e.g., an interferon, an interleukin, tumor necrosis factor (TNF), hyperthermia and cryotherapy, an agent to attenuate any adverse effect (e.g., an antiemetic), and any other approved biologic therapy or chemotherapy, e.g., a treatment regimen that uses drugs to stop the growth of cancer cells, either by killing the cells or by stopping them from dividing. Chemotherapy may be given by mouth, injection, or infusion, or on the skin, depending on the type and stage of the cancer being treated.

Nonlimiting exemplary antiproliferative compounds include an aromatase inhibitor; an anti-estrogen; an anti-androgen; a gonadorelin agonist; a topoisomerase I inhibitor; a topoisomerase II inhibitor; a microtubule active agent; an alkylating agent, e.g., temozolomide; a retinoid, a carontenoid, or a tocopherol; a cyclooxygenase inhibitor; an MMP inhibitor; an mTOR inhibitor; an antimetabolite; a platin compound; a methionine aminopeptidase inhibitor; a bisphosphonate; an antiproliferative antibody; a heparanase inhibitor; an inhibitor of Ras oncogenic isoforms; a telomerase inhibitor; a proteasome inhibitor; a compound used in the treatment of hematologic malignancies; a Flt-3 inhibitor; an Hsp90 inhibitor; a kinesin spindle protein inhibitor; a MEK inhibitor; an antitumor antibiotic; a nitrosourea; a compound targeting/decreasing protein or lipid kinase activity, a compound targeting/decreasing protein or lipid phosphatase activity, or any further anti-angiogenic compound.

Nonlimiting exemplary aromatase inhibitors include steroids, such as atamestane, exemestane, and formestane, and non-steroids, such as aminoglutethimide, roglethimide, pyridoglutethimide, trilostane, testolactone, ketokonazole, vorozole, fadrozole, anastrozole, and letrozole.

Nonlimiting anti-estrogens include tamoxifen, fulvestrant, raloxifene, and raloxifene hydrochloride. Anti-androgens include, but are not limited to, bicalutamide and apalutamide. Gonadorelin agonists include, but are not limited to, abarelix, goserelin, and goserelin acetate.

Nonlimiting exemplary topoisomerase I inhibitors include topotecan, gimatecan, irinotecan, camptothecin and its analogues, 9-nitrocamptothecin, and the macromolecular camptothecin conjugate PNU-166148. Topoisomerase II inhibitors include, but are not limited to, anthracyclines, such as doxorubicin, daunorubicin, epirubicin, idarubicin, and nemorubicin; anthraquinones, such as mitoxantrone and losoxantrone; and podophillotoxines, such as etoposide and teniposide.

Microtubule active agents include microtubule stabilizing, microtubule destabilizing compounds, and microtubulin polymerization inhibitors including, but not limited to, taxanes, such as paclitaxel and docetaxel; discodermolides; cochicine and epothilones and derivatives thereof.

Nonlimiting exemplary alkylating agents include cyclophosphamide, ifosfamide, melphalan, trabectedin, and nitrosoureas, such as carmustine and lomustine.

Nonlimiting exemplary matrix metalloproteinase inhibitors (“MMP inhibitors”) include collagen peptidomimetic and nonpeptidomimetic inhibitors, tetracycline derivatives, batimastat, marimastat, prinomastat, metastat, BMS-279251, BAY 12-9566, TAA211, MMI270B, and AAJ996.

Nonlimiting exemplary mTOR inhibitors include compounds that inhibit the mammalian target of rapamycin (mTOR) and possess antiproliferative activity such as sirolimus, everolimus, CCI-779, and ABT578.

Nonlimiting exemplary antimetabolites include 5-fluorouracil (5-FU), capecitabine, gemcitabine, DNA demethylating compounds, such as 5-azacytidine and decitabine, methotrexate and edatrexate, and folic acid antagonists, such as pemetrexed.

Nonlimiting exemplary platin compounds include carboplatin, cis-platin, cisplatinum, and oxaliplatin.

Nonlimiting exemplary methionine aminopeptidase inhibitors include bengamide or a derivative thereof and PPI-2458.

Nonlimiting exemplary bisphosphonates include etridonic acid, clodronic acid, tiludronic acid, pamidronic acid, alendronic acid, ibandronic acid, risedronic acid, and zoledronic acid.

Nonlimiting exemplary heparanase inhibitors include compounds that target, decrease, or inhibit heparin sulfate degradation, such as PI-88 and OGT2115.

Nonlimiting exemplary compounds which target, decrease, or inhibit the oncogenic activity of Ras include farnesyl transferase inhibitors, such as L-744832, DK8G557, tipifarnib, and lonafarnib.

Nonlimiting exemplary telomerase inhibitors include compounds that target, decrease, or inhibit the activity of telomerase, such as compounds that inhibit the telomerase receptor, such as telomestatin.

Nonlimiting exemplary proteasome inhibitors include compounds that target, decrease, or inhibit the activity of the proteasome including, but not limited to, bortezomib. In some embodiments, the proteasome inhibitor is carfilzomib or ixazomib.

Nonlimiting exemplary FMS-like tyrosine kinase inhibitors, which are compounds targeting, decreasing or inhibiting the activity of FMS-like tyrosine kinase receptors (Flt-3R), include gilteritinib, interferon, I-j-D-arabinofuransylcytosine (ara-c), and bisulfan; and ALK inhibitors, which are compounds that target, decrease, or inhibit anaplastic lymphoma kinase, include alectinib, brigatinib, and lorlatinib.

Nonlimiting exemplary Flt-3 inhibitors include PKC412, midostaurin, a staurosporine derivative, SU11248, MLN518, and gilteritinib.

Nonlimiting exemplary HSP90 inhibitors include compounds targeting, decreasing, or inhibiting the intrinsic ATPase activity of HSP90; or degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins, or antibodies that inhibit the ATPase activity of HSP90, such as 17-allylamino,17-demethoxygeldanamycin (17AAG), a geldanamycin derivative; other geldanamycin related compounds; radicicol and HDAC inhibitors.

Nonlimiting exemplary protein tyrosine kinase and/or serine and/or threonine kinase inhibitors or lipid kinase inhibitors, include a) a compound targeting, decreasing, or inhibiting the activity of the platelet-derived growth factor-receptors (PDGFR), such as a compound that targets, decreases, or inhibits the activity of PDGFR, including olaratumab and N-phenyl-2-pyrimidine-amine derivatives, such as imatinib, SUlOl, SU6668, and GFB-111; b) a compound targeting, decreasing, or inhibiting the activity of the fibroblast growth factor-receptors (FGFR), such as erdafitinib and lenvatinib; c) a compound targeting, decreasing, or inhibiting the activity of the insulin-like growth factor receptor I (IGF-IR), such as brigatinib; d) a compound targeting, decreasing, or inhibiting the activity of the vascular endothelial growth factor-receptors (VEGFR), such as lenvatinib; e) a compound targeting, decreasing, or inhibiting the activity of the Trk receptor tyrosine kinase family, or ephrin B4 inhibitors, such as larotrectinib; f) a compound targeting, decreasing, or inhibiting the activity of the Axl receptor tyrosine kinase family; g) a compound targeting, decreasing, or inhibiting the activity of the Ret receptor tyrosine kinase, such as alectinib; h) a compound targeting, decreasing, or inhibiting the activity of the Kit/SCFR receptor tyrosine kinase, such as imatinib; i) a compound targeting, decreasing, or inhibiting the activity of the c-Kit receptor tyrosine kinases, such as imatinib; j) a compound targeting, decreasing, or inhibiting the activity of members of the c-Abl family, their gene-fusion products (e.g. Bcr-Abl kinase) and mutants, such as an N-phenyl-2-pyrimidine-amine derivative, such as imatinib or nilotinib; PD180970; AG957; NSC 680410; PD173955; or dasatinib; k) a compound targeting, decreasing, or inhibiting the activity of members of the protein kinase C (PKC) and Raf family of serine/threonine kinases, members of the MEK, SRC, JAK, FAK, PDK1, PKB/Akt, and Ras/MAPK family members, and/or members of the cyclin-dependent kinase family (CDK), such as a staurosporine derivative disclosed in U.S. Pat. No. 5,093,330, such as midostaurin; examples of further compounds include UCN-01, safingol, BAY 43-9006, bryostatin 1, perifosine; ilmofosine; RO 318220 and RO 320432; GO 6976; Isis 3521; LY333531/LY379196; a isochinoline compound; a farnesyl transferase inhibitor; PD184352 or QAN697, or AT7519; abemaciclib; binimetinib; cobimetinib; encorafenib; neratinib; palbociclib; ribociclib; 1) a compound targeting, decreasing or inhibiting the activity of a protein-tyrosine kinase, such as acalabrutinib, imatinib mesylate or a tyrphostin, such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); m) a compound targeting, decreasing, or inhibiting the activity of the epidermal growth factor family of receptor tyrosine kinases (EGFR, ErbB2, ErbB3, ErbB4 as homo- or heterodimers) and their mutants, such as brigatinib, CP 358774, ZD 1839, ZM 105180; trastuzumab, cetuximab, gefitinib, erlotinib, osimertinib, dacomitinib, necitumumab, neratinib, OSI-774, Cl-1033, EKB-569, GW-2016, antibodies E.1, E2.4, E2.5, E6.2, E6.4, E2.11, E6.3 and E7.6.3, and 7H-pyrrolo-[2,3-d]pyrimidine derivatives; n) a compound targeting, decreasing or inhibiting the activity of a phosphatidylinositol 3-kinase (PI3K), such as alpelisib, copanlisib, and duvelisib; and o) a compound targeting, decreasing, or inhibiting the activity of the c-Met receptor.

Nonlimiting exemplary compounds that target, decrease, or inhibit the activity of a protein or lipid phosphatase include inhibitors of phosphatase 1, phosphatase 2A, or CDC25, such as okadaic acid or a derivative thereof.

Further anti-angiogenic compounds include compounds having another mechanism for their activity unrelated to protein or lipid kinase inhibition, e.g., thalidomide and TNP-470.

Additional, nonlimiting, exemplary chemotherapeutic compounds, one or more of which may be used in combination with a Cereblon Ligand include: avastin, daunorubicin, adriamycin, Ara-C, VP-16, teniposide, mitoxantrone, idarubicin, carboplatinum, PKC412, 6-mercaptopurine (6-MP), fludarabine phosphate, octreotide, SOM230, FTY720, 6-thioguanine, cladribine, 6-mercaptopurine, pentostatin, hydroxyurea, 2-hydroxy-1H-isoindole-1,3-dione derivatives, 1-(4-chloroanilino)-4-(4-pyridylmethyl)phthalazine or a pharmaceutically acceptable salt thereof, 1-(4-chloroanilino)-4-(4-pyridylmethyl)phthalazine succinate, angiostatin, endostatin, anthranilic acid amides, ZD4190, ZD6474, SU5416, SU6668, bevacizumab, rhuMAb, rhuFab, macugon; FLT-4 inhibitors, FLT-3 inhibitors, VEGFR-2 IgGI antibody, RPI 4610, porfimer sodium, anecortave, triamcinolone, hydrocortisone, 11-a-epihydrocotisol, cortex olone, 17a-hydroxyprogesterone, corticosterone, desoxycorticosterone, testosterone, estrone, dexamethasone, fluocinolone, a plant alkaloid, a hormonal compound and/or antagonist, a biological response modifier, such as a lymphokine or interferon, an antisense oligonucleotide or oligonucleotide derivative, shRNA, and siRNA.

A number of suitable optional therapeutic, e.g., anticancer, agents are contemplated for use in the therapeutic methods provided herein. Indeed, the methods provided herein can include, but are not limited to, administration of numerous optional therapeutic agents such as: agents that induce apoptosis; polynucleotides (e.g., anti-sense, ribozymes, siRNA); polypeptides (e.g., enzymes and antibodies); biological mimetics (e.g., gossypol or BH3 mimetics); agents that bind (e.g., oligomerize or complex) with a Bcl-2 family protein such as Bax; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-KB modulators; anti-CDK compounds; HDAC inhibitors; and the like. Numerous other examples of optional therapeutic agents such as chemotherapeutic compounds and anticancer therapies suitable for co-administration with the disclosed compounds are known to those skilled in the art.

In certain embodiments, anticancer agents comprise agents that induce or stimulate apoptosis. Agents that induce or stimulate apoptosis include, for example, agents that interact with or modify DNA, such as by intercalating, cross-linking, alkylating, or otherwise damaging or chemically modifying DNA. Agents that induce apoptosis include, but are not limited to, radiation (e.g., X-rays, gamma rays, UV); tumor necrosis factor (TNF)-related factors (e.g., TNF family receptor proteins, TNF family ligands, TRAIL, antibodies to TRAIL-R1 or TRAIL-R2); kinase inhibitors (e.g., epidermal growth factor receptor (EGFR) kinase inhibitor).

Additional anticancer agents include: vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, and AVASTIN); anti-estrogens (e.g., raloxifene and tamoxifen); anti-androgens (e.g., flutamide, apalutamide, bicalutamide, finasteride, aminoglutethamide, ketoconazole, and corticosteroids); BCL-2 inhibitors (e.g., venetoclax); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

In still other embodiments, the therapeutic methods provided herein include administering to a subject having cancer (a cancer patient) therapeutically effective amounts of a Cereblon Ligand, an immune checkpoint inhibitor, and at least one additional optional therapeutic agent, e.g., an anti-hyperproliferative or antineoplastic agent selected from alkylating agents, antimetabolites, and natural products (e.g., herbs and other plant and/or animal derived compounds).

Alkylating agents suitable for use in the present methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (DTIC; dimethyltriazenoimid-azolecarboxamide).

In some embodiments, antimetabolites suitable for use in the present methods include, but are not limited to: 1) folic acid analogs (e.g., methotrexate (amethopterin)); 2) pyrimidine analogs (e.g., fluorouracil (5-fluorouracil; 5-FU), floxuridine (fluorode-oxyuridine; FudR), and cytarabine (cytosine arabinoside)); and 3) purine analogs (e.g., mercaptopurine (6-mercaptopurine; 6-MP), thioguanine (6-thioguanine; TG), and pentostatin (2′-deoxycoformycin)).

In still further embodiments, chemotherapeutic agents suitable for use in the methods of the present disclosure include, but are not limited to: 1) vinca alkaloids (e.g., vinblastine (VLB), vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin (cis-DDP) and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide).

Any oncolytic agent that is routinely used in a cancer therapy context finds use in the therapeutic methods of the present disclosure. For example, the U.S. Food and Drug Administration (FDA) maintains a formulary of oncolytic agents approved for use in the United States. International counterpart agencies to the FDA maintain similar formularies. Those skilled in the art will appreciate that the “product labels” required on all U.S. approved chemotherapeutics describe approved indications, dosing information, toxicity data, and the like, for the exemplary agents.

Anticancer agents further include compounds which have been identified to have anticancer activity. Examples include, but are not limited to, 3-AP, 12-O-tetradecanoylphorbol-13-acetate, 17AAG, 852A, ABI-007, ABR-217620, ABT-751, ADI-PEG 20, AE-941, AG-013736, AGRO100, alanosine, AMG 706, antibody G250, antineoplastons, AP23573, apaziquone, APC8015, atiprimod, ATN-161, atrasenten, azacitidine, BB-10901, BCX-1777, bevacizumab, BG00001, bicalutamide, BMS 247550, bortezomib, bryostatin-1, buserelin, calaspargase pegol-mknl, calcitriol, CCI-779, CDB-2914, cefixime, cetuximab, CG0070, cilengitide, clofarabine, combretastatin A4 phosphate, CP-675,206, CP-724,714, CpG 7909, curcumin, daratumumab, decitabine, DENSPM, dinutuximab, doxercalciferol, E7070, E7389, ecteinascidin 743, efaproxiral, eflornithine, EKB-569, elotuzumab, enzastaurin, erlotinib, exisulind, fenretinide, flavopiridol, fludarabine, flutamide, fotemustine, FR901228, G17DT, galiximab, gefitinib, genistein, glasdegib, glufosfamide, GTI-2040, histrelin, HKI-272, homoharringtonine, HSPPC-96, hu14.18-interleukin-2 fusion protein, HuMax-CD4, iloprost, imiquimod, infliximab, inotuzumab ozogamicin, interleukin-12, IPI-504, irofulven, ixabepilone, lapatinib, lenalidomide, lestaurtinib, leuprolide, LMB-9 immunotoxin, lonafarnib, luniliximab, lutetium Lu 177 dotatate, mafosfamide, MB07133, MDX-010, MLN2704, mogamulizumab-kpkc, monoclonal antibody 3F8, monoclonal antibody J591, motexafin, moxetumomab pasudotox-tdfk, MS-275, MVA-MUC1-IL2, nilutamide, niraparib, nitrocamptothecin, nolatrexed dihydrochloride, nolvadex, NS-9, O6-benzylguanine, oblimersen sodium, ONYX-015, oregovomab, OSI-774, panitumumab, paraplatin, PD-0325901, pemetrexed, PHY906, pioglitazone, pirfenidone, pixantrone, polatuzumab vedotin-piiq, PV-341, PSC 833, PXD11, pyrazoloacridine, RF15777, RAD0, ranpirnase, rebeccamycin analogue, rhuAngiostatin protein, rhuMab 2C4, rosiglitazone, rubitecan, rucaparib, S-1, S-8184, satraplatin, SB-, 15992, SGN-0010, SGN-40, sonidegib, sorafenib, SR31747A, ST1571, SU011248, suberoylanilide hydroxamic acid, suramin, tagraxofusp-erzs, talabostat, talampanel, talazoparib, tariquidar, temsirolimus, TGFa-PE38 immunotoxin, thalidomide, thymalfasin, tipifarnib, tirapazamine, TLK286, trabectedin, trifluridine and tipiracil hydrochloride, trimetrexate glucuronate, TroVax, UCN-1, valproic acid, vinflunine, VNP40101M, volociximab, vorinostat, VX-680, ZD1839, ZD6474, zileuton, and zosuquidar trihydrochloride.

In some embodiments, the optional therapeutic agent comprises one of the anti-cancer drugs or anti-cancer drug combinations listed in Table I.

TABLE III

| | | | |
|---|---|---|---|
| Abemaciclib | Abiraterone | Abraxane | ABVD |
| | Acetate | (Paclitaxel | |
| | | Albumin- | |
| | | stabilized | |
| | | Nanoparticle | |
| | | Formulation) | |
| ABVE | ABVE-PC | AC | Acalabrutinib |
| AC-T | Actemra | Adcetris | ADE |
| | (Tocilizumab) | (Brentuximab | |
| | | Vedotin) | |
| Ado- | Adriamycin | Afatinib | Afinitor |
| Trastuzumab | (Doxorubicin | Dimaleate | (Everolimus) |
| Emtansine | Hydrochloride) | | |
| Akynzeo | Aldara | Aldesleukin | Alecensa |
| (Netupitant and | (Imiquimod) | | (Alectinib) |
| Palonosetron | | | |
| Hydrochloride) | | | |
| Alectinib | Alemtuzumab | Alimta | Aliqopa |
| | | (Pemetrexed | (Copanlisib |
| | | Disodium) | Hydrochloride) |
| Alkeran for | Alkeran Tablets | Aloxi | Alunbrig |
| Injection | (Melphalan) | (Palonosetron | (Brigatinib) |
| (Melphalan | | Hydrochloride) | |
| Hydrochloride) | | | |
| Ameluz | Amifostine | Aminolevulinic | Anastrozole |
| (Aminolevulinic | | Acid | |
| Acid) | | | |
| Apalutamide | Aprepitant | Aranesp | Aredia |
| | | (Darbepoetin | (Pamidronate |
| | | Alfa) | Disodium) |
| Arimidex | Aromasin | Arranon | Arsenic |
| (Anastrozole) | (Exemestane) | (Nelarabine) | Trioxide |
| Arzerra | Asparaginase | Atezolizumab | Avastin |
| (Ofatumumab) | Erwinia | | (Bevacizumab) |
| | chrysanthemi | | |
| Avelumab | Axicabtagene | Axitinib | Azacitidine |
| | Ciloleucel | | |
| Azedra | Bavencio | BEACOPP | Beleodaq |
| (Iobenguane | (Avelumab) | | (Belinostat) |
| I 131) | | | |
| Belinostat | Bendamustine | Bendeka | BEP |
| | Hydrochloride | (Bendamustine | |
| | | Hydrochloride) | |
| Besponsa | Bevacizumab | Bexarotene | Bicalutamide |
| (Inotuzumab | | | |
| Ozogamicin) | | | |
| BiCNU | Binimetinib | Bleomycin | Blinatumomab |
| (Carmustine) | | | |
| Blincyto | Bortezomib | Bosulif | Bosutinib |
| (Blinatumomab) | | (Bosutinib) | |
| Braftovi | Brentuximab | Brigatinib | BuMel |
| (Encorafenib) | Vedotin | | |
| Busulfan | Busulfex | Cabazitaxel | Cabometyx |
| | (Busulfan) | | (Cabozantinib- |
| | | | S-Malate) |
| Cabozantinib- | CAF | Calquence | Campath |
| S-Malate | | (Acalabrutinib) | (Alemtuzumab) |
| Camptosar | Capecitabine | CAPOX | Carac |
| (Irinotecan | | | (Fluorouracil-- |
| Hydrochloride) | | | Topical) |
| Carboplatin | CARBO- | Carfilzomib | Carmustine |
| | PLATIN- | | |
| | TAXOL | | |
| Carmustine | Casodex | CEM | Cemiplimab- |
| Implant | (Bicalutamide) | | rwlc |
| Ceritinib | Cerubidine | Cervarix | Cetuximab |
| | (Daunorubicin | (Recombinant | |
| | Hydrochloride) | HPV Bivalent | |
| | | Vaccine) | |
| CEV | Chlorambucil | CHLORAM- | CHOP |
| | | BUCIL- | |
| | | PREDNISONE | |
| Cisplatin | Cladribine | Clofarabine | Clolar |
| | | | (Clofarabine) |
| CMF | Cobimetinib | Cometriq | Copanlisib |
| | | (Cabozantinib- | Hydrochloride |
| | | S-Malate) | |
| COPDAC | Copiktra | COPP | COPP-ABV |
| | (Duvelisib) | | |
| Cosmegen | Cotellic | Crizotinib | CVP |
| (Dactinomycin) | (Cobimetinib) | | |
| Cyclo- | Cyramza | Cytarabine | Cytarabine |
| phosphamide | (Ramucirumab) | | Liposome |
| Cytosar-U | Dabrafenib | Dacarbazine | Dacogen |
| (Cytarabine) | | | (Decitabine) |
| Dacomitinib | Dactinomycin | Daratumumab | Darbepoetin |
| | | | Alfa |
| Darzalex | Dasatinib | Daunorubicin | Daunorubicin |
| (Daratumumab) | | Hydrochloride | Hydrochloride |
| | | | and |
| | | | Cytarabine |
| | | | Liposome |
| Decitabine | Defibrotide | Defitelio | Degarelix |
| | Sodium | (Defibrotide | |
| | | Sodium) | |
| Denileukin | Denosumab | DepoCyt | Dexamethasone |
| Diftitox | | (Cytarabine | |
| | | Liposome) | |
| Dexrazoxane | Dinutuximab | Docetaxel | Doxil |
| Hydrochloride | | | (Doxorubicin |
| | | | Hydrochloride |
| | | | Liposome) |
| Doxorubicin | Doxorubicin | Dox-SL | Durvalumab |
| Hydrochloride | Hydrochloride | (Doxorubicin | |
| | Liposome | Hydrochloride | |
| | | Liposome) | |
| Duvelisib | Efudex | Eligard | Elitek |
| | (Fluorouracil-- | (Leuprolide | (Rasburicase) |
| | Topical) | Acetate) | |
| Ellence | Elotuzumab | Eloxatin | Eltrombopag |
| (Epirubicin | | (Oxaliplatin) | Olamine |
| Hydrochloride) | | | |
| Emend | Empliciti | Enasidenib | Encorafenib |
| (Aprepitant) | (Elotuzumab) | Mesylate | |
| Enzalutamide | Epirubicin | EPOCH | Epoetin Alfa |
| | Hydrochloride | | |
| Epogen | Erbitux | Eribulin | Erivedge |
| (Epoetin Alfa) | (Cetuximab) | Mesylate | (Vismodegib) |
| Erleada | Erlotinib | Erwinaze | Ethyol |
| (Apalutamide) | Hydrochloride | (Asparaginase | (Amifostine) |
| | | Erwinia | |
| | | chrysanthemi) | |
| Etopophos | Etoposide | Etoposide | Evacet |
| (Etoposide | | Phosphate | (Doxorubicin |
| Phosphate) | | | Hydrochloride |
| | | | Liposome) |
| Everolimus | Evista | Evomela | Exemestane |
| | (Raloxifene | (Melphalan | |
| | Hydrochloride) | Hydrochloride) | |
| 5-FU | 5-FU | Fareston | Farydak |
| (Fluorouracil | (Fluorouracil-- | (Toremifene) | (Panobinostat |
| Injection) | Topical) | | lactate) |
| Faslodex | FEC | Femara | Filgrastim |
| (Fulvestrant) | | (Letrozole) | |
| Firmagon | Fludarabine | Fluoroplex | Fluorouracil |
| (Degarelix) | Phosphate | (Fluorouracil-- | Injection |
| | | Topical) | |
| Fluorouracil-- | Flutamide | FOLFIRI | FOLFIRI- |
| Topical | | | BEVACI- |
| | | | ZUMAB |
| FOLFIRI- | FOLFIRINOX | FOLFOX | Folotyn |
| CETUXIMAB | | | (Pralatrexate) |
| Fostamatinib | FU-LV | Fulvestrant | Fusilev |
| Disodium | | | (Leucovorin |
| | | | Calcium) |
| Gardasil | Gardasil 9 | Gazyva | Gefitinib |
| (Recombinant | (Recombinant | (Obinutuzumab) | |
| HPV | HPV Nonavalent | | |
| Quadrivalent | Vaccine) | | |
| Vaccine) | | | |
| Gemcitabine | GEMCITABINE- | GEMCITABINE- | Gemtuzumab |
| Hydrochloride | CISPLATIN | OXALIPLATIN | Ozogamicin |
| Gemzar | Gilotrif | Gleevec | Gliadel Wafer |
| (Gemcitabine | (Afatinib | (Imatinib | (Carmustine |
| Hydrochloride) | Dimaleate) | Mesylate) | Implant) |
| Glucarpidase | Goserelin | Granisetron | Granisetron |
| | Acetate | | Hydrochloride |
| Granix | Halaven | Hemangeol | Herceptin |
| (Filgrastim) | (Eribulin | (Propranolol | (Trastuzumab) |
| | Mesylate) | Hydrochloride) | |
| HPV Bivalent | HPV | HPV | Hycamtin |
| Vaccine, | Nonavalent | Quadrivalent | (Topotecan |
| Recombinant | Vaccine, | Vaccine, | Hydrochloride) |
| | Recombinant | Recombinant | |
| Hydrea | Hydroxyurea | Hyper-CVAD | Ibrance |
| (Hydroxyurea) | | | (Palbociclib) |
| Ibritumomab | Ibrutinib | ICE | Iclusig |
| Tiuxetan | | | (Ponatinib |
| | | | Hydrochloride) |
| Idarubicin | Idelalisib | Idhifa | Ifex |
| Hydrochloride | | (Enasidenib | (Ifosfamide) |
| | | Mesylate) | |
| Ifosfamide | IL-2 | Imatinib | Imbruvica |
| | (Aldesleukin) | Mesylate | (Ibrutinib) |
| Imfinzi | Imiquimod | Imlygic | Inlyta (Axitinib) |
| (Durvalumab) | | (Talimogene | |
| | | Laherparepvec) | |
| Inotuzumab | Interferon | Interleukin-2 | Intron A |
| Ozogamicin | Alfa-2b, | (Aldesleukin) | (Recombinant |
| | Recombinant | | Interferon Alfa- |
| | | | 2b) |
| Iobenguane | Ipilimumab | Iressa | Irinotecan |
| I 131 | | (Gefitinib) | Hydrochloride |
| Irinotecan | Istodax | Ivosidenib | Ixabepilone |
| Hydrochloride | (Romidepsin) | | |
| Liposome | | | |
| Ixazomib Citrate | Ixempra | Jakafi | JEB |
| | (Ixabepilone) | (Ruxolitinib | |
| | | Phosphate) | |
| Jevtana | Kadcyla (Ado- | Kepivance | Keytruda |
| (Cabazitaxel) | Trastuzumab | (Palifermin) | (Pembro- |
| | Emtansine) | | lizumab) |
| Kisqali | Kymriah | Kyprolis | Lanreotide |
| (Ribociclib) | (Tisagen- | (Carfilzomib) | Acetate |
| | lecleucel) | | |
| Lapatinib | Larotrectinib | Lartruvo | Lenalidomide |
| Ditosylate | Sulfate | (Olaratumab) | |
| Lenvatinib | Lenvima | Letrozole | Leucovorin |
| Mesylate | (Lenvatinib | | Calcium |
| | Mesylate) | | |
| Leukeran | Leuprolide | Levulan | Libtayo |
| (Chlorambucil) | Acetate | Kerastik | (Cemiplimab- |
| | | (Aminolevulinic | rwlc) |
| | | Acid) | |
| LipoDox | Lomustine | Lonsurf | Lorbrena |
| (Doxorubicin | | (Trifluridine and | (Lorlatinib) |
| Hydrochloride | | Tipiracil | |
| Liposome) | | Hydrochloride) | |
| Lorlatinib | Lumoxiti | Lupron | Lupron Depot |
| | (Moxetumomab | (Leuprolide | (Leuprolide |
| | Pasudotox-tdfk) | Acetate) | Acetate) |
| Lutathera | Lutetium (Lu | Lynparza | Marqibo |
| (Lutetium Lu | 177-Dotatate) | (Olaparib) | (Vincristine |
| 177-Dotatate) | | | Sulfate |
| | | | Liposome) |
| Matulane | Mechlorethamine | Megestrol | Mekinist |
| (Procarbazine | Hydrochloride | Acetate | (Trametinib) |
| Hydrochloride) | | | |
| Mektovi | Melphalan | Melphalan | Mercaptopurine |
| (Binimetinib) | | Hydrochloride | |
| Mesna | Mesnex (Mesna) | Methotrexate | Methyl- |
| | | | naltrexone |
| | | | Bromide |
| Midostaurin | Mitomycin C | Mitoxantrone | Moga- |
| | | Hydrochloride | mulizumab- |
| | | | kpkc |
| Moxetumomab | Mozobil | Mustargen | MVAC |
| Pasudotox-tdfk | (Plerixafor) | (Mechlor- | |
| | | ethamine | |
| | | Hydrochloride) | |
| Myleran | Mylotarg | Nanoparticle | Navelbine |
| (Busulfan) | (Gemtuzumab | Paclitaxel | (Vinorelbine |
| | Ozogamicin) | (Paclitaxel | Tartrate) |
| | | Albumin- | |
| | | stabilized | |
| | | Nanoparticle | |
| | | Formulation) | |
| Necitumumab | Nelarabine | Neratinib Maleate | Nerlynx |
| | | | (Neratinib |
| | | | Maleate) |
| Netupitant and | Neulasta | Neupogen | Nexavar |
| Palonosetron | (Pegfilgrastim) | (Filgrastim) | (Sorafenib |
| Hydrochloride | | | Tosylate) |
| Nilandron | Nilotinib | Nilutamide | Ninlaro |
| (Nilutamide) | | | (Ixazomib |
| | | | Citrate) |
| Niraparib | Nivolumab | Nplate | Obinutuzumab |
| Tosylate | | (Romiplostim) | |
| Monohydrate | | | |
| Odomzo | OEPA | Ofatumumab | OFF |
| (Sonidegib) | | | |
| Olaparib | Olaratumab | Omacetaxine | Oncaspar |
| | | Mepesuccinate | (Pegaspargase) |
| Ondansetron | Onivyde | Ontak | Opdivo |
| Hydrochloride | (Irinotecan | (Denileukin | (Nivolumab) |
| | Hydrochloride | Diftitox) | |
| | Liposome) | | |
| OPPA | Osimertinib | Oxaliplatin | Paclitaxel |
| Paclitaxel | PAD | Palbociclib | Palifermin |
| Albumin- | | | |
| stabilized | | | |
| Nanoparticle | | | |
| Formulation | | | |
| Palonosetron | Palonosetron | Pamidronate | Panitumumab |
| Hydrochloride | Hydrochloride | Disodium | |
| | and Netupitant | | |
| Panobinostat | Pazopanib | PCV | PEB |
| Lactate | Hydrochloride | | |
| Pegaspargase | Pegfilgrastim | Peginterferon | PEG-Intron |
| | | Alfa-2b | (Peginterferon |
| | | | Alfa-2b) |
| Pembrolizumab | Pemetrexed | Perjeta | Pertuzumab |
| | Disodium | (Pertuzumab) | |
| Plerixafor | Pomalidomide | Pomalyst | Ponatinib |
| | | (Pomalidomide) | Hydrochloride |
| Portrazza | Poteligeo | Pralatrexate | Prednisone |
| (Necitumumab) | (Mogamulizumab- | | |
| | kpkc) | | |
| Procarbazine | Procrit (Epoetin | Proleukin | Prolia |
| Hydrochloride | Alfa) | (Aldesleukin) | (Denosumab) |
| Promacta | Propranolol | Provenge | Purinethol |
| (Eltrombopag | Hydrochloride | (Sipuleucel-T) | (Mercapto- |
| Olamine) | | | purine) |
| Purixan | Radium 223 | Raloxifene | Ramucirumab |
| (Mercaptopurine) | Dichloride | Hydrochloride | |
| Rasburicase | R-CHOP | R-CVP | Recombinant |
| | | | Human |
| | | | Papillomavirus |
| | | | (HPV) Bivalent |
| | | | Vaccine |
| Recombinant | Recombinant | Recombinant | Regorafenib |
| Human | Human | Interferon | |
| Papillomavirus | Papillomavirus | Alfa-2b | |
| (HPV) | (HPV) | | |
| Nonavalent | Quadrivalent | | |
| Vaccine | Vaccine | | |
| Relistor | R-EPOCH | Retacrit | Revlimid |
| (Methyl- | | (Epoetin Alfa) | (Lenalidomide) |
| naltrexone | | | |
| Bromide) | | | |
| Rheumatrex | Ribociclib | R-ICE | Rituxan |
| (Methotrexate) | | | (Rituximab) |
| Rituxan Hycela | Rituximab | Rituximab and | Rolapitant |
| (Rituximab and | | Hyaluronidase | Hydrochloride |
| Hyaluronidase | | Human | |
| Human) | | | |
| Romidepsin | Romiplostim | Rubidomycin | Rubraca |
| | | (Daunorubicin | (Rucaparib |
| | | Hydrochloride) | Camsylate) |
| Rucaparib | Ruxolitinib | Rydapt | Sancuso |
| Camsylate | Phosphate | (Midostaurin) | (Granisetron) |
| Sclerosol | Siltuximab | Sipuleucel-T | Somatuline |
| Intrapleural | | | Depot |
| Aerosol (Talc) | | | (Lanreotide |
| | | | Acetate) |
| Sonidegib | Sorafenib | Sprycel | STANFORD V |
| | Tosylate | (Dasatinib) | |
| Sterile Talc | Steritalc (Talc) | Stivarga | Sunitinib Malate |
| Powder (Talc) | | (Regorafenib) | |
| Sustol | Sutent (Sunitinib | Sylatron | Sylvant |
| (Granisetron) | Malate) | (Peginterferon | (Siltuximab) |
| | | Alfa-2b) | |
| Synribo | Tabloid | TAC | Tafinlar |
| (Omacetaxine | (Thioguanine) | | (Dabrafenib) |
| Mepesuccinate) | | | |
| Tagrisso | Talc | Talimogene | Tamoxifen |
| (Osimertinib) | | Laherparepvec | Citrate |
| Tarabine PFS | Tarceva | Targretin | Tasigna |
| (Cytarabine) | (Erlotinib | (Bexarotene) | (Nilotinib) |
| | Hydrochloride) | | |
| Tavalisse | Taxol | Taxotere | Tecentriq |
| (Fostamatinib | (Paclitaxel) | (Docetaxel) | (Atezolizumab) |
| Disodium) | | | |
| Temodar | Temozolomide | Temsirolimus | Thalidomide |
| (Temozolomide) | | | |
| Thalomid | Thioguanine | Thiotepa | Tibsovo |
| (Thalidomide) | | | (Ivosidenib) |
| Tisagenlecleucel | Tocilizumab | Tolak | Topotecan |
| | | (Fluorouracil-- | Hydrochloride |
| | | Topical) | |
| Toremifene | Torisel | Totect | TPF |
| | (Temsirolimus) | (Dexrazoxane | |
| | | Hydrochloride) | |
| Trabectedin | Trametinib | Trastuzumab | Treanda |
| | | | (Bendamustine |
| | | | Hydrochloride) |
| Trexall | Trifluridine and | Trisenox | Tykerb |
| (Methotrexate) | Tipiracil | (Arsenic | (Lapatinib |
| | Hydrochloride | Trioxide) | Ditosylate) |
| Unituxin | Uridine | VAC | Valrubicin |
| (Dinutuximab) | Triacetate | | |
| Valstar | Vandetanib | VAMP | Varubi |
| (Valrubicin) | | | (Rolapitant |
| | | | Hydrochloride) |
| Vectibix | VeIP | Velcade | Vemurafenib |
| (Panitumumab) | | (Bortezomib) | |
| Venclexta | Venetoclax | Verzenio | Vidaza |
| (Venetoclax) | | (Abemaciclib) | (Azacitidine) |
| Vinblastine | Vincristine | Vincristine Sulfate | Vinorelbine |
| Sulfate | Sulfate | Liposome | Tartrate |
| VIP | Vismodegib | Vistogard | Vitrakvi |
| | | (Uridine | (Larotrectinib |
| | | Triacetate) | Sulfate) |
| Vizimpro | Voraxaze | Vorinostat | Votrient |
| (Dacomitinib) | (Glucarpidase) | | (Pazopanib |
| | | | Hydrochloride) |
| Vyxeos | Xalkori | Xeloda | XELIRI |
| (Daunorubicin | (Crizotinib) | (Capecitabine) | |
| Hydrochloride | | | |
| and Cytarabine | | | |
| Liposome) | | | |
| XELOX | Xgeva | Xofigo | Xtandi |
| | (Denosumab) | (Radium 223 | (Enzalutamide) |
| | | Dichloride) | |
| Yervoy | Yescarta | Yondelis | Zaltrap (Ziv- |
| (Ipilimumab) | (Axicabtagene | (Trabectedin) | Aflibercept) |
| | Ciloleucel) | | |
| Zarxio | Zejula (Niraparib | Zelboraf | Zevalin |
| (Filgrastim) | Tosylate | (Vemurafenib) | (Ibritumomab |
| | Monohydrate) | | Tiuxetan) |
| Zinecard | Ziv-Aflibercept | Zofran | Zoladex |
| (Dexrazoxane | | (Ondansetron | (Goserelin |
| Hydrochloride) | | Hydrochloride) | Acetate) |
| Zoledronic | Zolinza | Zometa | Zydelig |
| Acid | (Vorinostat) | (Zoledronic Acid) | (Idelalisib) |
| Zykadia | Zytiga | | |
| (Ceritinib) | (Abiraterone | | |
| | Acetate) | | |

The disclosure provides the following particular embodiments in connection with treating a disease in a subject.

Embodiment I. A method of treating a subject, the method comprising administering to the subject a therapeutically effective amount of a Cereblon Ligand or PROTAC Molecule, wherein the subject has cancer or other proliferative disorder, or an inflammatory disease.

Embodiment II. The method Embodiment I, wherein the subject has cancer.

Embodiment III. The method of Embodiment II, wherein the cancer is any one or more of the cancers of Table I.

Embodiment IV. The method of Embodiment II, wherein the cancer is selected from acute monocytic leukemia, acute myelogenous leukemia, chronic myelogenous leukemia, chronic lymphocytic leukemia mixed lineage leukemia, NUT midline carcinoma, multiple myeloma, small cell lung cancer, non-small cell lung cancer, neuroblastoma, Burkitt's lymphoma, cervical cancer, esophageal cancer, ovarian cancer, colorectal cancer, prostate cancer, breast cancer, bladder cancer, ovary cancer, glioma, sarcoma, esophageal squamous cell carcinoma, and papillary thyroid carcinoma.

Embodiment V. The method of Embodiment II, wherein the cancer is any one or more of the cancers of Table II, e.g, multiple myeloma.

Embodiment VI. The method of any one of Embodiments I-V further comprising administering a therapeutically effective amount of an optional therapeutic agent useful in the treatment of the disease or condition, e.g., an immune checkpoint inhibitor or other anticancer agent.

Embodiment VII. The method of any one of Embodiments I-VI, wherein a therapeutically effective amount of a Cereblon Ligand is administered to the subject.

Embodiment VIII. The method of any one of Embodiments I-VI, wherein a therapeutically effective amount of a PROTAC is administered to the subject.

Embodiment IX. A pharmaceutical composition comprising a Cereblon Ligand or PROTAC Molecule, and a pharmaceutically acceptable excipient for use in treating cancer or other proliferative disorder, or an inflammatory disease.

Embodiment X. The pharmaceutical composition of Embodiment IX for use in treating cancer.

Embodiment XI. The pharmaceutical composition of Embodiment X, wherein the cancer is any one or more of the cancers of Table I.

Embodiment XII. The pharmaceutical composition of Embodiment X, wherein the cancer is selected from acute monocytic leukemia, acute myelogenous leukemia, chronic myelogenous leukemia, chronic lymphocytic leukemia mixed lineage leukemia, NUT-midline carcinoma, multiple myeloma, small cell lung cancer, non-small cell lung cancer, neuroblastoma, Burkitt's lymphoma, cervical cancer, esophageal cancer, ovarian cancer, colorectal cancer, prostate cancer, breast cancer, bladder cancer, ovary cancer, glioma, sarcoma, esophageal squamous cell carcinoma, and papillary thyroid carcinoma.

Embodiment XIII. The pharmaceutical composition of Embodiment X, wherein the cancer is any one or more of the cancers of Table II.

Embodiment XIV. The pharmaceutical composition of any one of Embodiments IX-XIII comprising a Cereblon Ligand.

Embodiment XV. The pharmaceutical composition of any one of Embodiments IX-XIII comprising a PROTAC Molecule.

Embodiment XVI. A Cereblon Ligand or PROTAC Molecule for use in treatment of cancer or other proliferative disorder, or an inflammatory disease.

Embodiment XVII. The Cereblon Ligand or PROTAC Molecule of Embodiment XVI for use in treating cancer.

Embodiment XVIII. The Cereblon Ligand or PROTAC Molecule of Embodiment XVII, wherein the cancer is any one or more of the cancers of Table I.

Embodiment XIX. The Cereblon Ligand or PROTAC Molecule of Embodiment XVII, wherein the cancer is selected from acute monocytic leukemia, acute myelogenous leukemia, chronic myelogenous leukemia, chronic lymphocytic leukemia mixed lineage leukemia, NUT midline carcinoma, multiple myeloma, small cell lung cancer, non-small cell lung cancer, neuroblastoma, Burkitt's lymphoma, cervical cancer, esophageal cancer, ovarian cancer, colorectal cancer, prostate cancer, breast cancer, bladder cancer, ovary cancer, glioma, sarcoma, esophageal squamous cell carcinoma, and papillary thyroid carcinoma.

Embodiment XX. The Cereblon Ligand or PROTAC Molecule of Embodiment XVII, wherein the cancer is any one or more of the cancers of Table II.

Embodiment XXI. The Cereblon Ligand of any one of Embodiments XVI-XX for use in treatment of cancer or other proliferative disorder, or an inflammatory disease.

Embodiment XXII. The PROTAC Molecule of any one of Embodiments XVI-XX for use in treatment of cancer or other proliferative disorder, or an inflammatory disease.

Embodiment XXIII. Use of a Cereblon Ligand or PROTAC Molecule for the manufacture of a medicament for treatment of cancer or other proliferative disorder, or an inflammatory disease.

Embodiment XXIV. The use of Embodiment XXIII for the treatment of cancer.

Embodiment XXV. The use of Embodiment XXIV, wherein the cancer is any one or more of the cancers of Table I.

Embodiment XXVI. The use of Embodiment XXIII, wherein the cancer is selected from acute monocytic leukemia, acute myelogenous leukemia, chronic myelogenous leukemia, chronic lymphocytic leukemia mixed lineage leukemia, NUT midline carcinoma, multiple myeloma, small cell lung cancer, non-small cell lung cancer, neuroblastoma, Burkitt's lymphoma, cervical cancer, esophageal cancer, ovarian cancer, colorectal cancer, prostate cancer, breast cancer, bladder cancer, ovary cancer, glioma, sarcoma, esophageal squamous cell carcinoma, and papillary thyroid carcinoma.

Embodiment XXVII. The use of Embodiment XXIV, wherein the cancer is any one or more of the cancers of Table II.

Embodiment XXVIII. The use of the Cereblon Ligand of any one of Embodiments XXIII-XXVII for the manufacture of a medicament.

Embodiment XXIX. The use of the PROTAC Molecule of any one of Embodiments XXIII-XXVII, wherein the Cereblon Ligand is a compound of any one of Formulae II-IV for the manufacture of a medicament.

Embodiment XXX. A method of inhibiting CRBN ubiquitination within a cell of a subject in need thereof, the method comprising administering to the subject a Cereblon Ligand.

Methods of Synthesis

In some aspects, the present disclosure provides methods of preparing a Compound of Disclosure.

In some aspects, the present disclosure provides compounds obtainable by, or obtained by, or directly obtained by a method for preparing a Compound of Disclosure.

Exemplary non-limiting methods of making Compounds of the Disclosure and Intermediates of the Disclosure are provided in Synthetic Schemes 1-28. See below.

Those skilled in the art will recognize if a stereocenter exists in the compounds of the present dislosure (e.g., a compound of any of the formulae or any individual compounds disclosed herein). Accordingly, the present disclosure includes both possible stereoisomers (unless specified in the synthesis) and includes not only racemic compound but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-lnterscience, 1994).

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH, Inc. (Toronto, Canada), Bionet (Cornwall, U.K.), Chem Service Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc.

Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

In some embodiments, the biological assay may involve evaluation of CRBN binding activity, e.g., in Cereblon Binding Kit (Cisbio, #64BDCRBNPEG).

In some embodiments, the cell line is maintained and cultured, e.g., at room temperature for about 3 hours.

In some embodiments, the Fluorescence Resonance Energy Transfer (TR-FRET) measurements of the cell line is recorded and evaluated.

Pharmaceutical Compositions

In some aspects, the present disclosure provides pharmaceutical compositions comprising a Compound of Disclosure (e.g., a PROTAC Molecule), or a pharmaceutically acceptable salt, solvate, or stereoisomer thereof, and a pharmaceutically acceptable excipient.

In some embodiments, the pharmaceutically suitable or acceptable carrier is selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In some embodiments, the pharmaceutical composition is formulated for oral, topical (including buccal and sublingual), rectal, vaginal, transdermal, parenteral, intrapulmonary, intradermal, intrathecal and epidural and intranasal administration. Parenteral administration includes intramuscular, intravenous, intraarterial, intraperitoneal, or subcutaneous administration. In some embodiments, the pharmaceutical composition is formulated for intravenous injection, oral administration, inhalation, nasal administration, topical administration, or ophthalmic administration. In some embodiments, the pharmaceutical composition is formulated for oral administration. In some embodiments, the pharmaceutical composition is formulated for intravenous injection. In some embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In some embodiments, the pharmaceutical composition is formulated as a tablet.

Suitable doses and dosage regimens are determined by conventional range-finding techniques known to those of ordinary skill in the art. Generally, treatment is initiated with smaller dosages that are less than the optimum dose of the compound disclosed herein. Thereafter, the dosage is increased by small increments until the optimum effect under the circumstances is reached. By way of example only, the dose of the compound described herein for methods of treating a disease as described herein is about 0.001 to about 1 mg/kg body weight of the subject per day.

Kits of the Disclosure

In some embodiments, the present disclosure provides kits which comprise a Cereblon Ligand or PROTAC Molecule (or a composition comprising a Cereblon Ligand or PROTAC Molecule) packaged in a manner that facilitates their use to practice methods of the present disclosure. In some embodiments, the kit includes, for example, a Cereblon Ligand (or a composition comprising a Cereblon Ligand) packaged in a container, such as a sealed bottle or vessel, with a label affixed to the container or included in the kit that describes use of the compound or composition to practice the method of the disclosure, e.g., the method of any one of Embodiments I-VI. In some embodiments, the compound or composition is packaged in a unit dosage form. The kit further can include a device suitable for administering the composition according to the intended route of administration.

### EXEMPLARY EMBODIMENTS

Embodiment 1. A compound of Formula I or a pharmaceutically acceptable salt or solvate thereof, wherein:
(a) A¹ is selected from —CR^{2a}═ and —N═;
   A is —CR^{2b}═;
   A² is —CR^{2c}═;
   R^{2b} and R^{2c} are taken together to form a ring comprising a:
      (i) —(CH₂)ₘ—X—(CH₂)ₙ— radical;
      (ii) —C(═O)—N(R^{1d})—(CH₂)_{q}— radical;
      (iii) —(CH₂)ₜ—N(R^{1e})—(CH₂)₂—Y—(CH₂)ᵤ— radical;
      (iv) -E¹=E-E²=E³- radical;
      (v)=A⁴-N(R^{1g})—CR^{2k}=radical; or
      (vi) -E⁴=CR^{1j}-E⁵- radical;
   R^{2a} is selected from hydrogen, halo, C₁-C₃ alkyl, amino, and C₁-C₃ alkoxy; and
   R^{2d} is selected from hydrogen, halo, C₁-C₃ alkyl, amino, and C₁-C₃ alkoxy;
   A³ is selected from —CR^{2d}═ and —N═; or
(b) A¹ is —CR^{2a}═;
   A is —CR^{2b}═;
   R^{2a} and R^{2b} are taken together to form a ring comprising a:
      (i) —(CH₂)ₘ—X—(CH₂)ₙ— radical;
      (ii) —C(═O)—N(R^{1d})—(CH₂)_{q}— radical;
      (iii) —(CH₂)ₜ—N(R^{1e})—(CH₂)₂—Y—(CH₂)ᵤ— radical;
      (iv) -E¹=E-E²=E³- radical; or
      (v)=A⁴-N(R^{1g})—CR^{2k}=radical; or
      (vi) -E⁴=CR^{1j}-E⁵- radical;
   A² is selected from —CR^{2c}═ and —N═;
   A³ is selected from —CR^{2d}═ and —N═;
   R^{2e} is selected from hydrogen, halo, C₁-C₃ alkyl, amino, and C₁-C₃ alkoxy; and
   R^{2d} is selected from hydrogen, halo, C₁-C₃ alkyl, amino, and C₁-C₃ alkoxy;
   X is selected from —N(R^{1a})— and —CR^{1b}R^{1c}—
   R^{1a} is selected from hydrogen and Q-L-;
   R^{1b} is selected from hydrogen, —CHO, —C(═O)OH, hydroxy, (hydroxy)C₁-C₃ alkyl, amino, (amino)alkyl, and Q-L-;
   R^{1a} is hydrogen; or
   R^{1b} and R^{1c} taken together with the carbon atom to which they are attached form a —C(═O)—; or
   R^{1b} and R^{1c} taken together with the carbon atom to which they are attached form:

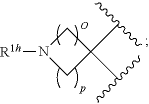

R^{1d} is selected from hydrogen and Q-L-;
R^{1e} is selected from hydrogen and Q-L-;
R^{1g} is selected from hydrogen and Q-L-;
R^{1h} is selected from hydrogen and Q-L-;
Y is selected from —O—, —S—, and —N(R^{1f})—
R^{1f} is selected from hydrogen and C₁-C₃ alkyl;
E¹ is selected from —CR^{2e}═ and —N═;
E is selected from —CR^{2f}═ and —N═;
E² is selected from —CR^{2g}═ and —N═;
E³ is selected from —CR^{2h}═ and —N═;
R^{2e}, R^{2f}, R^{2g}, and R^{2h} are independently selected from hydrogen, halo, hydroxy, amino, —CHO, C₁-C₃ alkyl, and C₁-C₃ alkoxy; or
R^{2e} is Q-L-; and, if present, R^{2f}, R^{2g}, and R^{2h} are independently selected from hydrogen, halo, hydroxy, amino, —CHO, C₁-C₃ alkyl, and C₁-C₃ alkoxy; or
R^{2f} is Q-L-; and, if present, R^{2e}, R^{2g}, and R^{2h} are independently selected from hydrogen, halo, hydroxy, amino, —CHO, C₁-C₃ alkyl, and C₁-C₃ alkoxy; or
R^{2g} is Q-L-; and, if present, R^{2e}, R^{2f}, and R^{2h} are independently selected from hydrogen, halo, hydroxy, amino, —CHO, C₁-C₃ alkyl, and C₁-C₃ alkoxy; or
R^{2h} is Q-L-; and, if present, R^{2e}, R^{2f}, and R^{2g} are independently selected from hydrogen, halo, hydroxy, amino, —CHO, C₁-C₃ alkyl, and C₁-C₃ alkoxy;
E⁴ is selected from ═C(H)— and ═N—;
E⁵ is selected from —O—, —S—, and —N(R^{2m})—;
R^{2m} is selected from hydrogen and C₁-C₄ alkyl;
R^{1j} is selected from hydrogen, C₁-C₄ alkyl, (hydroxy)alkyl, (heterocyclo)alkyl, and Q-L-;
A⁴ is selected from —CR^{2j}═ and —N═;
R^{2j} is selected from hydrogen and C₁-C₃ alkyl;
R^{2k} is selected from hydrogen and C₁-C₃ alkyl;
m is 1, 2, or 3;
n is 1, 2, or 3;
is 1, 2, or 3;
p is 1, 2, or 3;
q is 1 or 2;
t is 0 or 1;
u is 0 or 1;
R³ is selected from hydrogen, deuterium, fluoro, and C₁-C₃ alkyl;
Z and Z¹ are —C(═O)—; or
Z is —C(═O)— and Z¹ is —CR^{4a}R^{4b}—; or
Z is —CR^{4a}R^{4b}— and Z¹ is —C(═O)—; or
Z is —N═C(CH₃)— and Z¹ is —C(═O)—; or
Z is —C(═O)— and Z¹ is —N═C(CH₃)—; or
Z is a bond and Z¹ is —N(R²ⁿ)C(═O)—; or
Z is —N(R²ⁿ)C(═O) and Z is a bond;
R²ⁿ is selected from hydrogen and C₁-C₄ alkyl;
R^{4a} and R^{4b} are independently selected from hydrogen and C₁-C₃ alkyl; or
R^{4a} and R^{4b} taken together with the carbon to which they are attached form a C₃-C₆ cycloalkyl,
Q is a small molecule that binds to a target protein of interest;
L is -J¹-J²-J³-J⁴-J⁵-, wherein J¹ is attached to Q;
J¹ is selected from alkylenyl, cycloalkylenyl, and heterocyclenyl; or J¹ is absent;
J² is selected from —C(═O)—, —(CH₂)_{w}—, —CH═CH—, and —C≡C—;
w is 0, 1, 2, or 3;
J³ is selected from alkylenyl, heteroalkylenyl, cycloalkylenyl, heterocyclenyl, phenylenyl, and heteroarylenyl; or J³ is absent;
J⁴ is selected from alkylenyl, cycloalkylenyl, and heterocyclenyl; or J⁴ is absent;
J⁵ is selected from —O—, —N(H)—, —C≡C—, —(CH₂)ₓ— and —C(═O)—; and
x is 0, 1, 2, or 3,
with the proviso the compound of Formula I is not a compound of any of Formulae A-F, wherein:
   R³ is selected from hydrogen, fluoro, methyl, and deuterium;
   Z is selected from —C(═O)— and —CH₂—;
   R^{1b} is selected from —OH, —CH₂OH, —CHO, —C(═O)OH, and Q-L; and
   R^{1c} is hydrogen; or
   R^{1b} and R^{1c} taken together with the carbon atom to which they are attached form a —C(═O)—.

Embodiment 2. The compound of Embodiment 1 of Formula II or a pharmaceutically acceptable salt or solvate thereof.

Embodiment 3. The compound of Embodiment 1 of Formula III or a pharmaceutically acceptable salt or solvate thereof.

Embodiment 4. The compound of Embodiment 1 of Formula IV or a pharmaceutically acceptable salt or solvate thereof.

Embodiment 5. The compound of Embodiment 4 of Formula V or a pharmaceutically acceptable salt or solvate thereof.

Embodiment 6. The compound of Embodiment 4 of Formula VI or a pharmaceutically acceptable salt or solvate thereof.

Embodiment 7. The compound of any one of Embodiments 4-6, or a pharmaceutically acceptable salt or solvate thereof, wherein A¹ is —CR^{2a}═ and A³ is —CR^{2d}═.

Embodiment 8. The compound of Embodiment 7, or a pharmaceutically acceptable salt or solvate thereof, wherein R^{2a} is selected from hydrogen, —NH₂, fluoro, and methoxy.

Embodiment 9. The compound of Embodiments 7 or 8, or a pharmaceutically acceptable salt or solvate thereof, wherein R^{2d} is selected from hydrogen, —NH₂, fluoro, and methoxy.

Embodiment 10. The compound of any one of Embodiments 4-6, or a pharmaceutically acceptable salt or solvate thereof, wherein A¹ is —N═ and A³ is —CR^{2d}═.

Embodiment 11. The compound of Embodiment 10, or a pharmaceutically acceptable salt or solvate thereof, wherein R^{2d} is selected from hydrogen, —NH₂, fluoro, and methoxy.

Embodiment 12. The compound of any one of Embodiments 4-6, or a pharmaceutically acceptable salt or solvate thereof, wherein A¹ is —CR^{2a}═ and A³ is —N═.

Embodiment 13. The compound of Embodiment 12, or a pharmaceutically acceptable salt or solvate thereof, wherein R^{2a} is selected from hydrogen, —NH₂, fluoro, and methoxy.

Embodiment 14. The compound of Embodiment 1 of Formula VII or a pharmaceutically acceptable salt or solvate thereof.

Embodiment 15. The compound of Embodiment 14 of Formula VIII or a pharmaceutically acceptable salt or solvate thereof.

Embodiment 16. The compound of Embodiment 14 of Formula IX or a pharmaceutically acceptable salt or solvate thereof.

Embodiment 17. The compound of any one of Embodiments 14-16, or a pharmaceutically acceptable salt or solvate thereof, wherein A² is —CR^{2c}═ and A³ is —CR^{2d}═.

Embodiment 18. The compound of Embodiment 17, or a pharmaceutically acceptable salt or solvate thereof, wherein R^{2c} is selected from hydrogen, —NH₂, fluoro, and methoxy.

Embodiment 19. The compound of Embodiments 17 or 18, or a pharmaceutically acceptable salt or solvate thereof, wherein R^{2d} is selected from hydrogen, —NH₂, fluoro, and methoxy.

Embodiment 20. The compound of any one of Embodiments 14-16, or a pharmaceutically acceptable salt or solvate thereof, wherein A² is —N═ and A³ is —CR^{2d}═.

Embodiment 21. The compound of Embodiment 20, or a pharmaceutically acceptable salt or solvate thereof, wherein R^{2d} is selected from hydrogen, —NH₂, fluoro, and methoxy.

Embodiment 22. The compound of any one of Embodiments 14-16, or a pharmaceutically acceptable salt or solvate thereof, wherein A² is —CR^{2c}═ and A³ is —N═.

Embodiment 23. The compound of Embodiment 22, or a pharmaceutically acceptable salt or solvate thereof, wherein R^{2c} is selected from hydrogen, —NH₂, fluoro, and methoxy.

Embodiment 24. The compound of any one of Embodiments 4-23, or a pharmaceutically acceptable salt or solvate thereof, wherein m is 1.

Embodiment 25. The compound of any one of Embodiments 4-23, or a pharmaceutically acceptable salt or solvate thereof, wherein m is 2.

Embodiment 26. The compound of any one of Embodiments 4-23, or a pharmaceutically acceptable salt or solvate thereof, wherein m is 3.

Embodiment 27. The compound of any one of Embodiments 4-26, or a pharmaceutically acceptable salt or solvate thereof, wherein n is 1.

Embodiment 28. The compound of any one of Embodiments 4-26, or a pharmaceutically acceptable salt or solvate thereof, wherein n is 2.

Embodiment 29. The compound of any one of Embodiments 4-26, or a pharmaceutically acceptable salt or solvate thereof, wherein n is 3.

Embodiment 30. The compound of any one of Embodiments 4-29, or a pharmaceutically acceptable salt or solvate thereof, wherein X is —CR^{1b}R^{1c}—.

Embodiment 31. The compound of Embodiment 30, or a pharmaceutically acceptable salt or solvate thereof, wherein R^{1c} is hydrogen.

Embodiment 32. The compound of any one of Embodiments 30 or 31, or a pharmaceutically acceptable salt or solvate thereof, wherein R^{1b} is selected from hydroxy, —NH₂, —CHO, —C(═O)OH, and —CH₂OH.

Embodiment 33. The compound of any one of Embodiments 30 or 31, or a pharmaceutically acceptable salt or solvate thereof, wherein R^{1b} is Q-L-.

Embodiment 34. The compound of Embodiment 30, or a pharmaceutically acceptable salt or solvate thereof, wherein R^{1b} and R^{1c} taken together with the carbon atom to which they are attached form —C(═O)—.

Embodiment 35. The compound of any one of Embodiments 4-29 or a pharmaceutically acceptable salt or solvate thereof, wherein X is —N(R^{1a}).

Embodiment 36. The compound of Embodiment 35, a pharmaceutically acceptable salt or solvate thereof, wherein R^{1a} is hydrogen.

Embodiment 37. The compound of Embodiment 35, a pharmaceutically acceptable salt or solvate thereof, wherein R^{1a} is Q-L-.

Embodiment 38. The compound of Embodiment 1 of Formula X or a pharmaceutically acceptable salt or solvate thereof.

Embodiment 39. The compound of Embodiment 38 of Formula XI or a pharmaceutically acceptable salt or solvate thereof.

Embodiment 40. The compound of Embodiment 38 of Formula XII or a pharmaceutically acceptable salt or solvate thereof.

Embodiment 41. The compound of any one of Embodiments 38-40, or a pharmaceutically acceptable salt or solvate thereof, wherein A¹ is —CR^{2a}═ and A³ is —CR^{2d}═.

Embodiment 42. The compound of Embodiment 41, or a pharmaceutically acceptable salt or solvate thereof, wherein R^{2a} is selected from hydrogen, —NH₂, fluoro, and methoxy.

Embodiment 43. The compound of Embodiments 41 or 42, or a pharmaceutically acceptable salt or solvate thereof, wherein R^{2d} is selected from hydrogen, —NH₂, fluoro, and methoxy.

Embodiment 44. The compound of any one of Embodiments 38-40, or a pharmaceutically acceptable salt or solvate thereof, wherein A¹ is —N═ and A³ is —CR^{2d}═.

Embodiment 45. The compound of Embodiment 44, or a pharmaceutically acceptable salt or solvate thereof, wherein R^{2d} is selected from hydrogen, —NH₂, fluoro, and methoxy.

Embodiment 46. The compound of any one of Embodiments 38-40, or a pharmaceutically acceptable salt or solvate thereof, wherein A¹ is —CR^{2a}═ and A³ is —N═.

Embodiment 47. The compound of Embodiment 46, or a pharmaceutically acceptable salt or solvate thereof, wherein R^{2a} is selected from hydrogen, —NH₂, fluoro, and methoxy.

Embodiment 48. The compound of any one of Embodiments 38-47, or a pharmaceutically acceptable salt or solvate thereof, wherein q is 2.

Embodiment 49. The compound of any one of Embodiments 38-48, or a pharmaceutically acceptable salt or solvate thereof, wherein q is 1.

Embodiment 50. The compound of any one of Embodiments 38-49, or a pharmaceutically acceptable salt or solvate thereof, wherein R^{1d} is hydrogen.

Embodiment 51. The compound of any one of Embodiments 38-49, or a pharmaceutically acceptable salt or solvate thereof, wherein R^{1d} is Q-L-.

Embodiment 52. The compound of Embodiment 1 of Formula XIII or a pharmaceutically acceptable salt or solvate thereof.

Embodiment 53. The compound of Embodiment 52 of Formula XIV or a pharmaceutically acceptable salt or solvate thereof.

Embodiment 54. The compound of Embodiment 52 of Formula XV or a pharmaceutically acceptable salt or solvate thereof.

Embodiment 55. The compound of any one of Embodiments 52-54, or a pharmaceutically acceptable salt or solvate thereof, wherein A¹ is —CR^{2a}═ and A³ is —CR^{2d}═.

Embodiment 56. The compound of Embodiment 54, or a pharmaceutically acceptable salt or solvate thereof, wherein R^{2a} is selected from hydrogen, —NH₂, fluoro, and methoxy.

Embodiment 57. The compound of Embodiments 55 or 56, or a pharmaceutically acceptable salt or solvate thereof, wherein R^{2d} is selected from hydrogen, —NH₂, fluoro, and methoxy.

Embodiment 58. The compound of any one of Embodiments 52-54, or a pharmaceutically acceptable salt or solvate thereof, wherein A¹ is —N═ and A³ is —CR^{2d}═.

Embodiment 59. The compound of Embodiment 58, or a pharmaceutically acceptable salt or solvate thereof, wherein R^{2d} is selected from hydrogen, —NH₂, fluoro, and methoxy.

Embodiment 60. The compound of any one of Embodiments 52-54, or a pharmaceutically acceptable salt or solvate thereof, wherein A¹ is —CR^{2a}═ and A³ is —N═.

Embodiment 61. The compound of Embodiment 60, or a pharmaceutically acceptable salt or solvate thereof, wherein R^{2a} is selected from hydrogen, —NH₂, fluoro, and methoxy.

Embodiment 62. The compound of Embodiment 1 of Formula XVI or a pharmaceutically acceptable salt or solvate thereof.

Embodiment 63. The compound of Embodiment 62 of Formula XVII or a pharmaceutically acceptable salt or solvate thereof.

Embodiment 64. The compound of Embodiment 62 of Formula XVIII or a pharmaceutically acceptable salt or solvate thereof.

Embodiment 65. The compound of Embodiment 1 of Formula XIX or a pharmaceutically acceptable salt or solvate thereof.

Embodiment 66. The compound of Embodiment 65 of Formula XX or a pharmaceutically acceptable salt or solvate thereof.

Embodiment 67. The compound of Embodiment 65 of Formula XXI or a pharmaceutically acceptable salt or solvate thereof.

Embodiment 68. The compound of any one of Embodiments 62-67, or a pharmaceutically acceptable salt or solvate thereof, wherein A² is —CR^{2c}═ and A³ is —CR^{2d}═.

Embodiment 69. The compound of Embodiment 68, or a pharmaceutically acceptable salt or solvate thereof, wherein R^{2e} is selected from hydrogen, —NH₂, fluoro, and methoxy.

Embodiment 70. The compound of Embodiments 68 or 69, or a pharmaceutically acceptable salt or solvate thereof, wherein R^{2d} is selected from hydrogen, —NH₂, fluoro, and methoxy.

Embodiment 71. The compound of any one of Embodiments 62-67, or a pharmaceutically acceptable salt or solvate thereof, wherein A² is —N═ and A³ is —CR^{2d}═.

Embodiment 72. The compound of Embodiment 71, or a pharmaceutically acceptable salt or solvate thereof, wherein R^{2d} is selected from hydrogen, —NH₂, fluoro, and methoxy.

Embodiment 73. The compound of any one of Embodiments 62-67, or a pharmaceutically acceptable salt or solvate thereof, wherein A² is —CR^{2c}═ and A is —N═.

Embodiment 74. The compound of Embodiment 73, or a pharmaceutically acceptable salt or solvate thereof, wherein R^{2e} is selected from hydrogen, —NH₂, fluoro, and methoxy.

Embodiment 75. The compound of any one of Embodiments 62-74, or a pharmaceutically acceptable salt or solvate thereof, wherein R^{1e} is hydrogen.

Embodiment 76. The compound of any one of Embodiments 62-74, or a pharmaceutically acceptable salt or solvate thereof, wherein R^{1e} is Q-L-.

Embodiment 77. The compound of Embodiment 1 of Formula XXII or a pharmaceutically acceptable salt or solvate thereof.

Embodiment 78. The compound of Embodiment 77 of Formula XXIII or a pharmaceutically acceptable salt or solvate thereof.

Embodiment 79. The compound of Embodiment 77 of Formula XXIV or a pharmaceutically acceptable salt or solvate thereof.

Embodiment 80. The compound of any one of Embodiments 77-79, or a pharmaceutically acceptable salt or solvate thereof, wherein A¹ is —CR^{2a}═ and A³ is —CR^{2d}═.

Embodiment 81. The compound of Embodiment 80, or a pharmaceutically acceptable salt or solvate thereof, wherein R^{2a} is selected from hydrogen, —NH₂, fluoro, and methoxy.

Embodiment 82. The compound of Embodiments 80 or 81, or a pharmaceutically acceptable salt or solvate thereof, wherein R^{2d} is selected from hydrogen, —NH₂, fluoro, and methoxy.

Embodiment 83. The compound of any one of Embodiments 77-79, or a pharmaceutically acceptable salt or solvate thereof, wherein A¹ is —N═ and A³ is —CR^{2d}═.

Embodiment 84. The compound of Embodiment 83, or a pharmaceutically acceptable salt or solvate thereof, wherein R^{2d} is selected from hydrogen, —NH₂, fluoro, and methoxy.

Embodiment 85. The compound of any one of Embodiments 77-79, or a pharmaceutically acceptable salt or solvate thereof, wherein A¹ is —CR^{2a}═ and A³ is —N═.

Embodiment 86. The compound of Embodiment 85, or a pharmaceutically acceptable salt or solvate thereof, wherein R^{2a} is selected from hydrogen, —NH₂, fluoro, and methoxy.

Embodiment 87. The compound of any one of Embodiments 77-86, or a pharmaceutically acceptable salt or solvate thereof, wherein E¹ is —N═.

Embodiment 88. The compound of any one of Embodiments 77-86, or a pharmaceutically acceptable salt or solvate thereof, wherein E¹ is —CR^{2e}═ and R^{2e} is Q-L-.

Embodiment 89. The compound of any one of Embodiments 77-86, or a pharmaceutically acceptable salt or solvate thereof, wherein E is —N═.

Embodiment 90. The compound of any one of Embodiments 77-86, or a pharmaceutically acceptable salt or solvate thereof, wherein E is —CR^{2f}═ and R²¹ is Q-L-.

Embodiment 91. The compound of any one of Embodiments 77-86, or a pharmaceutically acceptable salt or solvate thereof, wherein E² is —N═.

Embodiment 92. The compound of any one of Embodiments 77-86, or a pharmaceutically acceptable salt or solvate thereof, wherein E² is —CR^{2g}═ and R^{2g} is Q-L-.

Embodiment 93. The compound of any one of Embodiments 77-86, or a pharmaceutically acceptable salt or solvate thereof, wherein E³ is —N═.

Embodiment 94. The compound of any one of Embodiments 77-86, or a pharmaceutically acceptable salt or solvate thereof, wherein E³ is —CR^{2h}═ and R^{2h} is Q-L-.

Embodiment 95. The compound of Embodiment 1 of Formula XXV or a pharmaceutically acceptable salt or solvate thereof.

Embodiment 96. The compound of Embodiment 95 of Formula XXVI or a pharmaceutically acceptable salt or solvate thereof.

Embodiment 97. The compound of Embodiment 95 of Formula XXVII or a pharmaceutically acceptable salt or solvate thereof.

Embodiment 98. The compound of any one of Embodiments 95-97, or a pharmaceutically acceptable salt or solvate thereof, wherein A¹ is —CR^{2a}═ and A³ is —CR^{2d}═.

Embodiment 99. The compound of Embodiment 98, or a pharmaceutically acceptable salt or solvate thereof, wherein R^{2a} is selected from hydrogen, —NH₂, fluoro, and methoxy.

Embodiment 100. The compound of Embodiments 98 or 99, or a pharmaceutically acceptable salt or solvate thereof, wherein R^{2d} is selected from hydrogen, —NH₂, fluoro, and methoxy.

Embodiment 101. The compound of any one of Embodiments 95-97, or a pharmaceutically acceptable salt or solvate thereof, wherein A¹ is —N═ and A³ is —CR^{2d}═.

Embodiment 102. The compound of Embodiment 101, or a pharmaceutically acceptable salt or solvate thereof, wherein R^{2d} is selected from hydrogen, —NH₂, fluoro, and methoxy.

Embodiment 103. The compound of any one of Embodiments 95-97, or a pharmaceutically acceptable salt or solvate thereof, wherein A¹ is —CR^{2a}═ and A³ is —N═.

Embodiment 104. The compound of Embodiment 103, or a pharmaceutically acceptable salt or solvate thereof, wherein R^{2a} is selected from hydrogen, —NH₂, fluoro, and methoxy.

Embodiment 105. The compound of any one of Embodiments 95-104, or a pharmaceutically acceptable salt or solvate thereof, wherein A⁴ is —N═.

Embodiment 106. The compound of any one of Embodiments 95-104, or a pharmaceutically acceptable salt or solvate thereof, wherein A⁴ is —CH═.

Embodiment 107. The compound of any one of Embodiment 95-106, a pharmaceutically acceptable salt or solvate thereof, wherein R^{2k} is hydrogen.

Embodiment 108. The compound of any one of Embodiment 95-107, a pharmaceutically acceptable salt or solvate thereof, wherein R¹⁹ is hydrogen.

Embodiment 109. The compound of any one of Embodiment 95-107, a pharmaceutically acceptable salt or solvate thereof, wherein R¹⁹ is Q-L.

Embodiment 110. The compound of Embodiment 1 of Formula XXVIII or a pharmaceutically acceptable salt or solvate thereof.

Embodiment 111. The compound of Embodiment 110 of Formula XXIX or a pharmaceutically acceptable salt or solvate thereof.

Embodiment 112. The compound of Embodiment 110 of Formula XXX or a pharmaceutically acceptable salt or solvate thereof.

Embodiment 113. The compound of any one of Embodiments 110-112, or a pharmaceutically acceptable salt or solvate thereof, wherein A¹ is —CR^{2a}═ and A³ is —CR^{2d}=.

Embodiment 114. The compound of Embodiment 113, or a pharmaceutically acceptable salt or solvate thereof, wherein R^{2a} is selected from hydrogen, —NH₂, fluoro, and methoxy.

Embodiment 115. The compound of Embodiments 113 or 114, or a pharmaceutically acceptable salt or solvate thereof, wherein R^{2d} is selected from hydrogen, —NH₂, fluoro, and methoxy.

Embodiment 116. The compound of any one of Embodiments 110-112, or a pharmaceutically acceptable salt or solvate thereof, wherein A¹ is —N═ and A³ is —CR^{2d}=.

Embodiment 117. The compound of Embodiment 116, or a pharmaceutically acceptable salt or solvate thereof, wherein R^{2d} is selected from hydrogen, —NH₂, fluoro, and methoxy.

Embodiment 118. The compound of any one of Embodiments 110-112, or a pharmaceutically acceptable salt or solvate thereof, wherein A¹ is —CR^{2a}═ and A³ is —N═.

Embodiment 119. The compound of Embodiment 118, or a pharmaceutically acceptable salt or solvate thereof, wherein R^{2a} is selected from hydrogen, —NH₂, fluoro, and methoxy.

Embodiment 120. The compound of any one of Embodiments 110-119, or a pharmaceutically acceptable salt or solvate thereof, wherein m is 1.

Embodiment 121. The compound of any one of Embodiments 110-119, or a pharmaceutically acceptable salt or solvate thereof, wherein m is 2.

Embodiment 122. The compound of any one of Embodiments 110-121, or a pharmaceutically acceptable salt or solvate thereof, wherein n is 1.

Embodiment 123. The compound of any one of Embodiments 110-121, or a pharmaceutically acceptable salt or solvate thereof, wherein n is 2.

Embodiment 124. The compound of any one of Embodiments 110-123, or a pharmaceutically acceptable salt or solvate thereof, wherein o is 1.

Embodiment 125. The compound of any one of Embodiments 110-123, or a pharmaceutically acceptable salt or solvate thereof, wherein o is 2.

Embodiment 126. The compound of any one of Embodiments 110-125, or a pharmaceutically acceptable salt or solvate thereof, wherein p is 1.

Embodiment 127. The compound of any one of Embodiments 110-125, or a pharmaceutically acceptable salt or solvate thereof, wherein p is 2.

Embodiment 128. The compound of any one of Embodiments 110-127, or a pharmaceutically acceptable salt or solvate thereof, wherein R^{1h} is hydrogen.

Embodiment 129. The compound of any one of Embodiments 110-127, or a pharmaceutically acceptable salt or solvate thereof, wherein R^{1h} is Q-L-.

Embodiment 130. A compound of Formula XXXI or a pharmaceutically acceptable salt or solvate thereof.

Embodiment 131. The compound of Embodiment 130 of Formula XXXII or a pharmaceutically acceptable salt or solvate thereof.

Embodiment 132. The compound of Embodiment 130 of Formula XXXIII or a pharmaceutically acceptable salt or solvate thereof.

Embodiment 133. The compound of any one of Embodiments 130-132, or a pharmaceutically acceptable salt or solvate thereof, wherein A¹ is —CR^{2a}═ and A³ is —CR^{2d}═.

Embodiment 134. The compound of Embodiment 133, or a pharmaceutically acceptable salt or solvate thereof, wherein R^{2a} is selected from hydrogen, —NH₂, fluoro, and methoxy.

Embodiment 135. The compound of Embodiments 133 or 134, or a pharmaceutically acceptable salt or solvate thereof, wherein R^{2d} is selected from hydrogen, —NH₂, fluoro, and methoxy.

Embodiment 136. The compound of any one of Embodiments 130-132, or a pharmaceutically acceptable salt or solvate thereof, wherein A¹ is —N═ and A³ is —CR^{2d}═.

Embodiment 137. The compound of Embodiment 136, or a pharmaceutically acceptable salt or solvate thereof, wherein R^{2d} is selected from hydrogen, —NH₂, fluoro, and methoxy.

Embodiment 138. The compound of any one of Embodiments 130-132, or a pharmaceutically acceptable salt or solvate thereof, wherein A¹ is —CR^{2a}═ and A³ is —N═.

Embodiment 139. The compound of Embodiment 138, or a pharmaceutically acceptable salt or solvate thereof, wherein R^{2a} is selected from hydrogen, —NH₂, fluoro, and methoxy.

Embodiment 140. A compound of Formula XXXIV or a pharmaceutically acceptable salt or solvate thereof.

Embodiment 141. The compound of Embodiment 140 of Formula XXXV or a pharmaceutically acceptable salt or solvate thereof.

Embodiment 142. The compound of Embodiment 140 of Formula XXXVI or a pharmaceutically acceptable salt or solvate thereof.

Embodiment 143. A compound of Formula XXXVII or a pharmaceutically acceptable salt or solvate thereof.

Embodiment 144. The compound of Embodiment 143 of Formula XXXVIII or a pharmaceutically acceptable salt or solvate thereof.

Embodiment 145. The compound of Embodiment 143 of Formula XXXIX or a pharmaceutically acceptable salt or solvate thereof.

Embodiment 145. The compound of any one of Embodiments 140-145, or a pharmaceutically acceptable salt or solvate thereof, wherein A² is —CR^{2c}═ and A³ is —CR^{2d}═.

Embodiment 146. The compound of Embodiment 145, or a pharmaceutically acceptable salt or solvate thereof, wherein R^{2e} is selected from hydrogen, —NH₂, fluoro, and methoxy.

Embodiment 147. The compound of Embodiments 145 or 146, or a pharmaceutically acceptable salt or solvate thereof, wherein R^{2d} is selected from hydrogen, —NH₂, fluoro, and methoxy.

Embodiment 148. The compound of any one of Embodiments 140-145, or a pharmaceutically acceptable salt or solvate thereof, wherein A² is —N═ and A³ is —CR^{2d}═.

Embodiment 149. The compound of Embodiment 148, or a pharmaceutically acceptable salt or solvate thereof, wherein R^{2d} is selected from hydrogen, —NH₂, fluoro, and methoxy.

Embodiment 150. The compound of any one of Embodiments 140-145, or a pharmaceutically acceptable salt or solvate thereof, wherein A² is —CR^{2c}═ and A is —N═.

Embodiment 151. The compound of Embodiment 150, or a pharmaceutically acceptable salt or solvate thereof, wherein R^{2e} is selected from hydrogen, —NH₂, fluoro, and methoxy.

Embodiment 152. The compound of any one of Embodiments 130-151, or a pharmaceutically acceptable salt or solvate thereof, wherein E⁴ is ═C(H)—.

Embodiment 153. The compound of any one of Embodiments 130-151, or a pharmaceutically acceptable salt or solvate thereof, wherein E⁴ is ═N—.

Embodiment 154. The compound of any one of Embodiments 130-153, or a pharmaceutically acceptable salt or solvate thereof, wherein E⁵ is —O—.

Embodiment 155. The compound of any one of Embodiments 130-153, or a pharmaceutically acceptable salt or solvate thereof, wherein E⁵ is —S—.

Embodiment 156. The compound of any one of Embodiments 130-153, or a pharmaceutically acceptable salt or solvate thereof, wherein E⁵ is selected from —NH— and —N(CH₃)—.

Embodiment 157. The compound of any one of Embodiments 130-153, or a pharmaceutically acceptable salt or solvate thereof, wherein R^{1j} is selected from hydrogen, C₁-C₄ alkyl, (hydroxy)alkyl, and (heterocyclo)alkyl;

Embodiment 157. The compound of any one of Embodiments 130-153, or a pharmaceutically acceptable salt or solvate thereof, wherein R^{1j} is Q-L-.

Embodiment 158. The compound of any one of Embodiments 1-157, or a pharmaceutically acceptable salt or solvate thereof, wherein R³ is hydrogen.

Embodiment 159. The compound of any one of Embodiments 1-158, or a pharmaceutically acceptable salt or solvate thereof, wherein Z and Z¹ are —C(═O)—.

Embodiment 160. The compound of any one of Embodiments 1-158, or a pharmaceutically acceptable salt or solvate thereof, wherein Z is —C(═O)— and Z¹ is —CR^{4a}R^{4b}—;

Embodiment 161. The compound of any one of Embodiments 1-158, or a pharmaceutically acceptable salt or solvate thereof, wherein Z is —CR^{4a}R^{4b}—; and Z¹ is —C(═O)—.

Embodiment 162. The compound of Embodiments 160 or 161, or a pharmaceutically acceptable salt or solvate thereof, wherein R^{4a} and R^{4b} are hydrogen,

Embodiment 163. The compound of any one of Embodiments 1-158, or a pharmaceutically acceptable salt or solvate thereof, wherein Z is —N═C(CH₃)— and Z¹ is —C(═O)—.

Embodiment 164. The compound of any one of Embodiments 1-158, or a pharmaceutically acceptable salt or solvate thereof, wherein Z is —C(═O)— and Z¹ is —N═C(CH₃)—.

Embodiment 165. The compound of any one of Embodiments 1-158, or a pharmaceutically acceptable salt or solvate thereof, wherein Z is a bond and Z¹ is —N(CH₃)C(═O)—.

Embodiment 166. The compound of any one of Embodiments 1-158, or a pharmaceutically acceptable salt or solvate thereof, wherein Z is —N(CH₃)C(═O) and Z is a bond.

Embodiment 167. The compound of Embodiment 1, or a pharmaceutically acceptable salt or solvate thereof, that is any one or more of the compounds of Table 1.

Embodiment 168. A pharmaceutical composition comprising the compound of any one of Embodiments 1-167, or a pharmaceutically acceptable salt or solvate thereof, and a pharmaceutically acceptable carrier.

Embodiment 169. A method of treating cancer in a subject in need thereof, the method comprising administering to the subject a therapeutically effective amount of the compound of any one of Embodiments 1-167, or a pharmaceutically acceptable salt or solvate thereof.

Embodiment 170. The method of Embodiment 169, wherein the cancer is any one or more of the cancers of Table I.

Embodiment 171. The method of Embodiments 169 or 170 further comprising administering a therapeutically effective amount of an optional therapeutic agent useful in the treatment of cancer.

Embodiment 172. The pharmaceutical composition of Embodiment 168 for use in treating cancer.

Embodiment 173. The pharmaceutical composition of Embodiment 172, wherein the cancer is any one or more of the cancers of Table I.

Embodiment 174. A compound of any one of Embodiments 1-167, or a pharmaceutically acceptable salt or solvate thereof, for use in treating of cancer.

Embodiment 175. The compound for use of Embodiment 174, wherein the cancer is any one or more of the cancers of Table I.

Embodiment 176. Use of a compound of any one of Embodiments 1-167, or a pharmaceutically acceptable salt or solvate thereof, for the manufacture of a medicament for treatment of cancer.

Embodiment 177. The use of Embodiment 176, wherein the cancer is any one or more of the cancers of Table I.

Embodiment 178. A method of inhibiting CRBN ubiquitination within a cell of a subject in need thereof, the method comprising administering to the subject a compound of any one of Embodiments 1-176, or a pharmaceutically acceptable salt or solvate thereof.

### Definitions

The term “a disease or condition wherein inhibition of CRBN ubiquitination provides a benefit” and the like pertains to a disease or condition in which CRBN ubiquitination is important or necessary, e.g., for the onset, progress, expression of that disease or condition, or a disease or a condition which is known to be treated by an CRBN ubiquitination inhibitor, e.g., thalidomide, lenalidomide, pomalidomide, and related analogs. Examples of such conditions include, but are not limited, cancer. One of ordinary skill in the art is readily able to determine whether a compound treats a disease or condition mediated by a CRBN ubiquitination inhibitor for any particular cell type, for example, by assays which conveniently can be used to assess the activity of particular compounds.

The terms “cereblon” or “CRBN” refers to a protein that is encoded by the CRBN gene in humans. Cereblon forms an E3 ubiquitin ligase complex with damaged DNA binding protein 1 (DDB1), Cullin-4A (CUL4A), and regulator of cullins 1 (ROC1). This complex ubiquitinates a number of other proteins. Angers et al., Nature 443:590-593 (2006)

The term “optional therapeutic agent” or “second therapeutic agent” refers to a therapeutic agent different from a Cereblon Ligand or PROTAC moleculesand that is known to treat the disease or condition of interest. For example when a cancer is the disease or condition of interest, the optional therapeutic agent can be a known chemotherapeutic drug, like taxol, or radiation, for example. In some embodiments, a Compound of the Disclosure and a second therapeutic agent are administered concurrently (e.g., simultaneously or sequentially). In some embodiments, a Compound of the Disclosure and a second therapeutic agent are administered in temporal proximity.

As used herein, the term “subject” includes human and non-human animals, as well as cell lines, cell cultures, tissues, and organs. In some embodiments, the subject is a mammal.

The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

The term “disease” or “condition” denotes disturbances and/or anomalies that as a rule are regarded as being pathological conditions or functions, and that can manifest themselves in the form of particular signs, symptoms, and/or malfunctions. Cereblon Ligands are inhibitors of CRBN ubiquitination and can be used in treating or preventing diseases and conditions wherein the inhibition of CRBN ubiquitination provides a benefit.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. The term “treat” and synonyms contemplate administering a therapeutically effective amount of a Cereblon Ligand to a subject in need of such treatment. The treatment can be orientated symptomatically, for example, to suppress symptoms. It can be effected over a short period, be oriented over a medium term, or can be a long-term treatment, for example within the context of a maintenance therapy.

As used herein, the terms “prevent,” “preventing,” and “prevention” refer to a method of preventing the onset of a disease or condition and/or its attendant symptoms or barring a subject from acquiring a disease. As used herein, “prevent,” “preventing,” and “prevention” also include delaying the onset of a disease and/or its attendant symptoms and reducing a subject's risk of acquiring a disease. The terms “prevent,” “preventing” and “prevention” may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition.

The term “therapeutically effective amount” or “effective dose” as used herein refers to an amount of the active ingredient(s) that is(are) sufficient, when administered by a method of the disclosure, to efficaciously deliver the active ingredient(s) for the treatment of condition or disease of interest to a subject in need thereof. In the case of a cancer or other proliferation disorder, the therapeutically effective amount of the agent may reduce (i.e., retard to some extent or stop) unwanted cellular proliferation; reduce the number of cancer cells; reduce the tumor size; inhibit (i.e., retard to some extent or stop) cancer cell infiltration into peripheral organs; inhibit (i.e., retard to some extent or stop) tumor metastasis; inhibit, to some extent, tumor growth; and/or relieve, to some extent, one or more of the symptoms associated with the cancer. To the extent the administered compound or composition prevents growth and/or kills existing cancer cells, it may be cytostatic and/or cytotoxic.

The term “container” means any receptacle and closure therefore suitable for storing, shipping, dispensing, and/or handling a pharmaceutical product.

The term “insert” means information accompanying a pharmaceutical product that provides a description of how to administer the product, along with the safety and efficacy data required to allow the physician, pharmacist, and subject to make an informed decision regarding use of the product. The package insert generally is regarded as the “label” for a pharmaceutical product.

“Concurrent administration,” “administered in combination,” “simultaneous administration,” and similar phrases mean that two or more agents are administered concurrently to the subject being treated. By “concurrently,” it is meant that each agent is administered either simultaneously or sequentially in any order at different points in time.

However, if not administered simultaneously, it is meant that they are administered to an individual in a sequence and sufficiently close in time so as to provide the desired therapeutic effect and can act in concert. For example, a Compound of the Disclosure can be administered at the same time or sequentially in any order at different points in time as a second therapeutic agent. A Compound of the Disclosure and the second therapeutic agent can be administered separately, in any appropriate form and by any suitable route. When a Compound of the Disclosure and the second therapeutic agent are not administered concurrently, it is understood that they can be administered in any order to a subject in need thereof. For example, a Compound of the Disclosure can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent treatment modality (e.g., radiotherapy), a subject in need thereof. In various embodiments, a Compound of the Disclosure and the second therapeutic agent are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In some embodiments, the components of the combination therapies are administered at about 1 minute to about 24 hours apart.

As used herein, the term “temporal proximity” refers to that administration of one therapeutic agent (e.g., a Compound of the Disclosure) occurs within a time period before or after the administration of another therapeutic agent (e.g., a second therapeutic agent), such that the therapeutic effect of the one therapeutic agent overlaps with the therapeutic effect of the other therapeutic agent. In some embodiments, the therapeutic effect of the one therapeutic agent completely overlaps with the therapeutic effect of the other therapeutic agent. In some embodiments, “temporal proximity” means that administration of one therapeutic agent occurs within a time period before or after the administration of another therapeutic agent, such that there is a synergistic effect between the one therapeutic agent and the other therapeutic agent. “Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

The use of the terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

The term “halo” as used herein by itself or as part of another group refers to —Cl, —F, —Br, or —I.

The term “nitro” as used herein by itself or as part of another group refers to —NO₂.

The term “cyano” as used herein by itself or as part of another group refers to —CN.

The term “hydroxy” as herein used by itself or as part of another group refers to —OH.

The term “alkyl” as used herein by itself or as part of another group refers to a straight- or branched-chain aliphatic hydrocarbon containing one to twelve carbon atoms, i.e., a C₁-C₁₂ alkyl, or the number of carbon atoms designated, e.g., a C₁ alkyl such as methyl, a C₂ alkyl such as ethyl, etc. In some embodiments, the alkyl is a C₁-C₁₀ alkyl. In some embodiments, the alkyl is a C₁-C₆ alkyl. In some embodiments, the alkyl is a C₁-C₄ alkyl. In some embodiments, the alkyl is a C₁-C₃ alkyl, i.e., methyl, ethyl, propyl, or isopropyl. Non-limiting exemplary C₁-C₁₂ alkyl groups include methyl, ethyl, propyl, isopropyl, butyl, sec-butyl, tert-butyl, iso-butyl, 3-pentyl, hexyl, heptyl, octyl, nonyl, and decyl.

The term “optionally substituted alkyl” as used herein by itself or as part of another group refers to an alkyl group that is either unsubstituted or substituted with one, two, or three substituents, wherein each substituent is independently nitro, haloalkoxy, aryloxy, aralkyloxy, alkylthio, sulfonamido, alkylcarbonyl, arylcarbonyl, alkylsulfonyl, arylsulfonyl, ureido, guanidino, carbamate, carboxy, alkoxycarbonyl, carboxyalkyl, —N(R^{50a})C(═O)R^{50b}, —N(R^{50a})S(═O)₂R^{50c}, —C(═O)R⁵¹, —S(═O)R⁵², or —S(═O)₂R⁵³; wherein:
R^{50a} is hydrogen or alkyl;
R^{50b} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;
R^{50c} is alkyl, haloalkyl, optionally substituted cycloalkyl, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;
R⁵¹ is haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, or optionally substituted heteroaryl;
R⁵² is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl; and
R⁵³ is haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, or optionally substituted heteroaryl. Non-limiting exemplary optionally substituted alkyl groups include —CH(CO₂Me)CH₂CO₂Me and —CH(CH₃)CH₂N(H)C(═O)O(CH₃)₃.

The term “alkenyl” as used herein by itself or as part of another group refers to an alkyl group containing one, two, or three carbon-to-carbon double bonds. In some embodiments, the alkenyl group is a C₂-C₆ alkenyl group. In some embodiments, the alkenyl group is a C₂-C₄ alkenyl group. In some embodiments, the alkenyl group has one carbon-to-carbon double bond. Non-limiting exemplary alkenyl groups include ethenyl, propenyl, isopropenyl, butenyl, sec-butenyl, pentenyl, and hexenyl.

The term “optionally substituted alkenyl” as used herein by itself or as part of another refers to an alkenyl group that is either unsubstituted or substituted with one, two or three substituents, wherein each substituent is independently halo, nitro, cyano, hydroxy, amino (e.g., alkylamino, dialkylamino), haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted cycloalkyl, alkenyl, alkynyl, optionally substituted aryl, optionally substituted heteroaryl, or optionally substituted heterocyclo. Non-limiting exemplary optionally substituted alkenyl groups include —CH═CHPh.

The term “alkynyl” as used herein by itself or as part of another group refers to an alkyl group containing one, two, or three carbon-to-carbon triple bonds. In some embodiments, the alkynyl is a C₂-C₆ alkynyl. In some embodiments, the alkynyl is a C₂-C₄ alkynyl. In some embodiments, the alkynyl has one carbon-to-carbon triple bond. Non-limiting exemplary alkynyl groups include ethynyl, propynyl, butynyl, 2-butynyl, pentynyl, and hexynyl groups.

The term “optionally substituted alkynyl” as used herein by itself or as part of another group refers to an alkynyl group that is either unsubstituted or substituted with one, two or three substituents, wherein each substituent is independently halo, nitro, cyano, hydroxy, amino, e.g., alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted cycloalkyl, alkenyl, alkynyl, optionally substituted aryl, optionally substituted heteroaryl, or optionally substituted heterocyclo. Non-limiting exemplary optionally substituted alkynyl groups include —C≡CPh and —CH(Ph)C≡CH.

The term “haloalkyl” as used herein by itself or as part of another group refers to an alkyl group substituted by one or more fluorine, chlorine, bromine, and/or iodine atoms. In some embodiments, the alkyl is substituted by one, two, or three fluorine and/or chlorine atoms.

In some embodiments, the alkyl is substituted by one, two, or three fluorine atoms. In some embodiments, the alkyl is a C₁-C₆ alkyl. In some embodiments, the alkyl is a C₁-C₄ alkyl.

In some embodiments, the alkyl group is a C₁ or C₂ alkyl. Non-limiting exemplary haloalkyl groups include fluoromethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

The terms “hydroxyalkyl” or “(hydroxy)alkyl” as used herein by themselves or as part of another group refer to an alkyl group substituted with one, two, or three hydroxy groups. In some embodiments, the alkyl is a C₁-C₆ alkyl. In some embodiments, the alkyl is a C₁-C₄ alkyl. In some embodiments, the alkyl is a C₁ or C₂ alkyl. In some embodiments, the hydroxyalkyl is a monohydroxyalkyl group, i.e., substituted with one hydroxy group. In some embodiments, the hydroxyalkyl group is a dihydroxyalkyl group, i.e., substituted with two hydroxy groups. Non-limiting exemplary (hydroxyl)alkyl groups include hydroxymethyl, hydroxyethyl, hydroxypropyl and hydroxybutyl groups, such as 1-hydroxyethyl, 2-hydroxyethyl, 1,2-dihydroxyethyl, 2-hydroxypropyl, 3-hydroxypropyl, 3-hydroxybutyl, 4-hydroxybutyl, 2-hydroxy-1-methylpropyl, and 1,3-dihydroxyprop-2-yl.

The term “alkoxy” as used herein by itself or as part of another group refers to an alkyl group or alkenyl group attached to a terminal oxygen atom. In some embodiments, the alkyl is a C₁-C₆ alkyl and resulting alkoxy is thus referred to as a “C₁-C₆ alkoxy.” In some embodiments, the alkyl is a C₁-C₄ alkyl group. Non-limiting exemplary alkoxy groups include methoxy, ethoxy, and tert-butoxy.

The term “haloalkoxy” as used herein by itself or as part of another group refers to a haloalkyl group attached to a terminal oxygen atom. In some embodiments, the haloalkyl group is a C₁-C₆ haloalkyl. In some embodiments, the haloalkyl group is a C₁-C₄ haloalkyl group. Non-limiting exemplary haloalkoxy groups include fluoromethoxy, difluoromethoxy, trifluoromethoxy, and 2,2,2-trifluoroethoxy.

The term “alkylthio” as used herein by itself or as part of another group refers to an alkyl group attached to a terminal sulfur atom. In some embodiments, the alkyl group is a C₁-C₄ alkyl group. Non-limiting exemplary alkylthio groups include —SCH₃, and —SCH₂CH₃.

The terms “alkoxyalkyl” or “(alkoxy)alkyl” as used herein by themselves or as part of another group refers to an alkyl group substituted with one alkoxy group. In some embodiments, the alkoxy is a C₁-C₆ alkoxy. In some embodiments, the alkoxy is a C₁-C₄ alkoxy. In some embodiments, the alkyl is a C₁-C₆ alkyl. In some embodiments, the alkyl is a C₁-C₄ alkyl. Non-limiting exemplary alkoxyalkyl groups include methoxymethyl, methoxyethyl, methoxypropyl, methoxybutyl, ethoxymethyl, ethoxyethyl, ethoxypropyl, ethoxybutyl, propoxymethyl, iso-propoxymethyl, propoxyethyl, propoxypropyl, butoxymethyl, tert-butoxymethyl, isobutoxymethyl, sec-butoxymethyl, and pentyloxymethyl.

The term “heteroalkyl” as used by itself or part of another group refers to unsubstituted straight- or branched-chain aliphatic hydrocarbons containing from three to twenty chain atoms, i.e., 3- to 20-membered heteroalkyl, or the number of chain atoms designated, wherein at least one —CH₂— is replaced with at least one of —O—, —N(H)—, —N(C₁-C₄ alkyl)-, or —S—. The —O—, —N(H)—, —N(C₁-C₄ alkyl)-, or —S— can independently be placed at any interior position of the aliphatic hydrocarbon chain so long as each —O—, —N(H)—, —N(C₁-C₄ alkyl)-, and —S— group is separated by at least two —CH₂— groups. In some embodiments, one —CH₂— group is replaced with one —O— group. In some embodiments, two —CH₂— groups are replaced with two —O— groups. In some embodiments, three —CH₂— groups are replaced with three —O— groups. In some embodiments, four —CH₂— groups are replaced with four —O— groups. Non-limiting exemplary heteroalkyl groups include —CH₂OCH₃, —CH₂OCH₂CH₂CH₃, —CH₂CH₂CH—₂OCH₃, —CH₂CH₂OCH₂CH₂OCH₂CH₃, —CH₂CH₂OCH₂CH₂OCH₂CH₂OCH₂CH₃.

The term “cycloalkyl” as used herein by itself or as part of another group refers to saturated and partially unsaturated, e.g., containing one or two double bonds, monocyclic, bicyclic, or tricyclic aliphatic hydrocarbons containing three to twelve carbon atoms, i.e., a C₃₋₁₂ cycloalkyl, or the number of carbons designated, e.g., a C₃ cycloalkyl such a cyclopropyl, a C₄ cycloalkyl such as cyclobutyl, etc. In some embodiments, the cycloalkyl is bicyclic, i.e., it has two rings. In some embodiments, the cycloalkyl is monocyclic, i.e., it has one ring. In some embodiments, the cycloalkyl is a C₃-8 cycloalkyl. In some embodiments, the cycloalkyl is a C₃-6 cycloalkyl, i.e., cyclopropyl, cyclobutyl, cyclopentyl, or cyclohexyl. In some embodiments, the cycloalkyl is a C₅ cycloalkyl, i.e., cyclopentyl. In some embodiments, the cycloalkyl is a C₆ cycloalkyl, i.e., cyclohexyl. Non-limiting exemplary C₃₋₁₂ cycloalkyl groups include cyclopropyl, cyclobutyl, cyclopentyl, cyclohexyl, cycloheptyl, cyclooctyl, norbornyl, decalin, adamantyl, cyclohexenyl, and spiro[3.3]heptane.

The term “optionally substituted cycloalkyl” as used herein by itself or as part of another group refers to a cycloalkyl group that is either unsubstituted or substituted with one, two, or three substituents, wherein each substituent is independently halo, nitro, cyano, hydroxy, amino (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{50a})C(═O)R^{50b}, —N(R^{50a})S(═O)₂R^{50c}, —C(═O)R⁵¹, —S(═O)R⁵², —S(═O)₂R⁵³, or —OR⁵⁴, wherein R^{50a}, R^{50b}, R^{50c}, R⁵², R⁵¹, and R⁵³ are as defined in connection with the term “optionally substituted alkyl” and R⁵⁴ is (hydroxy)alkyl or (amino)alkyl. The term optionally substituted cycloalkyl also includes cycloalkyl groups having fused optionally substituted aryl or optionally substituted heteroaryl groups such as

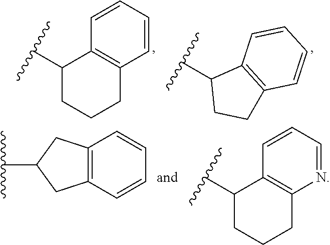

Non-limiting exemplary optionally substituted cycloalkyl groups include:

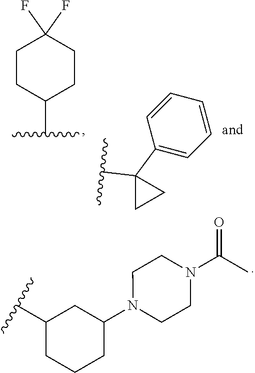 [SMILES: *C1CCC(F)(F)CC1 ; *C1(c2ccccc2)CC1 ; *C1CCCC(N2CCN(C(C)=O)CC2)C1]

The term “heterocyclo” as used herein by itself or as part of another group refers to saturated and partially unsaturated, e.g., containing one or two double bonds, monocyclic, bicyclic, or tricyclic groups (e.g., fused or spiro) containing three to fourteen ring members, i.e., a 3- to 14-membered heterocyclo, comprising one, two, three, or four heteroatoms. Each heteroatom is independently oxygen, sulfur, or nitrogen. Each sulfur atom is independently oxidized to give a sulfoxide, i.e., S(═O), or sulfone, i.e., S(═O)₂.

The term heterocyclo includes groups wherein one or more —CH₂— groups is replaced with one or more —C(═O)— groups, including cyclic ureido groups such as imidazolidinyl-2-one, cyclic amide groups such as pyrrolidin-2-one or piperidin-2-one, and cyclic carbamate groups such as oxazolidinyl-2-one.

The term heterocyclo also includes groups having fused optionally substituted aryl or optionally substituted heteroaryl groups such as indoline, indolin-2-one, 2,3-dihydro-1H-pyrrolo[2,3-c]pyridine, 2,3,4,5-tetrahydro-1H-benzo[d]azepine, or 1,3,4,5-tetrahydro-2H-benzo[d]azepin-2-one.

In some embodiments, the heterocyclo group is a 4- to 8-membered cyclic group containing one ring and one or two oxygen atoms, e.g., tetrahydrofuran or tetrahydropyran, or one or two nitrogen atoms, e.g., pyrrolidine, piperidine, or piperazine, or one oxygen and one nitrogen atom, e.g., morpholine, and, optionally, one —CH₂— group is replaced with one —C(═O)— group, e.g., pyrrolidin-2-one or piperazin-2-one. In some embodiments, the heterocyclo group is a 5- to 8-membered cyclic group containing one ring and one or two nitrogen atoms and, optionally, one —CH₂— group is replaced with one —C(═O)— group. In some embodiments, the heterocyclo group is a 5- or 6-membered cyclic group containing one ring and one or two nitrogen atoms and, optionally, one —CH₂— group is replaced with one —C(═O)— group. In some embodiments, the heterocyclo group is a 8- to 12-membered cyclic group containing two rings and one or two nitrogen atoms. The heterocyclo can be linked to the rest of the molecule through any available carbon or nitrogen atom. Non-limiting exemplary heterocyclo groups include:

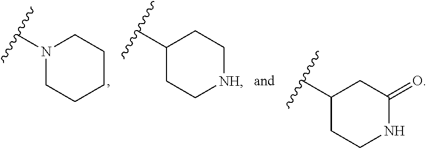

The term “optionally substituted heterocyclo” as used herein by itself or part of another group refers to a heterocyclo group that is either unsubstituted or substituted with one to four substituents, wherein each substituent is independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{50a})C(═O)Rob, —N(R^{50a})S(═O)₂R^{50c}, —C(═O)R⁵¹, —S(═O)R⁵², —S(═O)₂R⁵³, or —OR⁵⁴, wherein R^{50a}, R^{50b}, R^{50c}, R⁵², R⁵¹, R⁵³, and R⁵⁴ are as defined in connection with the term “optionally substituted cycloalkyl.” Substitution may occur on any available carbon or nitrogen atom of the heterocyclo group. Non-limiting exemplary optionally substituted heterocyclo groups include:

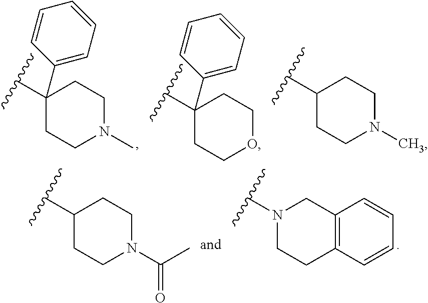

The term “aryl” as used herein by itself or as part of another group refers to an aromatic ring system having six to fourteen carbon atoms, i.e., C₆-C₁₄ aryl. Non-limiting exemplary aryl groups include phenyl (abbreviated as “Ph”), naphthyl, phenanthryl, anthracyl, indenyl, azulenyl, biphenyl, biphenylenyl, and fluorenyl groups. In some embodiments, the aryl group is phenyl or naphthyl. In some embodiments, the aryl group is phenyl.

The term “optionally substituted aryl” as used herein by itself or as part of another group refers to aryl that is either unsubstituted or substituted with one to five substituents, wherein the substituents are each independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{50a})C(═O)R^{50b}, —N(R^{50a})S(═O)₂R^{50c}, —C(═O)R⁵¹, —S(═O)R⁵², —S(═O)₂R⁵³, or —OR⁵⁴, wherein R^{50a}, R^{50b}, R^{50c}, R⁵², R⁵¹, R⁵³, and R⁵⁴ are as defined in connection with the term “optionally substituted cycloalkyl.”

In some embodiments, the optionally substituted aryl is an optionally substituted phenyl. In some embodiments, the optionally substituted phenyl has four substituents. In some embodiments, the optionally substituted phenyl has three substituents. In some embodiments, the optionally substituted phenyl has two substituents. In some embodiments, the optionally substituted phenyl has one substituent. Non-limiting exemplary optionally substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 2,6-di-fluorophenyl, 2,6-di-chlorophenyl, 2-methyl, 3-methoxyphenyl, 2-ethyl, 3-methoxyphenyl, 3,4-di-methoxyphenyl, 3,5-di-fluorophenyl 3,5-di-methylphenyl, 3,5-dimethoxy, 4-methylphenyl, 2-fluoro-3-chlorophenyl, 3-chloro-4-fluorophenyl, and 2-phenylpropan-2-amine. The term optionally substituted aryl includes aryl groups having fused optionally substituted cycloalkyl groups and fused optionally substituted heterocyclo groups. Non-limiting examples include: 2,3-dihydro-1H-inden-1-yl, 1,2,3,4-tetrahydronaphthalen-1-yl, 1,3,4,5-tetrahydro-2H-benzo[c]azepin-2-yl, 1,2,3,4-tetrahydroisoquinolin-1-yl, and 2-oxo-2,3,4,5-tetrahydro-1H-benzo[d]azepin-1-yl.

The term “heteroaryl” as used herein by itself or as part of another group refers to monocyclic and bicyclic aromatic ring systems having five to 14 fourteen ring members, i.e., a 5- to 14-membered heteroaryl, comprising one, two, three, or four heteroatoms. Each heteroatom is independently oxygen, sulfur, or nitrogen. In some embodiments, the heteroaryl has three heteroatoms. In some embodiments, the heteroaryl has two heteroatoms. In some embodiments, the heteroaryl has one heteroatom. In some embodiments, the heteroaryl is a 5- to 10-membered heteroaryl. In some embodiments, the heteroaryl has 5 ring atoms, e.g., thienyl, a 5-membered heteroaryl having four carbon atoms and one sulfur atom. In some embodiments, the heteroaryl has 6 ring atoms, e.g., pyridyl, a 6-membered heteroaryl having five carbon atoms and one nitrogen atom. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, O-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In some embodiments, the heteroaryl is chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) and isoxazolyl (e.g., isoxazol-3-yl, isoxazol-4-yl, and isoxazol-5-yl). The term heteroaryl also includes N-oxides. A non-limiting exemplary N-oxide is pyridyl N-oxide.

The term “optionally substituted heteroaryl” as used herein by itself or as part of another group refers to a heteroaryl that is either unsubstituted or substituted with one to four substituents, wherein the substituents are independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{50a})C(═O)Rob, —N(R^{50a})S(═O)₂R^{50c}, —C(═O)R⁵¹, —S(═O)R⁵², —S(═O)₂R⁵³, or —OR⁵⁴, wherein R^{50a}, R^{50b}, R^{50c}, R⁵², R⁵¹, R⁵³, and R⁵⁴ are as defined in connection with the term “optionally substituted cycloalkyl.”

In some embodiments, the optionally substituted heteroaryl has two substituents. In some embodiments, the optionally substituted heteroaryl has one substituent. Any available carbon or nitrogen atom can be substituted.

The term “aryloxy” as used herein by itself or as part of another group refers to an optionally substituted aryl attached to a terminal oxygen atom. A non-limiting exemplary aryloxy group is PhO—.

The term “aralkyloxy” as used herein by itself or as part of another group refers to an aralkyl attached to a terminal oxygen atom. A non-limiting exemplary aralkyloxy group is PhCH₂O—.

The term “carboxyalkyl” as used herein by itself or as part of another group refers to an alkyl substituted with one carboxy group. In some embodiments, the alkyl is a C₁-C₄ alkyl. Non-limiting exemplary carboxyalkyl groups include —CH₂CO₂H and —CH₂CH₂CO₂H.

The term “(cyano)alkyl” as used herein by itself or as part of another group refers to an alkyl substituted with one, two, or three cyano groups. In some embodiments, the alkyl is substituted with one cyano group. In some embodiments, the alkyl is a C₁-C₆ alkyl. In some embodiments, the alkyl is a C₁-C₄ alkyl. Non-limiting exemplary (cyano)alkyl groups include —CH₂CH₂CN and —CH₂CH₂CH₂CN.

The term “(cycloalkyl)alkyl” as used herein by itself or as part of another group refers to an alkyl substituted with one or two optionally substituted cycloalkyl groups. In some embodiments, the cycloalkyl group(s) is an optionally substituted C₃-C₆ cycloalkyl. In some embodiments, the alkyl is a C₁-C₆ alkyl. In some embodiments, the alkyl is a C₁-C₄ alkyl. In some embodiments, the alkyl is a C₁ or C₂ alkyl. In some embodiments, the alkyl is substituted with one optionally substituted cycloalkyl group. In some embodiments, the alkyl is substituted with two optionally substituted cycloalkyl groups. Non-limiting exemplary (cycloalkyl)alkyl groups include:

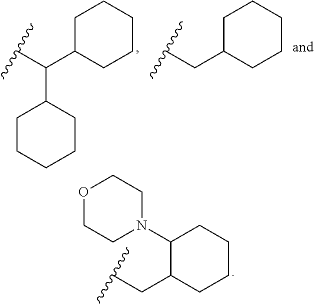

The term “sulfonamido” as used herein by itself or as part of another group refers to a radical of the formula —SO₂NR^{54a}R^{54b}, wherein R^{54a} and R^{54b} are each independently hydrogen, alkyl, optionally substituted cycloalkyl, optionally substituted heterocyclo, optionally substituted aryl, or optionally substituted heteroaryl; or R^{54a} and R^{54b} taken together with the nitrogen to which they are attached form a 3- to 8-membered optionally substituted heterocyclo group. Non-limiting exemplary sulfonamido groups include —SO₂NH₂, —SO₂N(H)CH₃, and —SO₂N(H)Ph.

The term “alkylcarbonyl” as used herein by itself or as part of another group refers to a carbonyl group, i.e., —C(═O)—, substituted by an alkyl group. In some embodiments, the alkyl is a C₁-C₄ alkyl. A non-limiting exemplary alkylcarbonyl group is —COCH₃.

The term “arylcarbonyl” as used herein by itself or as part of another group refers to a carbonyl group, i.e., —C(═O)—, substituted by an optionally substituted aryl group.

A non-limiting exemplary arylcarbonyl group is —COPh.

The term “alkylsulfonyl” as used herein by itself or as part of another group refers to a sulfonyl group, i.e., —SO₂—, substituted by an alkyl group. A non-limiting exemplary alkylsulfonyl group is —SO₂CH₃.

The term “arylsulfonyl” as used herein by itself or as part of another group refers to a sulfonyl group, i.e., —SO₂—, substituted by an optionally substituted aryl group. A non-limiting exemplary arylsulfonyl group is —SO₂Ph.

The term “mercaptoalkyl” as used herein by itself or as part of another group refers to an alkyl substituted by a —SH group.

The term “carboxy” as used by itself or as part of another group refers to a radical of the formula —C(═O)OH.

The term “ureido” as used herein by itself or as part of another group refers to a radical of the formula —NR^{51a}—C(═O)—NR^{51b}R^{51c}, wherein R^{51a} is hydrogen or alkyl; and R^{51b} and R^{51c} are each independently hydrogen, alkyl, optionally substituted cycloalkyl, optionally substituted heterocyclo, optionally substituted aryl, or optionally substituted heteroaryl, or R^{51b} and R⁵¹, taken together with the nitrogen to which they are attached form a 4- to 8-membered optionally substituted heterocyclo group. Non-limiting exemplary ureido groups include —NH—C(C═O)—NH₂ and —NH—C(C═O)—NHCH₃.

The term “guanidino” as used herein by itself or as part of another group refers to a radical of the formula —NR^{52a}—C(═NR⁵³)—NR^{52b}R^{52c}, wherein R^{52a} is hydrogen or alkyl; R^{52b} and R^{53c} are each independently hydrogen, alkyl, optionally substituted cycloalkyl, optionally substituted heterocyclo, optionally substituted aryl, or optionally substituted heteroaryl; or R^{52b} and R^{52c} taken together with the nitrogen to which they are attached form a 4- to 8-membered optionally substituted heterocyclo group; and R⁵³ is hydrogen, alkyl, cyano, alkylsulfonyl, alkylcarbonyl, carboxamido, or sulfonamido. Non-limiting exemplary guanidino groups include —NH—C(C═NH)—NH₂, —NH—C(C═NCN)—NH₂, and —NH—C(C═NH)—NHCH₃.

The term “(heterocyclo)alkyl” as used herein by itself or as part of another group refers to an alkyl substituted with one, two, or three optionally substituted heterocyclo groups. In some embodiments, the alkyl is substituted with one optionally substituted 5- to 8-membered heterocyclo group. In some embodiments, alkyl is a C₁-C₆ alkyl. In some embodiments, alkyl is a C₁-C₄ alkyl. The heterocyclo group can be linked to the alkyl group through a carbon or nitrogen atom. Non-limiting exemplary (heterocyclo)alkyl groups include:

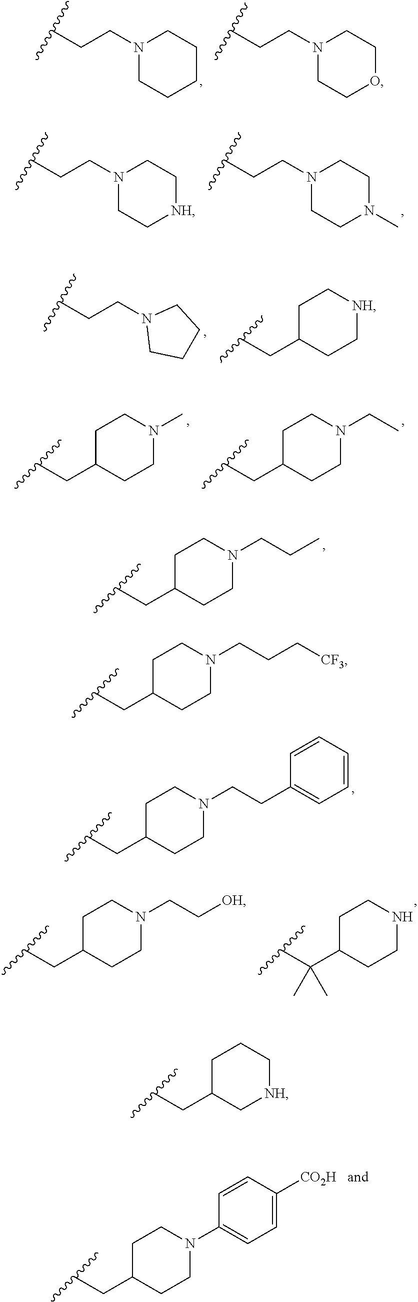

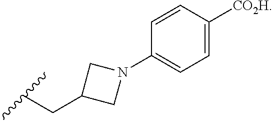 [SMILES: *CC1CN(c2ccc(C(=O)O)cc2)C1]

The term “carbamate” as used herein by itself or as part of another group refers to a radical of the formula —NR^{54a}—C(═O)—OR^{54b}, wherein R^{54a} is hydrogen or alkyl, and R^{54b} is hydrogen, alkyl, optionally substituted cycloalkyl, optionally substituted heterocyclo, optionally substituted aryl, or optionally substituted heteroaryl. A non-limiting exemplary carbamate group is —NH—(C═O)OtBu.

The term “(heteroaryl)alkyl” as used herein by itself or as part of another group refers to an alkyl substituted with one or two optionally substituted heteroaryl groups. In some embodiments, the alkyl group is substituted with one optionally substituted 5- to 14-membered heteroaryl group. In some embodiments, the alkyl group is substituted with two optionally substituted 5- to 14-membered heteroaryl groups. In some embodiments, the alkyl group is substituted with one optionally substituted 5- to 9-membered heteroaryl group. In some embodiments, the alkyl group is substituted with two optionally substituted 5- to 9-membered heteroaryl groups. In some embodiments, the alkyl group is substituted with one optionally substituted 5- or 6-membered heteroaryl group. In some embodiments, the alkyl group is substituted with two optionally substituted 5- or 6-membered heteroaryl groups. In some embodiments, the alkyl group is a C₁-C₆ alkyl. In some embodiments, the alkyl group is a C₁-C₄ alkyl. In some embodiments, the alkyl group is a C₁ or C₂ alkyl. Non-limiting exemplary (heteroaryl)alkyl groups include:

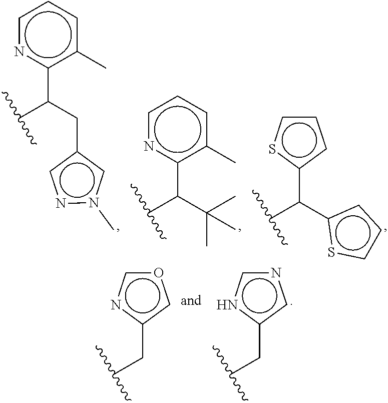

The terms “aralkyl” or “(aryl)alkyl” as used herein by themselves or as part of another group refers to an alkyl substituted with one, two, or three optionally substituted aryl groups. In some embodiments, the alkyl is substituted with one optionally substituted aryl group. In some embodiments, the alkyl is substituted with two optionally substituted aryl groups. In some embodiments, the aryl is an optionally substituted phenyl or optionally substituted naphthyl. In some embodiments, the aryl is an optionally substituted phenyl. In some embodiments, the alkyl is a C₁-C₆ alkyl. In some embodiments, the alkyl is a C₁-C₄ alkyl. In some embodiments, the alkyl is a C₁ or C₂ alkyl. Non-limiting exemplary (aryl)alkyl groups include benzyl, phenethyl, —CHPh₂, and —CH(4-F-Ph)₂.

The term “amido” as used herein by itself or as part of another group refers to a radical of formula —C(═O)NR^{60a}R^{60b}, wherein R^{60a} and R^{60b} are each independently hydrogen, optionally substituted alkyl, optionally substituted alkenyl, optionally substituted alkynyl, haloalkyl, (alkoxy)alkyl, (hydroxy)alkyl, (cyano)alkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl; or R^{60a} and R^{60b} taken together with the nitrogen to which they are attached from a 4- to 8-membered optionally substituted heterocyclo group. In some embodiments, R^{60a} and R^{60b} are each independently hydrogen or C₁-C₆ alkyl.

The term “amino” as used by itself or as part of another group refers to a radical of the formula —NR^{55a}R^{55b}, wherein R^{55a} and R^{55b} are independently hydrogen, optionally substituted alkyl, haloalkyl, (hydroxy)alkyl, (alkoxy)alkyl, (amino)alkyl, heteroalkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl.

In some embodiments, the amino is —NH₂.

In some embodiments, the amino is an “alkylamino,” i.e., an amino group wherein R^{55a} is C₁₋₆ alkyl and R^{55b} is hydrogen. In some embodiments, R^{55a} is C₁-C₄ alkyl. Non-limiting exemplary alkylamino groups include —N(H)CH₃ and —N(H)CH₂CH₃.

In some embodiments, the amino is a “dialkylamino,” i.e., an amino group wherein R^{55a} and R^{55b} are each independently C₁₋₆ alkyl. In some embodiments, R^{55a} and R^{55b} are each independently C₁-C₄ alkyl. Non-limiting exemplary dialkylamino groups include —N(CH₃)₂ and —N(CH₃)CH₂CH(CH₃)₂.

In some embodiments, the amino is a “hydroxyalkylamino,” i.e., an amino group wherein R^{55a} is (hydroxyl)alkyl and R^{55b} is hydrogen or C₁-C₄ alkyl.

In some embodiments, the amino is a “cycloalkylamino,” i.e., an amino group wherein R^{55a} is optionally substituted cycloalkyl and R^{55b} is hydrogen or C₁-C₄ alkyl.

In some embodiments, the amino is a “aralkylamino,” i.e., an amino group wherein R^{55a} is aralkyl and R^{55b} is hydrogen or C₁-C₄ alkyl. Non-limiting exemplary aralkylamino groups include —N(H)CH₂Ph, —N(H)CHPh₂, and —N(CH₃)CH₂Ph.

In some embodiments, the amino is a “(cycloalkyl)alkylamino,” i.e., an amino group wherein R^{55a} is (cycloalkyl)alkyl and R^{55b} is hydrogen or C₁-C₄ alkyl. Non-limiting exemplary (cycloalkyl)alkylamino groups include:

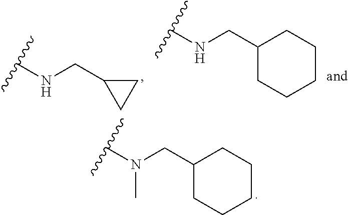

In some embodiments, the amino is a “(heterocyclo)alkylamino,” i.e., an amino group wherein R^{55a} is (heterocyclo)alkyl and R^{55b} is hydrogen or C₁-C₄ alkyl. Non-limiting exemplary (heterocyclo)alkylamino groups include:

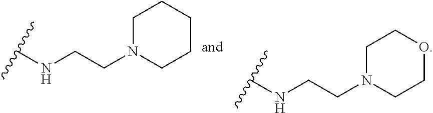 [SMILES: *NCCN1CCCCC1 ; *NCCN1CCOCC1]

The term “(amino)alkyl” as used herein by itself or as part of another group refers to an alkyl substituted with one amino group. In some embodiments, the amino group is —NH₂. In some embodiments, the amino group is an alkylamino. In some embodiments, the amino group is a dialkylamino. In some embodiments, the alkyl is a C₁-C₆ alkyl. In some embodiments, the alkyl is a C₁-C₄ alkyl. Non-limiting exemplary (amino)alkyl groups include —CH₂NH₂, CH₂CH₂N(H)CH₃, —CH₂CH₂N(CH₃)₂, CH₂N(H)cyclopropyl, —CH₂N(H)cyclobutyl, and —CH₂N(H)cyclohexyl, and —CH₂CH₂CH₂N(H)CH₂Ph and —CH₂CH₂CH₂N(H)CH₂(4-CF₃-Ph).

In the present disclosure, the term “alkylenyl” as used herein by itself or part of another group refers to a divalent form of an alkyl group, wherein the alkyl group is either unsubstituted or substituted with one or two groups independently selected from optionally substituted phenyl and optionally substituted 5- or 6-membered heteroaryl. In some embodiments, the alkylenyl is a divalent form of a C₁₋₁₂ alkyl, i.e., a C₁-C₁₂ alkylenyl. In some embodiments, the alkylenyl is a divalent form of a C₁₋₁₀ alkyl, i.e., a C₁-C₁₀ alkylenyl. In some embodiments, the alkylenyl is a divalent form of a C₁₋₈ alkyl, i.e., a C₁-C₈ alkylenyl. In some embodiments, the alkylenyl is a divalent form of an unsubstituted C₁₋₆ alkyl, i.e., a C₁-C₆ alkylenyl. In some embodiments, the alkylenyl is a divalent form of an unsubstituted C₁₋₄ alkyl, i.e., a C₁-C₄ alkylenyl. In some embodiments, the alkylenyl is a divalent form of a C₁₋₄ alkyl substituted with one or two optionally substituted phenyl groups. Non-limiting exemplary alkylenyl groups include —CH₂—, —CH₂CH₂—, —CH(Ph)-, —CH(Ph)CH₂—, —CH₂CH₂CH₂—, —CH(Ph)CH₂CH₂—, —CH₂(CH₂)₂CH₂—, —CH(CH₂)₃CH₂—, and —CH₂(CH₂)₄CH₂—.

The term “heteroalkylenyl” as used herein by itself or part of another group refers to a divalent form of a heteroalkyl group. In some embodiments, the heteroalkylenyl is a divalent form of a 3- to 20-membered heteroalkyl, i.e., a 3- to 20-membered heteroalkylenyl. In some embodiments, the heteroalkylenyl is a divalent form of a 3- to 10-membered heteroalkyl, i.e., a 3- to 10-membered heteroalkylenyl. In some embodiments, the heteroalkylenyl is a divalent form of a 3- to 8-membered heteroalkyl, i.e., a 3- to 8-membered heteroalkylenyl. In some embodiments, the heteroalkylenyl is a divalent form of a 3- to 6-membered heteroalkyl, i.e., a 3- to 6-membered heteroalkylenyl. In some embodiments, the heteroalkylenyl is a divalent form of a 3- or 4-membered heteroalkyl, i.e., a 3- or 4-membered heteroalkylenyl. In some embodiments, the heteroalkylenyl is a radical of the formula —(CH₂CH₂O)ᵤ₁— wherein u₁ is 1, 2, 3, 4, 5, or 6. Non-limiting exemplary heteroalkylenyl groups include —CH₂OCH₂—, —CH₂CH₂OCH₂CH₂O—, —CH₂OCH₂CH₂CH₂—, and —CH₂CH₂OCH₂CH₂OCH₂CH₂O—.

The term “heterocyclenyl” as used herein by itself or part of another group refers to a divalent form of an optionally substituted heterocyclo group. In some embodiments, the heterocyclenyl is a divalent form of a 4- to 14-membered heterocyclo group, i.e., a 4- to 14-membered heterocyclenyl. In some embodiments, the heterocyclenyl is a divalent form of a 4- to 10-membered heterocyclo group, i.e., a 4- to 10-membered heterocyclenyl. In some embodiments, the heterocyclenyl is a divalent form of a 4- to 8-membered heterocyclo group, i.e., a 4- to 8-membered heterocyclenyl. In some embodiments, the heterocyclenyl is a divalent form of an optionally substituted azetidine. In some embodiments, the heterocyclenyl is a divalent form of an optionally substituted piperidinyl. In some embodiments, the heterocyclenyl is a divalent form of an optionally substituted piperazinyl. Non-limiting exemplary heterocyclenyl groups include:

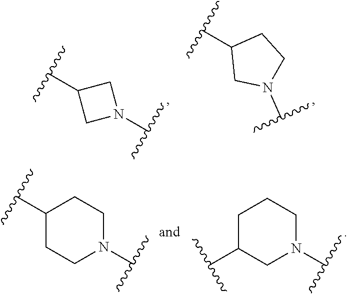 [SMILES: *C1CN(*)C1 ; *C1CCN(*)C1 ; *C1CCN(*)CC1 ; *C1CCCN(*)C1]

In some embodiments, the heterocyclenyl is a spiroheterocyclenyl.

The term “spiroheterocyclenyl” as used herein by itself or part of another group refers to a divalent form of a spiroheterocyclo. Non-limiting exemplary spiroheterocyclenyl groups include:

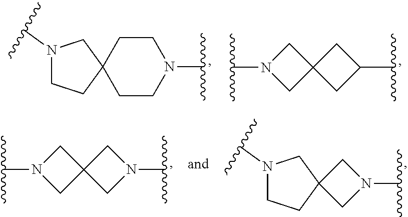

The term “cycloalkylenyl” as used herein by itself or part of another group refers to a divalent form of an optionally substituted C₄-C₆ cycloalkyl group. In some embodiments, the cycloalkylenyl is a 4-membered cycloalkylenyl. In some embodiments, the cycloalkylenyl is a 5-membered cycloalkylenyl. In some embodiments, the cycloalkylenyl is a 6-membered cycloalkylenyl. Non-limiting exemplary groups include:

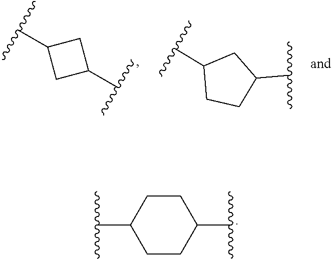 [SMILES: *C1CC(*)C1 ; *C1CCC(*)C1 ; *C1CCC(*)CC1]

The term “phenylenyl” as used herein by itself or part of another group refers to a divalent form of an optionally substituted phenyl group. Non-limiting examples include:

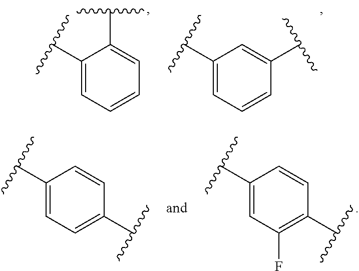 [SMILES: *c1ccccc1* ; *c1cccc(*)c1 ; *c1ccc(*)cc1 ; *c1ccc(*)c(F)c1]

The term “heteroarylenyl” as used herein by itself or part of another group refers to a divalent form of an optionally substituted heteroaryl group, e.g., a 5- to 9-membered heteroarylenyl. In some embodiments, the heteroarylenyl is a 6-membered heteroarylenyl, e.g., heteroarylenyl derived from pyridine. In some embodiments, the heteroarylenyl is a bicyclic 9-membered heteroarylenyl. Exemplary non-limiting exemplary heteroarylenyl groups include:

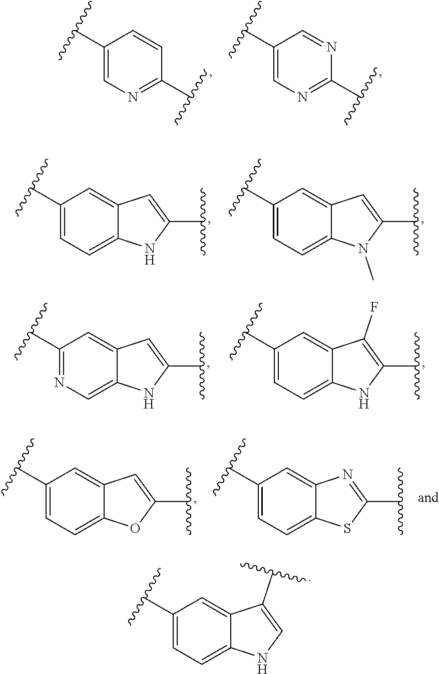

The present disclosure encompasses any of the Cereblon Ligands being isotopically-labelled (i.e., radiolabeled) by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H (or deuterium (D)), ³H, ¹¹C, ¹³C, ¹⁴C, ¹⁵N, ¹⁸O, ¹⁷O, ³¹P, ³²P, ³⁵S, ¹⁸F, and ³⁶Cl, respectively, e.g., ³H, ¹¹C, and ¹⁴C. In some embodiments, provided is a composition wherein substantially all of the atoms at a position within the Cereblon Ligand are replaced by an atom having a different atomic mass or mass number. In some embodiments, provided is a composition wherein a portion of the atoms at a position within the Cereblon Ligand are replaced, i.e., the Cereblon Ligand is enriched at a position with an atom having a different atomic mass or mass number. For example, in some particular embodiments, the hydrogen atom at R³ in any one of Formulae I-IV can be replaced with a deuterium atom.

When a position of any one of Formulae I-IV, e.g., R³, is designated specifically as “H” or “hydrogen,” the position is understood to have hydrogen at its natural abundance isotopic composition.

When a position of any one of Formulae I-IV, e.g., R³, is designated specifically as “D” or “deuterium,” the position is understood to have deuterium at an abundance that is at least about 1000 times greater than the natural abundance of deuterium, which is about 0.015%.

Isotopically-labelled Cereblon Ligands can be prepared by methods known in the art.

Cereblon Ligands may contain one or more asymmetric centers and may thus give rise to enantiomers, diastereomers, and other stereoisomeric forms. The present disclosure encompasses the use of all such possible forms, as well as their racemic and resolved forms and mixtures thereof. The individual enantiomers can be separated according to methods known in the art in view of the present disclosure. When the compounds described herein contain olefinic double bonds or other centers of geometric asymmetry, and unless specified otherwise, it is intended that they include both E and Z geometric isomers. All tautomers are also encompassed by the present disclosure.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive. In some embodiments, Cereblon Ligands are racemic.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as |R−S|*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography or optical polarimetry.

The term “about,” as used herein, includes the recited number±10%. Thus, “about 10” means 9 to 11.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

### Example 1

The synthesis of 3-(1-oxo-1,3,5,6,7,8-hexahydro-2H-pyrrolo[3,4-g]isoquinolin-2-yl)piperidine-2,6-dione is shown in Scheme 1. (S)-3-(1-Oxo-1,3,5,6,7,8-hexahydro-2H-pyrrolo[3,4-g]isoquinolin-2-yl)piperidine-2,6-dione and (R)-3-(1-oxo-1,3,5,6,7,8-hexahydro-2H-pyrrolo[3,4-g]isoquinolin-2-yl)piperidine-2,6-dione can also be prepared according to Scheme 1 using (S)-3-aminopiperidine-2,6-dione HCl or (R)-3-aminopiperidine-2,6-dione HCl instead of 3-aminopiperidine-2,6-dione HCl.

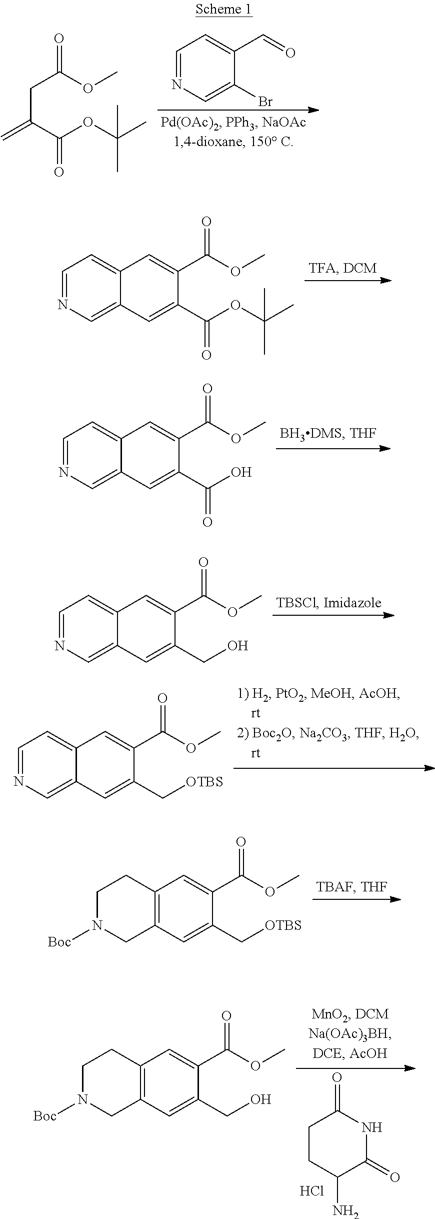

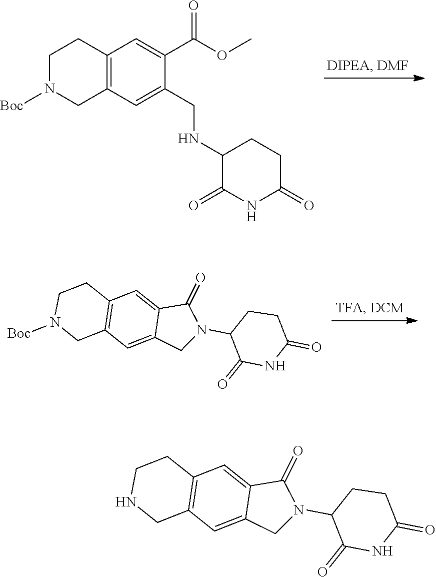 [SMILES: COC(=O)c1cc2c(cc1CNC1CCC(=O)NC1=O)CN(C(=O)OC(C)(C)C)CC2 ; CC(C)(C)OC(=O)N1CCc2cc3c(cc2C1)CN(C1CCC(=O)NC1=O)C3=O ; O=C1CCC(N2Cc3cc4c(cc3C2=O)CCNC4)C(=O)N1]

### Example 2

The synthesis of 3-(3-oxo-1,3,5,6,7,8-hexahydro-2H-pyrrolo[3,4-g]isoquinolin-2-yl)piperidine-2,6-dione is shown in Scheme 2. (S)-3-(3-Oxo-1,3,5,6,7,8-hexahydro-2H-pyrrolo[3,4-g]isoquinolin-2-yl)piperidine-2,6-dione and (R)-3-(3-oxo-1,3,5,6,7,8-hexahydro-2H-pyrrolo[3,4-g]isoquinolin-2-yl)piperidine-2,6-dione can also be prepared according to Scheme 2 using (S)-3-aminopiperidine-2,6-dione HCl or (R)-3-aminopiperidine-2,6-dione HCl instead of 3-aminopiperidine-2,6-dione HCl.

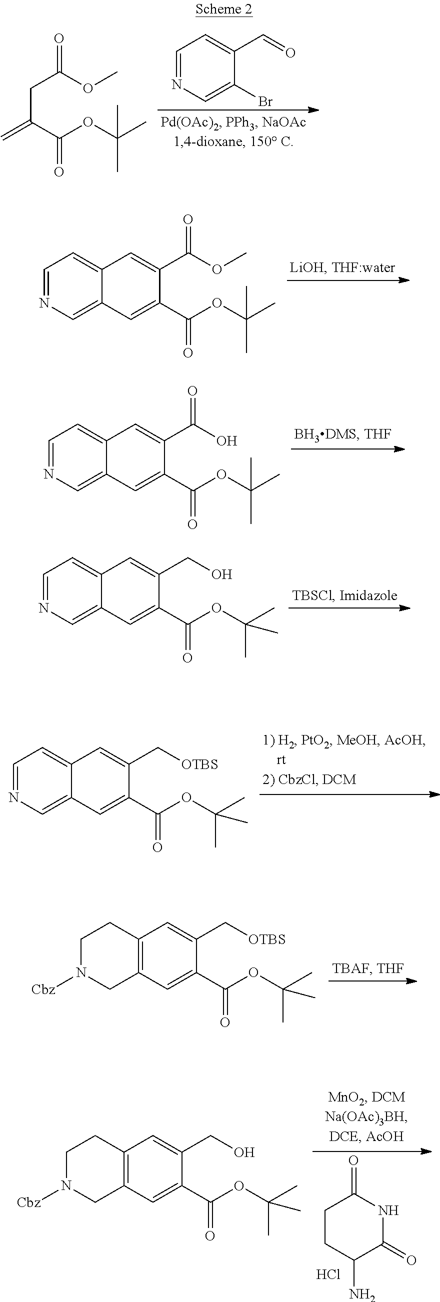

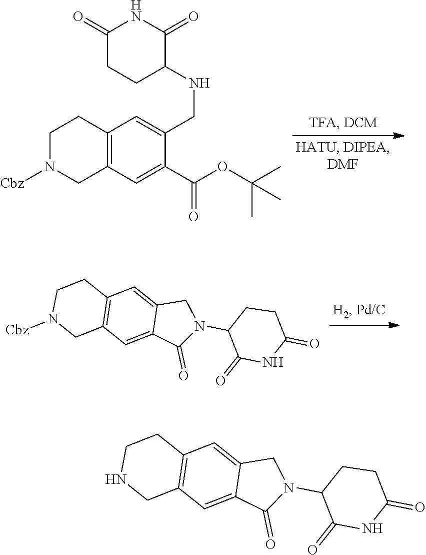 [SMILES: CC(C)(C)OC(=O)c1cc2c(cc1CNC1CCC(=O)NC1=O)CCN(C(=O)OCc1ccccc1)C2 ; O=C1CCC(N2Cc3cc4c(cc3C2=O)CN(C(=O)OCc2ccccc2)CC4)C(=O)N1 ; O=C1CCC(N2Cc3cc4c(cc3C2=O)CNCC4)C(=O)N1]

### Example 3

The synthesis of 2-(2,6-dioxopiperidin-3-yl)-7,8-dihydropyrrolo[3,4-e]isoindole-1,3(2H,6H)-dione is shown in Scheme 3. (S)-2-(2,6-Dioxopiperidin-3-yl)-7,8-dihydropyrrolo[3,4-e]isoindole-1,3(2H,6H)-dione and (R)-2-(2,6-dioxopiperidin-3-yl)-7,8-dihydropyrrolo[3,4-e]isoindole-1,3(2H,6H)-dione can also be prepared according to Scheme 3 using (S)-3-aminopiperidine-2,6-dione or (R)-3-aminopiperidine-2,6-dione instead of 3-aminopiperidine-2,6-dione.

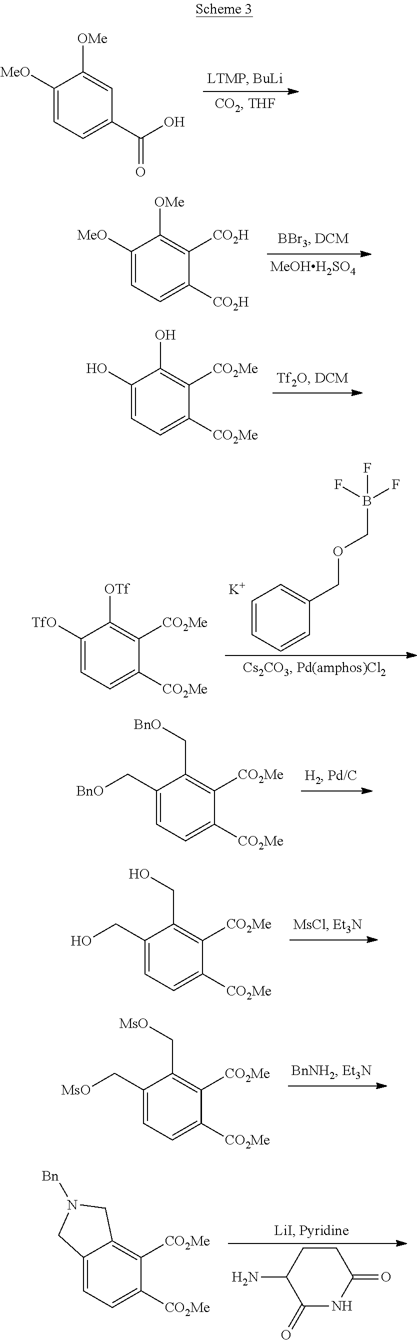

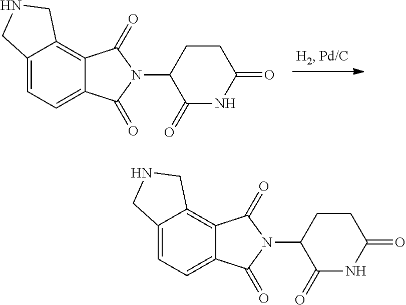

¹H NMR of 2-(2,6-dioxopiperidin-3-yl)-7,8-dihydropyrrolo[3,4-e]isoindole-1,3(2H,6H)-dione (400 MHz, CDCl₃) δ 7.99 (s, 1H), 7.71 (d, J=7.6 Hz, 1H), 7.52 (d, J=7.6 Hz, 1H), 5.05 (s, 2H), 4.98 (dd, J=12.4, 5.3 Hz, 1H), 3.74 (q, J=5.3 Hz, 2H), 3.06-2.95 (m, 2H), 2.95-2.70 (m, 2H), 2.24-2.11 (m, 1H).

### Example 4

The synthesis of 2-(2,6-dioxopiperidin-3-yl)-6,7,8,9-tetrahydro-1H-pyrrolo[3,4-h]isoquinoline-1,3(2H)-dione is shown is Scheme 4. (S)-2-(2,6-Dioxopiperidin-3-yl)-6,7,8,9-tetrahydro-1H-pyrrolo[3,4-h]isoquinoline-1,3(2H)-dione and (R)-2-(2,6-dioxopiperidin-3-yl)-6,7,8,9-tetrahydro-1H-pyrrolo[3,4-h]isoquinoline-1,3(2H)-dione can also be prepared according to Scheme 4 using (S)-3-aminopiperidine-2,6-dione or (R)-3-aminopiperidine-2,6-dione instead of 3-aminopiperidine-2,6-dione.

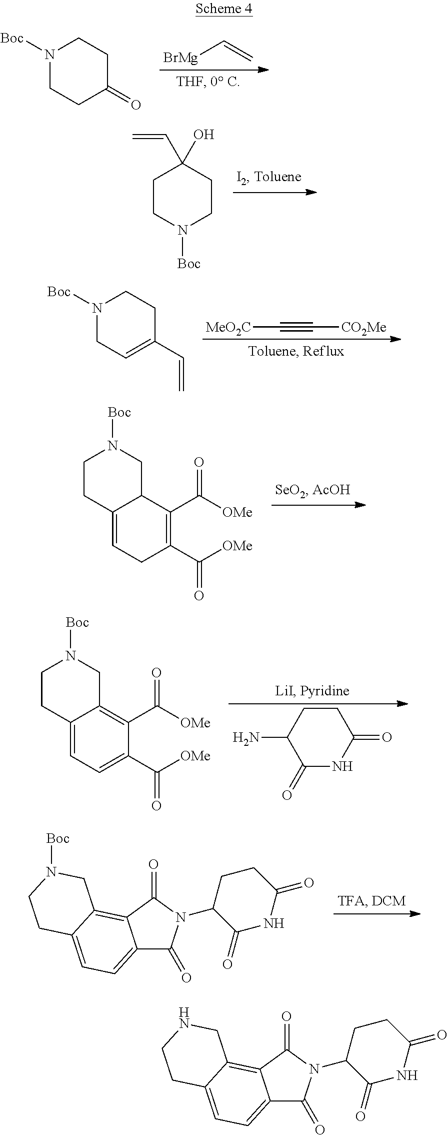

### Example 5

The synthesis of 2-(azetidin-3-ylmethyl)-6-(2,6-dioxopiperidin-3-yl)-2,3,6,7-tetrahydropyrrolo[3,4-f]isoindole-1,5-dione is shown in Scheme 5. (S)-2-(Azetidin-3-ylmethyl)-6-(2,6-dioxopiperidin-3-yl)-2,3,6,7-tetrahydropyrrolo[3,4-f]isoindole-1,5-dione and (R)-2-(azetidin-3-ylmethyl)-6-(2,6-dioxopiperidin-3-yl)-2,3,6,7-tetrahydropyrrolo[3,4-f]isoindole-1,5-dione can also be prepared according to Scheme 5 using (S)-3-aminopiperidine-2,6-dione or (R)-3-aminopiperidine-2,6-dione instead of 3-aminopiperidine-2,6-dione

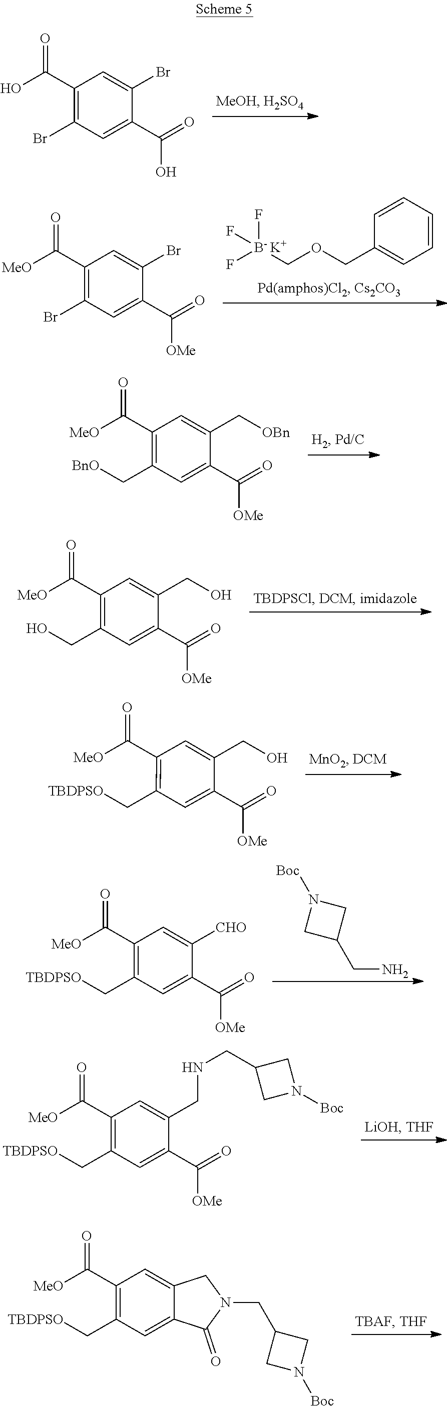

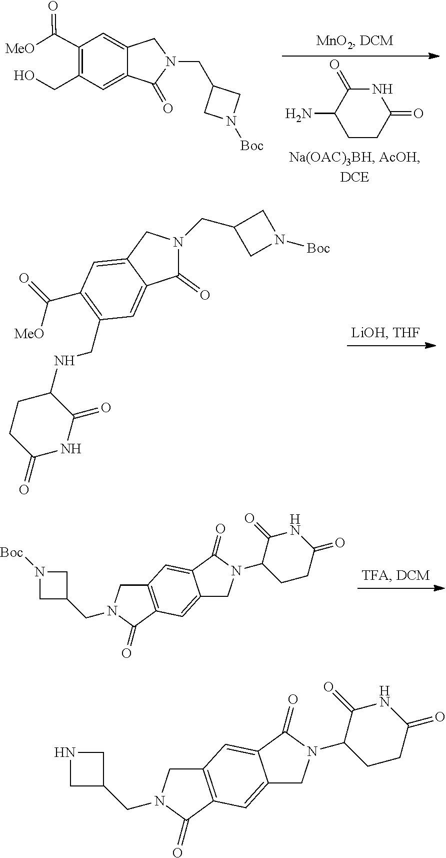 [SMILES: COC(=O)c1cc2c(cc1CO)C(=O)N(CC1CN(C(=O)OC(C)(C)C)C1)C2 ; NC1CCC(=O)NC1=O ; COC(=O)c1cc2c(cc1CNC1CCC(=O)NC1=O)C(=O)N(CC1CN(C(=O)OC(C)(C)C)C1)C2 ; CC(C)(C)OC(=O)N1CC(CN2Cc3cc4c(cc3C2=O)CN(C2CCC(=O)NC2=O)C4=O)C1 ; O=C1CCC(N2Cc3cc4c(cc3C2=O)CN(CC2CNC2)C4=O)C(=O)N1]

### Alternative Scheme

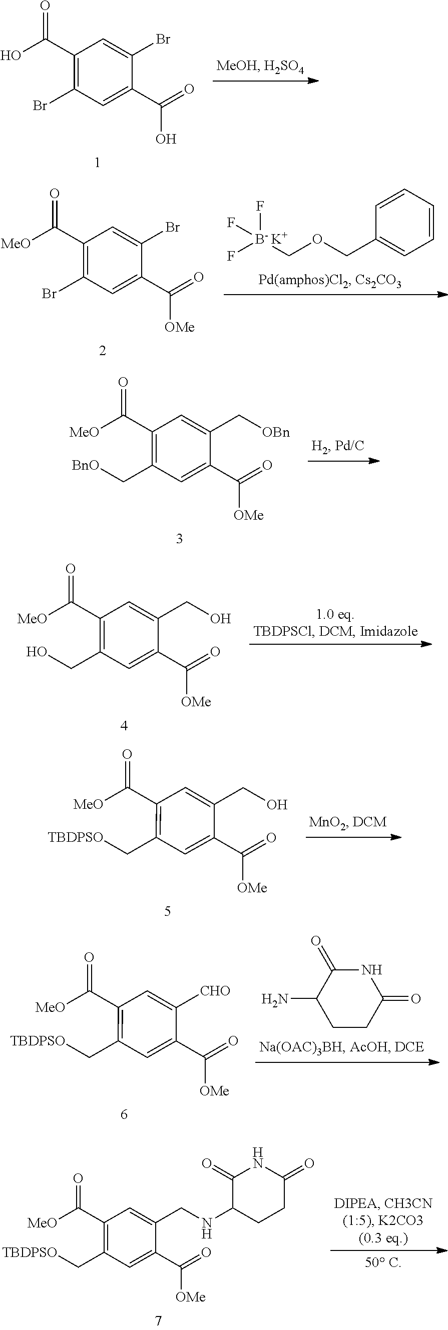

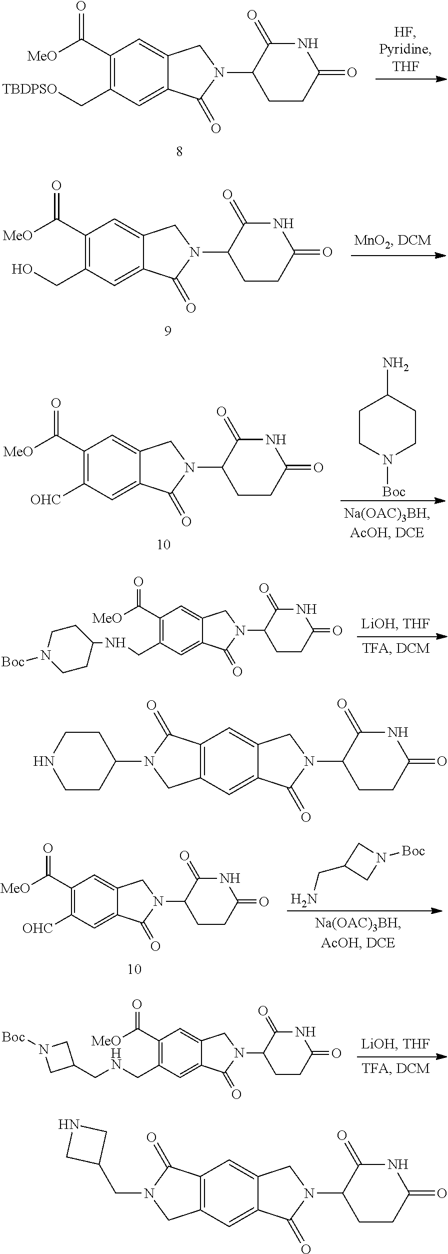

¹H NMR of compound 10 (400 MHz, CDCl₃) δ 10.60 (s, 1H), 8.46 (s, 1H), 8.08 (s, 1H), 7.97 (s, 1H), 5.27 (dd, J=13.3, 5.2 Hz, 1H), 4.68 (d, J=16.8 Hz, 1H), 4.47 (d, J=16.9 Hz, 1H), 4.05 (s, 3H), 3.04-2.78 (m, 2H), 2.50-2.36 (m, 1H), 2.36-2.24 (in, 1H).

### Example 6

The synthesis of 2-(azetidin-3-ylmethyl)-6-(2,6-dioxopiperidin-3-yl)-2,3,5,6-tetrahydropyrrolo[3,4-f]isoindole-1,7-dione is shown in Scheme 6. (S)-2-(Azetidin-3-ylmethyl)-6-(2,6-dioxopiperidin-3-yl)-2,3,5,6-tetrahydropyrrolo[3,4-f]isoindole-1,7-dione and (R)-2-(azetidin-3-ylmethyl)-6-(2,6-dioxopiperidin-3-yl)-2,3,5,6-tetrahydropyrrolo[3,4-f]isoindole-1,7-dione can also be prepared according to Scheme 6 using (S)-3-aminopiperidine-2,6-dione or (R)-3-aminopiperidine-2,6-dione instead of 3-aminopiperidine-2,6-dione.

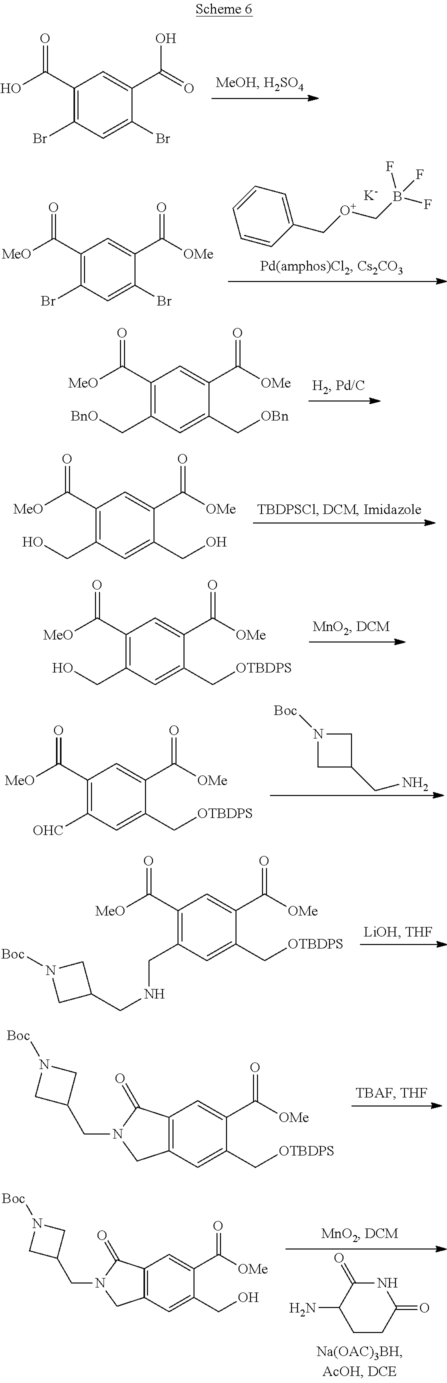

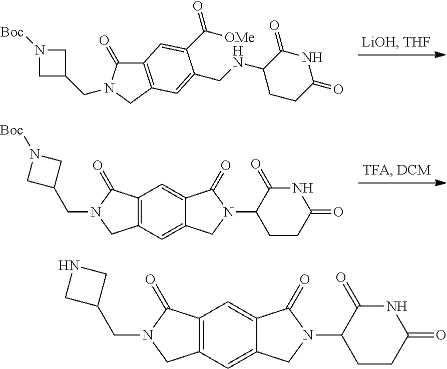 [SMILES: COC(=O)c1cc2c(cc1CNC1CCC(=O)NC1=O)CN(CC1CN(C(=O)OC(C)(C)C)C1)C2=O ; CC(C)(C)OC(=O)N1CC(CN2Cc3cc4c(cc3C2=O)C(=O)N(C2CCC(=O)NC2=O)C4)C1 ; O=C1CCC(N2Cc3cc4c(cc3C2=O)C(=O)N(CC2CNC2)C4)C(=O)N1]

### Alternative Scheme

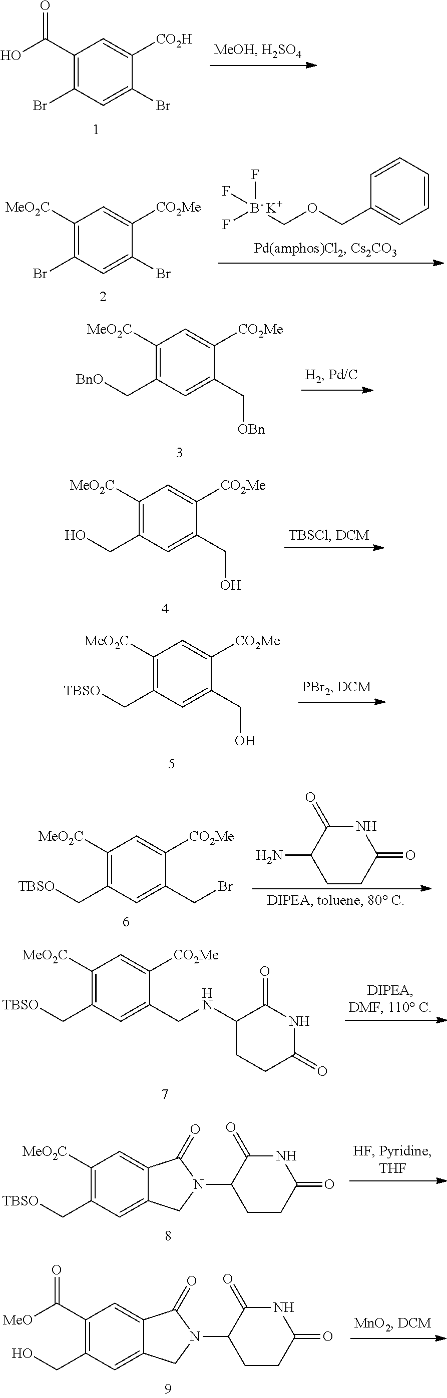

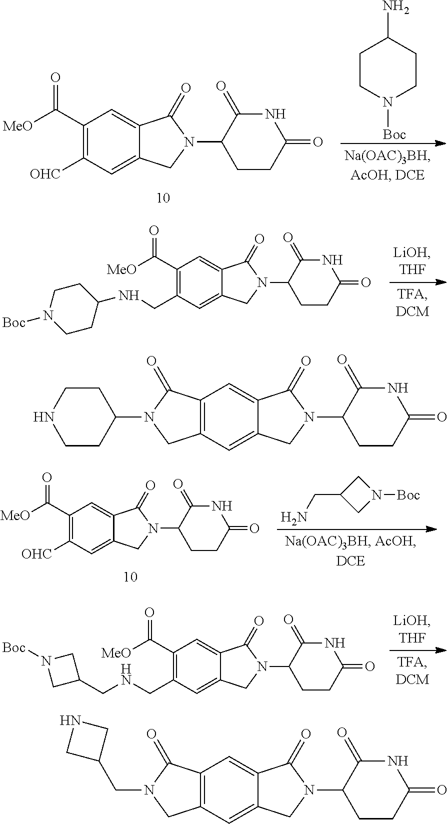

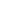

¹H NMR of compound 10 (400 MHz, CDCl₃) δ 10.75 (s, 1H), 8.56 (s, 1H), 8.07 (d, J=0.8 Hz, 1H), 7.94 (brs, 1H), 5.26 (dd, J=13.3, 5.2 Hz, 1H), 4.65 (d, J=16.8 Hz, 1H), 4.48 (d, J=16.9 Hz, 1H), 4.05 (s, 3H), 3.05-2.80 (m, 2H), 2.42 (qd, J=13.2, 4.8 Hz, 1H), 2.34-2.23 (m, 1H).

### Example 7

The synthesis of 3-(6-hydroxy-1-oxo-3,5,6,7-tetrahydrocyclopenta[f]isoindol-2(1H)-yl)piperidine-2,6-dione is shown in Scheme 7. (S)-3-(6-Hydroxy-1-oxo-3,5,6,7-tetrahydrocyclopenta[f]isoindol-2(1H)-yl)piperidine-2,6-dione and (R)-3-(6-hydroxy-1-oxo-3,5,6,7-tetrahydrocyclopenta[f]isoindol-2(1H)-yl)piperidine-2,6-dione can also be prepared according to Scheme 7 using (S)-3-aminopiperidine-2,6-dione or (R)-3-aminopiperidine-2,6-dione instead of 3-aminopiperidine-2,6-dione.

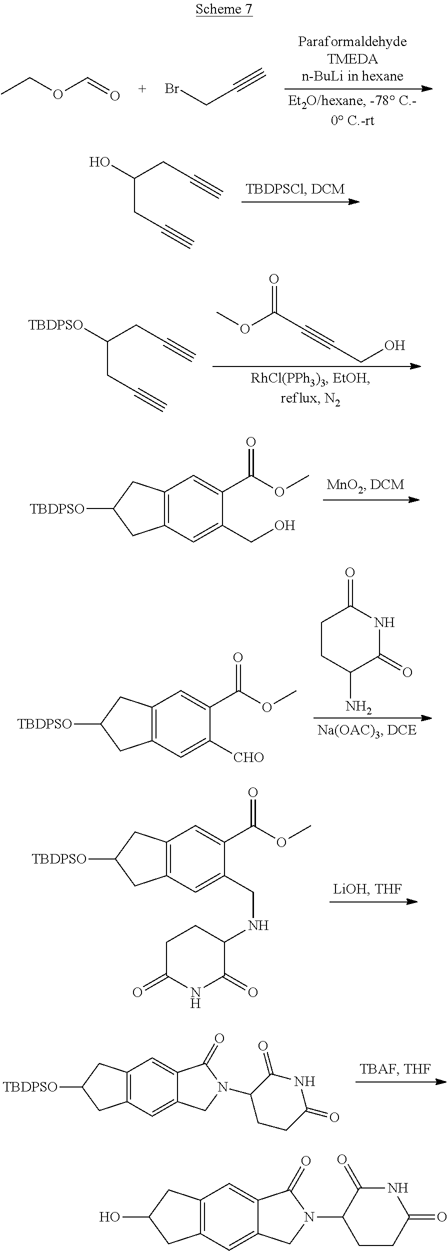

### Example 8

The synthesis of 2-(2,6-dioxopiperidin-3-yl)-1-oxo-1,2,3,5,6,7-hexahydrocyclopenta[f]isoindole-6-carbaldehyde is shown in Scheme 8. (S)-2-(2,6-Dioxopiperidin-3-yl)-1-oxo-1,2,3,5,6,7-hexahydrocyclopenta[f]isoindole-6-carbaldehyde and (R)-2-(2,6-dioxopiperidin-3-yl)-1-oxo-1,2,3,5,6,7-hexahydrocyclopenta[f]isoindole-6-carbaldehyde can also be prepared according to Scheme 8 using (S)-3-aminopiperidine-2,6-dione or (R)-3-aminopiperidine-2,6-dione instead of 3-aminopiperidine-2,6-dione.

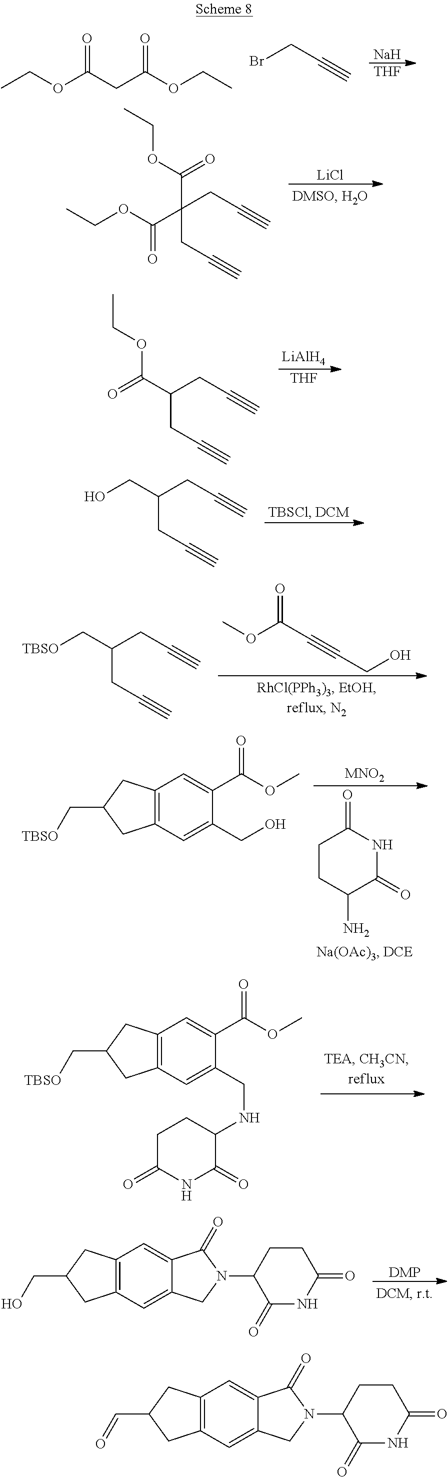

### Example 9

The synthesis of 8-(2,6-dioxopiperidin-3-yl)-2,3,4,5-tetrahydro-7H-[1,4]oxazepino[6,7-f]isoindole-7,9(8H)-dione is shown in Scheme 9. (S)-8-(2,6-Dioxopiperidin-3-yl)-2,3,4,5-tetrahydro-7H-[1,4]oxazepino[6,7-f]isoindole-7,9(8H)-dione and (R)-8-(2,6-dioxopiperidin-3-yl)-2,3,4,5-tetrahydro-7H-[1,4]oxazepino[6,7-f]isoindole-7,9(8H)-dione can also be prepared according to Scheme 9 using (S)-3-aminopiperidine-2,6-dione or (R)-3-aminopiperidine-2,6-dione instead of 3-aminopiperidine-2,6-dione.

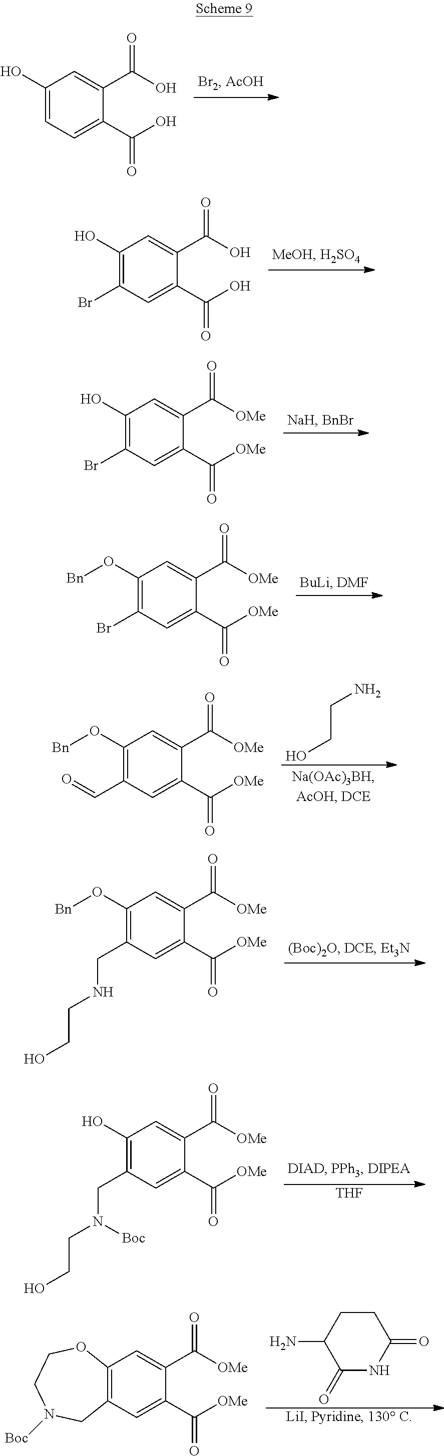

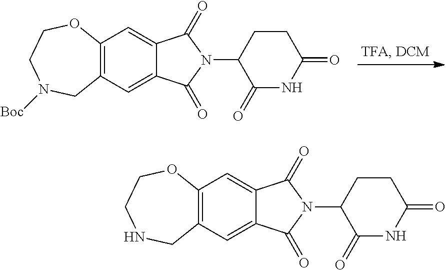 [SMILES: CC(C)(C)OC(=O)N1CCOc2cc3c(cc2C1)C(=O)N(C1CCC(=O)NC1=O)C3=O ; O=C1CCC(N2C(=O)c3cc4c(cc3C2=O)OCCNC4)C(=O)N1]

### Example 10

The synthesis of 2-(6-oxopiperidin-3-yl)pyrrolo[3,4-f]isoindole-1,3(2H,6H)-dione is shown in Scheme 10.

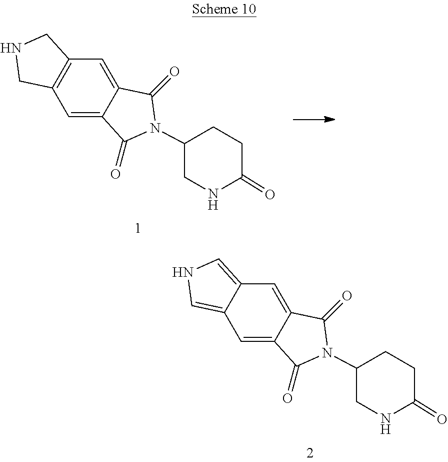 [SMILES: O=C1CCC(N2C(=O)c3cc4c(cc3C2=O)CNC4)CN1 ; O=C1CCC(N2C(=O)c3cc4c[nH]cc4cc3C2=O)CN1]

Compound 1 (1.0 eq) was dissolved in dioxane (10×) and water (5×), and DDQ (1.3 eq) was added. The reaction mixture was stirred at 60° C. for 2 h. All the solvent was removed and the residue was purified by Combiflash with DCM and MeOH to give 2 in 90% yield.

### Example 11

The synthesis of 2-(6-oxopiperidin-3-yl)-3,6-dihydropyrrolo[3,4-f]isoindol-1(2H)-one is shown in Scheme 11.

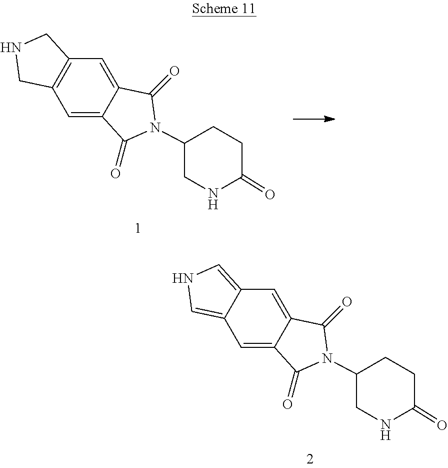 [SMILES: O=C1CCC(N2C(=O)c3cc4c(cc3C2=O)CNC4)CN1 ; O=C1CCC(N2C(=O)c3cc4c[nH]cc4cc3C2=O)CN1]

Compound 1 (1.0 eq) was dissolved in dioxane (10×) and water (5×), and DDQ (1.3 eq) was added. The reaction mixture was stirred at 60° C. for 2 h. All the solvent was removed and the residue was purified by Combiflash with DCM and MeOH to give 2 in 80% yield.

### Example 12

The synthesis of 6-(2,6-dioxopiperidin-3-yl)-2-(piperidin-4-ylmethyl)-5H-oxazolo[4,5-f]isoindole-5,7(6H)-dione is shown in Scheme 12. (S)-6-(2,6-Dioxopiperidin-3-yl)-2-(piperidin-4-ylmethyl)-5H-oxazolo[4,5-f]isoindole-5,7(6H)-dione and (R)-6-(2,6-dioxopiperidin-3-yl)-2-(piperidin-4-ylmethyl)-5H-oxazolo[4,5-f]isoindole-5,7(6H)-dione can also be prepared according to Scheme 12 using (S)-3-aminopiperidine-2,6-dione or (R)-3-aminopiperidine-2,6-dione instead of 3-aminopiperidine-2,6-dione.

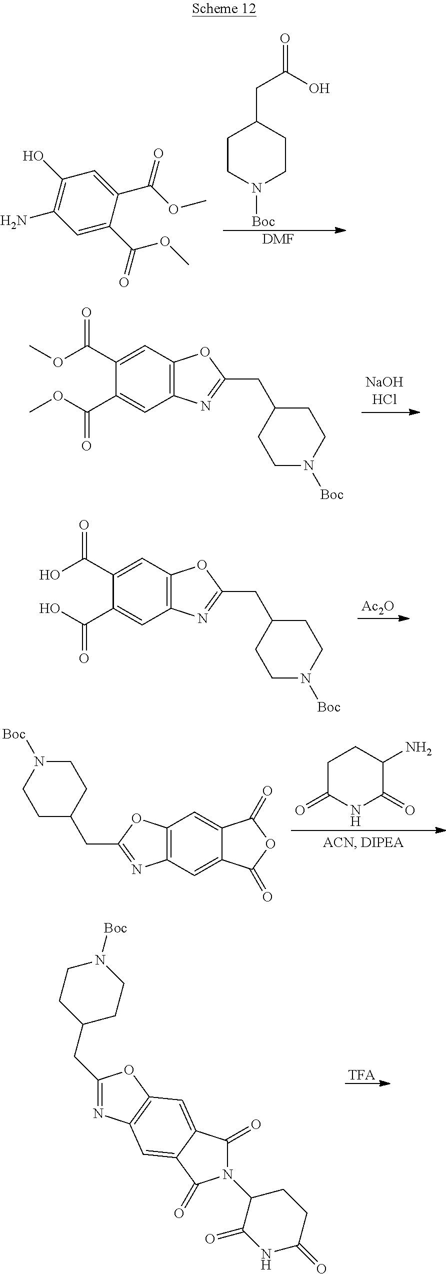

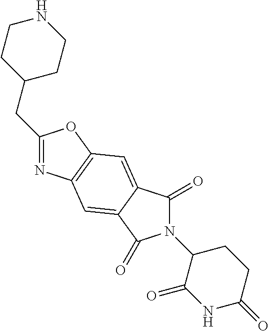 [SMILES: O=C1CCC(N2C(=O)c3cc4nc(CC5CCNCC5)oc4cc3C2=O)C(=O)N1]

### Example 13

The synthesis of 3-(3-methyl-2-oxo-3,5,6,7-tetrahydroimidazo[4,5-f]isoindol-1(2H)-yl)piperidine-2,6-dione is shown in Schemes 13A and 13B. (S)-3-(3-Methyl-2-oxo-3,5,6,7-tetrahydroimidazo[4,5-f]isoindol-1(2H)-yl)piperidine-2,6-dione and (R)-3-(3-methyl-2-oxo-3,5,6,7-tetrahydroimidazo[4,5-f]isoindol-1(2H)-yl)piperidine-2,6-dione can also be prepared according to Scheme 13A or 13B using (S)-3-bromopiperidine-2,6-dione or (R)-3-bromopiperidine-2,6-dione instead of 3-bromopiperidine-2,6-dione.

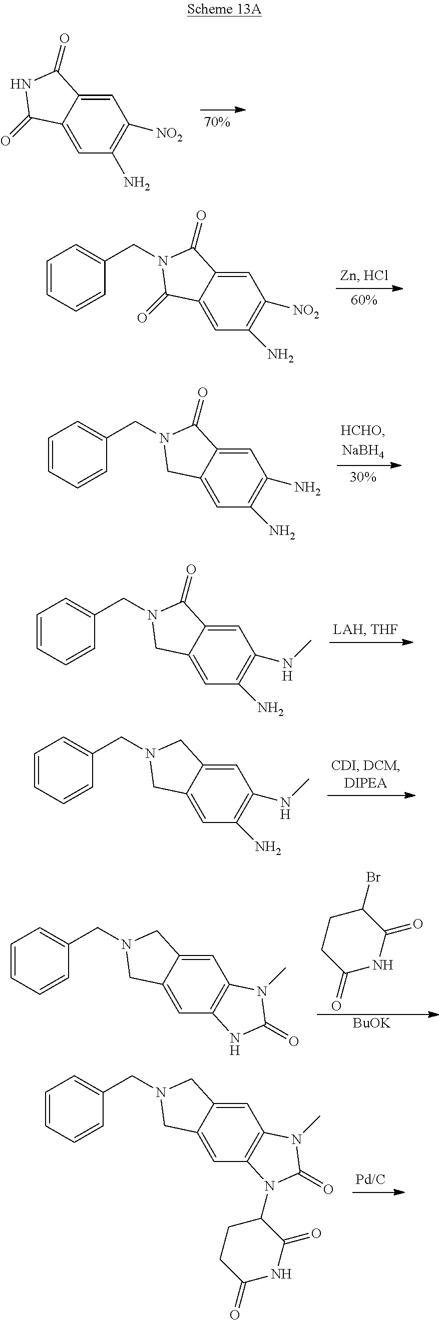

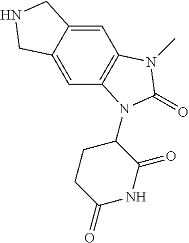 [SMILES: Cn1c(=O)n(C2CCC(=O)NC2=O)c2cc3c(cc21)CNC3]

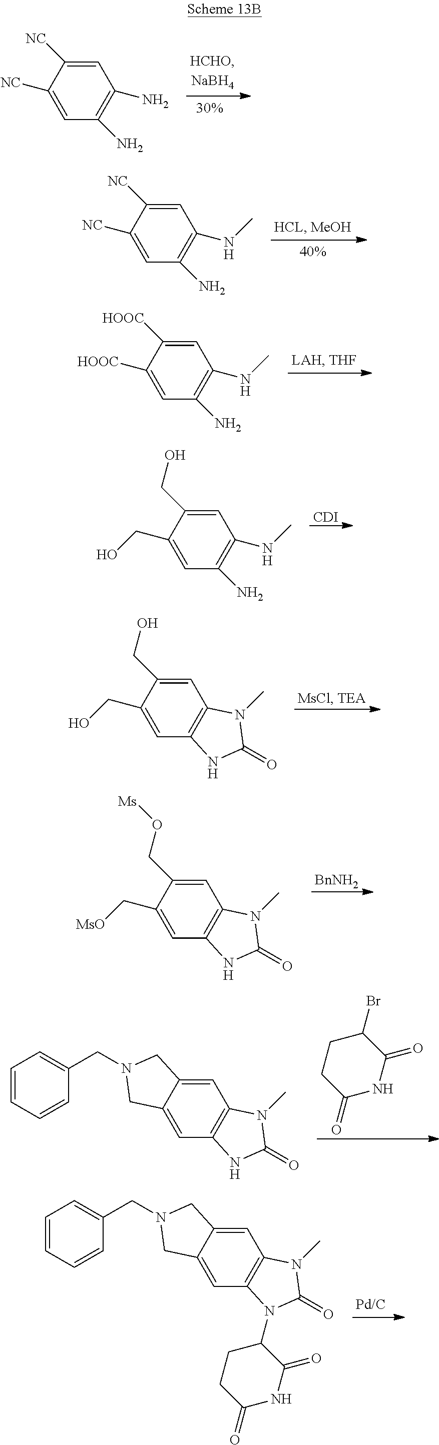

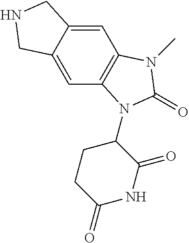 [SMILES: Cn1c(=O)n(C2CCC(=O)NC2=O)c2cc3c(cc21)CNC3]

### Example 14

The synthesis of tert-butyl 1-(2,6-dioxopiperidin-3-yl)-3-methyl-2-oxo-1,2,3,5,7,8-hexahydro-6H-imidazo[4,5-g]isoquinoline-6-carboxylate is shown in Schemes 14A and 14B. The synthesis of tert-butyl 1-(2,6-dioxopiperidin-3-yl)-3-methyl-2-oxo-1,2,3,6,7,9-hexahydro-8H-imidazo[4,5-h]isoquinoline-8-carboxylate is shown in Schemes 14A and 14C.

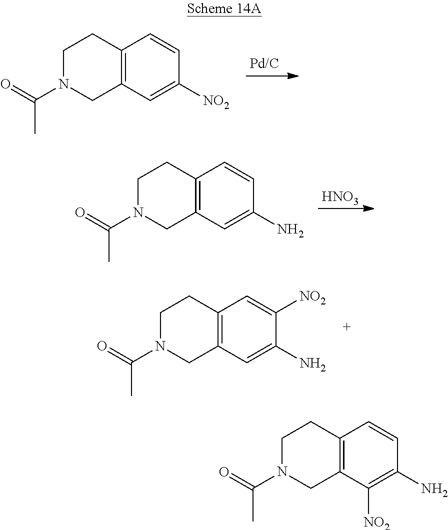

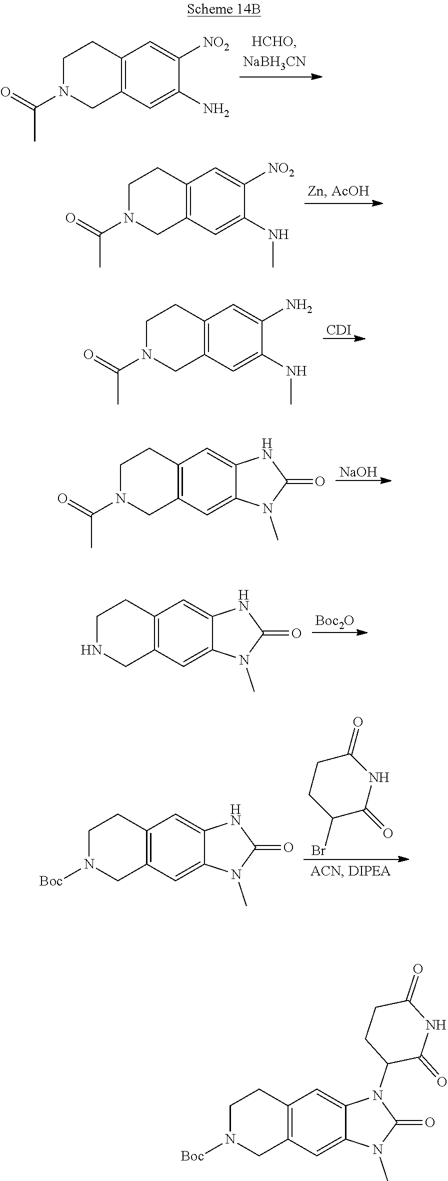

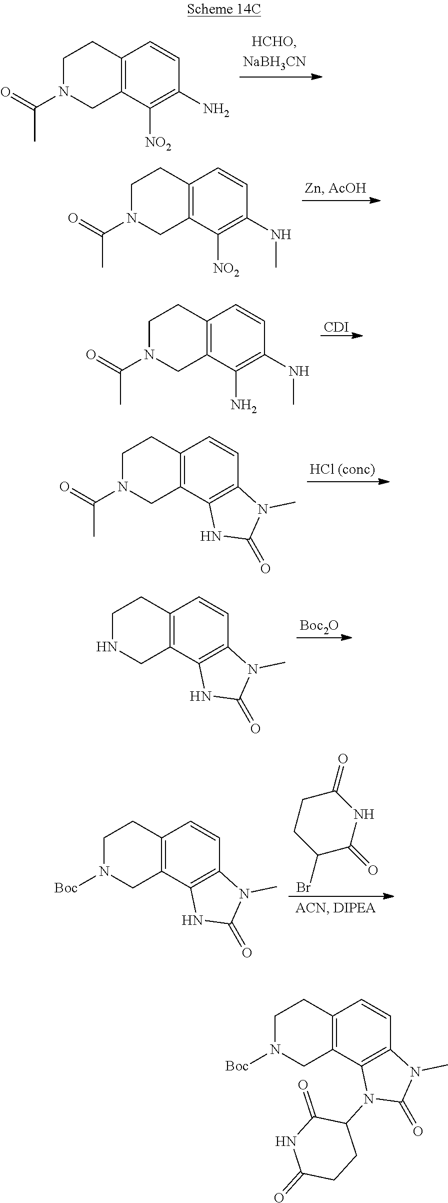

### Example 15

The synthesis of 3-(7-methyl-6-oxo-2-(piperidin-4-ylmethyl)-6,7-dihydro-5H-imidazo[4′,5′:4,5]benzo[1,2-d]oxazol-5-yl)piperidine-2,6-dione is shown in Scheme 15A and the synthesis 3-(5-methyl-6-oxo-2-(piperidin-4-ylmethyl)-5,6-dihydro-7H-imidazo[4′,5′:4,5]benzo[1,2-d]oxazol-7-yl)piperidine-2,6-dione is shown in Scheme 15B.

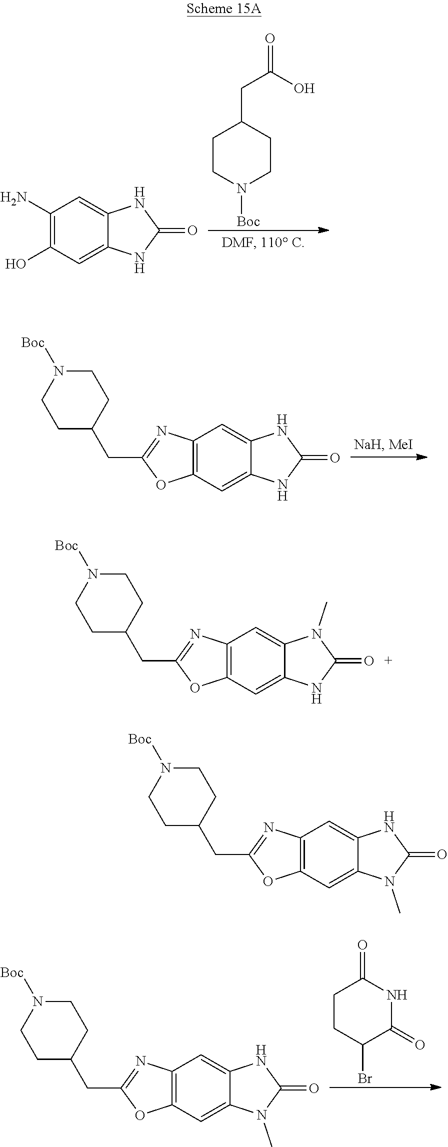

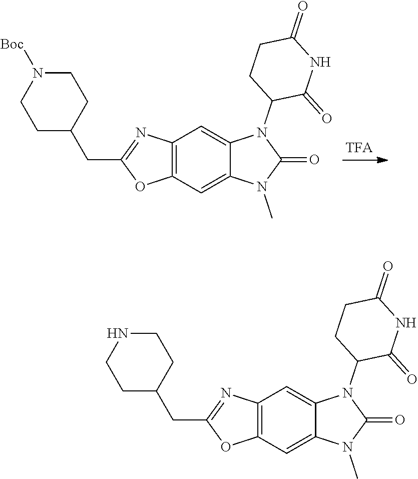 [SMILES: Cn1c(=O)n(C2CCC(=O)NC2=O)c2cc3nc(CC4CCN(C(=O)OC(C)(C)C)CC4)oc3cc21 ; Cn1c(=O)n(C2CCC(=O)NC2=O)c2cc3nc(CC4CCNCC4)oc3cc21]

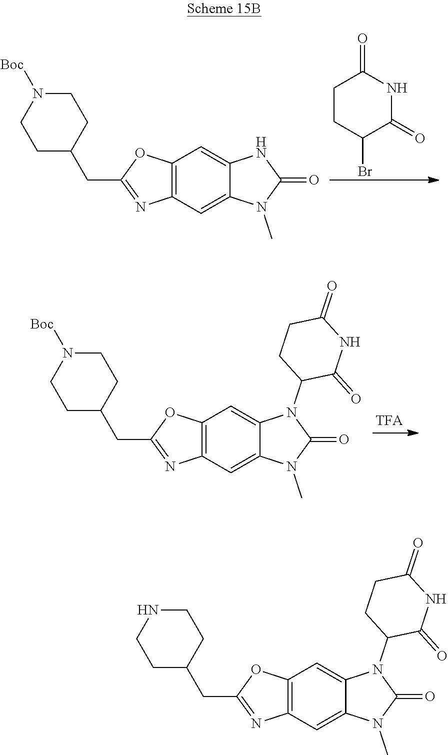 [SMILES: Cn1c(=O)[nH]c2cc3oc(CC4CCN(C(=O)OC(C)(C)C)CC4)nc3cc21 ; O=C1CCC(Br)C(=O)N1 ; Cn1c(=O)n(C2CCC(=O)NC2=O)c2cc3oc(CC4CCN(C(=O)OC(C)(C)C)CC4)nc3cc21 ; Cn1c(=O)n(C2CCC(=O)NC2=O)c2cc3oc(CC4CCNCC4)nc3cc21]

### Example 16

The synthesis of 3-(2-methyl-4-oxo-6,7,8,9-tetrahydropyrido[3,4-g]quinazolin-3(4H)-yl)piperidine-2,6-dione is shown in Scheme 16. (S)-3-(2-methyl-4-oxo-6,7,8,9-tetrahydropyrido[3,4-g]quinazolin-3(4H)-yl)piperidine-2,6-dione and (R)-3-(2-methyl-4-oxo-6,7,8,9-tetrahydropyrido[3,4-g]quinazolin-3(4H)-yl)piperidine-2,6-dione can also be prepared according to Scheme 16 using (S)-3-aminopiperidine-2,6-dione or (R)-3-aminopiperidine-2,6-dione instead of 3-aminopiperidine-2,6-dione.

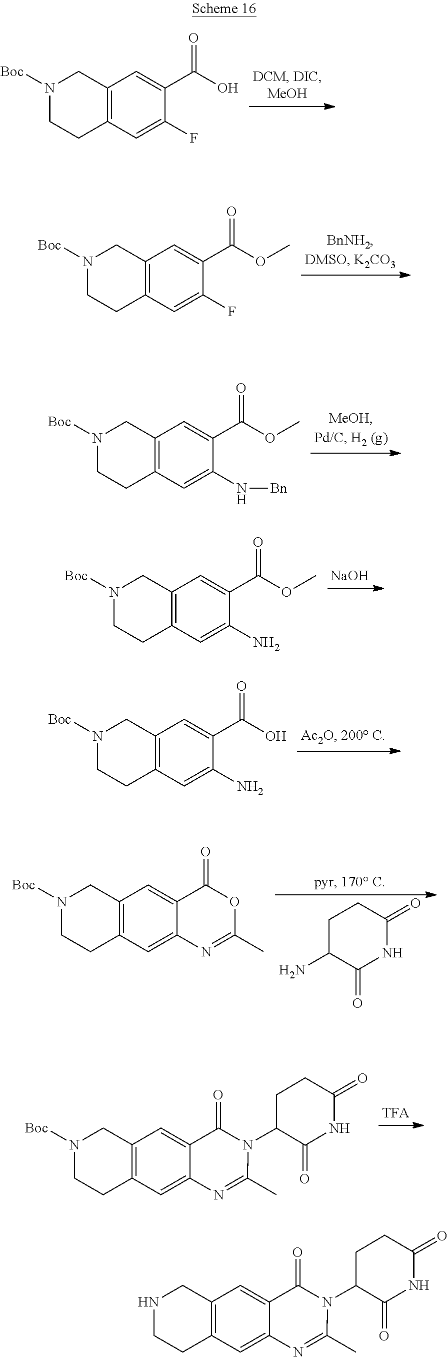

### Example 17

The synthesis of 3-(1-oxo-1,3,6,7,8,9-hexahydro-2H-pyrrolo[3,4-f]isoquinolin-2-yl)piperidine-2,6-dione is shown in Scheme 17.

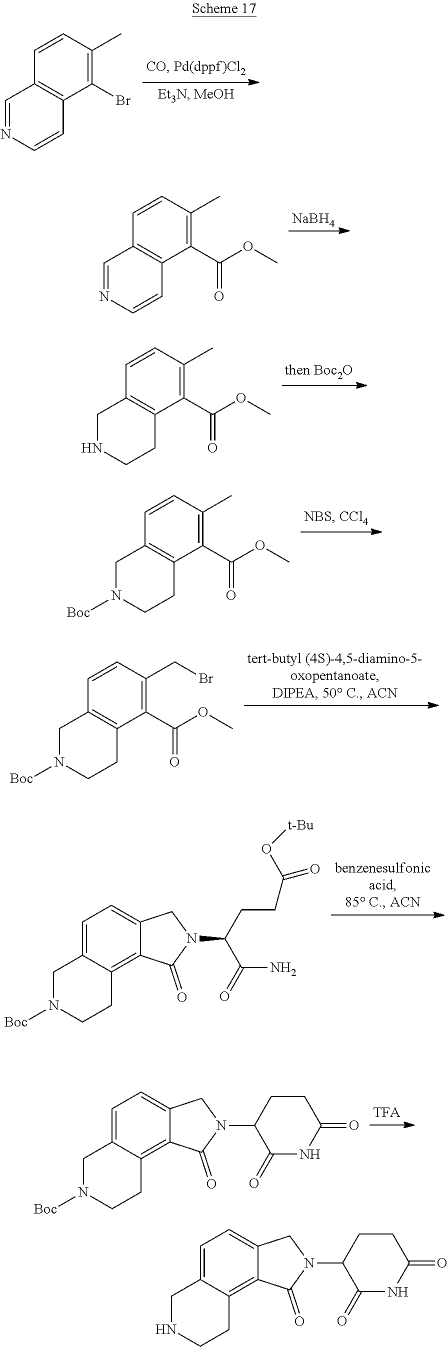

### Example 18

The synthesis of 6-amino-2-(2,6-dioxopiperidin-3-yl)-6,7-dihydrocyclopenta[f]isoindole-1,3(2H,5H)-dione is shown in Scheme 18. (S)-6-Amino-2-(2,6-dioxopiperidin-3-yl)-6,7-dihydrocyclopenta[f]isoindole-1,3(2H,5H)-dione and (R)-6-amino-2-(2,6-dioxopiperidin-3-yl)-6,7-dihydrocyclopenta[f]isoindole-1,3(2H,5H)-dione can also be prepared according to Scheme 18 using (S)-3-aminopiperidine-2,6-dione or (R)-3-aminopiperidine-2,6-dione instead of 3-aminopiperidine-2,6-dione.

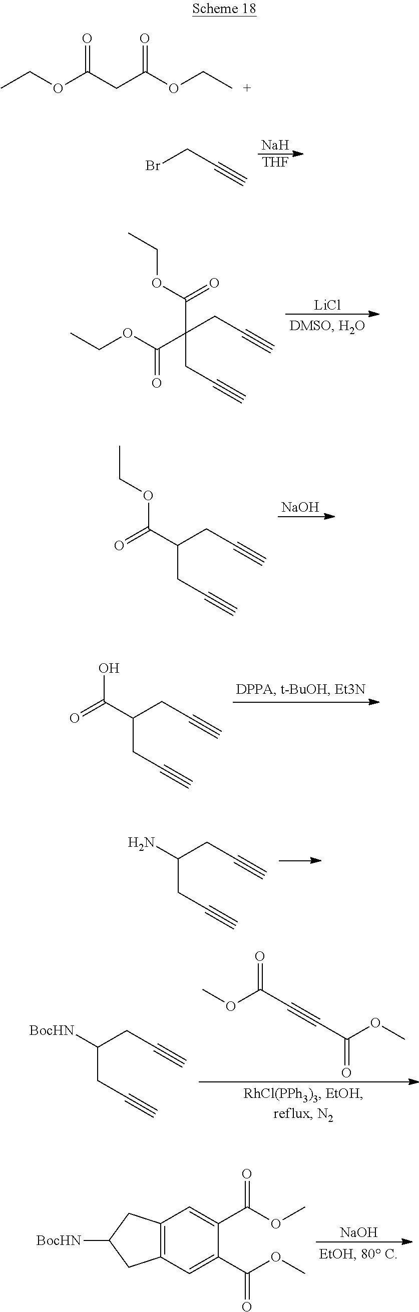

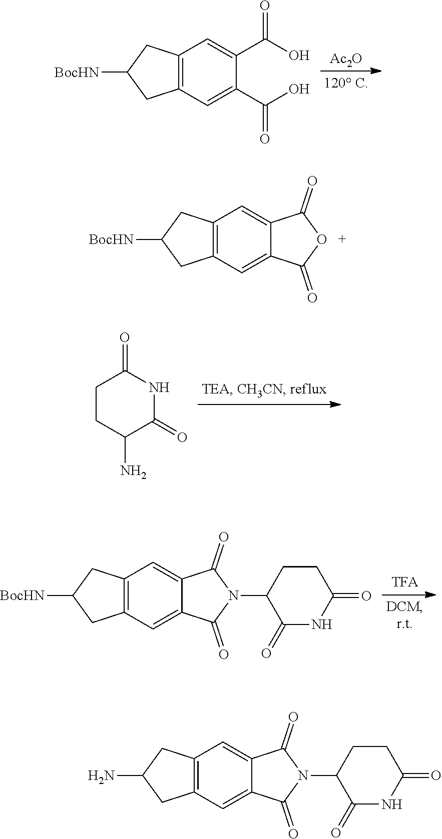

### Example 19

The synthesis of 3-(6-amino-1-oxo-3,5,6,7-tetrahydrocyclopenta[f]isoindol-2(1H)-yl)piperidine-2,6-dione is shown in Scheme 19. (S)-3-(6-Amino-1-oxo-3,5,6,7-tetrahydrocyclopenta[f]isoindol-2(1H)-yl)piperidine-2,6-dione and (R)-3-(6-amino-1-oxo-3,5,6,7-tetrahydrocyclopenta[f]isoindol-2(1H)-yl)piperidine-2,6-dione can also be prepared according to Scheme 19 using (S)-3-aminopiperidine-2,6-dione or (R)-3-aminopiperidine-2,6-dione instead of 3-aminopiperidine-2,6-dione.

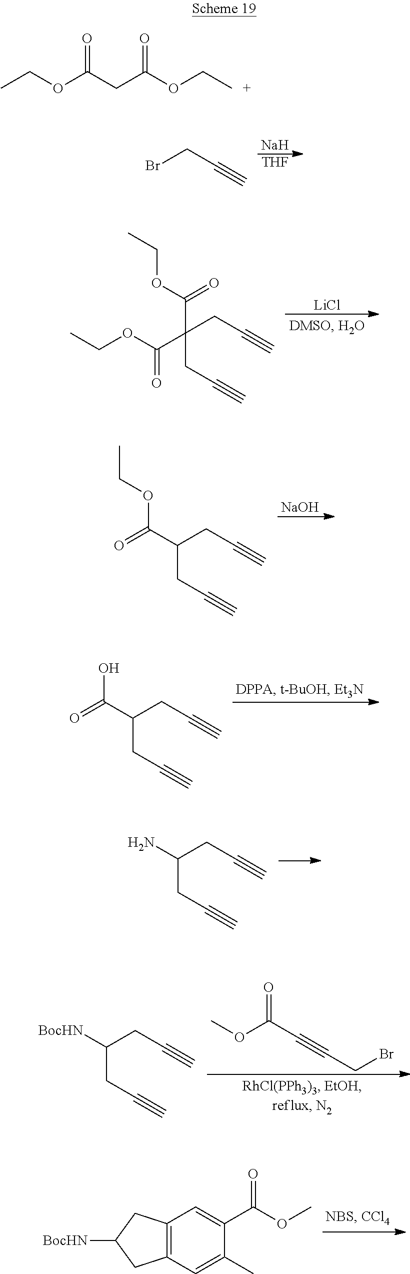

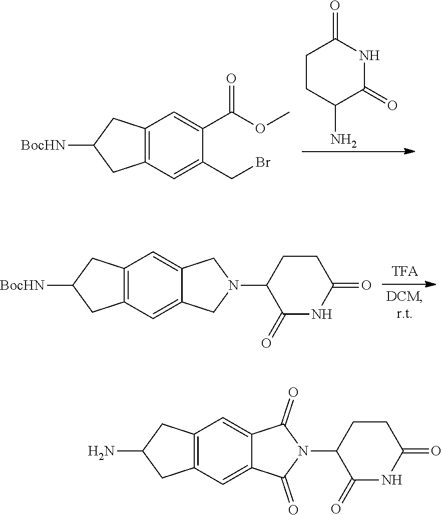

### Example 20

The synthesis of 3-(3-oxo-1,3,6,7,8,9-hexahydro-2H-pyrrolo[3,4-f]isoquinolin-2-yl)piperidine-2,6-dione is shown in Scheme 20. (S)-3-(3-Oxo-1,3,6,7,8,9-hexahydro-2H-pyrrolo[3,4-f]isoquinolin-2-yl)piperidine-2,6-dione and (R)-3-(3-oxo-1,3,6,7,8,9-hexahydro-2H-pyrrolo[3,4-f]isoquinolin-2-yl)piperidine-2,6-dione can also be prepared according to Scheme 20 using (S)-3-aminopiperidine-2,6-dione or (R)-3-aminopiperidine-2,6-dione instead of 3-aminopiperidine-2,6-dione

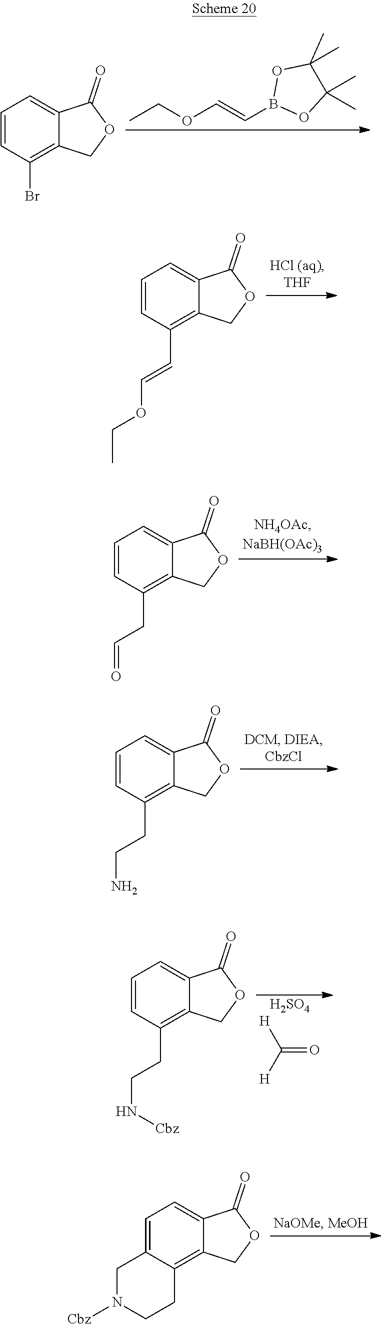

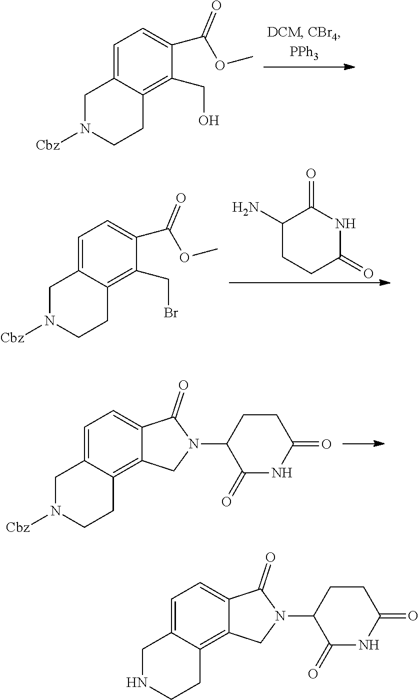 [SMILES: COC(=O)c1ccc2c(c1CO)CCN(C(=O)OCc1ccccc1)C2 ; COC(=O)c1ccc2c(c1CBr)CCN(C(=O)OCc1ccccc1)C2 ; NC1CCC(=O)NC1=O ; O=C1CCC(N2Cc3c(ccc4c3CCN(C(=O)OCc3ccccc3)C4)C2=O)C(=O)N1 ; O=C1CCC(N2Cc3c(ccc4c3CCNC4)C2=O)C(=O)N1]

### Example 21

The synthesis of 3-(3-oxo-1,3,6,7,8,9-hexahydro-2H-pyrrolo[3,4-h]isoquinolin-2-yl)piperidine-2,6-dione is shown in Scheme 21. (S)-3-(3-Oxo-1,3,6,7,8,9-hexahydro-2H-pyrrolo[3,4-h]isoquinolin-2-yl)piperidine-2,6-dione and (R)-3-(3-oxo-1,3,6,7,8,9-hexahydro-2H-pyrrolo[3,4-h]isoquinolin-2-yl)piperidine-2,6-dione can also be prepared according to Scheme 21 using (S)-3-aminopiperidine-2,6-dione or (R)-3-aminopiperidine-2,6-dione instead of 3-aminopiperidine-2,6-dione

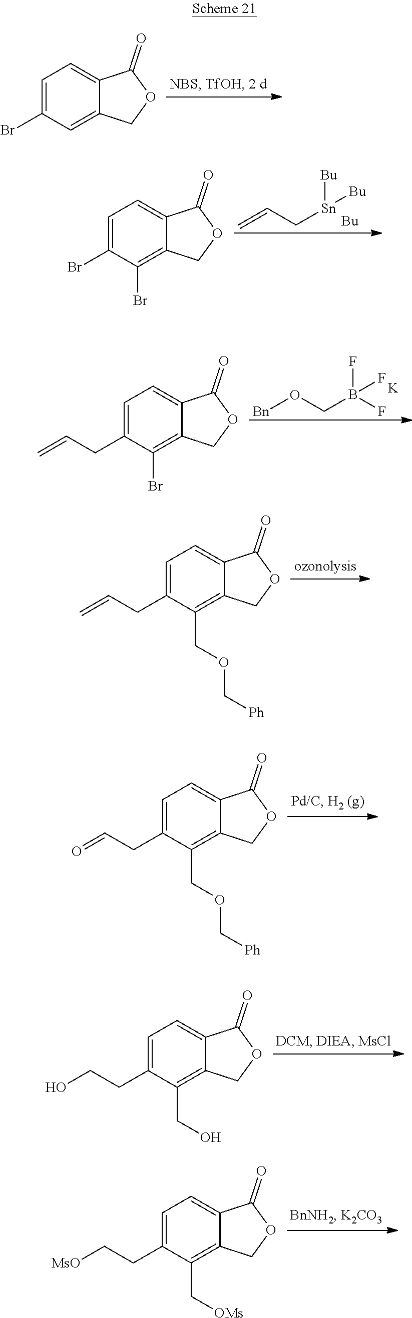

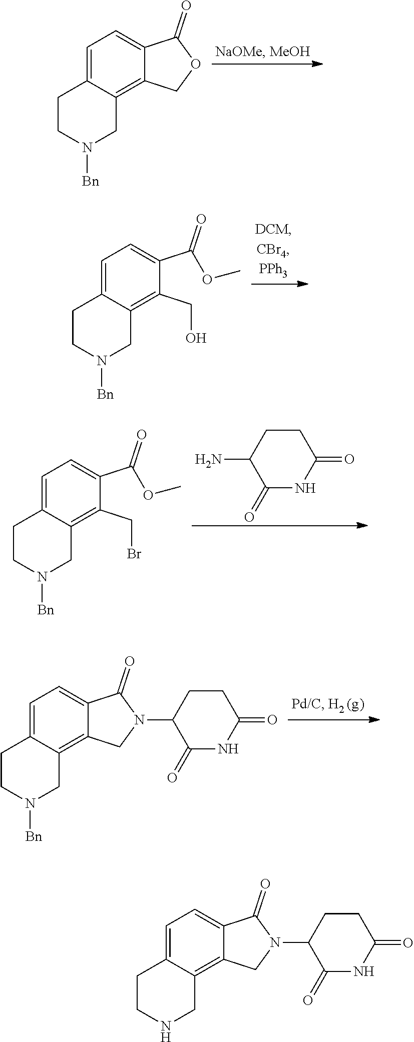

### Example 22

The synthesis of 3-(2-methyl-4-oxo-4,6,7,8-tetrahydro-3H-pyrrolo[3,4-g]quinazolin-3-yl)piperidine-2,6-dione is shown in Schemes 22A and 22B. (S)-3-(2-Methyl-4-oxo-4,6,7,8-tetrahydro-3H-pyrrolo[3,4-g]quinazolin-3-yl)piperidine-2,6-dione and (R)-3-(2-methyl-4-oxo-4,6,7,8-tetrahydro-3H-pyrrolo[3,4-g]quinazolin-3-yl)piperidine-2,6-dione can also be prepared according to Scheme 22 using (S)-3-aminopiperidine-2,6-dione or (R)-3-aminopiperidine-2,6-dione instead of 3-aminopiperidine-2,6-dione.

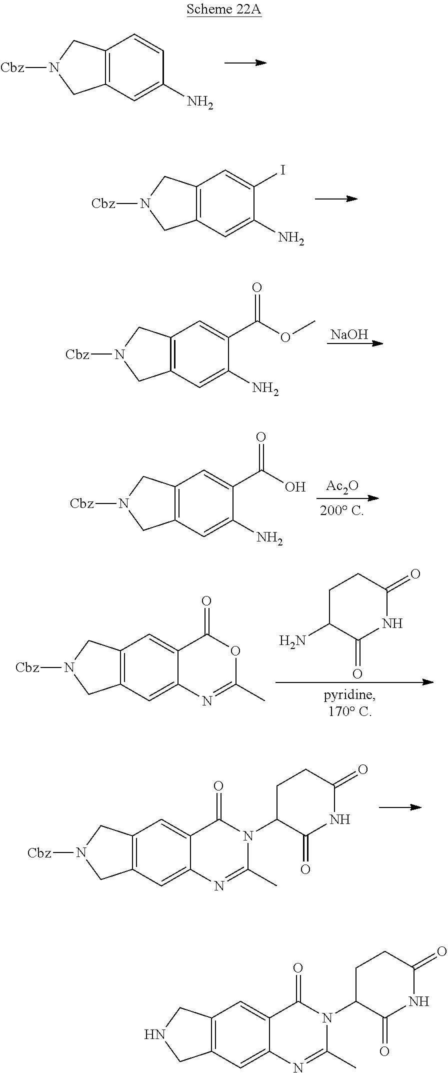

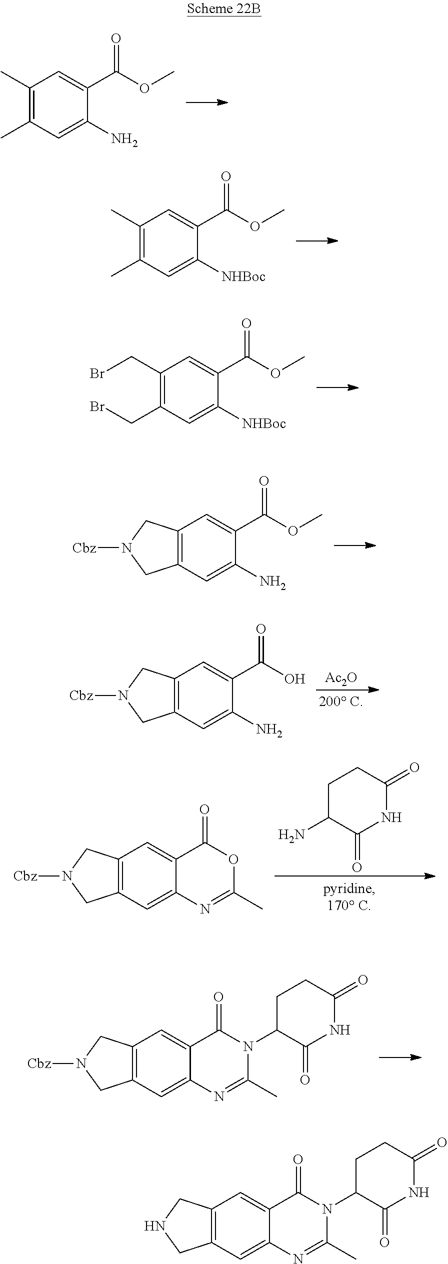

### Example 23

The synthesis of 3-(2-methyl-4-oxo-6,7,8,9-tetrahydropyrido[4,3-g]quinazolin-3(4H)-yl)piperidine-2,6-dione is shown in Scheme 23. (S)-3-(2-Methyl-4-oxo-6,7,8,9-tetrahydropyrido[4,3-g]quinazolin-3(4H)-yl)piperidine-2,6-dione and (R)-3-(2-methyl-4-oxo-6,7,8,9-tetrahydropyrido[4,3-g]quinazolin-3(4H)-yl)piperidine-2,6-dione can also be prepared according to Scheme 23 using (S)-3-aminopiperidine-2,6-dione or (R)-3-aminopiperidine-2,6-dione instead of 3-aminopiperidine-2,6-dione.

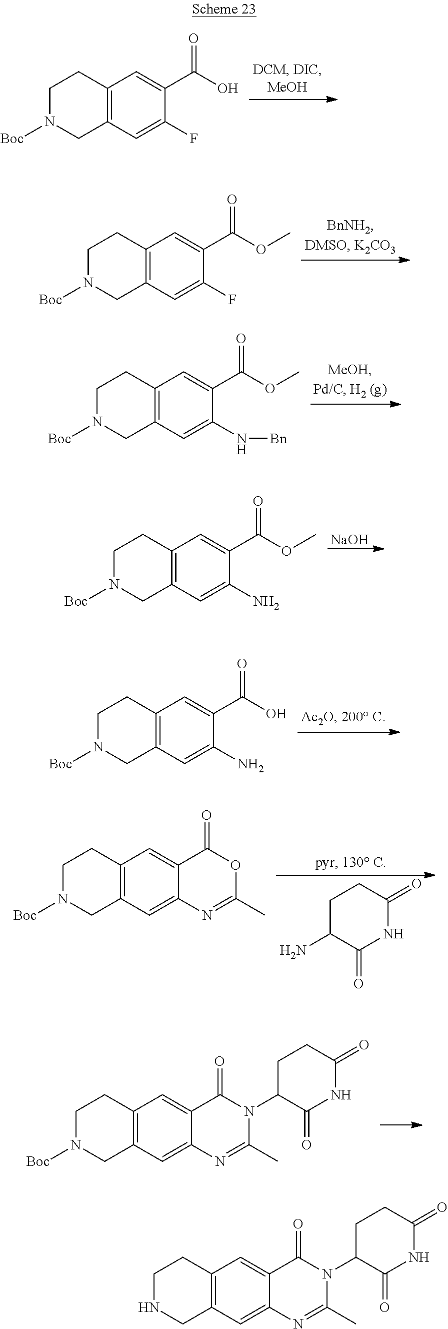

### Example 24

The synthesis of 2′-(2,6-dioxopiperidin-3-yl)-5′,7′-dihydro-1′H-spiro[azetidine-3,6′-cyclopenta[f]isoindole]-1′,3′(2′H)-dione and 3-(1′-oxo-5′,7′-dihydro-1′H-spiro[azetidine-3,6′-cyclopenta[f]isoindol]-2′(3′H)-yl)piperidine-2,6-dione is shown in Scheme 24. (S)-2′-(2,6-Dioxopiperidin-3-yl)-5′,7′-dihydro-1′H-spiro[azetidine-3,6′-cyclopenta[f]isoindole]-1′,3′(2′H)-dione, (S)-3-(1′-oxo-5′,7′-dihydro-1′H-spiro[azetidine-3,6′-cyclopenta[f]isoindol]-2′(3′H)-yl)piperidine-2,6-dione, (R)-2′-(2,6-dioxopiperidin-3-yl)-5′,7′-dihydro-1′H-spiro[azetidine-3,6′-cyclopenta[f]isoindole]-1′,3′(2′H)-dione, and (R)-3-(1′-oxo-5′,7′-dihydro-1′H-spiro[azetidine-3,6′-cyclopenta[f]isoindol]-2′(3′H)-yl)piperidine-2,6-dione can also be prepared according to Scheme 24 using (S)-3-aminopiperidine-2,6-dione or (R)-3-aminopiperidine-2,6-dione instead of 3-aminopiperidine-2,6-dione.

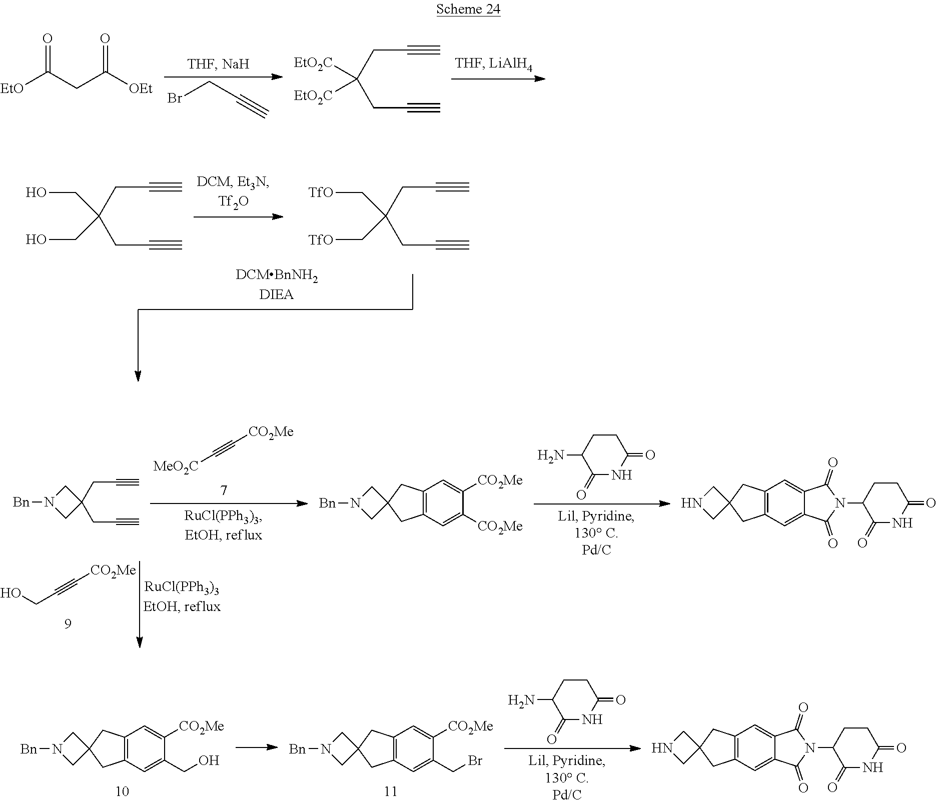

### Alternative Scheme

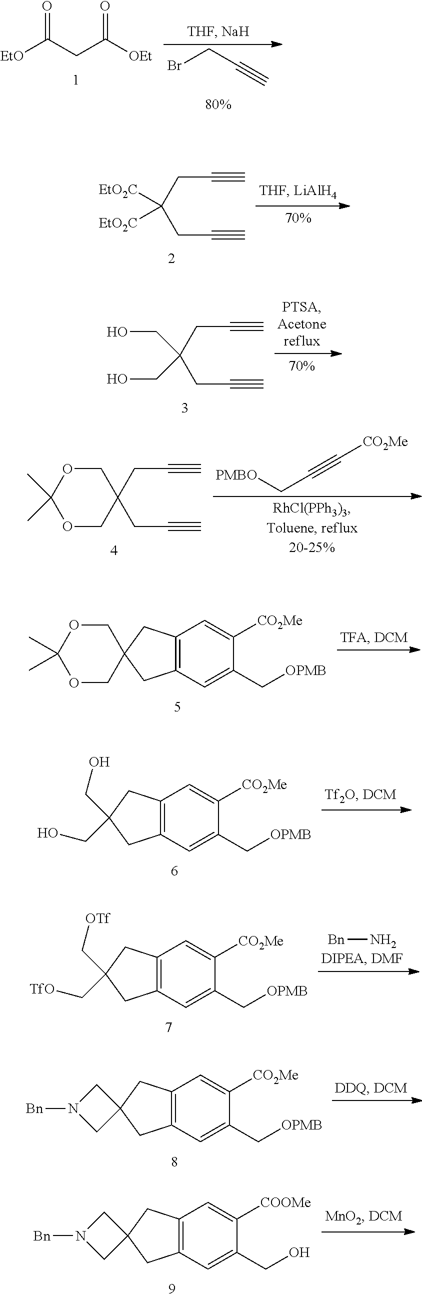

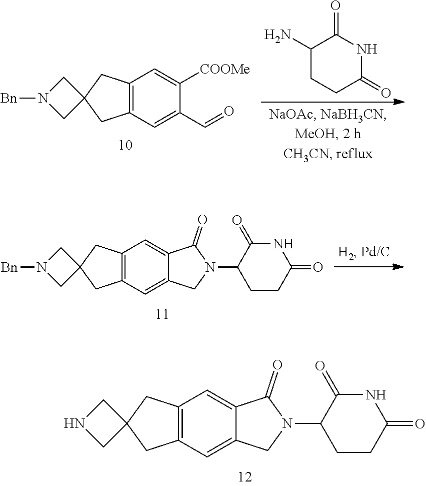 [SMILES: COC(=O)c1cc2c(cc1C=O)CC1(C2)CN(Cc2ccccc2)C1 ; NC1CCC(=O)NC1=O ; O=C1CCC(N2Cc3cc4c(cc3C2=O)CC2(C4)CN(Cc3ccccc3)C2)C(=O)N1 ; O=C1CCC(N2Cc3cc4c(cc3C2=O)CC2(CNC2)C4)C(=O)N1]

¹H NMR of compound 12 (400 MHz, Methanol-d₄) δ 7.65 (s, 1H), 7.44 (s, 1H), 5.15 (dd, J=13.4, 5.2 Hz, 1H), 4.45 (d, J=7.3 Hz, 2H), 3.91 (d, J=3.8 Hz, 4), 3.26 (d, J=5.9 Hz, 4H), 2.92 (ddd, J=17.6, 13.4, 5.3 Hz, 1H), 2.80 (ddd, J=17.7, 4.7, 2.5 Hz, 1H), 2.50 (qd, J=13.2, 4.7 Hz, 1H), 2.18 (dtd, J=12.9, 5.3, 2.5 Hz, 1H).

### Example 25

The synthesis of 3-(3-methyl-2-oxo-3,6,7,8-tetrahydroimidazo[4,5-e]isoindol-1(2H)-yl)piperidine-2,6-dione and 3-(1-methyl-2-oxo-1,6,7,8-tetrahydroimidazo[4,5-e]isoindol-3(2H)-yl)piperidine-2,6-dione is shown is Schemes 25A and 25B, respectively.

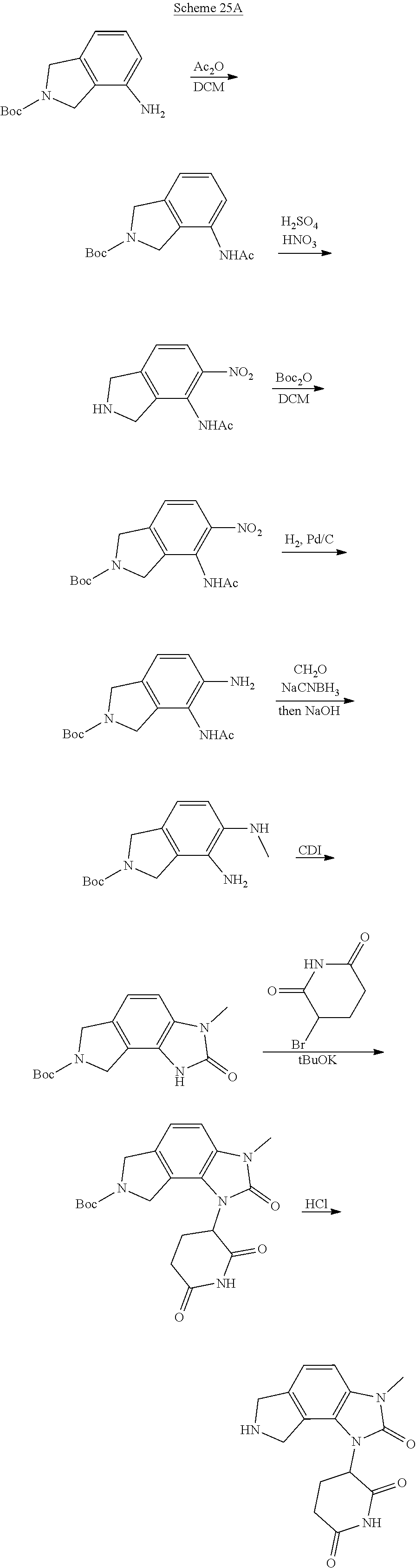

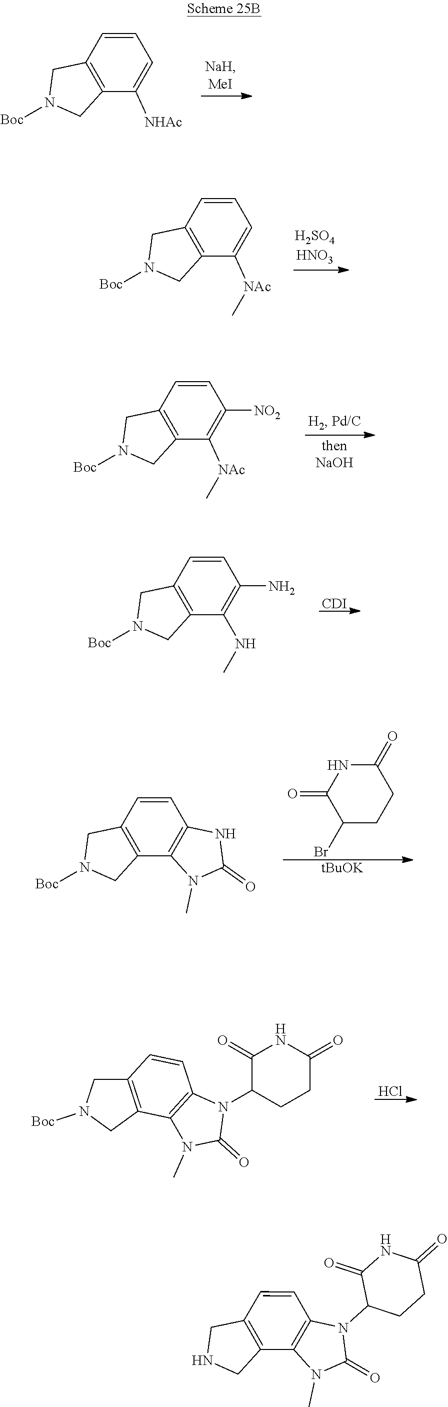

### Example 26

The synthesis of 2′-(2,6-dioxopiperidin-3-yl)-5′,7′-dihydro-1′H-spiro[azetidine-3,6′-cyclopenta[f]isoindole]-1′,3′(2′H)-dione is shown is Scheme 26

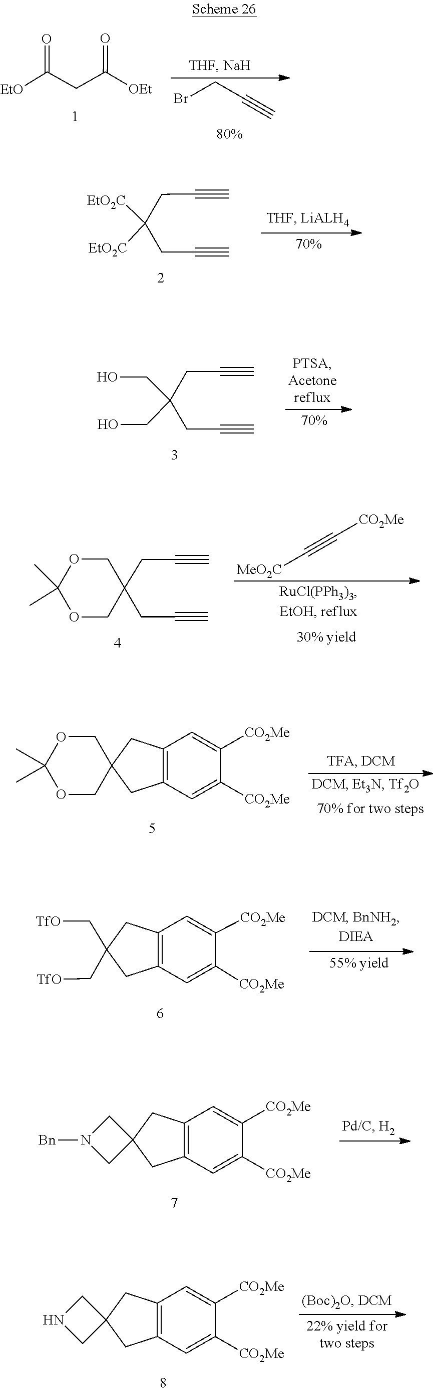

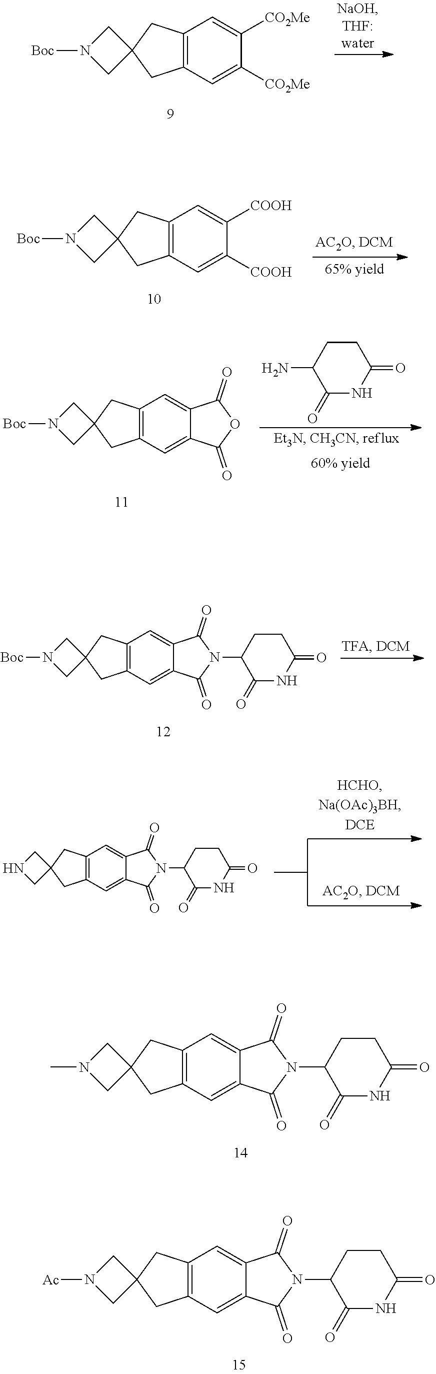

¹H NMR of compound 13 (400 MHz, Methanol-d₄) δ 7.76 (s, 2H), 5.14 (dd, J=12.6, 5.5 Hz, 1H), 4.15 (s, 4H), 3.47 (s, 4H), 2.97-2.62 (m, 3H), 2.20-2.09 (m, 1H).

### Example 27

The synthesis of 2-(2,6-dioxopiperidin-3-yl)-4-methoxy-6,7-dihydropyrrolo[3,4-f]isoindole-1,3(2H,5H)-dione is shown is Scheme 27

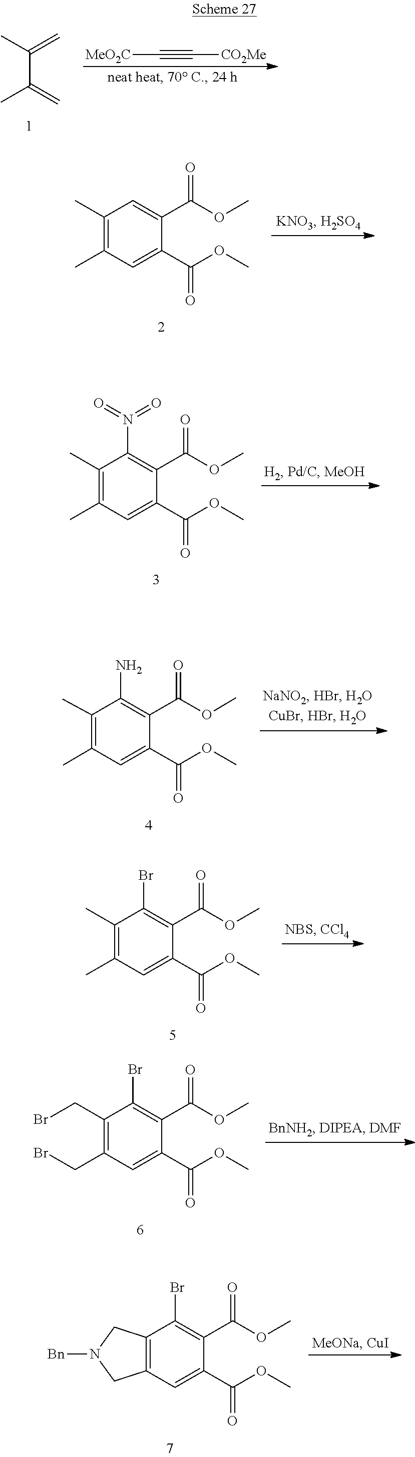

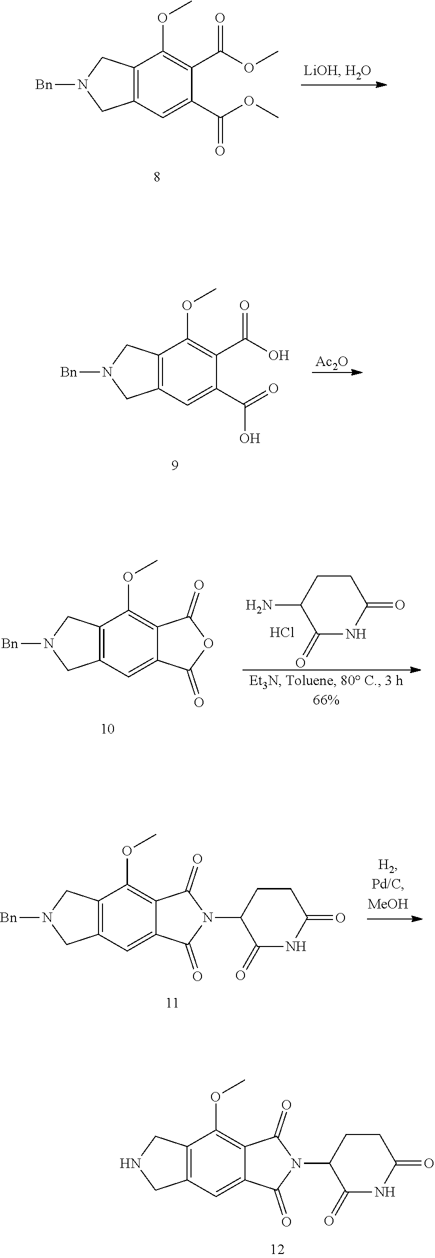

¹H NMR of compound 12 (400 MHz, Methanol-d₄) δ 7.62 (s, 1H), 5.17 (dd, J=12.5, 5.5 Hz, 1H), 4.75 (d, J=19.0 Hz, 2H), 4.33 (s, 2H), 3.33 (s, 3H), 2.96-2.83 (m, 1H), 2.83-2.68 (m, 2H), 2.21-2.11 (m, 1H).

### Example 28

The synthesis of 3-(4-methoxy-1-oxo-3,5,6,7-tetrahydropyrrolo[3,4-f]isoindol-2(1H)-yl)piperidine-2,6-dione is shown is Scheme 28

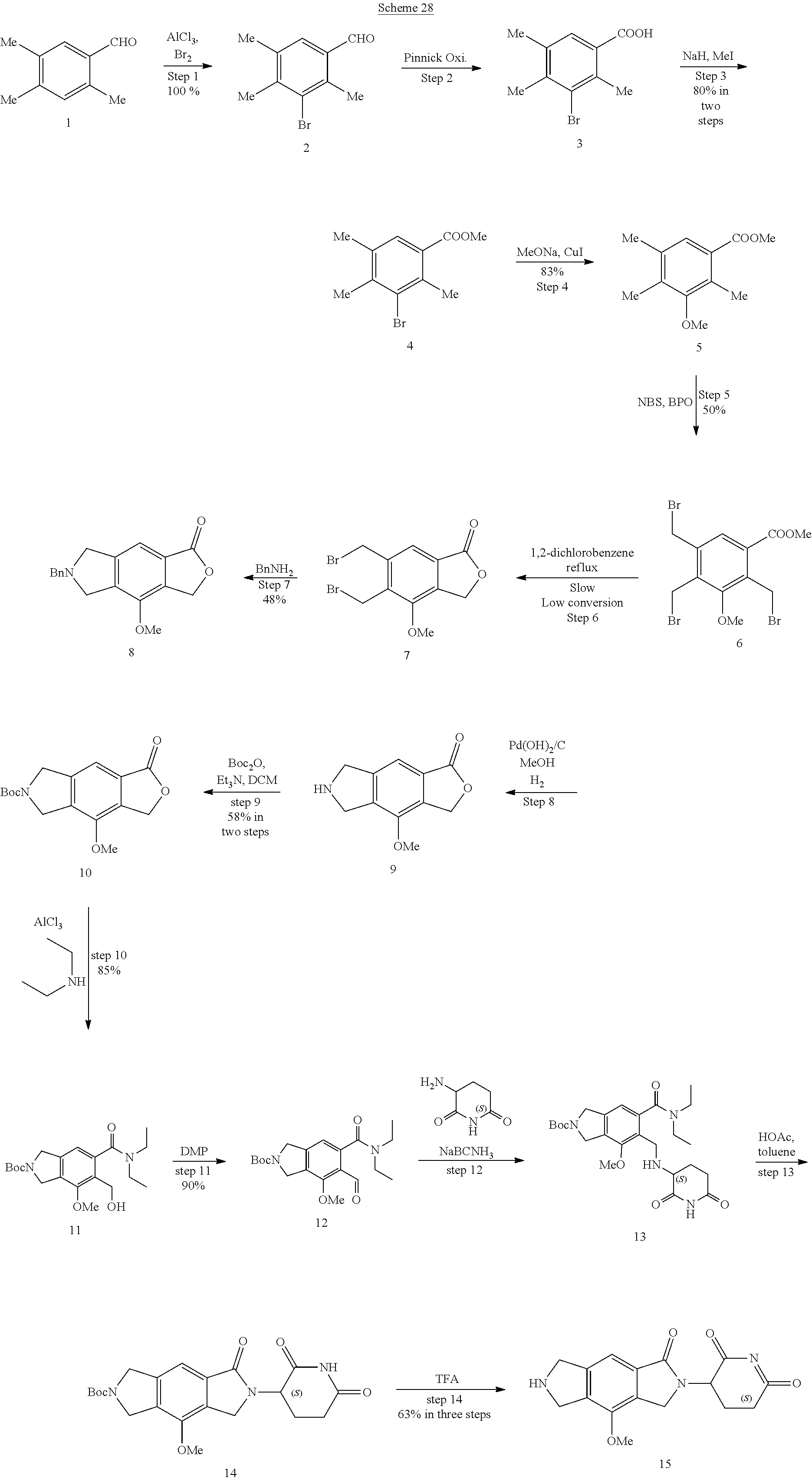

Detailed Procedures:

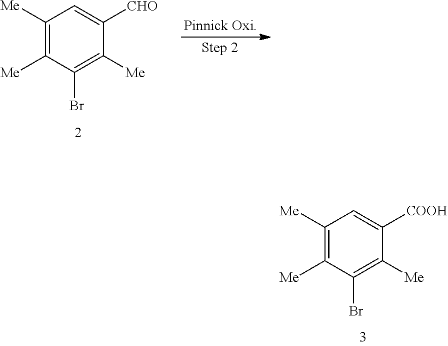 [SMILES: Cc1cc(C=O)c(C)c(Br)c1C ; Cc1cc(C(=O)O)c(C)c(Br)c1C]

To a solution of 2 (1 equiv) in t-BuOH/H2O=3:1 (c2=0.25 mol/L). NaH2PO4 (5 equiv) and Isoamylene (10 equiv) was added into the flask at room temperature. Then NaClO (3 equiv) was added into the flask. Quenched with saturated NaHSO3, The organic phase was separated. EA was added to the mixture, the resulting mixture was washed by brine. The combined organic phase was dried by MgSO4. Filtered and concentrated in vacuum. The residue was used for next step without further purification.

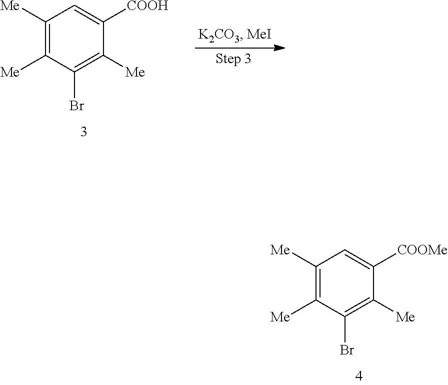 [SMILES: Cc1cc(C(=O)O)c(C)c(Br)c1C ; COC(=O)c1cc(C)c(C)c(Br)c1C]

To a solution of 3 (1 equiv) in acetone (c₃=0.5 mol/L). K₂CO₃ (3 equiv) and Mel (1.2 equiv) was added into the flask at room temperature. Quenched with saturated NaHCO₃. EA was added to the mixture, The organic phase was separated. the resulting mixture was washed by brine. The combined organic phase was dried by MgSO4. Filtered and concentrated in vacuum. The residue was purified by silica gel chromatography, the combination yield of two steps is 80%.

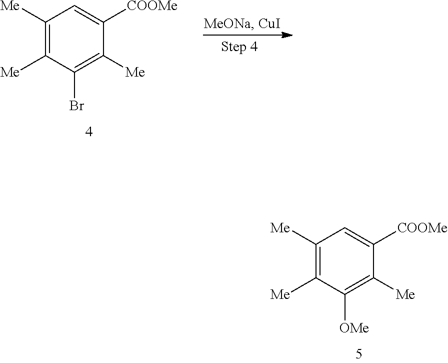 [SMILES: COC(=O)c1cc(C)c(C)c(Br)c1C ; COC(=O)c1cc(C)c(C)c(OC)c1C]

To a solution of 4 (1 equiv) in DMF (c₄=0.25 mol/L). MeONa (25 wt % in methanol, 2 equiv) and CuI (1 equiv), EA (1 equiv) was added into the flask. Heat to 100° C. for 6 h. Quenched with 3M HCl. EA was added to the mixture, The organic phase was separated. the resulting mixture was washed by brine. The combined organic phase was dried by MgSO4. Filtered and concentrated in vacuum. The residue was purified by silica gel chromatography, yield 83%.

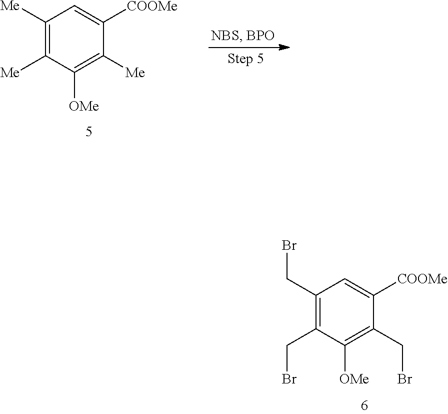 [SMILES: COC(=O)c1cc(C)c(C)c(OC)c1C ; COC(=O)c1cc(CBr)c(CBr)c(OC)c1CBr]

5 (1.0 equiv.) and N-bromosuccinimide (3.2 equiv.) were dissolved in 2-dichloroethane (c₅=0.5 mol/L), the reaction mixture was heated to 80° C. and benzoyl peroxide (0.02 equiv.) was added in one portion. Heating continued for 8 h. TLC (PE:EA=5:1, Rf=0.4) checked that 5 was consumed. The reaction mixture was cooled to ambient temperature, then filtered and the filtrates were concentrated. The residue was purified by silica gel chromatography to give 6 at 50% yield.

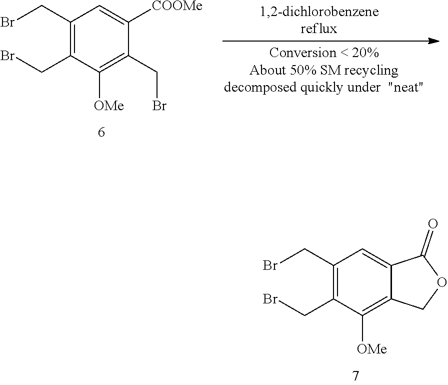 [SMILES: COC(=O)c1cc(CBr)c(CBr)c(OC)c1CBr ; COc1c(CBr)c(CBr)cc2c1COC2=O]

6 (1.0 equiv.) was dissolved in 1,2-dichlorobenzene (c₆=0.05 mol/L), the reaction mixture was refluxed for 24 hours. TLC (PE:EA=1:1, Rf=0.2) checked that 7 was appeared. The reaction mixture was cooled to ambient temperature, then was purified by silica gel chromatography straightly to give 7 at 10% yield, about 50% SM can be recycled.

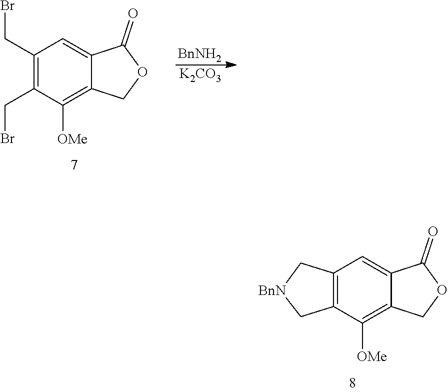

To a solution of 7 (1 equiv) in MeCN (c₇=0.2 mol/L). K₂CO₃ (3 equiv) and BnNH₂ (1 equiv) was added into the flask at room temperature in 5 portions. Reaction was quenched with saturated NaHCO₃. EA was added to the mixture, The organic phase was separated. the resulting mixture was washed by brine. The combined organic phase was dried by MgSO4. Filtered and concentrated in vacuum. The residue was purified by silica gel chromatography to give 8 at 48% yield.

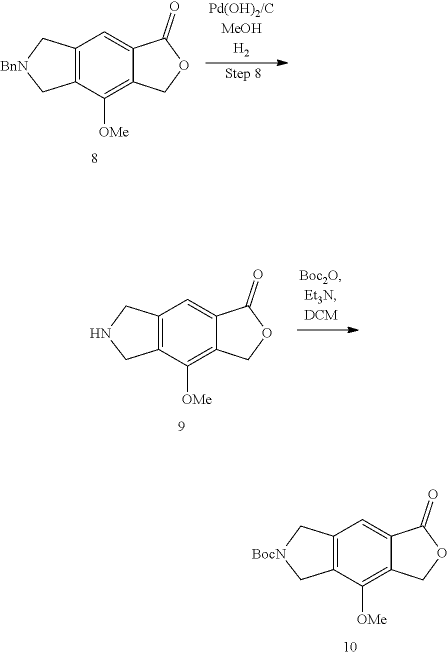 [SMILES: COc1c2c(cc3c1COC3=O)CN(Cc1ccccc1)C2 ; COc1c2c(cc3c1COC3=O)CNC2 ; COc1c2c(cc3c1COC3=O)CN(C(=O)OC(C)(C)C)C2]

To a solution of 8 (1 equiv) in MeOH (c₈=0.05 mol/L). Pd(OH)₂/C (¼ of 8's weight) was added into the flask. Hydrogenation with H₂ balloon for 3 h. Reaction was detected by UPLM-MS. Pd(OH)₂/C was filtered by cilite. concentrated in vacuum to get 9 without more purification. 9 was dissolved in DCM (c₉=0.1 mol/L). Et₃N (1.5 equiv) and Boc₂O (1.2 equiv) were added into the flask. Reaction was detected by UPLM-MS. Concentrated and purified by silica gel chromatography to give 10 at 58% yield.

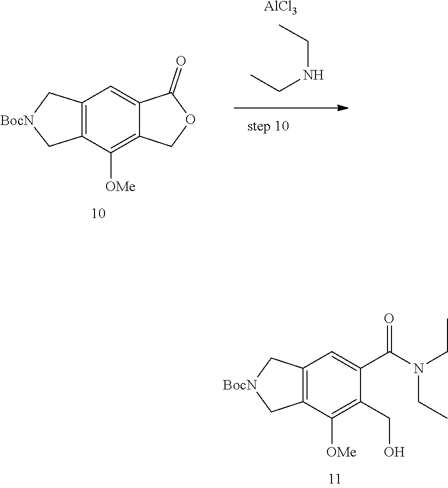

To a suspension of aluminium trichloride (1.3 equiv.) in DCM (C_{AlCl3}=0.5 mol/L), diethylamine (2.5 equiv.) was added at 0° C. and the mixture was stirred for additional 30 min. A solution of 10 (1.0 eq.) in DCM (c₁₀=1 mol/L), was added and the resulting mixture was stirred at 25° C. for 1 h. UPLM-MS(11: M+H:379, M+H-H₂O:361) checked that 10 was consumed. The reaction mixture was poured into 300 mL saturated aqueous NH₄Cl. The organic layers were combined and washed with 200 mL saturated aqueous NH₄Cl, dried over anhydrous MgSO₄, and concentrated in vacuum. The obtained residue was purified by silica gel chromatography to give 11 (85% yield).

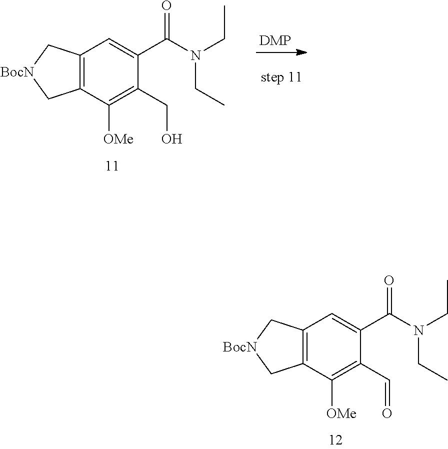 [SMILES: CCN(CC)C(=O)c1cc2c(c(OC)c1CO)CN(C(=O)OC(C)(C)C)C2 ; CCN(CC)C(=O)c1cc2c(c(OC)c1C=O)CN(C(=O)OC(C)(C)C)C2]

To a solution of 11 (1 equiv) in DCM (c₁=0.1 mol/L). DMP (1.1 equiv) was added into the flask at room temperature. Reaction was detected by UPLM-MS. Quenched with saturated NaHCO₃. EA was added to the mixture, The organic phase was separated. the resulting mixture was washed by brine. The combined organic phase was dried by MgSO₄. Filtered and concentrated in vacuum. The residue was purified by silica gel chromatography to give 12 (90%).

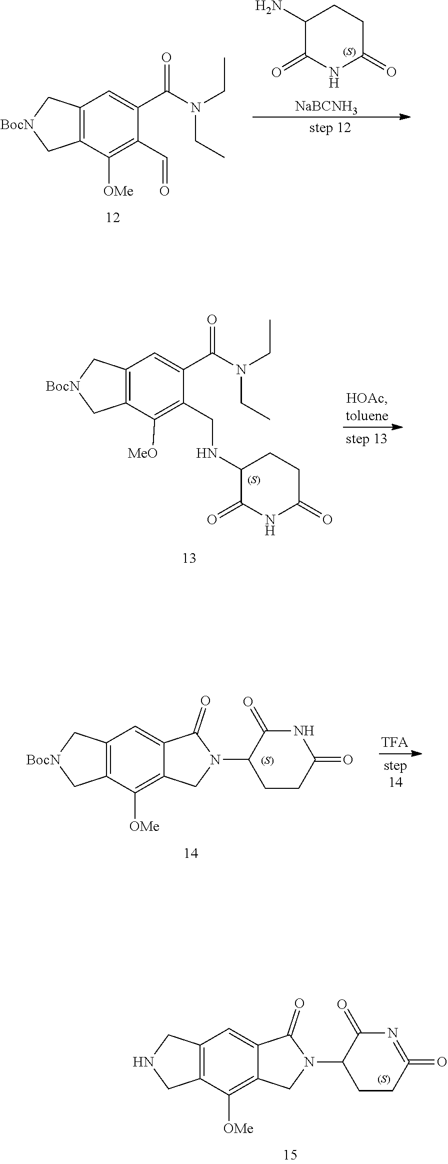

To a solution of 12 (1 equiv) in MeOH (c₁=0.2 mol/L). NaOAc (1.0 equiv) and (S)-3-Amino-piperidine-2,6-dione hydrochloride NaOAc (1.0 equiv), NaCNBH₃ (1.0 equiv) was added into the flask at room temperature. Reaction was detected by UPLM-MS (about 3 hours). Remove solvent under vacuum. The residue was dissolved in toluene. HOAc (15 equiv) was added into flask. The reaction was heated at 110° C. and stirred for 12 hour. Reaction was detected by UPLM-MS, all 13 is change into 14. 14 was purified by HPLC (TFA condition). 1.0 equiv TFA was added and concentrated 14 to get de-Boc 15 (63% yield in three steps).

### Example 29. Biological Activities of the Exemplary Compounds

Procedure for Cereblon Binding Assay. The binding to cereblon (CRBN) was determined using the Cereblon Binding Kit (Cisbio, #64BDCRBNPEG) following the manufacturer's instruction. Briefly, serially diluted compounds were incubated with GST-tagged wild-type human CRBN protein, XL665-labelled Thalidomide and Europium Cryptate labelled GST antibody at room temperature for about 3 hours. Time Resolved Fluorescence Resonance Energy Transfer (TR-FRET) measurements were acquired on a CALRIOstar plate reader with MARS data analysis software (BMG Labtech), with the following settings: 665/10 nm and 620/10 nm emission, 60 s delay and 400 s integration. The TR-FRET ratio was taken as the 665/620 nm intensity ratio. The readings were normalized to the control (0.5%) and the ICso was calculated by nonlinear regression (four parameters sigmoid fitted with variable slope) analysis using the GraphPad Prism 8 software.

Binding Activity: The recorded binding activity is summarized in Table A (“++++”≤0.5 μM, 0.5 μM<“+++”≤1.0 μM, 1.0 μM<“++”≤10 μM, and 10 μM<“+”).

TABLE A

| | Binding Activity |
|---|---|
| Compound No. | (IC50) |
| Lenalidomide | +++ |
| 20 | ++ |
| 217 | + |
| 218 | ++ |
| 219 | ++ |
| 220 | ++++ |
| 221 | +++ |
| 222 | ++++ |
| 223 | ++ |
| 224 | ++++ |
| 225 | ++++ |
| 226 | ++++ |
| 227 | +++ |
| 228 | ++++ |
| 229 | +++ |
| 230 | +++ |
| 231 | +++ |
| 232 | +++ |
| 233 | ++ |
| 234 | +++ |
| 235 | +++ |
| 236 | ++++ |
| 237 | ++++ |
| 238 | ++ |
| 239 | ++++ |
| 240 | ++++ |
| 241 | +++ |
| 242 | ++ |
| 243 | +++ |
| 244 | ++++ |
| 245 | ++++ |
| 246 | ++ |
| 247 | +++ |
| 248 | ++++ |
| 249 | +++ |
| 250 | ++++ |
| 251 | +++ |
| 252 | ++++ |
| 253 | ++++ |
| 254 | ++++ |
| 255 | ++ |
| 256 | ++++ |
| 257 | ++ |
| 258 | ++ |
| 259 | +++ |
| 260 | ++++ |
| 261 | ++ |
| 262 | ++ |
| 263 | +++ |
| 264 | ++++ |
| 265 | +++ |
| 266 | ++++ |
| 267 | ++ |
| 268 | +++ |
| 269 | ++++ |
| 270 | +++ |
| 271 | ++++ |
| 272 | ++++ |
| 273 | ++ |
| 274 | ++ |
| 275 | +++ |

### EQUIVALENTS

It is to be understood that the foregoing embodiments and exemplifications are not intended to be limiting in any respect to the scope of the disclosure, and that the claims presented herein are intended to encompass all embodiments and exemplifications whether or not explicitly presented herein.

All patents, patent applications, and publications cited herein are fully incorporated by reference herein in their entirety.

What is claimed is:

1. A compound of Formula I: 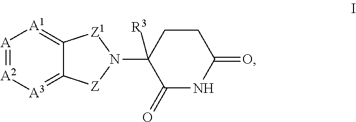
or a pharmaceutically acceptable salt or solvate thereof, wherein:
(a) A¹ is selected from —CR^{2a}═ and —N═;
A is —CR^{2b}═;
A² is —CR^{2c}═;
R^{2b} and R^{2c} are taken together to form a ring comprising a:
(i) —(CH₂)ₘ—X—(CH₂)ₙ— radical;
(ii) —C(═O)—N(R^{1d})—(CH₂)_{q}— radical;
(iii) —(CH₂)ₜ—N(R^{1e})—(CH₂)₂—Y—(CH₂)ᵤ— radical;
(iv) -E¹=E-E²=E³- radical;
(v)=A⁴-N(R^{1g})—CR^{2k}=radical; or
(vi) -E⁴=CR^{1j}-E⁵- radical;
R^{2a} is selected from hydrogen, halo, C₁-C₃ alkyl, amino, and C₁-C₃ alkoxy; and
R^{2d} is selected from hydrogen, halo, C₁-C₃ alkyl, amino, and C₁-C₃ alkoxy;
A³ is selected from —CR^{2d}═ and —N═; or
(b) A¹ is —CR^{2a}═;
A is —CR^{2b}═;
R^{2a} and R^{2b} are taken together to form a ring comprising a:
(i) —(CH₂)ₘ—X—(CH₂)ₙ— radical;
(ii) —C(═O)—N(R^{1d})—(CH₂)_{q}— radical;
(iii) —(CH₂)ₜ—N(R^{1e})—(CH₂)₂—Y—(CH₂)ᵤ— radical;
(iv) -E¹=E-E²=E³- radical;
(v)=A⁴-N(R^{1g})—CR^{2k}=radical; or
(vi) -E⁴=CR^{1j}-E⁵- radical;
A² is selected from —CR^{2c}═ and —N═;
A³ is selected from —CR^{2d}═ and —N═;
R^{2c} is selected from hydrogen, halo, C₁-C₃ alkyl, amino, and C₁-C₃ alkoxy; and
R^{2d} is selected from hydrogen, halo, C₁-C₃ alkyl, amino, and C₁-C₃ alkoxy;
X is selected from —N(R^{1a})— and —CR^{1b}R^{1c}—
R^{1a} is selected from hydrogen, C₁-C₃ alkyl, C₁-C₃ haloalkyl, cycloalkyl, aryl, heterocyclo, heteroaryl, (cycloalkyl)alkyl, (aryl)alkyl, (heterocyclo)alkyl, (heteroaryl)alkyl, —C(═O)-(cycloalkyl), —C(═O)-(aryl), —C(═O)-(heterocyclo), —C(═O)-(heteroaryl), and Q-L-, wherein the C₁-C₃ alkyl, C₁-C₃ haloalkyl, cycloalkyl, aryl, heterocyclo, heteroaryl, (cycloalkyl)alkyl, (aryl)alkyl, (heterocyclo)alkyl, (heteroaryl)alkyl, —C(═O)-(cycloalkyl), —C(═O)-(aryl), —C(═O)-(heterocyclo), —C(═O)-(heteroaryl) is optionally substituted with one or more R^{1aS};
each R^{1aS} is independently selected from oxo, halo, C₁-C₃ alkyl, C₁-C₃ haloalkyl, C₁-C₃ alkoxy, —C(═O)—OH, —C(═O)—(C₁-C₃ alkyl), —C(═O)O—(C₁-C₄ alkyl), —NH₂, —NH(C₁-C₃ alkyl), —N(C₁-C₃ alkyl)₂, cycloalkyl, aryl, heterocyclo, heteroaryl, (cycloalkyl)alkyl, (aryl)alkyl, (heterocyclo)alkyl, (heteroaryl)alkyl, —C(═O)-(cycloalkyl), —C(═O)-(aryl), —C(═O)-(heterocyclo), and —C(═O)-(heteroaryl), wherein the C₁-C₃ alkyl, C₁-C₃ haloalkyl, C₁-C₃ alkoxy, cycloalkyl, aryl, heterocyclo, heteroaryl, (cycloalkyl)alkyl, (aryl)alkyl, (heterocyclo)alkyl, (heteroaryl)alkyl, —C(═O)-(cycloalkyl), —C(═O)-(aryl), —C(═O)-(heterocyclo), —C(═O)-(heteroaryl) is optionally substituted with one or more R^{1a}ss;
each R^{1aSS} is independently selected from oxo, halo, C₁-C₃ alkyl, C₁-C₃ haloalkyl, C₁-C₃ alkoxy, —C(═O)—OH, —C(═O)—(C₁-C₃ alkyl), —NH₂, —NH(C₁-C₃ alkyl), and —N(C₁-C₃ alkyl)₂;
R^{1b} is selected from hydrogen, —CHO, —C(═O)OH, hydroxy, (hydroxy)C₁-C₃ alkyl, amino, (amino)alkyl, (heterocyclo)alkyl, and Q-L-, wherein the (heterocyclo)alkyl is optionally substituted with one or more R^{1b}s;
each R^{1bS} is independently (aryl)alkyl optionally substituted with one or more halo;
R^{1c} is hydrogen; or
R^{1b} and R^{1c} taken together with the carbon atom to which they are attached form a —C(═O)—; or
R^{1b} and R^{1c} taken together with the carbon atom to which they are attached form: 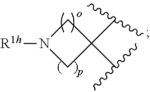
R^{1d} is selected from hydrogen, heterocyclo, (heterocyclo)alkyl, and Q-L-, wherein the heterocyclo or (heterocyclo)alkyl is optionally substituted with one or more R^{1aS};
each R^{1aS} is independently selected from C₁-C₃ alkyl, —C(═O)—(C₁-C₃ alkyl), and (aryl)alkyl, wherein the (aryl)alkyl is optionally substituted with one or more halo;
R^{1e} is selected from hydrogen and Q-L-;
R^{1g} is selected from hydrogen and Q-L-;
R^{1h} is selected from hydrogen, (aryl)alkyl, and Q-L-, wherein the (aryl)alkyl is optionally substituted with one or more halo;
Y is selected from —O—, —S—, and —N(R^{1f})—
R^{1f} is selected from hydrogen and C₁-C₃ alkyl;
E¹ is selected from —CR^{2e}═ and —N═;
E is selected from —CR^{2f}═ and —N═;
E² is selected from —CR^{2g}═ and —N═;
E³ is selected from —CR^{2h}═ and —N═;
R^{2e}, R^{2f}, R^{2g}, and R^{2h} are independently selected from hydrogen, halo, hydroxy, amino, —CHO, C₁-C₃ alkyl, and C₁-C₃ alkoxy; or
R^{2e} is Q-L-; and, if present, R^{2f}, R^{2g}, and R^{2h} are independently selected from hydrogen, halo, hydroxy, amino, —CHO, C₁-C₃ alkyl, and C₁-C₃ alkoxy; or
R^{2f} is Q-L-; and, if present, R^{2e}, R^{2g}, and R^{2h} are independently selected from hydrogen, halo, hydroxy, amino, —CHO, C₁-C₃ alkyl, and C₁-C₃ alkoxy; or
R^{2g} is Q-L-; and, if present, R^{2e}, R^{2f}, and R^{2h} are independently selected from hydrogen, halo, hydroxy, amino, —CHO, C₁-C₃ alkyl, and C₁-C₃ alkoxy; or
R^{2h} is Q-L-; and, if present, R^{2e}, R^{2f}, and R^{2g} are independently selected from hydrogen, halo, hydroxy, amino, —CHO, C₁-C₃ alkyl, and C₁-C₃ alkoxy;
E⁴ is selected from ═C(H)— and ═N—;
E⁵ is selected from —O—, —S—, and —N(R^{2m})—;
R^{2m} is selected from hydrogen and C₁-C₄ alkyl;
R^{1j} is selected from hydrogen, C₁-C₄ alkyl, (hydroxy)alkyl, (heterocyclo)alkyl, and Q-L;
A⁴ is selected from —CR^{2j}═ and —N═;
R^{2j} is selected from hydrogen and C₁-C₃ alkyl;
R^{2k} is selected from hydrogen and C₁-C₃ alkyl;
m is 1, 2, or 3;
n is 1, 2, or 3;
is 1, 2, or 3;
p is 1, 2, or 3;
q is 1 or 2;
t is 0 or 1;
u is 0 or 1;
R³ is selected from hydrogen, deuterium, fluoro, and C₁-C₃ alkyl;
Z and Z¹ are —C(═O)—; or
Z is —C(═O)— and Z¹ is —CR^{4a}R^{4b}—; or
Z is —CR^{4a}R^{4b}— and Z¹ is —C(═O)—; or
Z is —N═C(CH₃)— and Z¹ is —C(═O)—; or
Z is —C(═O)— and Z¹ is —N═C(CH₃)—; or
Z is a bond and Z¹ is —N(R²ⁿ)C(═O)—; or
Z is —N(R²ⁿ)C(═O) and Z is a bond;
R²ⁿ is selected from hydrogen and C₁-C₄ alkyl;
R^{4a} and R^{4b} are independently selected from hydrogen and C₁-C₃ alkyl; or
R^{4a} and R^{4b} taken together with the carbon to which they are attached form a C₃-C₆ cycloalkyl,
Q is a small molecule that binds to a target protein of interest;
L is -J¹-J²-J³-J⁴-J⁵-, wherein J¹ is attached to Q;
J¹ is selected from alkylenyl, cycloalkylenyl, and heterocyclenyl; or J¹ is absent;
J² is selected from —C(═O)—, —(CH₂)_{w}—, —CH═CH—, and —C≡C—;
w is 0, 1, 2, or 3;
J³ is selected from alkylenyl, heteroalkylenyl, cycloalkylenyl, heterocyclenyl, phenylenyl, and heteroarylenyl; or J³ is absent;
J⁴ is selected from alkylenyl, cycloalkylenyl, and heterocyclenyl; or J⁴ is absent;
J⁵ is selected from —O—, —N(H)—, —C≡C—, —(CH₂)ₓ— and —C(═O)—; and
x is 0, 1, 2, or 3.

2. The compound of claim 1, or a pharmaceutically acceptable salt or solvate thereof, wherein:
(a) A¹ is selected from —CR^{2a}═ and —N═;
A is —CR^{2b}—;
A² is —CR^{2c}═;
R^{2b} and R^{2e} are taken together to form a ring comprising a:
(i) —(CH₂)ₘ—X—(CH₂)ₙ— radical;
(ii) —C(═O)—N(R^{1d})—(CH₂)_{q}— radical;
(iii) —(CH₂)ₜ—N(R^{1e})—(CH₂)₂—Y—(CH₂)ᵤ— radical;
(iv) -E¹=E-E²=E³- radical;
(v)=A⁴-N(R^{1g})—CR^{2k}=radical; or
(vi) -E⁴=CR^{1j}-E⁵- radical;
R^{2a} is selected from hydrogen, halo, C₁-C₃ alkyl, amino, and C₁-C₃ alkoxy; and
R^{2d} is selected from hydrogen, halo, C₁-C₃ alkyl, amino, and C₁-C₃ alkoxy;
A³ is selected from —CR^{2d}═ and —N═; or
(b) A¹ is —CR^{2a}—;
A is —CR^{2b}—;
R^{2a} and R^{2b} are taken together to form a ring comprising a:
(i) —(CH₂)ₘ—X—(CH₂)ₙ— radical;
(ii) —C(═O)—N(R^{1d})—(CH₂)_{q}— radical;
(iii) —(CH₂)ₜ—N(R^{1e})—(CH₂)₂—Y—(CH₂)ᵤ— radical;
(iv) -E=E-E²=E³- radical; or
(v)=A⁴-N(R^{1g})—CR^{2k}=radical; or
(vi) -E⁴=CR^{1j}-E⁵- radical;
A² is selected from —CR^{2c}═ and —N═;
A³ is selected from —CR^{2d}═ and —N═;
R^{2e} is selected from hydrogen, halo, C₁-C₃ alkyl, amino, and C₁-C₃ alkoxy; and
R^{2d} is selected from hydrogen, halo, C₁-C₃ alkyl, amino, and C₁-C₃ alkoxy;
X is selected from —N(R^{1a})— and —CR^{1b}R^{1c}—
R^{1a} is selected from hydrogen and Q-L-;
R^{1b} is selected from hydrogen, —CHO, —C(═O)OH, hydroxy, (hydroxy)C₁-C₃ alkyl, amino, (amino)alkyl, and Q-L-;
R^{1c} is hydrogen; or
R^{1b} and R^{1c} taken together with the carbon atom to which they are attached form a —C(═O)—; or
R^{1b} and R^{1c} taken together with the carbon atom to which they are attached form: 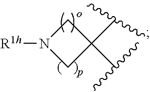
R^{1d} is selected from hydrogen and Q-L-;
R^{1e} is selected from hydrogen and Q-L-;
R^{1g} is selected from hydrogen and Q-L-;
R^{1h} is selected from hydrogen and Q-L-;
Y is selected from —O—, —S—, and —N(R^{1f})—
R^{1f} is selected from hydrogen and C₁-C₃ alkyl;
E¹ is selected from —CR^{2e}═ and —N═;
E is selected from —CR^{2f}═ and —N═;
E² is selected from —CR^{2g}═ and —N═;
E³ is selected from —CR^{2h}═ and —N═;
R^{2e}, R^{2f}, R^{2g}, and R^{2h} are independently selected from hydrogen, halo, hydroxy, amino, —CHO, C₁-C₃ alkyl, and C₁-C₃ alkoxy; or
R^{2e} is Q-L-; and, if present, R^{2f}, R^{2g}, and R^{2h} are independently selected from hydrogen, halo, hydroxy, amino, —CHO, C₁-C₃ alkyl, and C₁-C₃ alkoxy; or
R^{2f} is Q-L-; and, if present, R^{2e}, R^{2g}, and R^{2h} are independently selected from hydrogen, halo, hydroxy, amino, —CHO, C₁-C₃ alkyl, and C₁-C₃ alkoxy; or
R^{2g} is Q-L-; and, if present, R^{2e}, R^{2f}, and R^{2h} are independently selected from hydrogen, halo, hydroxy, amino, —CHO, C₁-C₃ alkyl, and C₁-C₃ alkoxy; or
R^{2h} is Q-L-; and, if present, R^{2e}, R^{2f}, and R^{2g} are independently selected from hydrogen, halo, hydroxy, amino, —CHO, C₁-C₃ alkyl, and C₁-C₃ alkoxy;
E⁴ is selected from ═C(H)— and ═N—;
E⁵ is selected from —O—, —S—, and —N(R^{2m})—;
R^{2m} is selected from hydrogen and C₁-C₄ alkyl;
R^{1j} is selected from hydrogen, C₁-C₄ alkyl, (hydroxy)alkyl, (heterocyclo)alkyl, and Q-L;
A⁴ is selected from —CR^{2j}═ and —N═;
R^{2j} is selected from hydrogen and C₁-C₃ alkyl;
R^{2k} is selected from hydrogen and C₁-C₃ alkyl;
m is 1, 2, or 3;
n is 1, 2, or 3;
is 1, 2, or 3;
p is 1, 2, or 3;
q is 1 or 2;
t is 0 or 1;
u is 0 or 1;
R³ is selected from hydrogen, deuterium, fluoro, and C₁-C₃ alkyl;
Z and Z¹ are —C(═O)—; or
Z is —C(═O)— and Z¹ is —CR^{4a}R^{4b}—; or
Z is —CR^{4a}R^{4b}— and Z¹ is —C(═O)—; or
Z is —N═C(CH₃)— and Z¹ is —C(═O)—; or
Z is —C(═O)— and Z¹ is —N═C(CH₃)—; or
Z is a bond and Z¹ is —N(R²ⁿ)C(═O)—; or
Z is —N(R²ⁿ)C(═O) and Z is a bond;
R²ⁿ is selected from hydrogen and C₁-C₄ alkyl;
R^{4a} and R^{4b} are independently selected from hydrogen and C₁-C₃ alkyl; or
R^{4a} and R^{4b} taken together with the carbon to which they are attached form a C₃-C₆ cycloalkyl,
Q is a small molecule that binds to a target protein of interest;
L is -J¹-J²-J³-J⁴-J⁵-, wherein J¹ is attached to Q;
J¹ is selected from alkylenyl, cycloalkylenyl, and heterocyclenyl; or J¹ is absent;
J² is selected from —C(═O)—, —(CH₂)_{w}—, —CH═CH—, and —C≡C—;
w is 0, 1, 2, or 3;
J³ is selected from alkylenyl, heteroalkylenyl, cycloalkylenyl, heterocyclenyl, phenylenyl, and heteroarylenyl; or J³ is absent;
J⁴ is selected from alkylenyl, cycloalkylenyl, and heterocyclenyl; or J⁴ is absent;
J⁵ is selected from —O—, —N(H)—, —C≡C—, —(CH₂)ₓ— and —C(═O)—; and
x is 0, 1, 2, or 3.

3. The compound of claim 1, wherein the compound is not a compound of any one of Formulae A-F: 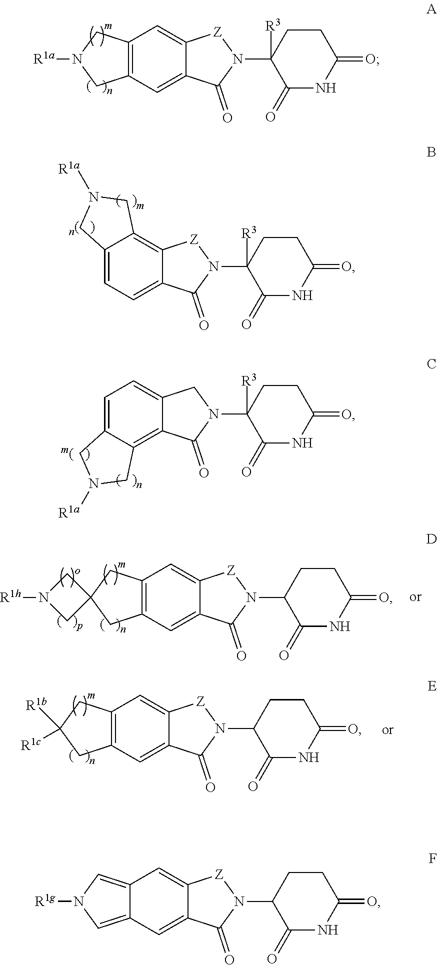
wherein:
R³ is selected from hydrogen, fluoro, methyl, and deuterium;
Z is selected from —C(═O)— and —CH₂—;
R^{1b} is selected from —OH, —CH₂OH, —CHO, —C(═O)OH, and Q-L; and
R^{1c} is hydrogen; or
R^{1b} and R^{1c} taken together with the carbon atom to which they are attached form a —C(═O)—.

4. The compound of any one of the preceding claims, being of any one of Formulae II-VI: 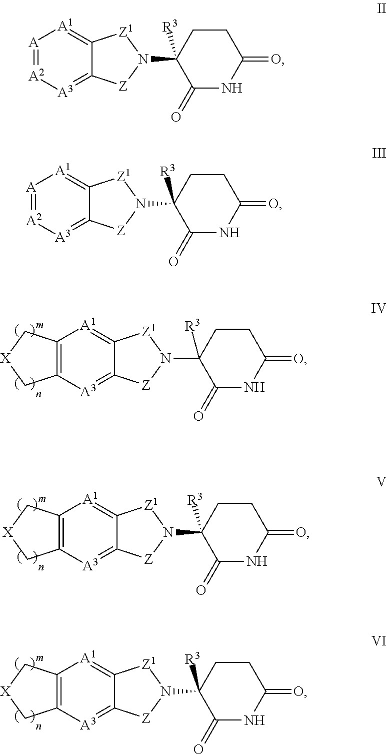
or a pharmaceutically acceptable salt or solvate thereof;
optionally, wherein:
(i) A¹ is —CR^{2a}═ and A³ is —CR^{2d}═;
optionally, R^{2a} is selected from hydrogen, —NH₂, fluoro, and methoxy; and
optionally, R^{2d} is selected from hydrogen, —NH₂, fluoro, and methoxy;
(ii) A¹ is —N═ and A³ is —CR^{2d}═;
optionally, R^{2d} is selected from hydrogen, —NH₂, fluoro, and methoxy; or
(iii) A¹ is —CR^{2a}═ and A³ is —N═;
optionally, R^{2a} is selected from hydrogen, —NH₂, fluoro, and methoxy.

5. The compound of any one of the preceding claims, being or any one of Formulae VII-IX: 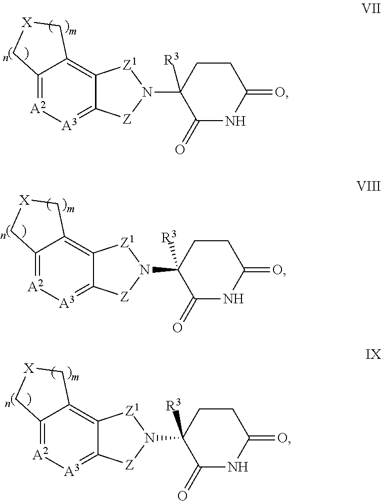
or a pharmaceutically acceptable salt or solvate thereof;
optionally, wherein:
(i) A² is —CR^{2c}═ and A³ is —CR^{2d}═;
optionally, R^{2e} is selected from hydrogen, —NH₂, fluoro, and methoxy; and
optionally, R^{2d} is selected from hydrogen, —NH₂, fluoro, and methoxy;
(ii) A² is —N═ and A³ is —CR^{2d}═;
optionally, R^{2d} is selected from hydrogen, —NH₂, fluoro, and methoxy; or
(iii) A² is —CR^{2c}═ and A³ is —N═;
optionally, R^{2c} is selected from hydrogen, —NH₂, fluoro, and methoxy.

6. The compound of any one of the preceding claims, or a pharmaceutically acceptable salt or solvate thereof, wherein X is —CR^{1b}R^{1c}—;
optionally, wherein:
(i) R^{1c} is hydrogen, and R^{1b} is selected from hydroxy, —NH₂, —CHO, —C(═O)OH, and —CH₂OH;
(ii) R^{1c} is hydrogen, and R^{1b} is Q-L-; or
(iii) R^{1b} and R^{1c} taken together with the carbon atom to which they are attached form —C(═O)—.

7. The compound of any one of the preceding claims, or a pharmaceutically acceptable salt or solvate thereof, wherein X is —N(R^{1a})═;
optionally, wherein:
(i) R^{1a} is hydrogen.
(ii) R^{1a} is Q-L-.

8. The compound of any one of the preceding claims, being of any one of Formulae X-XII: 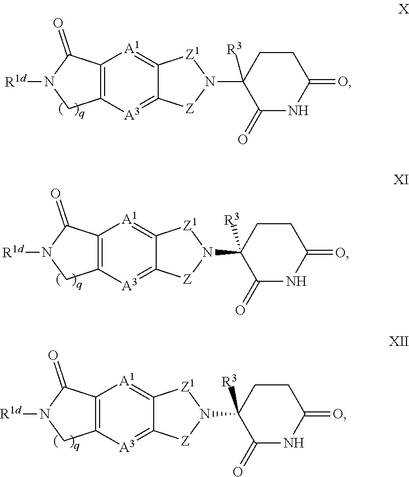
or a pharmaceutically acceptable salt or solvate thereof;
optionally, wherein:
(i) A¹ is —CR^{2a}═ and A³ is —CR^{2d}═.
optionally, R^{2a} is selected from hydrogen, —NH₂, fluoro, and methoxy; and
optionally, R^{2d} is selected from hydrogen, —NH₂, fluoro, and methoxy;
(ii) A¹ is —N═ and A³ is —CR^{2d}═;
optionally, R^{2d} is selected from hydrogen, —NH₂, fluoro, and methoxy; or
(iii) A¹ is —CR^{2a}═ and A³ is —N═;
optionally, R^{2a} is selected from hydrogen, —NH₂, fluoro, and methoxy; and
optionally, wherein R^{1d} is hydrogen or Q-L-.

9. The compound of any one of the preceding claims, being of any one of Formula XIII-XV: 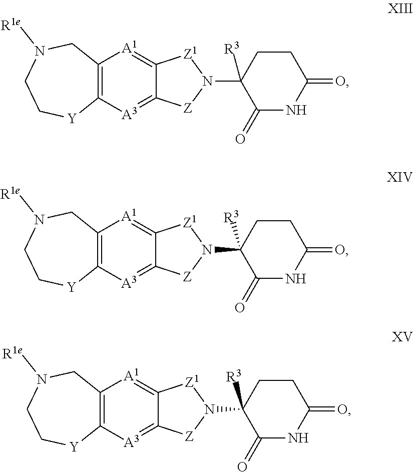
or a pharmaceutically acceptable salt or solvate thereof;
optionally, wherein:
(i) A¹ is —CR^{2a}═ and A³ is —CR^{2d}═;
optionally, R^{2a} is selected from hydrogen, —NH₂, fluoro, and methoxy; and
optionally, R^{2d} is selected from hydrogen, —NH₂, fluoro, and methoxy; or
(ii) A¹ is —N═ and A³ is —CR^{2a}═;
optionally, R^{2d} is selected from hydrogen, —NH₂, fluoro, and methoxy; or
(iii) A¹ is —CR^{2a}═ and A³ is —N═;
optionally, R^{2a} is selected from hydrogen, —NH₂, fluoro, and methoxy.

10. The compound of any one of the preceding claims, being of any one of Formulae XVI-XXI: 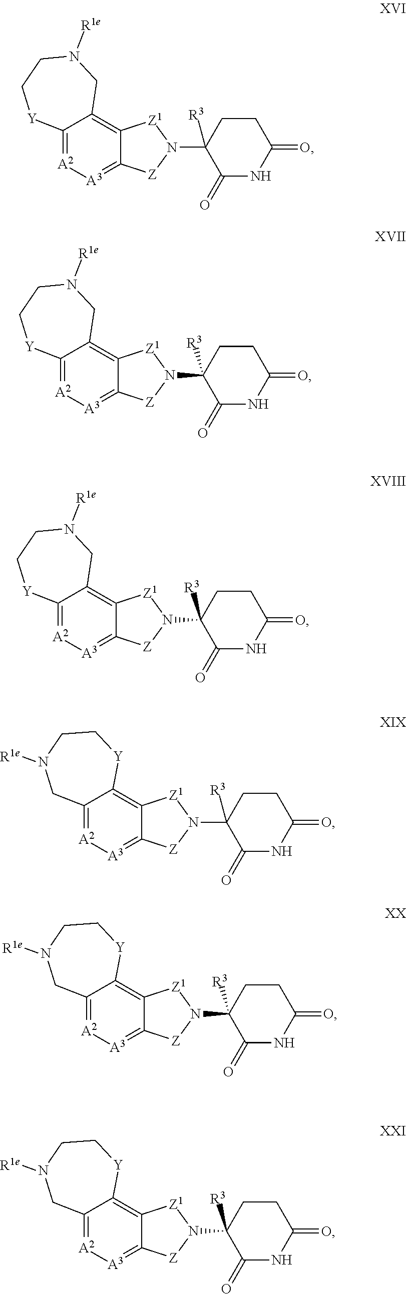
or a pharmaceutically acceptable salt or solvate thereof;
optionally, wherein:
(i) A² is —CR^{2c}═ and A³ is —CR^{2d}═;
optionally, R^{2e} is selected from hydrogen, —NH₂, fluoro, and methoxy; and
optionally, R^{2d} is selected from hydrogen, —NH₂, fluoro, and methoxy;
(ii) A² is —N═ and A³ is —CR^{2d}═;
optionally, R^{2d} is selected from hydrogen, —NH₂, fluoro, and methoxy; or
(iii) A² is —CR^{2c}═ and A is —N═;
optionally, R^{2c} is selected from hydrogen, —NH₂, fluoro, and methoxy; and
optionally, wherein R^{1e} is hydrogen or Q-L-.

11. The compound of any one of the preceding claims, being of any one of Formulae XXII-XXIV: 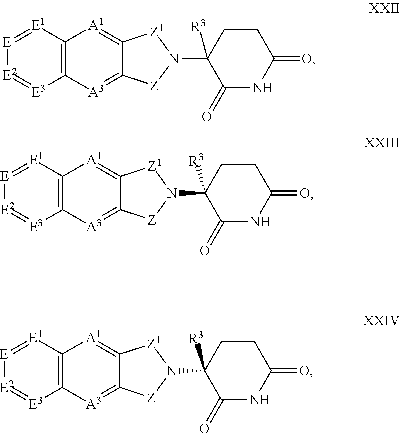
or a pharmaceutically acceptable salt or solvate thereof;
optionally, wherein:
(i) A¹ is —CR^{2a}═ and A³ is —CR^{2d}═;
optionally, R^{2a} is selected from hydrogen, —NH₂, fluoro, and methoxy; and
optionally, R^{2d} is selected from hydrogen, —NH₂, fluoro, and methoxy;
(ii) A¹ is —N═ and A³ is —CR^{2d}═;
optionally, R^{2d} is selected from hydrogen, —NH₂, fluoro, and methoxy; or
(iii) A¹ is —CR^{2a}═ and A³ is —N═;
optionally, R²ⁱ is selected from hydrogen, —NH₂, fluoro, and methoxy; and
optionally, wherein E² is —N═; or E² is —CR^{2g}═ and R^{2g} is Q-L-; and
optionally, wherein E is —N═; or E is —CR^{2h}═ and R² is Q-L-; and
optionally, wherein E² is —N—; or E² is —CR^{2g}═ and R is Q-L-; and
optionally, wherein E³ is —N═; or E³ is —CR^{2h}═ and R^{2h} is Q-L-.

12. The compound of any one of the preceding claims, being of any one of Formulae XXV-XXVII: 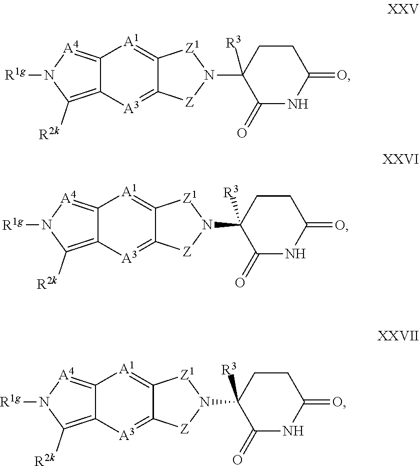
or a pharmaceutically acceptable salt or solvate thereof;
optionally, wherein:
(i) A¹ is —CR^{2a}═ and A³ is —CR^{2d}═;
optionally, R^{2a} is selected from hydrogen, —NH₂, fluoro, and methoxy; or
optionally, R^{2d} is selected from hydrogen, —NH₂, fluoro, and methoxy;
(ii) A¹ is —N═ and A³ is —CR^{2d}═;
optionally, R^{2d} is selected from hydrogen, —NH₂, fluoro, and methoxy; or
(iii) A¹ is —CR^{2a}═ and A³ is —N═;
optionally, R^{2a} is selected from hydrogen, —NH₂, fluoro, and methoxy; and
optionally, wherein A⁴ is —N═; or A⁴ is —CH═; and
optionally, wherein R^{2k} is hydrogen; and
optionally, wherein R¹⁹ is hydrogen; or R^{1g} is Q-L.

13. The compound of any one of the preceding claims, being of any one of Formulae XXVIII-XXX: 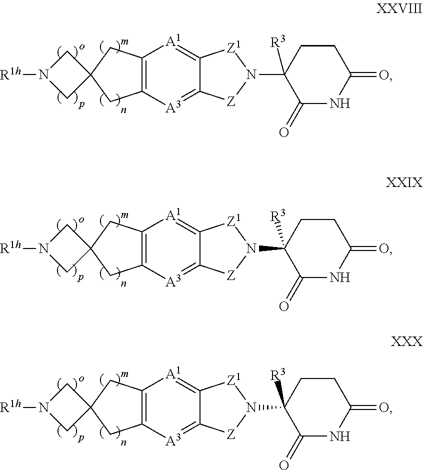
or a pharmaceutically acceptable salt or solvate thereof;
optionally, wherein:
(i) A¹ is —CR^{2a}═ and A³ is —CR²d_;
optionally, R^{2a} is selected from hydrogen, —NH₂, fluoro, and methoxy; or
optionally, R^{2d} is selected from hydrogen, —NH₂, fluoro, and methoxy;
(ii) A¹ is —N═ and A³ is —CR^{2d}_;
optionally, R^{2d} is selected from hydrogen, —NH₂, fluoro, and methoxy; or
(iii) A¹ is —CR^{2a}═ and A³ is —N═;
optionally, R^{2a} is selected from hydrogen, —NH₂, fluoro, and methoxy; and
optionally, wherein m is 1; or m is 2; and
optionally, wherein n is 1; or n is 2; and
optionally, wherein o is 1; or o is 2; and
optionally, wherein p is 1; or p is 2; and
optionally, wherein R^{1h} is hydrogen; or R^{1h} is Q-L-.

14. The compound of any one of the preceding claims, being of any one of Formulae XXXI-XXXIII: 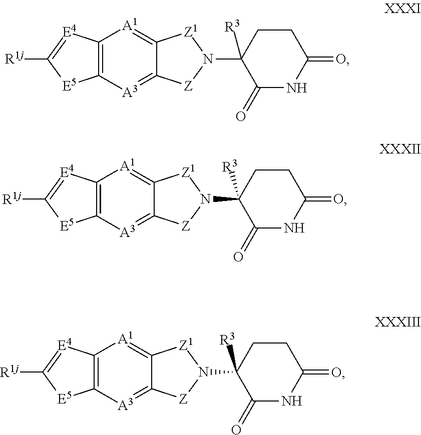
or a pharmaceutically acceptable salt or solvate thereof;
optionally, wherein:
(i) A¹ is —CR^{2a}═ and A³ is —CR^{2d}═;
optionally, R^{2a} is selected from hydrogen, —NH₂, fluoro, and methoxy; and
optionally, R^{2d} is selected from hydrogen, —NH₂, fluoro, and methoxy;
(ii) A¹ is —N═ and A³ is —CR^{2d}═;
optionally, R^{2d} is selected from hydrogen, —NH₂, fluoro, and methoxy; or
(iii) A¹ is —CR^{2a}═ and A³ is —N═;
optionally, R^{2a} is selected from hydrogen, —NH₂, fluoro, and methoxy.

15. The compound of any one of the preceding claims, being of any one of Formulae XXXIV-XXXIX: 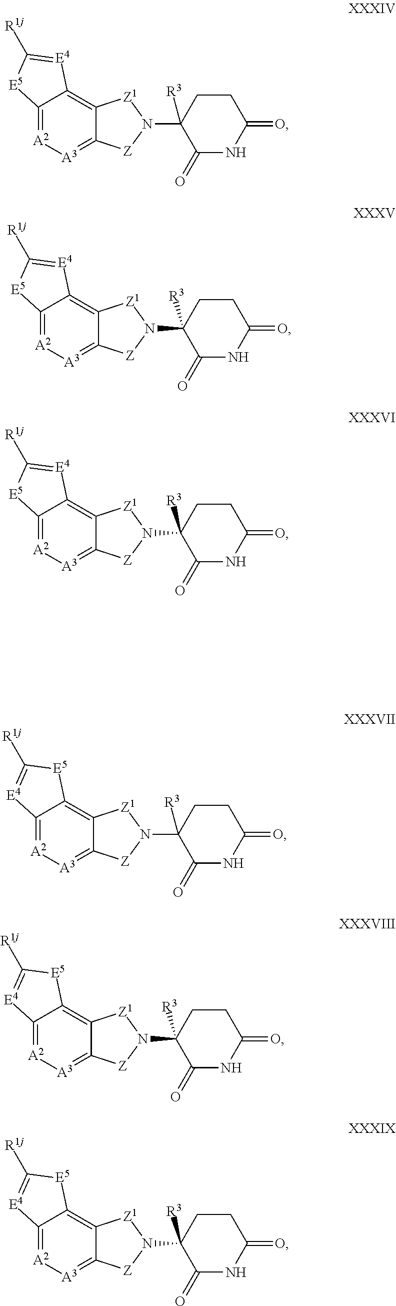
or a pharmaceutically acceptable salt or solvate thereof;
optionally, wherein:
(i) A² is —CR^{2c}═ and A³ is —CR²d_;
optionally, R^{2c} is selected from hydrogen, —NH₂, fluoro, and methoxy; and
optionally, R^{2d} is selected from hydrogen, —NH₂, fluoro, and methoxy;
(ii) A² is —N═ and A³ is —CR^{2d}_;
optionally, R^{2d} is selected from hydrogen, —NH₂, fluoro, and methoxy; or
(iii) A² is —CR^{2c}═ and A is —N═;
optionally, R^{2c} is selected from hydrogen, —NH₂, fluoro, and methoxy; and
optionally, wherein E⁴ is ═C(H)—; or E⁴ is ═N—; and
optionally, wherein E⁵ is —O—; E⁵ is —S—; or E⁵ is selected from —NH— and —N(CH₃)—.

16. The compound of any one of the preceding claims, or a pharmaceutically acceptable salt or solvate thereof, wherein:
(i) R^{1j} is selected from hydrogen, C₁-C₄ alkyl, (hydroxy)alkyl, and (heterocyclo)alkyl; or
(ii) R^{1j} is Q-L-.

17. The compound of any one of the preceding claims, or a pharmaceutically acceptable salt or solvate thereof, wherein R³ is hydrogen.

18. The compound of any one of the preceding claims, or a pharmaceutically acceptable salt or solvate thereof, wherein:
(i) Z and Z¹ are —C(═O)—;
(ii) Z is —C(═O)— and Z¹ is —CR^{4a}R^{4b}—;
(iii) Z is —CR^{4a}R^{4b}—; and Z¹ is —C(═O)—;
optionally, R^{4a} and R^{4b} are hydrogen;
(iv) Z is —N═C(CH₃)— and Z¹ is —C(═O)—;
(v) Z is —C(═O)— and Z¹ is —N═C(CH₃)—;
(vi) Z is a bond and Z¹ is —N(CH₃)C(═O)—; or
(vii) Z is —N(CH₃)C(═O) and Z is a bond.

19. The compound of any one of the preceding claims, or a pharmaceutically acceptable salt or solvate thereof, being selected from the compounds described in Table 1, and pharmaceutically acceptable salts and solvates thereof.

20. A pharmaceutical composition comprising the compound of any one of the preceding claims, or a pharmaceutically acceptable salt or solvate thereof, and a pharmaceutically acceptable carrier.

21. A method of inhibiting CRBN ubiquitination in a subject, comprising administering to the subject the compound of any one of the preceding claims or a pharmaceutically acceptable salt or solvate thereof.

22. Use of the compound of any one of the preceding claims or a pharmaceutically acceptable salt or solvate thereof in the manufacture of a medicament for inhibiting CRBN ubiquitination in a subject.

23. The compound of any one of the preceding claims or a pharmaceutically acceptable salt or solvate thereof for use in inhibiting CRBN ubiquitination in a subject.

24. A method of degrading a protein in a subject, comprising administering to the subject the compound of any one of the preceding claims or a pharmaceutically acceptable salt or solvate thereof.

25. Use of the compound of any one of the preceding claims or a pharmaceutically acceptable salt or solvate thereof in the manufacture of a medicament for degrading a protein in a subject.

26. The compound of any one of the preceding claims or a pharmaceutically acceptable salt or solvate thereof for use in degrading a protein in a subject.

27. A method of treating or preventing a disease in a subject in need thereof, comprising administering to the subject the compound of any one of the preceding claims or a pharmaceutically acceptable salt or solvate thereof

28. Use of the compound of any one of the preceding claims or a pharmaceutically acceptable salt or solvate thereof in the manufacture of a medicament for treating or preventing a disease in a subject.

29. The compound of any one of the preceding claims or a pharmaceutically acceptable salt or solvate thereof for use in treating or preventing a disease in a subject.

30. The method, use, or compound for use in any one of the preceding claims, wherein the subject is a mammal.

31. The method, use, or compound for use in any one of the preceding claims, wherein the subject is a human.

32. The method, use, or compound for use in any one of the preceding claims, wherein the disease is associated with degradation of a protein.

33. The method, use, or compound for use in any one of the preceding claims, wherein the disease is a cancer;
optionally, the cancer is selected from the cancers described in Table I.
